(19) United States
(12) Patent Application Publication
(10) Pub. No.: US 2024/0109887 A1
(43) Pub. Date: Apr. 4, 2024

(54) NOVEL SUBSTITUTED BICYCLIC AZA-HETEROCYCLES AS SOS1 INHIBITORS

(71) Applicants: Hongjian ZHANG, Plainsboro, NJ (US); Viva Star Biosciences (Suzhou) Co., Ltd., Suzhou (CN)

(72) Inventors: Hongjian ZHANG, Plainsboro, NJ (US); Jingjing JI, Shanghai (CN); Peihua SUN, Shanghai (CN)

(73) Assignee: Viva Star Biosciences (Suzhou) Co., Ltd., Suzhou (CN)

(21) Appl. No.: 18/280,177

(22) PCT Filed: Mar. 1, 2022
(86) PCT No.: PCT/US2022/018372

§ 371 Date: Sep. 1, 2023

(30) Foreign Application Priority Data
Mar. 2, 2021 (WO) PCT/CN2021/078596

(51) Int. Cl. C07D 471/04

(52) U.S. Cl. CPC C07D 471/04

(57) ABSTRACT

This application relates to novel substituted bicyclic aza-heterocycles and analogues, their manufacture, pharmaceutical compositions comprising them, and their use as medicaments for treating a disease associated with mammalian RAS (Rat sarcoma virus) family proteins.

### CROSS-REFERENCE TO RELATED APPLICATIONS

This application claims priority to PCT International Application No. PCT/CN2021/078596, filed Mar. 2, 2021, the entire contents of which are incorporated herein by reference.

### FIELD

This application relates to novel substituted bicyclic aza-heterocycles and analogues, their manufacture, pharmaceutical compositions comprising them, and their use as medicaments for treating a disease associated with mammalian RAS (Rat sarcoma virus) family proteins.

### BACKGROUND

RAS (Rat sarcoma virus) proteins belong to a superfamily of small GTPases and act as a “binary switch” in cellular signal transduction. As depicted in the diagram in FIG. 1 [Biomark Cancer, 2016; 8(Suppl 1): 27-35.], RAS proteins exert their effects via the GDP/GTP cycle (the nucleotide exchange cycle). GEFs (Guanine nucleotide exchange factors) stimulate GDP/GTP exchange, leading to the formation of RAS-GTP complex, which interacts with and turns on the downstream effector pathways. Because cellular concentrations of GTP are much higher than GDP (up to 10-fold higher), the active RAS-GTP is thus in the RAS-On state as a net result. In contrast, GAPs (GTPase activating proteins) enhance the hydrolysis of GTP, leading to the formation of RAS-GDP complex which is unable to engage downstream effectors, and resulting in a RAS-Off or inactive state. Mutations in RAS genes promote the formation of the RAS-GTP complex and an overtly “active state” drives tumor cell proliferation and survival, leading to poor prognosis of several cancer types due to altered tumor metabolism and drug resistance.

RAS genes encode four highly homologous RAS proteins (83 to 85% amino acid sequence identity): HRAS (Harvey rat sarcoma viral oncogene homolog), NRAS (Neuroblastoma RAS viral oncogene homolog), KRAS4A (Kirsten rat sarcoma viral oncogene homologue 4A), and KRAS4B [Cell. 2017 Jun. 29; 170(1):17-33; Sci. Signal. 2020, 13, eaay 6013]. Since the discovery of mutationally activated human RAS genes as important oncogenes, mutations in KRAS, HRAS and NRAS have been linked to poor prognosis of different forms of human cancers [Ras history, Small GTPases, 2010, 1:1, 2-27, DOI: 10.4161/sgtp.1.1.12178]. To date, activating mutations in RAS proteins are found in approximately 24% of all cancers, among which KRAS mutation is a major driver in 90% of pancreatic ductal adenocarcinoma (PDAC), 43% of colorectal cancer (CRC), and 26% of nonsmall-cell lung cancer (NSCLC), affecting pathways involved in cell proliferation, differentiation, and apoptosis [Cell. 2017 Jun. 29; 170(1):17-33; Oncogene. 2018 May; 37(18):2444-2455]. Because of high cellular concentrations of GTP and a small/shallow binding pocket in the RAS-GTP complex, direct targeting of the GTP binding site via competitive inhibition has not been fruitful in the discovery and development of RAS inhibitors (particularly KRAS inhibitors) as antitumor agents.

The RAS signaling pathway is associated with multiple upstream initiators (such as RTKs, receptor tyrosine kinases), downstream effectors, and binding partners (chaperon proteins), etc. As illustrated in the diagram shown in FIG. 2 [Oncotarget. 2014 Sep. 15; 5 (17):7285-302; Sci. Signal. 2020, 13, eaay6013], SOS (Son of sevenless, a GEF) plays a critical role in converting RAS-GDP (Off) to RAS-GTP (On), thus activating downstream effector RAF/MEK/ERK and/or PI3K/AKT/mTOR pathways and promoting tumor cell proliferation and survival [Oncotarget. 2014 Sep. 15; 5 (17):7285-302; Cell. 2017 Jun. 29; 170(1):17-33]. As such, the RAS:SOS interaction and/or various stages of RAS signaling have been explored to identify tumor vulnerabilities for therapeutic intervention of various RAS mutation driven cancers [Biochemical Journal (2019) 476 365-374; Sci Signal. 2020 Mar. 24; 13(624): eaay 6013].

SOS1 (Son of sevenless homolog 1) is the most studied GEF that is involved in RAS signaling. The RAS:SOS1 interaction, particularly KRAS:SOS1 interaction, has been explored as a key vulnerability in KRAS mutation driven cancers. It has been shown that SOS1-mediated cross-activation of oncogenic RAS is essential for tumorigenesis and the depletion of SOS1 reduced the proliferation rate and survival of tumor cells harboring KRAS mutations [Nat Commun. 2012; 3:1168. doi: 10.1038/ncomms 2173.]. Studies have also demonstrated that the RAS:SOS1 interaction can be disrupted/inhibited by small molecules, as exemplified by BAY-293 and BI-2852 [Proc Natl Acad Sci USA. 2019 Feb. 12; 116(7):2551-2560; Proc Natl Acad Sci USA. 2019 Aug. 6; 116(32):15823-15829]. BAY-293, a selective inhibitor of the KRAS:SOS1 interaction, prevents the formation of the KRAS:SOS1 complex and blocks the reloading of KRAS with GTP, leading to antiproliferative activity [Proc Natl Acad Sci USA. 2019 Feb. 12; 116(7):2551-2560]. In comparison, BI-2852, a KRAS inhibitor that binds with nanomolar affinity to a pocket between switch I and II on RAS, blocks all GEF, GAP, and effector interactions with KRAS, leading to inhibition of downstream signaling and antiproliferative effect, suggesting the switch I/II pocket of RAS is druggable by small molecules [Proc Natl Acad Sci USA. 2019 Aug. 6; 116(32):15823-15829]. The above findings clearly demonstrate the essential role of SOS1 as a GEF in KRAS mutant cancers and that the KRAS:SOS1 interaction in RAS signaling can be targeted for therapeutic intervention.

In addition to cancers, aberrant activities of RAS proteins are associated with various diseases, generally termed RASopathies [J Hum Genet. 2016 January; 61(1):33-9.]. In contrast to RAS mutation driven cancers, RASopathies are a group of clinical syndromes caused by hyperactivation of the RAS/MAPK pathway [Cell. 2017 Jun. 29; 170(1):17-33]. Evidence has implied that somatic mutations in RAS and SOS genes play an important role in the pathogenesis of these developmental disorders including neurofibromatosis type 1, Noonan syndrome, Noonan syndrome with multiple lentigines, Costello syndrome, cardiofaciocutaneous syndrome, and capillary malformation-arteriovenous syndrome [Ann Oncol. 2020 Mar. 30; S0923-7534(20)36378-X.]. Therefore, a RAS:SOS inhibitor may be used to treat not only RAS mutation driven cancers but also RASopathies caused by aberrant RAS signaling activities. Furthermore, a recent study demonstrates that knocking down the expression of KRAS using antisense oligonucleotides reduces fibrosis by 50% and prevents the loss of renal function in a mouse model of chronic folic acid nephropathy [Sci Rep. 2019 Sep. 30; 9(1):14010], suggesting that targeting KRAS expression and activity may be used for treatment of renal dysfunction associated with fibrosis.

### SUMMARY

The present technology provides novel RAS:SOS (e.g. KRAS:SOS1) small molecules inhibitors which disrupt the interaction between RAS and SOS and prevent the activation of RAS proteins particularly KRAS. The inhibition of the RAS:SOS (e.g. KRAS:SOS1) complex formation results in significant inhibitory effects on downstream effector pathways, leading to profound reduction in proliferation and survival of RAS or KRAS mutation driven cancers. The described RAS:SOS (e.g. KRAS:SOS1) inhibitors may be used to treat various RAS mutation driven cancers such as PDAC, NSCLC, CRC, as well as RASopathies. In addition, small molecule compounds disclosed herein show good pharmaceutical properties including solubility, ADME (absorption, distribution, metabolism, and excretion), pharmacokinetics, CYP inhibition and other safety profiles. Finally, RAS:SOS (e.g., KRAS:SOS1) inhibitors may be used for in-pathway combinations, with inhibitors of upstream RTKs or SHP2 and downstream RAF, MEK, ERK, PI3K, AKT, or mTOR, to achieve better antitumor efficacy.

In one aspect, the present technology relates to a compound of Formulae (I)-(IV),

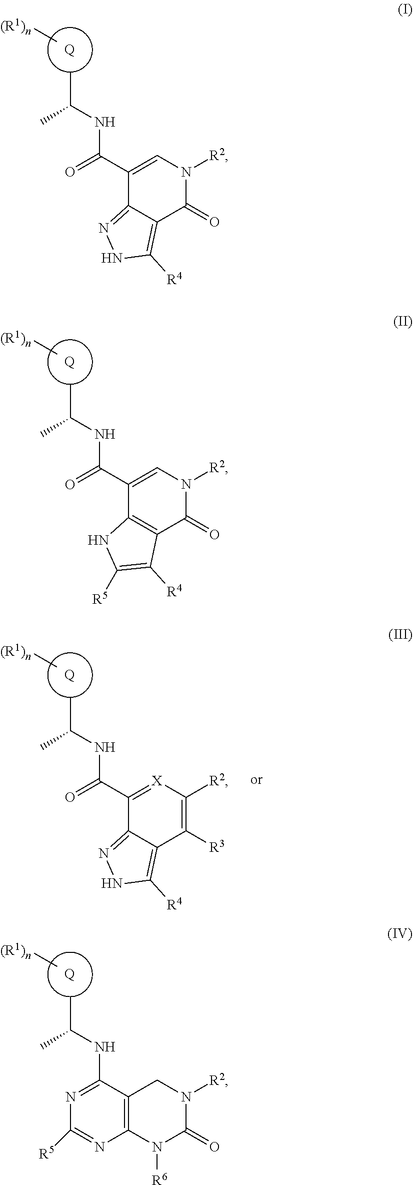

or a pharmaceutically acceptable salt, tautomer or stereoisomer thereof,
wherein:
Q at each occurrence is independently a ring selected from phenyl or a 5- or 6-membered heteroaryl group, wherein the heteroaryl group comprises at least one carbon atom and 1-4 additional heteroatoms independently selected from nitrogen, oxygen and sulfur;
X is CH or N;
R¹ at each occurrence is independently hydrogen, halogen, C₁₋₆alkyl, haloC₁₋₆alkyl, C₁₋₆alkenyl, C₁₋₆alkynyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, —S(O)ₜ—C₁₋₆alkyl, —S(O)ₜ—NR^{a}R^{b}, phenyl, or 3-7-membered heterocyclyl, wherein the phenyl and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b}, or two adjacent R¹ groups, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄ alkyl-NR^{a}R^{b}, and oxo group (═O);
R² at each occurrence is independently hydrogen, halogen, CN, —OR^{a}, —NR^{a}R^{b}, C₁₋₆alkyl, haloC₁₋₆alkyl, C₂₋₆alkenyl, C₂₋₆alkynyl, C₃₋₇cycloalkyl, 3-7-membered heterocylyl, phenyl, or 5-6-membered heteroaryl, wherein each of the C₁₋₆alkyl, haloC₁₋₆alkyl, C₂₋₆alkenyl, C₂₋₆alkynyl, C₃₋₇cycloalkyl, 3-7-membered heterocylyl, phenyl, and 5-6-membered heteroaryl is optionally substituted with 1-5 R⁸;
R³ at each occurrence is independently hydrogen, halogen, C₁₋₆alkyl, haloC₁₋₆alkyl, C₁₋₆alkoxy, haloC₁₋₆alkoxy, C₁₋₆alkyl-OH, CN, C₃₋₇cycloalkyl, C₃₋₇cycloalkyl-OH, C₃₋₇cycloalkoxy, —NH₂, —NHC₁₋₄alkyl, —N(C₁₋₄alkyl)₂, or 3-7-membered cyclic amine;
R⁴ at each occurrence is independently hydrogen, halogen, C₁₋₆alkyl, haloC₁₋₆alkyl, C₁₋₆alkoxy, haloC₁₋₆alkoxy, CN, NH₂, C₃₋₇cycloalkyl or C₃₋₇cycloalkoxy;
R⁵ at each occurrence is independently hydrogen, C₁₋₄alkyl, or haloC₁₋₄alkyl;
R⁶ at each occurrence is independently hydrogen, C₁₋₆alkyl, haloC₁₋₆alkyl, or C₃₋₇cycloalkyl;
R⁸ at each occurrence is independently hydrogen, halogen, C₁₋₄alkyl, haloC₁₋₄alkyl, C₁₋₄alkoxy, C₂₋₄alkenyl, C₂₋₄alkynyl, C₃₋₇cycloalkyl, C₃₋₇cycloalkoxy, 3-7-membered heterocyclyl, phenyl, 5-6-membered heteroaryl, —OR^{a}, —SR^{a}, S(O)ₜR^{a}, —S(O)ₜNR^{a}R^{b}, —OC(O)—R^{a}, —NR^{a}R^{b}, —C(O)R^{a}, —C(O)OR^{a}, —OC(O)NR^{a}R^{b}₂, —C(O)NR^{a}R^{b}, —N(R^{a})C(O)OR^{a}, —N(R^{a})C(O)R^{a}, —N(R^{a})C(O)NR^{a}R^{b}, —N(R^{a})C(NR^{a})NR^{a}R^{b}, —N(R^{a})S(O)ₜNR^{a}R^{b}, —P(═O)(R^{a})(R^{b}), —O—P(═O)(OR^{a})(OR^{b}), or oxo group (═O);
R^{a} and R^{b} at each occurrence are independently hydrogen, C₁₋₆alkyl, haloC₁₋₆alkyl, C₁₋₆alkyl-OH, C₁₋₆alkoxy, C₃₋₇cycloalkyl, 3-7-membered heterocyclyl, C₁₋₆alkyl-NH₂, C₁₋₆alkyl-NHC₁₋₄alkyl, C₁₋₆alkyl-N(C₁₋₄alkyl)₂, or C₁₋₆alkyl-(3-7-membered cyclic amine), wherein each of the foregoing groups may be optionally substituted by one to three substituents independently selected from the group consisting of C₁₋₄alkyl, haloC₁₋₄alkyl, halogen, OH, NH₂, C₁₋₄alkoxy, haloC₁₋₄alkoxy, CN, and —C(O)C₁₋₄alkyl; or R^{a} and R^{b}, together with the nitrogen atom to which they are attached, form a saturated or unsaturated heterocyclic ring containing from three to seven ring atoms, which ring may optionally contain additional one or two heteroatoms independently selected from the group consisting of nitrogen, oxygen and sulfur and may be optionally substituted by from one to three substituents independently selected from the group consisting of C₁₋₄alkyl, —C(O)C₁₋₄alkyl, phenyl and benzyl;
n at each occurrence is independently 1, 2 or 3, and
t at each occurrence is independently 1 or 2.

The technology also relates to a pharmaceutical composition comprising a compound of Formulae (I)-(IV), its manufacture and use as medicaments for treating or preventing a disease or condition mediated by mammalian Ras family proteins. Accordingly, the compounds of Formulae (I)-(IV) are useful for treating or preventing cancer, including breast cancer, leukemia, prostate cancer, ovarian cancer, pancreatic cancer, colon cancer, lung cancer, endometrial cancer, thyroid cancer, cholangiocarcinoma, multiple myeloma, melanoma, uterine cancer, head and neck squamous cell carcinoma, diffuse large B cell lymphoma, esophageal cancer, hepatocellular cancer, glioblastoma, renal cancer, sarcoma, bladder cancer, urothelial cancer, gastric cancer, or cervical cancer. Additionally, the compounds of Formulae (I)-(IV) are useful for treating or preventing RASopathies.

### BRIEF DESCRIPTION OF DRAWINGS

FIG. 1 schematically illustrates how RAS proteins exert their effects via the GDP/GTP cycle (the nucleotide exchange cycle), and lead to RAS-GDP (off) and RAS-GTP (on) states. Mutations in RAS genes that promote formation of the latter can drive tumor cell proliferation and survival

FIG. 2 schematically illustrates how SOS (Son of sevenless, a GEF) plays a critical role in converting RAS-GDP (Off) to RAS-GTP (On), thus activating downstream effector RAF/MEK/ERK and/or PI3K/AKT/mTOR pathways and promoting tumor cell proliferation and survival.

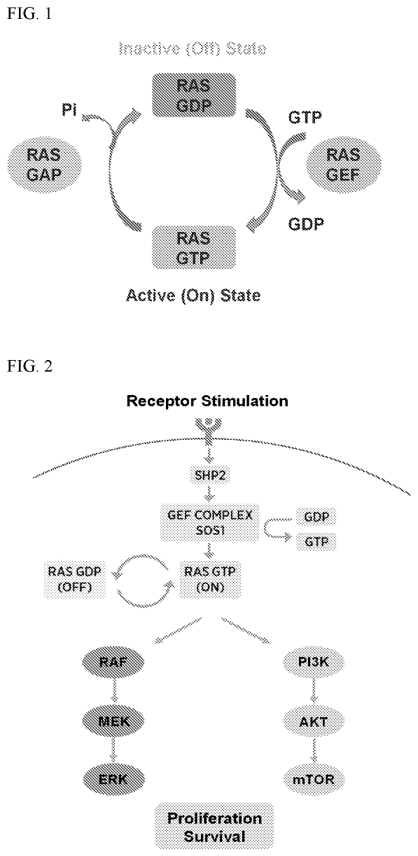

### DETAILED DESCRIPTION

In one aspect, the present technology provides compounds, and their pharmaceutically acceptable forms, including, but not limited to, salts, hydrates, solvates, isomers, sterioisomers, enantiomers, prodrugs, and isotopically labeled derivatives thereof.

In another aspect, the present technology provides methods of treating and/or managing various diseases and disorders, which comprises administering to a patient a therapeutically effective amount of a compound provided herein, or a pharmaceutically acceptable form (e.g., salts, hydrates, solvates, isomers, sterioisomers, enantiomers, prodrugs, and isotopically labeled derivatives) thereof. Non-limiting examples of diseases and disorders are described herein.

In another aspect, the present technology provides methods of preventing various diseases and disorders, which comprises administering to a patient in need of such prevention a prophylactically effective amount of a compound provided herein, or a pharmaceutically acceptable form (e.g., salts, hydrates, solvates, isomers, sterioisomers, prodrugs, and isotopically labeled derivatives) thereof. Non-limiting examples of diseases and disorders are described herein.

In another aspect, a compound provided herein, or a pharmaceutically acceptable form (e.g., salts, hydrates, solvates, isomers, sterioisomers, prodrugs, and isotopically labeled derivatives) thereof, can be administered in combination with another drug (“second active agent”) or treatment. Second active agents include small molecules and large molecules (e.g., proteins and antibodies).

Also provided herein are pharmaceutical compositions (e.g., single unit dosage forms) that can be used in the methods provided herein. In one embodiment, pharmaceutical compositions comprise a compound provided herein, or a pharmaceutically acceptable form (e.g., salts, hydrates, solvates, isomers, sterioisomers, prodrugs, and isotopically labeled derivatives) thereof, and optionally one or more second active agents.

While specific embodiments have been discussed, the specification is illustrative only and not restrictive. Many variations of this disclosure will become apparent to those skilled in the art upon review of this specification.

Unless defined otherwise, all technical and scientific terms used herein have the same meaning as is commonly understood by one of ordinary skill in the art to which this specification pertains.

### Definitions

As used in the specification and claims, the singular form “a”, “an” and “the” includes plural references unless the context clearly dictates otherwise.

As used herein, “agent” or “biologically active agent” or “second active agent” refers to a biological, pharmaceutical, or chemical compound or another moiety. Non-limiting examples include simple or complex organic or inorganic molecules, a peptide, a protein, an oligonucleotide, an antibody, an antibody derivative, an antibody fragment, a vitamin, a vitamin derivative, a carbohydrate, a toxin, or a chemotherapeutic compound, and metabolites thereof. Various compounds can be synthesized, for example, small molecules and oligomers (e.g., oligopeptides and oligonucleotides), and synthetic organic compounds based on various core structures. In addition, various natural sources can provide active compounds, such as plant or animal extracts, and the like. A skilled artisan can readily recognize that there is no limit as to the structural nature of the agents of this disclosure.

“Administration” of a disclosed compound encompasses the delivery to a subject of a compound as described herein, or a prodrug or other pharmaceutically acceptable derivative thereof, using any suitable formulation or route of administration, as discussed herein.

The term “co-administration,” “administered in combination with,” and their grammatical equivalents, as used herein, encompasses administration of two or more agents to the subject so that both agents and/or their metabolites are present in the subject at the same time. Co-administration includes simultaneous administration in separate compositions, administration at separate times in separate compositions, or administration in a composition in which both agents are present.

The term “effective amount” or “therapeutically effective amount” refers to that amount of a compound or pharmaceutical composition described herein that is sufficient to affect the intended application including, but not limited to, disease treatment, as illustrated below. In some embodiments, the amount is that effective for detectable inhibition of SOS1, which, for example, can be determined in a KRAS:SOS1 binding assay. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a response in target cells, e.g., reduction of cell migration. The specific dose will vary depending on, for example, the compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

All methods described herein can be performed in any suitable order unless otherwise indicated herein or otherwise clearly contradicted by context.

As used herein, the terms “treatment”, “treating”, “palliating” “managing” and “ameliorating” are used interchangeably herein. These terms refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a disease, or to a patient reporting one or more of the physiological symptoms of a disease, even though a diagnosis of this disease may not have been made.

The terms “preventing” and “prophylaxis” as used herein refer to administering a pharmaceutical compound or medicament or a composition including the pharmaceutical compound or medicament to a subject before a disease, disorder, or condition fully manifests itself, to forestall the appearance and/or reduce the severity of one or more symptoms of the disease, disorder or condition. The person of ordinary skill in the art recognizes that the term “prevent” is not an absolute term. In the medical art it is understood to refer to the prophylactic administration of a drug to diminish the likelihood or seriousness of a disease, disorder or condition, or a symptom thereof, and this is the sense that such terms are used in this disclosure.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The term “in vivo” refers to an event that takes place in a subject's body. In vivo also includes events occurring in rodents, such as rats, mice, guinea pigs, and the like.

The term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. For example, pharmaceutically acceptable salts include adipate, alginate, ascorbate, aspartate, benzenesulfonate, besylate, benzoate, bisulfate, borate, butyrate, camphorate, camphor sulfonate, citrate, cyclopentane propionate, digluconate, dodecyl sulfate, ethane sulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In some embodiments, the pharmaceutically acceptable form is a prodrug. As used herein, the term “prodrug” refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elselvier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁₋₈)alkyl, (C₁₋₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 10 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as [3-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a disclosed compound contains an alcohol functional group, a prodrug can be formed by the replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, α-amino(C₁₋₄)alkanoyl, arylacyl, and α-aminoacyl, or α-aminoacyl-α-aminoacyl, where each α-aminoacyl group is independently selected from the naturally occurring L-amino acids, —P(O)(OH)₂, —P(O)(O(C₁₋₆)alkyl)₂ or glycosyl (the radical resulting from the removal of a hydroxyl group of the hemiacetal form of a carbohydrate).

If a disclosed compound incorporates an amine functional group, a prodrug can be formed by the replacement of a hydrogen atom in the amine group with a group such as R-carbonyl, RO-carbonyl, NRR′-carbonyl where R and R′ are each independently selected from (C₁₋₁₀)alkyl, (C₃₋₇)cycloalkyl, benzyl, a natural α-aminoacyl or natural α-aminoacyl-natural-α-aminoacyl, —C(OH)C(O)OY¹ wherein Y¹ is H, (C₁₋₆)alkyl or benzyl; —C(OY²)Y³ wherein Y² is (C₁₋₄)alkyl and Y³ is (C₁₋₆)alkyl, carboxy(C₁₋₆)alkyl, amino(C₁₋₄)alkyl or mono-N— or di-N,N—(C₁₋₆)alkylaminoalkyl; and —C(Y⁴)Y⁵ wherein Y⁴ is H or methyl and Y⁵ is mono-N— or di-N—(C_{1 6})alkylamino, morpholino, piperidin-1-yl or pyrrolidin-1-yl.

In some embodiments, the disclosed compounds may encompass an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

Geometric isomers can be represented by the symbol [symbol] which denotes a bond that can be a single, double or triple bond as described herein. Provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or“trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

“Enantiomers” are a pair of stereoisomers that are non-superimposable mirror images of each other. A mixture of a pair of enantiomers in any proportion can be known as a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

In some embodiments, an enantiomer is provided partly or substantially free of the corresponding enantiomer, and may be referred to as “optically enriched,” “enantiomerically enriched,” “enantiomerically pure,” and “non-racemic,” as used interchangeably herein. The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer.

ee=(90−10)/100=80%.

Thus, a composition containing 90% of one enantiomer and 10% of the other enantiomer is said to have an enantiomeric excess of 80%. In some embodiments, compositions described herein contain an enantiomeric excess of at least about 50%, at least about 60%, at least about 70%, at least about 80%, at least about 90%, at least about 95%, at least about 98%, at least about 99%, or at least about 99.5% of the S enantiomer, or a range between and including any two of the foregoing values (e.g., 50-99.5% ee). In other words, the compositions contain an enantiomeric excess of the S enantiomer over the R enantiomer. In other embodiments, some compositions described herein contain an enantiomeric excess of at least about 50%, at least about 60%, at least about 70%, at least about 80%, at least about 90%, at least about 95%, at least about 98%, at least about 99%, or at least about 99.5% of the R enantiomer or a range between any two of the foregoing values (e.g., 50-99.5% ee). In other words, the compositions contain an enantiomeric excess of the R enantiomer over the S enantiomer. Where the enrichment of one enantiomer is much greater than about 80% by weight, the compositions are referred to as “substantially enantiomerically enriched,” “substantially enantiomerically pure” or a “substantially non-racemic” preparation.

Thus, a composition containing 90% of one enantiomer and 10% of the other enantiomer is said to have an enantiomeric excess of 80%. In some embodiments, compositions described herein contain an enantiomeric excess of at least about 50%, at least about 60%, at least about 70%, at least about 80%, at least about 90%, at least about 95%, at least about 98%, at least about 99%, or at least about 99.5% of the S enantiomer, or a range between and including any two of the foregoing values (e.g., 50-99.5% ee). In other words, the compositions contain an enantiomeric excess of the S enantiomer over the R enantiomer. In other embodiments, some compositions described herein contain an enantiomeric excess of at least about 50%, at least about 60%, at least about 70%, at least about 80%, at least about 90%, at least about 95%, at least about 98%, at least about 99%, or at least about 99.5% of the R enantiomer or a range between any two of the foregoing values (e.g., 50-99.5% ee). In other words, the compositions contain an enantiomeric excess of the R enantiomer over the S enantiomer. Where the enrichment of one enantiomer is much greater than about 80% by weight, the compositions are referred to as “substantially enantiomerically enriched,” “substantially enantiomerically pure” or a “substantially non-racemic” preparation.

Optical isomers can be obtained by resolution of the racemic mixtures according to conventional processes, e.g., by formation of diastereoisomeric salts, by treatment with an optically active acid or base. Examples of appropriate acids include, but are not limited to, tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound. Optically active compounds can also be obtained by using active starting materials. In some embodiments, these isomers can be in the form of a free acid, a free base, an ester or a salt.

In any embodiments, the pharmaceutically acceptable form is a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The disclosure also embraces pharmaceutically acceptable forms that are “isotopically labeled derivatives” which are compounds that are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, fluorine and chlorine, such as ²H, ³H, ¹³C ¹⁴C, ¹⁵N, ¹⁸O, ¹⁷O, ³¹P, ³²p ³⁵S, ¹⁸F, and ³⁶Cl, respectively. Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure. In some embodiments, radiolabeled compounds are useful for studying metabolism and/or tissue distribution of the compounds or to alter the rate or path of metabolism or other aspects of biological functioning.

“Pharmaceutically acceptable carrier” or “pharmaceutically acceptable excipient” includes any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. The use of such media and agents for pharmaceutically active substances is well known in the art. Except insofar as any conventional media or agent is incompatible with the active ingredient, its use in the therapeutic compositions as disclosed herein is contemplated. Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of compounds described herein.

Definitions of specific functional groups and chemical terms are described in more detail below. The chemical elements are identified in accordance with the Periodic Table of the Elements, CAS version, Handbook of Chemistry and Physics, 75th ed., inside cover, and specific functional groups are generally defined as described therein. Additionally, general principles of organic chemistry, as well as specific functional moieties and reactivity, are described in Organic Chemistry, Thomas Sorrell, University Science Books, Sansalito, 1999; Smith and March March's Advanced Organic Chemistry, 5th ed., John Wiley & Sons, Inc., NewYork, 2001; Larock, Comprehensive Organic Transformations, VCH Publishers, Inc., NewYork, 1989; and Carruthers, Some Modern Methods of Organic Synthesis, 3rd ed., Cambridge University Press, Cambridge, 1987.

Recitation of ranges of values herein merely serve as a shorthand method of referring individually to each separate value and sub-range falling within the range, unless otherwise indicated herein, and each separate value and sub-range is incorporated into the specification as if it were individually recited herein. For example, “C₁₋₆ alkyl” will be understood to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C_{4- 6}, C₄₋₅, and C₅₋₆ alkyl. Likewise, 1-4 substituents will be understood to encompass 1, 2, 3, 4, 1-2, 1-3, 1-4, 2-3, 2-4 or 3-4 substituents.

“Alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1, 2, 3, 4 5, 6, 7, 8, 9, or 10 carbon atoms, although the present definition also covers the occurrence of the term “alkyl” where no numerical range is designated. In some embodiments, alkyl groups have 1 to 10, 1 to 8, 1 to 6, or 1 to 3 carbon atoms. Representative saturated straight chain alkyls include, but are not limited to, methyl, ethyl, n-propyl, n-butyl, n-pentyl, and n-hexyl groups; while saturated branched alkyls include, but are not limited to, isopropyl, sec-butyl, isobutyl, tert-butyl, isopentyl, 2-methylbutyl, 3-methylbutyl, 2-methylpentyl, 3-methylpentyl, 4-methylpentyl, 2methylhexyl, 3-methylhexyl, 4-methylhexyl, 5-methylhexyl, 2,3-dimethylbutyl, and the like. The alkyl is attached to the parent molecule by a single bond. Unless stated otherwise in the specification, an alkyl group may be optionally substituted by one or more of substituents disclosed herein. In a non-limiting embodiment, a substituted alkyl can be selected from fluoromethyl, difluoromethyl, trifluoromethyl, 2-fluoroethyl, 3-fluoropropyl, hydroxymethyl, 2-hydroxyethyl, 3hydroxypropyl, benzyl, and phenethyl.

“Alkenyl” refers to a straight or branched hydrocarbon chain radical group consisting solely of carbon and hydrogen atoms, containing at least one double bond, and having from two to ten carbon atoms (i.e., C₂₋₁₀ alkenyl). Whenever it appears herein, a numerical range such as “2 to 10” refers to each integer in the given range; e.g., “2 to 10 carbon atoms” means that the alkenyl group can consist of 2 carbon atoms, 3 carbon atoms, etc., up to and including 10 carbon atoms. In any embodiments, an alkenyl comprises two to eight carbon atoms. In other embodiments, an alkenyl comprises two to six carbon atoms (e.g., C₂₋₆ alkenyl). The alkenyl is attached to the parent molecular structure by a single bond, for example, ethenyl (i.e., vinyl), prop-1-enyl (i.e., allyl), but-1-enyl, pent-1-enyl, penta-1,4-dienyl, and the like. The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), 2-methylprop-2-enyl (C₄), butadienyl (C₄) and the like. Examples of C₂₋₆ alkenyl groups include the aforementioned C₂₋₄ alkenyl groups as well as pentenyl (C₅), pentadienyl (C₅), hexenyl (C₆), 2,3-dimethyl-2-butenyl (C₆) and the like. Additional examples of alkenyl include heptenyl (C₇), octenyl (C₈), octatrienyl (C₈) and the like. Unless stated otherwise in the specification, an alkenyl group may be optionally substituted by one or more of substituents disclosed herein.

“Alkynyl” refers to a straight or branched hydrocarbon chain radical group consisting solely of carbon and hydrogen atoms, containing at least one triple bond, having from two to ten carbon atoms (i.e., C₂₋₁₀ alkynyl). Whenever it appears herein, a numerical range such as “2 to 10” refers to each integer in the given range; e.g., “2 to 10 carbon atoms” means that the alkynyl group can consist of 2 carbon atoms, 3 carbon atoms, etc., up to and including 10 carbon atoms. In any embodiments, an alkynyl comprises two to eight carbon atoms. In other embodiments, an alkynyl has two to six carbon atoms (e.g., C₂₋₆ alkynyl). The alkynyl is attached to the parent molecular structure by a single bond, for example, ethynyl, propynyl, butynyl, pentynyl, 3-methyl-4-pentynyl, hexynyl, and the like. Unless stated otherwise in the specification, an alkynyl group may be optionally substituted by one or more of substituents disclosed herein.

“Alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, tbutoxy, pentoxy, cyclopropyloxy, cyclohexyloxy and the like. “Lower alkoxy” refers to alkoxy groups containing one to six carbons. In some embodiments, C₁₋₄alkoxy is an alkoxy group which encompasses both straight and branched chain alkyls of from 1 to 4 carbon atoms. Unless stated otherwise in the specification, an alkoxy group may be optionally substituted by one or more of substituents disclosed herein. The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

“Aromatic” or “aryl” refers to a radical with 6 to 14 ring atoms (e.g., C₆₋₁₄ aromatic or C₆14 aryl) which has at least one ring having a conjugated pi electron system which is carbocyclic (e.g., phenyl, fluorenyl, and naphthyl). In some embodiments, the aryl is a C₆₋₁₀ aryl group. For example, bivalent radicals formed from substituted benzene derivatives and having the free valences at ring atoms are named as substituted phenylene radicals. In other embodiments, bivalent radicals derived from univalent polycyclic hydrocarbon radicals whose names end in “-yl” by removal of one hydrogen atom from the carbon atom with the free valence are named by adding “idene” to the name of the corresponding univalent radical, e.g., a naphthyl group with two points of attachment is termed naphthylidene. Whenever it appears herein, a numerical range such as “6 to 14 aryl” refers to each integer in the given range; e.g., “6 to 14 ring atoms” means that the aryl group can consist of 6 ring atoms, 7 ring atoms, etc., up to and including 14 ring atoms. The term includes monocyclic or fused-ring polycyclic (i.e., rings which share adjacent pairs of ring atoms) groups. Polycyclic aryl groups include bicycles, tricycles, tetracycles, and the like. In a multi-ring group, only one ring is required to be aromatic, so groups such as indanyl are encompassed by the aryl definition. Non-limiting examples of aryl groups include phenyl, phenalenyl, naphthalenyl, tetrahydronaphthyl, phenanthrenyl, anthracenyl, fluorenyl, indolyl, indanyl, and the like. Unless stated otherwise in the specification, an aryl group may be optionally substituted by one or more of substituents disclosed herein.

“Cycloalkyl” and “carbocyclyl” each refer to a monocyclic or polycyclic radical that contains only carbon and hydrogen, and can be saturated or partially unsaturated. Partially unsaturated cycloalkyl groups can be termed “cycloalkenyl” if the carbocycle contains at least one double bond, or “cycloalkynyl” if the carbocycle contains at least one triple bond. Cycloalkyl groups include groups having from 3 to 13 ring atoms (i.e., C₃₋₁₃ cycloalkyl). Whenever it appears herein, a numerical range such as “3 to 10” refers to each integer in the given range; e.g., “3 to 13 carbon atoms” means that the cycloalkyl group can consist of 3 carbon atoms, 4 carbon atoms, 5 carbon atoms, etc., up to and including 13 carbon atoms. The term “cycloalkyl” also includes bridged and spiro-fused cyclic structures containing no heteroatoms. The term also includes monocyclic or fused-ring polycyclic (i.e., rings which share adjacent pairs of ring atoms) groups. Polycyclic aryl groups include bicycles, tricycles, tetracycles, and the like. In some embodiments, “cycloalkyl” can be a C₃₋₈ cycloalkyl radical. In some embodiments, “cycloalkyl” can be a C₃₋₅ cycloalkyl radical. Illustrative examples of cycloalkyl groups include, but are not limited to the following moieties: C₃₋₆ carbocyclyl groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ carbocyclyl groups include norbornyl (C₇). Examples of C₃₋₈ carbocyclyl groups include the aforementioned C₃₋₇ carbocyclyl groups as well as cycloheptyl(C₇), cycloheptadienyl (C₇), cycloheptatrienyl (C₇), cyclooctyl (C₈), bicyclo[2.2.1]heptanyl, bicyclo[2.2.2]octanyl, and the like. Examples of C₃₋₁₃ carbocyclyl groups include the aforementioned C₃₋₈ carbocyclyl groups as well as octahydro-1H indenyl, decahydronaphthalenyl, spiro[4.5]decanyl and the like. Unless stated otherwise in the specification, a cycloalkyl group may be optionally substituted by one or more of substituents disclosed herein. The terms “cycloalkenyl” and “cycloalkynyl” mirror the above description of “cycloalkyl” wherein the prefix “alk” is replaced with “alken” or “alkyn” respectively, and the parent “alkenyl” or “alkynyl” terms are as described herein. For example, a cycloalkenyl group can have 3 to 13 ring atoms, such as 5 to 8 ring atoms. In some embodiments, a cycloalkynyl group can have 5 to 13 ring atoms.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof, preferably substituted with one, two, or three halo groups. For example, the terms “fluoroalkyl” and “fluoroalkoxy” include haloalkyl and haloalkoxy groups, respectively, in which the halo is fluorine, such as, but not limited to, trifluoromethyl, difluoromethyl, 2,2,2trifluoroethyl, 1-fluoromethyl-2-fluoroethyl, —O—CHF₂, and the like. Each of the alkyl, alkenyl, alkynyl and alkoxy groups are as defined herein and can be optionally further substituted as defined herein.

“Heteroaryl” or, alternatively, “heteroaromatic” refers to a refers to a radical of a 5-18 membered monocyclic or polycyclic (e.g., bicyclic, tricyclic, tetracyclic and the like) aromatic ring system (e.g., having 6, 10 or 14 π electrons shared in a cyclic array) having one or more ring carbon atoms and 1-6 ring heteroatoms provided in the aromatic ring system, wherein each heteroatom is independently selected from nitrogen, oxygen, phosphorous and sulfur (“5-18 membered heteroaryl”). Heteroaryl polycyclic ring systems can include one or more heteroatoms in one or both rings. Whenever it appears herein, a numerical range such as “5 to 18” refers to each integer in the given range; e.g., “5 to 18 ring atoms” means that the heteroaryl group can consist of 5 ring atoms, 6 ring atoms, etc., up to and including 18 ring atoms. In some instances, a heteroaryl can have 5 to 14 ring atoms. In some embodiments, the heteroaryl has, for example, bivalent radicals derived from univalent heteroaryl radicals whose names end in “-yl” by removal of one hydrogen atom from the atom with the free valence are named by adding “-ene” to the name of the corresponding univalent radical, e.g., a pyridyl group with two points of attachment is a pyridylene.

For example, an N-containing “heteroaromatic” or “heteroaryl” moiety refers to an aromatic group in which at least one of the skeletal atoms of the ring is a nitrogen atom. One or more heteroatom(s) in the heteroaryl radical can be optionally oxidized. One or more nitrogen atoms, if present, can also be optionally quaternized. Heteroaryl also includes ring systems substituted with one or more nitrogen oxide (—O—) substituents, such as pyridinyl N-oxides. The heteroaryl is attached to the parent molecular structure through any atom of the ring(s).

“Heteroaryl” also includes ring systems wherein the heteroaryl ring, as defined above, is fused with one or more aryl groups wherein the point of attachment to the parent molecular structure is either on the aryl or on the heteroaryl ring, or wherein the heteroaryl ring, as defined above, is fused with one or more cycloalkyl or heterocyclyl groups wherein the point of attachment to the parent molecular structure is on the heteroaryl ring. For polycyclic heteroaryl groups wherein one ring does not contain a heteroatom (e.g., indolyl, quinolinyl, carbazolyl and the like), the point of attachment to the parent molecular structure can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having one or more ring carbon atoms and 1-4 ring heteroatoms provided in the aromatic ring system, wherein each heteroatom is independently selected from nitrogen, oxygen, phosphorous, and sulfur (“5-10 membered heteroaryl”). In some embodiments, a heteroaryl group is a 5-8 membered aromatic ring system having one or more ring carbon atoms and 1-4 ring heteroatoms provided in the aromatic ring system, wherein each heteroatom is independently selected from nitrogen, oxygen, phosphorous, and sulfur (“5-8 membered heteroaryl”). In some embodiments, a heteroaryl group is a 5-6 membered aromatic ring system having one or more ring carbon atoms and 1-4 ring heteroatoms provided in the aromatic ring system, wherein each heteroatom is independently selected from nitrogen, oxygen, phosphorous, and sulfur (“5-6 membered heteroaryl”). In some embodiments, the 5-6 membered heteroaryl has 1-3 ring heteroatoms independently selected from nitrogen, oxygen, phosphorous, and sulfur. In some embodiments, the 5-6 membered heteroaryl has 1-2 ring heteroatoms independently selected from nitrogen, oxygen, phosphorous, and sulfur. In some embodiments, the 5-6 membered heteroaryl has 1 ring heteroatom selected from nitrogen, oxygen, phosphorous, and sulfur.

Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4] oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzopyranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno [2,3-d]pyrimidinyl, 5,6-dihydrobenzo[h]quinazolinyl, 5,6-dihydrobenzo[h]cinnolinyl, 6,7-dihydro-5H benzo[6,7]cyclohepta[1,2-c]pyridazinyl, dibenzofuranyl, dibenzothiophenyl, furanyl, furazanyl, furanonyl, furo [3,2-c]pyridinyl, 5,6,7,8,9,10-hexahydrocycloocta[d] pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo [4,5] thieno [2,3-d]pyrimdinyl, 6,7,8,9-tetrahydro-5H-cyclohepta[4,5]thieno [2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno [2,3-c]pridinyl, and thiophenyl (i.e., thienyl). Unless stated otherwise in the specification, a heteroaryl group may be optionally substituted by one or more of substituents disclosed herein.

“Heterocyclyl”, “heterocycloalkyl” or “heterocarbocyclyl” each refer to any 3 to 18-membered non-aromatic radical monocyclic or polycyclic moiety comprising at least one carbon atom and at least one heteroatom selected from nitrogen, oxygen, phosphorous and sulfur. A heterocyclyl group can be a monocyclic, bicyclic, tricyclic or tetracyclic ring system, wherein the polycyclic ring systems can be a fused, bridged or spiro ring system. Heterocyclyl polycyclic ring systems can include one or more heteroatoms in one or both rings. A heterocyclyl group can be saturated or partially unsaturated. Partially unsaturated heterocycloalkyl groups can be termed “heterocycloalkenyl” if the heterocyclyl contains at least one double bond, or “heterocycloalkynyl” if the heterocyclyl contains at least one triple bond. Whenever it appears herein, a numerical range such as “5 to 18” refers to each integer in the given range; e.g., “5 to 18 ring atoms” means that the heterocyclyl group can consist of 5 ring atoms, 6 ring atoms, etc., up to and including 18 ring atoms. For example, bivalent radicals derived from univalent heterocyclyl radicals whose names end in “-yl” by removal of one hydrogen atom from the atom with the free valence are named by adding “-ene” to the name of the corresponding univalent radical, e.g., a piperidine group with two points of attachment is a piperidylene.

An N-containing heterocyclyl moiety refers to a non-aromatic group in which at least one of the ring atoms is a nitrogen atom. The heteroatom(s) in the heterocyclyl radical can be optionally oxidized. One or more nitrogen atoms, if present, can be optionally quaternized. Heterocyclyl also includes ring systems substituted with one or more nitrogen oxide (—O—) substituents, such as piperidinyl N-oxides. The heterocyclyl is attached to the parent molecular structure through any atom of any of the ring(s).

“Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment to the parent molecular structure is on the heterocyclyl ring. In some embodiments, a heterocyclyl group is a 5-14 membered non-aromatic ring system having one or more ring carbon atoms and 1-4 ring heteroatoms, wherein each heteroatom is independently selected from nitrogen, oxygen, phosphorous and sulfur (“5-14 membered heterocyclyl”). In some embodiments, a heterocyclyl group is a 3-10 membered non-aromatic ring system having one or more ring carbon atoms and 1-4 ring heteroatoms, wherein each heteroatom is independently selected from nitrogen, oxygen, phosphorous and sulfur (“3-10 membered heterocyclyl”). In some embodiments, a heterocyclyl group is a 5-8 membered non-aromatic ring system having one or more ring carbon atoms and 1-4 ring heteroatoms, wherein each heteroatom is independently selected from nitrogen, oxygen, phosphorous and sulfur (“5-8 membered heterocyclyl”). In some embodiments, a heterocyclyl group is a 5-6 membered non-aromatic ring system having one or more ring carbon atoms and 1-4 ring heteroatoms, wherein each heteroatom is independently selected from nitrogen, oxygen, phosphorous and sulfur (“5-6 membered heterocyclyl”). In some embodiments, the 5-6 membered heterocyclyl has 1-3 ring heteroatoms independently selected from nitrogen, oxygen phosphorous and sulfur. In some embodiments, the 5-6 membered heterocyclyl has 1-2 ring heteroatoms independently selected from nitrogen, oxygen, phosphorous and sulfur. In some embodiments, the 5-6 membered heterocyclyl has 1 ring heteroatom selected from nitrogen, oxygen, phosphorous and sulfur.

“Heterocyclyl” may include one or more ketone group (—C(═O)—) as part of the ring. Examples of a ketone-containing heterocycle include, without limitation, pyridin-2(1H)-one, pyrazin-2(1H)-one, pyrimidin-2(1H)-one, pyrimidin-4(3H)-one, pyridazin-3(2H)-one, pyridin-4(1H)-one, imidazolidin-2-one, 1,3-dihydro-2H-imidazol-2-one, 2,4-dihydro-3H-1,2,4-triazol-3-one, oxazol-2(3H)-one, and oxazolidin-2-one. A ketone-containing heterocyclyl is obtainable by removing a hydrogen atom from its corresponding ketone-containing heterocycle at any available N—H or C—H position.

Exemplary 3-membered heterocyclyls containing 1 heteroatom include, without limitation, azirdinyl, oxiranyl, and thiorenyl. Exemplary 4-membered heterocyclyls containing 1 heteroatom include, without limitation, azetidinyl, oxetanyl and thietanyl. Exemplary 5-membered heterocyclyls containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyls containing 2 heteroatoms include, without limitation, dioxolanyl, oxathiolanyl, thiazolidinyl, and dithiolanyl. Exemplary 5-membered heterocyclyls containing 3 heteroatoms include, without limitation, triazolinyl, diazolonyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6 membered heterocyclyl groups containing 2 heteroatoms include, without limitation, piperazinyl, morpholinyl, thiomorpholinyl, dithianyl, dioxanyl, and triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, benzoxanyl, benzopyrrolidinyl, benzopiperidinyl, benzoxolanyl, benzothiolanyl, benzothianyl, tetrahydroindolyl, tetrahydroquinolinyl, tetrahydroisoquinolinyl, decahydroquinolinyl, decahydroisoquinolinyl, 3-1H-benzimidazol-2-one, (1-substituted)-2-oxo-benzimidazol-3-yl, octahydrochromenyl, octahydroisochromenyl, decahydronaphthyridinyl, decahydro-1,8-naphthyridinyl, octahydropyrrolo[3,2-b]pyrrole, phenanthridinyl, indolinyl, phthalimidyl, naphthalimidyl, chromanyl, chromenyl, 1H-benzo[e] [1,4]diazepinyl, 1,4,5,7-tetrahydropyrano[3,4-b]pyrrolyl, 5,6-dihydro-4H-furo[3,2-b]pyrrolyl, 6,7-dihydro-5H-furo [3,2-b]pyranyl, 5,7-dihydro-4H-thieno [2,3-c]pyranyl, 2,3-dihydro-1H-pyrrolo[2,3-b]pyridinyl, hydrofuro[2,3-b]pyridinyl, 4,5,6,7 tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

Unless stated otherwise in the specification, a heterocyclyl group may be optionally substituted by one or more of substituents disclosed herein.

Where substituent groups are specified by their conventional chemical formulae, written from left to right, they equally encompass the chemically identical substituents that would result from writing the structure from right to left, e.g., —CH₂O— is equivalent to —OCH₂—.

A “leaving group or atom” is any group or atom that will, under the reaction conditions, cleave from the starting material, thus promoting reaction at a specified site. Suitable non-limiting examples of such groups unless otherwise specified include halogen atoms, mesyloxy, p-nitrobenzensulphonyloxy, trifluoromethyloxy, and tosyloxy groups.

“Protecting group” has the meaning conventionally associated with it in organic synthesis, i.e., a group that selectively blocks one or more reactive sites in a multifunctional compound such that a chemical reaction can be carried out selectively on another unprotected reactive site and such that the group can readily be removed after the selective reaction is complete. Non-limiting embodiments of functional groups that can be masked with a protecting group include an amine, hydroxy, thiol, carboxylic acid, and aldehyde. For example, a hydroxy protected form is where at least one of the hydroxy groups present in a compound is protected with a hydroxy protecting group. A variety of protecting groups are disclosed, for example, Greene's Protective Groups in Organic Synthesis, Fifth Edition, Wiley (2014), incorporated herein by reference in its entirety. For additional background information on protecting group methodologies (materials, methods and strategies for protection and deprotection) and other synthetic chemistry transformations useful in producing the compounds described herein, see in R. Larock, Comprehensive organic Transformations, VCH Publishers (1989); Greene's Protective Groups in Organic Synthesis, Fifth Edition, Wiley (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995). These references are incorporated herein by reference in their entirety.

The terms “substituted” or “substitution” mean that at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. Unless otherwise indicated, a “substituted” group can have a substituent at one or more substitutable positions of the group, and when more than one position in any given structure is substituted, the substituent is either the same or different at each position. Substituents include one or more group(s) individually and independently selected from acyl, alkyl, alkenyl, alkynyl, alkoxy, alkylaryl, cycloalkyl, aralkyl, aryl, aryloxy, amino, amido, amidino, imino, azide, carbonate, carbamate, carbonyl, heteroalkyl, heteroaryl, heteroarylalkyl, heterocycloalkyl, hydroxy, cyano, halo, haloalkoxy, haloalkyl, ester, ether, mercapto, thio, alkylthio, arylthio, thiocarbonyl, nitro, oxo, phosphate, phosphonate, phosphinate, silyl, sulfinyl, sulfonyl, sulfonamidyl, sulfoxyl, sulfonate, urea, —Si(R^{a})₃, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜN(R^{a})₂ (where t is 1 or 2), —P(═O)(R^{a})(R^{a}), or —O—P(═O)(OR^{a})₂ where each R^{a} is independently hydrogen, alkyl, haloalkyl, carbocyclyl, carbocyclylalkyl, aryl, aralkyl, heterocycloalkyl, heterocycloalkylalkyl, heteroaryl or heteroarylalkyl, and each of these moieties (other than hydrogen) can be optionally substituted with one or more substituents (up to six, valence permitting) independently selected from OH, NH₂, oxo, halo, nitro, COOH, C(O)NH₂ or cyano. For example, a cycloalkyl substituent can have a halide substituted at one or more ring carbons, and the like. The protecting groups that can form the protective derivatives of the above substituents are known to those of skill in the art and can be found in references such as Greene and Wuts, above.

Suitable substituents include, but are not limited to, haloalkyl and trihaloalkyl, alkoxyalkyl, halophenyl, -M-heteroaryl, -M-heterocycle, -M-aryl, -M-OR^{a}, -M-SR^{a}, -M-N(R^{a})₂, -M-OC(O)N(R^{a})₂, -M-C(═NR^{a})N(R^{a})₂, -M-C(═NR^{a})OR^{a}, -M-P(O)(R^{a})₂, Si(R^{a})₃, -M-NR^{a}C(O)R^{a}, -M-NR^{a}C(O)OR^{a}, -M-C(O)R^{a}, -M-C(═S)R^{a}, -M-C(═S)NR^{a}R^{a}, -M-C(O)N(R^{a})₂, -M-C(O)NR^{a}-M-N(R^{a})₂, -M-NR^{a}C(NR^{a})N(R^{a})₂, -M-NR^{a}C(S)N(R^{a})₂, -M-S(O)₂R^{a}, -M C(O)R^{a}, -M-OC(O)R^{a}, -MC(O)SR^{a}, -M-S(O)₂N(R^{a})₂, —C(O)-M-C(O)R^{a}, -MCO₂R^{a}, -MC(═O)N(R^{a})₂, -M-C(═NH)N(R^{a})₂, and -M-OC(═NH)N(R^{a})₂ (wherein M is a C₁₋₆ alkyl group).

When a ring system (e.g., cycloalkyl, heterocyclyl, aryl, or heteroaryl) is substituted with several substituents varying within an expressly defined range, it is understood that the total number of substituents does not exceed the normal available valencies under the existing conditions. Thus, for example, a phenyl ring substituted with “p” substituents (where “p” ranges from 0 to 5) can have 0 to 5 substituents, whereas it is understood that a pyridinyl ring substituted with “p” substituents has several substituents ranging from 0 to 4. The maximum number of substituents that a group in the disclosed compounds can have can be easily determined. The substituted group encompasses only those combinations of substituents and variables that result in a stable or chemically feasible compound. A stable compound or chemically feasible compound is one that, among other factors, has stability sufficient to permit its preparation and detection. In some embodiments, disclosed compounds are sufficiently stable that they are not substantially altered when kept at a temperature of 40° C. or less, in the absence of moisture (e.g., less than about 10%, less than about 5%, less than about 2%, less than about 1%, or less than about 0.5%) or other chemically reactive conditions, for e.g., at least about 3 days, at least about a week, at least about 2 weeks, at least about 4 weeks, or at least about 6 weeks.

The terms “combine, combining, to combine, combination” refer to the action of adding at least one chemical substance to another chemical substance(s) either sequentially or simultaneously. In some embodiments, bringing these chemical substances together can result in transformation of the initial chemical substances into one or more different chemical substances. This transformation can occur through one or more chemical reactions, e.g., where covalent bonds are formed, broken, rearranged and the like. A non-limiting example can include hydrolysis of an ester into an alcohol and carboxylic acid which can result from the combination of the ester with a suitable base. In another non-limiting example, an aryl fluoride can be combined with an amine to provide an aryl amine through a substitution process. These terms also include changes in association of charged chemical substances and creation of charged chemical substances, such as, but not limited to, N-oxide formation, acid addition salt formation, basic addition salt formation, and the like. These terms include the creation and/or transformation of radical chemical substances and isotopically labeled chemical substances.

The terms “convert, converting, to convert, conversion” refer to a subset of “combination” and its grammatical equivalents, where the action of one or more reagents transforms one or more functional groups on a chemical substance to another functional group(s). For example, a conversion includes, but is not limited to, transforming a nitro functional group on a chemical substance to an amine with a reducing agent. Conversions also include changes in charged chemical substances, radical chemical substances and isotopically labeled chemical substances. However, the term “convert” does not include alteration of conserved bonds in disclosed genuses and compounds.

Compounds

In one aspect, the present technology relates to a compound of Formulae (I)-(IV),

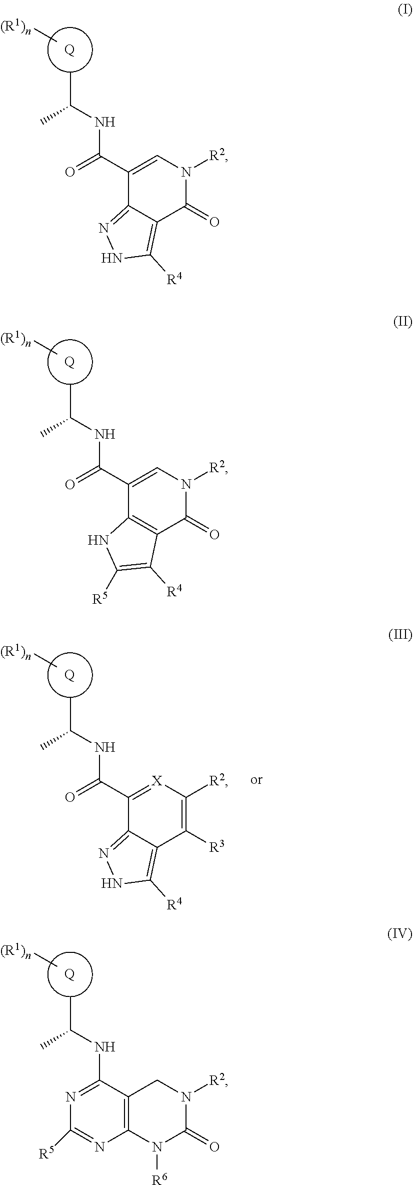

or a pharmaceutically acceptable salt, tautomer or stereoisomer thereof,
wherein:
Q at each occurrence is independently a ring selected from phenyl or a 5- or 6-membered heteroaryl group, wherein the heteroaryl group comprises at least one carbon atom and 1-4 additional heteroatoms independently selected from nitrogen, oxygen and sulfur;
X is CH or N;
R¹ at each occurrence is independently hydrogen, halogen, C₁₋₆alkyl, haloC₁₋₆alkyl, C₁₋₆alkenyl, C₁₋₆alkynyl, —NR^{a}R^{b}, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, —S(O)ₜ—C₁₋₆alkyl, —S(O)ₜ—NR^{a}R^{b}, phenyl, or 3-7-membered heterocyclyl, wherein the phenyl and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b}, or two adjacent R¹ groups, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁. 4alkyl-NR^{a}R^{b}, and oxo group (═O);
R² at each occurrence is independently hydrogen, halogen, CN, —OR^{a}, —NR^{a}R^{b}, C₁₋₆alkyl, haloC₁₋₆alkyl, C₂₋₆alkenyl, C₂₋₆alkynyl, C₃₋₇cycloalkyl, 3-7-membered heterocylyl, phenyl, or 5-6-membered heteroaryl, wherein each of the C₁₋₆alkyl, haloC₁₋₆alkyl, C₂₋₆alkenyl, C₂₋₆alkynyl, C₃₋₇cycloalkyl, 3-7-membered heterocylyl, phenyl, and 5-6-membered heteroaryl is optionally substituted with 1-5 R⁸;
R³ at each occurrence is independently hydrogen, halogen, C₁₋₆alkyl, haloC₁₋₆alkyl, C₁₋₆alkoxy, haloC₁₋₆alkoxy, C₁₋₆alkyl-OH, CN, C₃₋₇cycloalkyl, C₃₋₇cycloalkyl-OH, C₃₋₇cycloalkoyx, —NH₂, —NHC₁₋₄alkyl, —N(C₁₋₄alkyl)₂, or 3-7-membered cyclic amine;
R⁴ at each occurrence is independently hydrogen, halogen, C₁₋₆alkyl, haloC₁₋₆alkyl, C₁₋₆alkoxy, haloC₁₋₆alkoxy, CN, NH₂, C₃₋₇cycloalkyl or C₃₋₇cycloalkoxy;
R⁵ at each occurrence is independently hydrogen, C₁₋₄alkyl, or haloC₁₋₄alkyl;
R⁶ at each occurrence is independently hydrogen, C₁₋₆alkyl, haloC₁₋₆alkyl, or C₃₋₇cycloalkyl;
R⁸ at each occurrence is independently hydrogen, halogen, C₁₋₄alkyl, haloC₁₋₄alkyl, C₁₋₄alkoxy, C₂₋₄alkenyl, C₂₋₄alkynyl, C₃₋₇cycloalkyl, C₃₋₇cycloalkoxy, 3-7-membered heterocyclyl, phenyl, 5-6-membered heteroaryl, —OR^{a}, —SR^{a}, S(O)ₜR^{a}, —S(O)ₜNR^{a}R^{b}, —OC(O)—R^{a}, —NR^{a}R^{b}, —C(O)R^{a}, —C(O)OR^{a}, —OC(O)NR^{a}R^{b}₂, —C(O)NR^{a}R^{b}, —N(R^{a})C(O)OR^{a}, —N(R^{a})C(O)R^{a}, —N(R^{a})C(O)NR^{a}R^{b}, —N(R^{a})C(NR^{a})NR^{a}R^{b}, —N(R^{a})S(O)ₜNR^{a}R^{b}, —P(═O)(R^{a})(R^{b}), —O—P(═O)(OR^{a})(OR^{b}), or oxo group (═O);
R^{a} and R^{b} at each occurrence are independently hydrogen, C₁₋₆alkyl, haloC₁₋₆alkyl, C₁₋₆alkyl-OH, C₁₋₆alkoxy, C₃₋₇cycloalkyl, 3-7-membered heterocyclyl, C₁₋₆alkyl-NH₂, C₁₋₆alkyl-NHC₁₋₄alkyl, C₁₋₆alkyl-N(C₁₋₄alkyl)₂, or C₁₋₆alkyl-(3-7-membered cyclic amine), wherein each of the foregoing groups may be optionally substituted by one to three substituents which may be the same or different selected from the group consisting of C₁₋₄alkyl, haloC₁₋₄alkyl, halogen, OH, NH₂, C₁₋₄alkoxy, haloC₁₋₄alkoxy, CN, and —C(O)C₁₋₄alkyl; or R^{a} and R^{b}, together with the nitrogen atom to which they are attached, form a saturated or unsaturated heterocyclic ring containing from three to seven ring atoms, which ring may optionally contain additional one or two heteroatoms independently selected from the group consisting of nitrogen, oxygen and sulfur and may be optionally substituted by from one to three substituents independently selected from the group consisting of C₁₋₄alkyl, —C(O)C₁₋₄alkyl, phenyl and benzyl;
n at each occurrence is independently 1, 2 or 3, and
t at each occurrence is independently 1 or 2.

In some embodiments, the present technology relates to a compound of Formula (I):

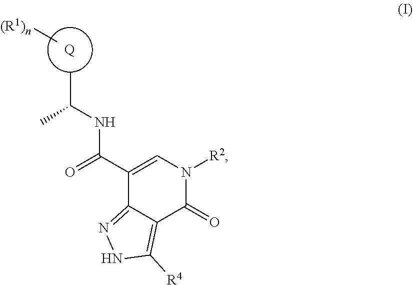

or a pharmaceutically acceptable salt, tautomer or stereoisomer thereof, wherein Q, R¹, R², R⁴, R⁸, R^{a}, R^{b}, n and t are each as defined above, or may have any of the values disclosed herein.

In some embodiments, the present technology relates to a compound of Formula (II):

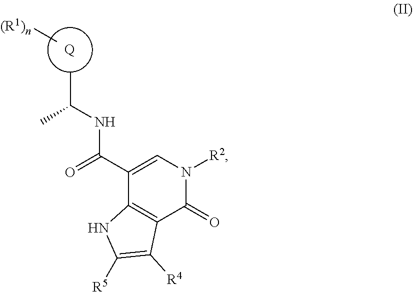

or a pharmaceutically acceptable salt, tautomer or stereoisomer thereof,
wherein Q, R¹, R², R⁴, R⁵, R⁸, R^{a}, R^{b}, n and t are each as defined above, or may have any of the values disclosed herein.

In some embodiments, the present technology relates to a compound of Formula (III):

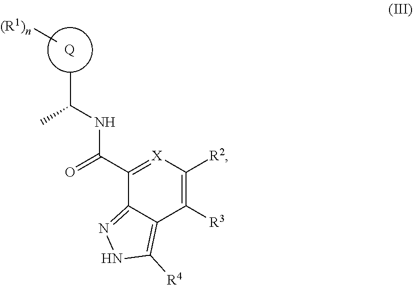

or a pharmaceutically acceptable salt, tautomer or stereoisomer thereof,
wherein Q, X, R¹, R², R³, R⁴, R⁸, R^{a}, R^{b}, n and t are each as defined above, or may have any of the values disclosed herein.

In some embodiments, the present technology relates to a compound of Formula (IV):

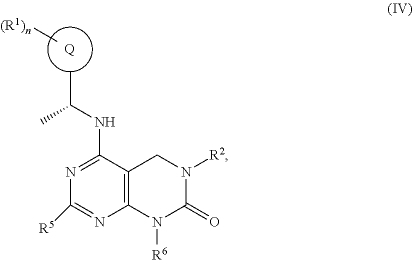

or a pharmaceutically acceptable salt, tautomer or stereoisomer thereof,
wherein Q, R¹, R², R⁵, R⁶, R⁸, R^{a}, R^{b}, n and t are each as defined above, or may have any of the values disclosed herein.

In any embodiments of the present compounds (including but not limited to compounds of Formulae I, II, III, and IV), Q at each occurrence is phenyl. In some embodiments, Q at each occurrence is independently a 5-membered heteroaryl group. In some embodiments, Q at each occurrence is independently a 6-membered heteroaryl group. In some embodiments, Q at each occurrence is thiophenyl. In some embodiments, Q at each occurrence is pyridinyl.

In any embodiments of the present compounds, R¹ at each occurrence is independently hydrogen, halogen, C₁₋₆alkyl, haloC₁₋₆alkyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy or —S(O)ₜ—C₁₋₆alkyl. In some embodiments, R¹ at each occurrence is independently phenyl or 3-7-membered heterocyclyl, wherein the phenyl and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜNR^{a}R^{b}, —NR^{a}R^{b} and C₁₋₄alkyl-NR^{a}R^{b}. In some embodiments, two adjacent R¹ groups at each occurrence, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b}, and oxo group (═O). In some embodiments, two adjacent R¹ groups at each occurrence, together with the carbon atoms to which they are attached, form a 5-6-membered carbocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b} and oxo group (═O). In some embodiments, two adjacent R¹ groups at each occurrence, together with the carbon atoms to which they are attached, form a 5-6-membered heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b} and oxo group (═O).

In any embodiments of the present compounds, Q together with its substituents (R¹)ₙ

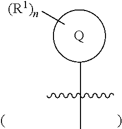

at each occurrence independently has the structure of

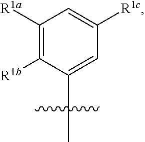

wherein:
R^{1a} at each occurrence is independently hydrogen, halogen, C₁₋₆alkyl, haloC₁₋₆alkyl, C₁₋₆alkenyl, C₁₋₆alkynyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, —S(O)ₜ—C₁₋₆alkyl, —S(O)ₜ—NR^{a}R^{b}, or 3-7-membered heterocyclyl, wherein the 3-7-membered heterocyclyl is optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b};
R^{1b} at each occurrence is independently hydrogen, halogen, C₁₋₄alkyl or C₃₋₆cycloalkyl;
or R^{1a} and R^{1b}, at each occurrence, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b}, and oxo group (═O); and
R^{1c} at each occurrence is independently hydrogen, halogen, NH₂, or C₁₋₄alkyl.

In some embodiments, the compound of Formula (I) includes a compound of Formula (Ia),

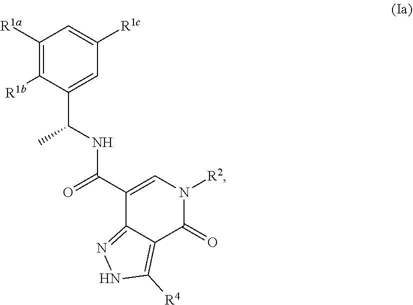

wherein R^{1a}, R^{1b}, R^{1c}, R², and R⁴ are each defined as above, or may each have any of the values disclosed herein, including but not limited to groups incorporating R^{a}, R^{b}, R⁸ and t as defined herein.

In some embodiments, the compound of Formula (II) includes a compound of Formula (IIa),

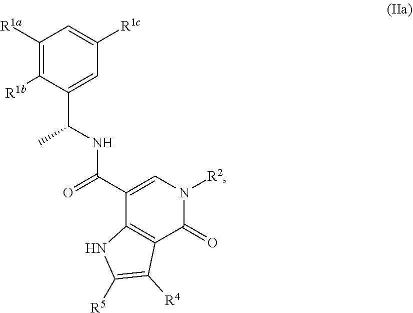

wherein R^{1a}, R^{1b}, R^{1c}, R², R⁴, and R⁵ are each defined as above, or may each have any of the values disclosed herein, including but not limited to groups incorporating R^{a}, R^{b}, R⁸, and t as defined herein.

In some embodiments, the compound of Formula (III) includes a compound of Formula (IIIa),

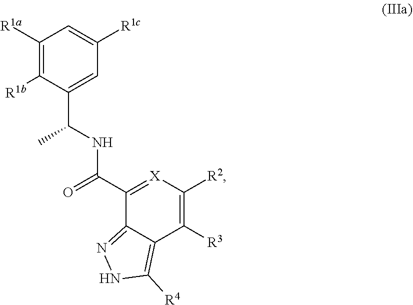

wherein R^{1a}, R^{1b}, R^{1c}, R², R³, and R⁵ are each defined as above, or may each have any of the values disclosed herein, including but not limited to groups incorporating R^{a}, R^{b}, R⁸, and t as defined herein.

In some embodiments, the compound of Formula (IV) includes a compound of Formula (IVa),

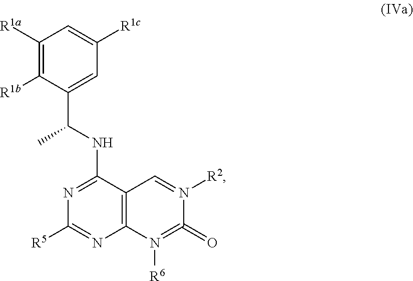

wherein R^{1a}, R^{1b}, R^{1c}, R², R⁵, and R⁶ are each defined as above, or may each have any of the values disclosed herein, including but not limited to groups incorporating R^{a}, R^{b}, R⁸, and t as defined herein.

In any embodiments, R^{1a} at each occurrence is independently hydrogen, halogen, C₁₋₆alkyl, haloC₁₋₆alkyl, C₁₋₆alkenyl, C₁₋₆alkynyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, or —S(O)ₜ—C₁₋₆alkyl. In certain embodiments, R^{1a} at each occurrence is independently 3-7-membered heterocyclyl optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b}. In some embodiments, R^{1a} at each occurrence is independently CHF₂, CH₂F, CF₃, CF₂CH₃ or CF₂CH₂OH. In some embodiments, R^{1a} and R^{1b}, at each occurrence, together with the carbon atoms to which they are attached, form a 5- or 6-membered carbocyclic ring optionally substituted with 1-3 halogen. In some embodiments, Ria and R^{1b}, at each occurrence, together with the carbon atoms to which they are attached, form a carbocyclic ring having the structure of

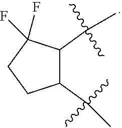 [SMILES: *C1CCC(F)(F)C1*]

In any embodiments, R^{1b} at each occurrence is independently hydrogen. In some embodiments, R^{1b} at each occurrence is independently halogen. In some embodiments, R^{1b} at each occurrence is independently C₁₋₄alkyl. In some embodiments, R^{1b} at each occurrence is independently F or methyl.

In any embodiments, R^{1c} at each occurrence is independently hydrogen. In some embodiments, R^{1c} at each occurrence is independently halogen. In some embodiments, R^{1c} at each occurrence is independently NH₂. In some embodiments, R^{1c} at each occurrence is independently C₁₋₄alkyl.

In any embodiments, Q together with its substituents (R¹)ₙ,

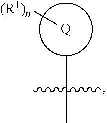

at each occurrence independently has the structure of

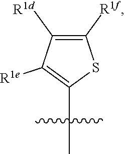

wherein:
R^{1d} and R^{1e} at each occurrence are independently hydrogen, halogen, C₁₋₄alkyl, or haloC₁₋₄alkyl; and Rif at each occurrence is independently phenyl, 5-6-membered heteroaryl, or 3-7-membered heterocyclyl, wherein the phenyl, 5-6-membered heteroaryl, and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b} and C₁₋₄alkyl-NR^{a}R^{b}.

In some embodiments, the compound of Formula (I) includes a compound of Formula (Ib),

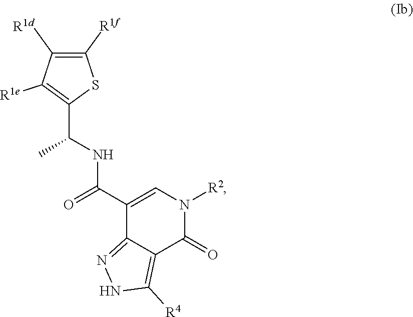

wherein R^{1d}, R^{1e}, R^{1f}, R², and R⁴ are each defined as above, or may each have any of the values disclosed herein, including but not limited to groups incorporating R^{a}, R^{b}, R⁸ and t as defined herein.

In some embodiments, the compound of Formula (II) includes a compound of Formula (IIb),

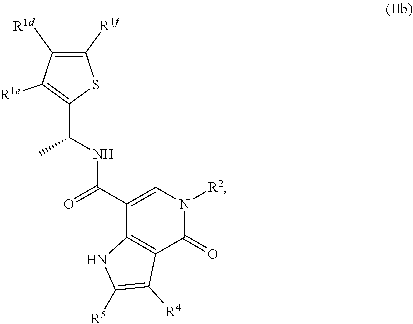

wherein R^{1d}, R^{1e}, R¹, R², R⁴, and R⁵ are each defined as above, or may each have any of the values disclosed herein, including but not limited to groups incorporating R^{a}, R^{b}, R⁸, and t as defined herein.

In some embodiments, the compound of Formula (III) includes a compound of Formula (IIIb),

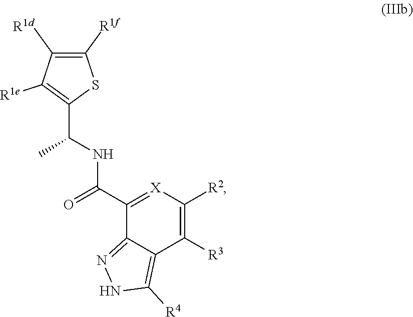

wherein R^{1d}, R^{1e}, R¹, R², R³, and R⁵ are each defined as above, or may each have any of the values disclosed herein, including but not limited to groups incorporating R^{a}, R^{b}, R⁸, and t as defined herein.

In some embodiments, the compound of Formula (IV) includes a compound of Formula (IVb),

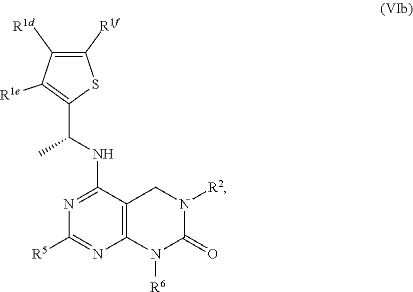

wherein R^{1d}, R^{1e}, R^{1f}, R², R⁵, and R⁶ are each defined as above, or may each have any of the values disclosed herein, including but not limited to groups incorporating R^{a}, R^{b}, R⁸, and t as defined herein.

In any embodiments, R^{1d} and R^{1e} at each occurrence are independently hydrogen or methyl. In some embodiments, R^{1d} and R^{1e} at each occurrence are independently hydrogen.

In any embodiments, R^{1f} at each occurrence is independently phenyl optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b} and C₁₋₄alkyl-NR^{a}R^{b}. In some embodiments, R^{f}f at each occurrence is independently phenyl optionally substituted with C₁₋₄alkyl-NR^{a}R^{b}. In some embodiments, R^{f}f at each occurrence is independently phenyl optionally substituted with C₁₋₄alkyl-NH—C₁₋₄alkyl. In some embodiments, R^{f}f at each occurrence is independently phenyl optionally substituted with C₁₋₃alkyl-NH—C₁₋₃alkyl. In some embodiments, R^{f}f at each occurrence is independently phenyl optionally substituted with C₁₋₂alkyl-NH—C₁₋₂alkyl. In some embodiments, R^{1f} at each occurrence is independently phenyl optionally substituted with C₁₋₂alkyl-N(C₁₋₂alkyl)-C₁₋₂alkyl.

In any embodiments, R² at each occurrence is independently hydrogen, halogen, CN, —OR^{a}, —NR^{a}R^{b}, C₁₋₆alkyl, or haloC₁₋₆alkyl, wherein each of the C₁₋₆alkyl and haloC₁₋₆alkyl is optionally substituted with 1-5 R⁸. In some embodiments, R² at each occurrence is independently C₃₋₇cycloalkyl optionally substituted with 1-5 R⁸. For example, R² may be cyclopropyl or cyclobutyl. In some embodiments, R² at each occurrence is independently 3-7-membered heterocylyl optionally substituted with 1-5 R⁸. For example, R² may be tetrahydrofuranyl, tetrahydropyranyl, piperidinyl, piperazinyl, or morpoholinyl. In some embodiments, R² at each occurrence is independently phenyl optionally substituted with 1-5 R⁸. In some embodiments, R² at each occurrence is independently 5-membered heteroaryl optionally substituted with 1-5 R⁸. In some embodiments, R² at each occurrence is independently 6-membered heteroaryl optionally substituted with 1-5 R⁸.

In any embodiments, R³ at each occurrence is independently hydrogen, halogen, C₁₋₆alkyl, haloC₁₋₆alkyl, or CN. In some embodiments, R³ at each occurrence is independently C₁₋₆alkoxy, haloC₁₋₆alkoxy, or C₁₋₆alkyl-OH. In some embodiments, R³ at each occurrence is independently C₃₋₇cycloalkyl or C₃₋₇cycloalkyl-OH. In some embodiments, R³ at each occurrence is independently —NH₂, —NHC₁₋₄alkyl, —N(C₁₋₄alkyl)₂, or 3-7-membered cyclic amine.

In any embodiments, X is CH. In some embodiments, X is N.

In any embodiments, R⁴ at each occurrence is independently hydrogen. In some embodiments, R⁴ at each occurrence is independently halogen. In some embodiments, R⁴ at each occurrence is independently C₁₋₆alkyl. In some embodiments, R⁴ at each occurrence is independently methyl. In some embodiments, R⁴ at each occurrence is independently ethyl. In some embodiments, R⁴ at each occurrence is independently propyl. In some embodiments, R⁴ at each occurrence is independently iso-propyl.

In any embodiments, R⁵ at each occurrence is independently hydrogen or C₁₋₄alkyl. In some embodiments, R⁵ at each occurrence is independently hydrogen. In some embodiments, R⁵ at each occurrence is independently methyl. In some embodiments, R⁵ at each occurrence is independently ethyl. In some embodiments, R⁵ at each occurrence is independently propyl. In some embodiments, R⁵ at each occurrence is independently iso-propyl.

In any embodiments, R⁶ at each occurrence is independently hydrogen or C₁₋₄alkyl. In some embodiments, R⁶ at each occurrence is independently hydrogen. In some embodiments, R⁶ at each occurrence is independently C₁₋₄alkyl. In some embodiments, R⁶ at each occurrence is independently methyl. In some embodiments, R⁶ at each occurrence is independently ethyl. In some embodiments, R⁶ at each occurrence is independently propyl. In some embodiments, R⁶ at each occurrence is independently iso-propyl.

In any embodiments, R⁸ at each occurrence is independently hydrogen, halogen, C₁₋₄alkyl, haloC₁₋₄alkyl, or C₁₋₄alkoxy. In some embodiments, R⁸ at each occurrence is independently C₃₋₇cycloalkyl or 3-7-membered heterocyclyl. In some embodiments, R⁸ at each occurrence is independently phenyl or 5-6-membered heteroaryl. In some embodiments, R⁸ at each occurrence is independently —OR^{a}, —SR^{a}, —S(O)ₜR^{a}, —S(O)ₜ—NR^{a}R^{b}, —OC(O)—R^{a}, —NR^{a}R^{b}, —C(O)R^{a}, —C(O)OR^{a}, —OC(O)NR^{a}R^{b}, —C(O)NR^{a}R^{b}, —N(R^{a})C(O)OR^{a}, —N(R^{a})C(O)R^{a}, —N(R^{a})C(O)NR^{a}R^{b}, —N(R^{a})C(NR^{a})NR^{a}R^{b}, —N(R^{a})S(O)ₜNR^{a}R^{b}, —P(═O)(R^{a})(R^{b}), —O—P(═O)(OR^{a})(OR^{b}), or oxo group (═O). In some embodiments, R⁸ at each occurrence is independently oxo group (═O). In some embodiments, R⁸ at each occurrence is independently —NR^{a}R^{b}. In some embodiments, R⁸ at each occurrence is independently —NR^{a}R^{b}, wherein said —NR^{a}R^{b} is independently NH₂, —NHC₁₋₄alkyl, —N(C₁₋₄alkyl)₂, or 3-7-membered cyclic amine. In some embodiments, R⁸ at each occurrence is independently —S(O)ₜ—C₁₋₄alkyl.

In any embodiments, the compound is selected from:

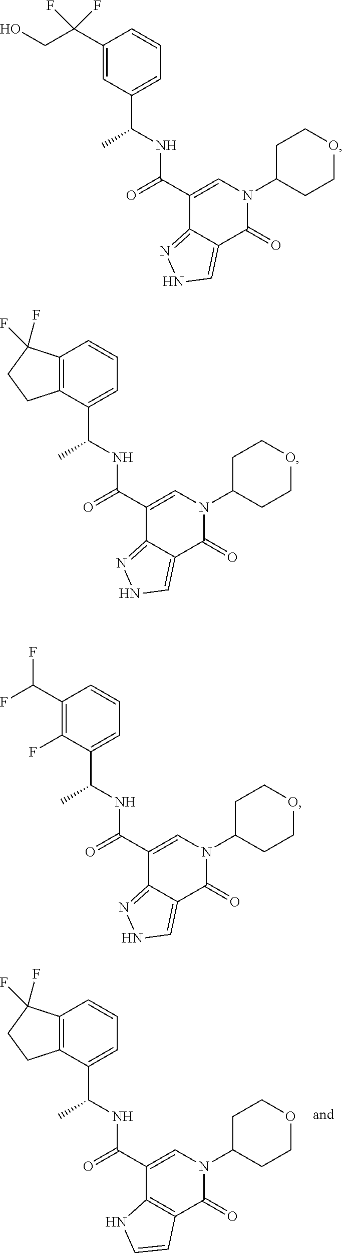 [SMILES: C[C@@H](NC(=O)c1cn(C2CCOCC2)c(=O)c2c[nH]nc12)c1cccc(C(F)(F)CO)c1 ; C[C@@H](NC(=O)c1cn(C2CCOCC2)c(=O)c2c[nH]nc12)c1cccc2c1CCC2(F)F ; C[C@@H](NC(=O)c1cn(C2CCOCC2)c(=O)c2c[nH]nc12)c1cccc(C(F)F)c1F ; C[C@@H](NC(=O)c1cn(C2CCOCC2)c(=O)c2cc[nH]c12)c1cccc2c1CCC2(F)F]

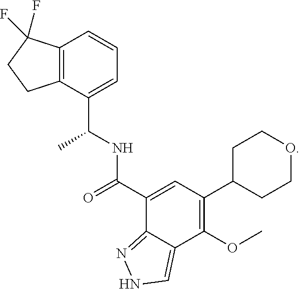 [SMILES: COc1c(C2CCOCC2)cc(C(=O)N[C@H](C)c2cccc3c2CCC3(F)F)c2n[nH]cc12]

In any embodiments, the compound is selected from:

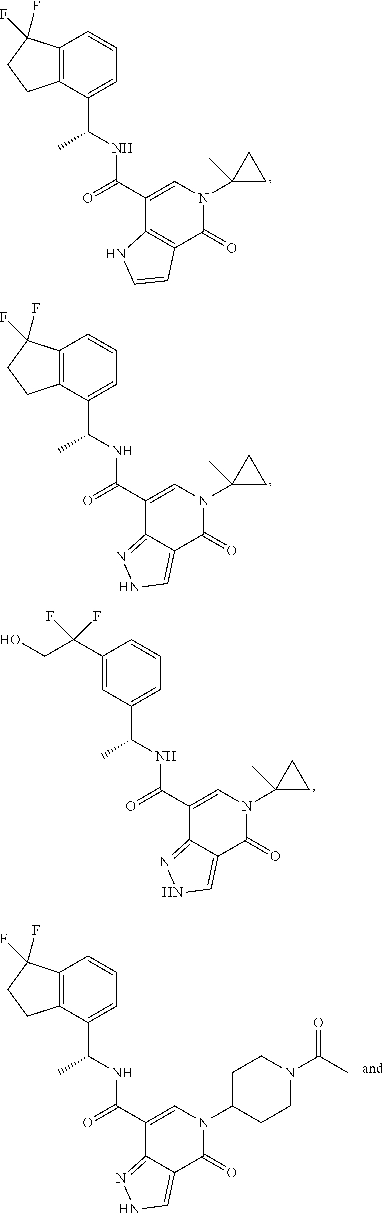 [SMILES: C[C@@H](NC(=O)c1cn(C2(C)CC2)c(=O)c2cc[nH]c12)c1cccc2c1CCC2(F)F ; C[C@@H](NC(=O)c1cn(C2(C)CC2)c(=O)c2c[nH]nc12)c1cccc2c1CCC2(F)F ; C[C@@H](NC(=O)c1cn(C2(C)CC2)c(=O)c2c[nH]nc12)c1cccc(C(F)(F)CO)c1 ; CC(=O)N1CCC(n2cc(C(=O)N[C@H](C)c3cccc4c3CCC4(F)F)c3n[nH]cc3c2=O)CC1]

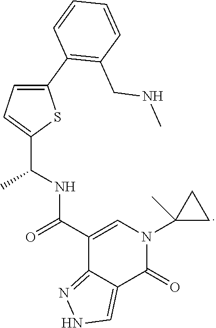 [SMILES: CNCc1ccccc1-c1ccc([C@@H](C)NC(=O)c2cn(C3(C)CC3)c(=O)c3c[nH]nc23)s1]

In some embodiments, the present technology relates to a compound of Formulae (I), (Ia), (Ib), (II), (IIa), (IIb), (III), (IIIa), (IIIb), (IV), (IVa), and (IVb), including each exemplified compound, wherein at least one hydrogen (H) is replaced with deuterium (D). Enriching for deuterium may afford certain therapeutic advantages, such as increasing in vivo half-life or reducing dosage requirements, or may provide a compound useful as a standard for characterization of biological samples. In some other embodiments, a compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In another aspect, the present technology relates to a pharmaceutical composition comprising a compound disclosed herein (including but not limited to a compound of Formulae (I), (Ia), (Ib), (II), (IIa), (IIb), (III), (IIIa), (IIIb), (IV), (IVa), and/or (IVb)), and a pharmaceutically acceptable carrier.

In yet another aspect, the present technology relates to a method for treating or preventing a disease or condition mediated by mammalian Ras family proteins in a subject in need thereof, comprising administering an effective amount of a compound disclosed herein (including but not limited to a compound of Formulae (I), (Ia), (Ib), (II), (IIa), (IIb), (III), (IIIa), (IIIb), (IV), (IVa), and/or (IVb)) to the subject.

In yet another aspect, the present technology relates to a method for treating or preventing cancer in a subject in need thereof, comprising administering an effective amount of a compound disclosed herein (including but not limited to a compound of Formulae (I), (Ia), (Ib), (II), (IIa), (IIb), (III), (IIIa), (IIIb), (IV), (IVa), and/or (IVb)) to the subject. In some embodiments, the cancer is breast cancer, leukemia, prostate cancer, ovarian cancer, pancreatic cancer, colon cancer, lung cancer, endometrial cancer, thyroid cancer, cholangiocarcinoma, multiple myeloma, melanoma, uterine cancer, head and neck squamous cell carcinoma, diffuse large B cell lymphoma, esophageal cancer, hepatocellular cancer, glioblastoma, renal cancer, sarcoma, bladder cancer, urothelial cancer, gastric cancer, or cervical cancer.

In yet another aspect, the present technology relates to a method for treating or preventing RASopathies caused by aberrant RAS signaling activities in a subject in need thereof, comprising administering an effective amount of a compound disclosed herein (including but not limited to a compound of Formulae (I), (Ia), (Ib), (II), (IIa), (IIb), (III), (IIIa), (IIIb), (IV), (IVa), and/or (IVb)) to the subject.

In yet another aspect, the present technology relates to a method for disorder or condition in a subject in need thereof, comprising administering an effective amount of a compound disclosed herein (including but not limited to a compound of Formulae (I), (Ia), (Ib), (II), (IIa), (IIb), (III), (IIIa), (IIIb), (IV), (IVa), and/or (IVb)) to the subject, wherein the disorder or condition is selected from neurofibromatosis type 1, Noonan syndrome, Noonan syndrome with multiple lentigines, Costello syndrome, cardiofaciocutaneous syndrome, and capillary malformation-arteriovenous syndrome.

In yet another aspect, the present technology relates to a method for treating or preventing renal dysfunction associated with fibrosis in a subject in need thereof, comprising administering an effective amount of a compound disclosed herein (including but not limited to a compound of Formulae (I), (Ia), (Ib), (II), (IIa), (IIb), (III), (IIIa), (IIIb), (IV), (IVa), and/or (IVb)) to the subject.

In yet another aspect, the present technology relates to a process of making a compound of Formulae (I), (Ia), (Ib), (II), (IIa), (IIb), (III), (IIIa), (IIIb), (IV), (IVa), and (IVb), including each exemplified compound and intermediate described herein.

### EXAMPLES

General Synthetic Methods

The compounds of the present technology can be synthesized using the methods described herein, together with synthetic methods known in the art of synthetic organic chemistry, or by variations thereon as appreciated by those skilled in the art. Preferred methods include, but are not limited to, those exemplary schemes and working examples described below. All substituents are as defined hereinabove unless otherwise indicated. The reactions are performed in a solvent or solvent mixture appropriate to the reagents and materials employed and suitable for the transformations proposed. This will sometimes require a judgment to modify the order of synthetic steps or to select on particular process scheme over another in order to obtain a desired compound of the technology.

It will be recognized that another major consideration in the planning of any synthetic route in this field is the judicious choice of the protecting group used for protection of the reactive functional groups present in the compounds described in this technology. An authoritative account describing the many alternatives to the trained practitioner is by Greene et al., Greene's Protective Groups in Organic Synthesis, Fifth Edition, Wiley (2014). It will also be recognized that the compound names referred to in the descriptions of Schemes 1-5 are for convenience only, and do not necessarily reflect the actual chemical names of those compounds.

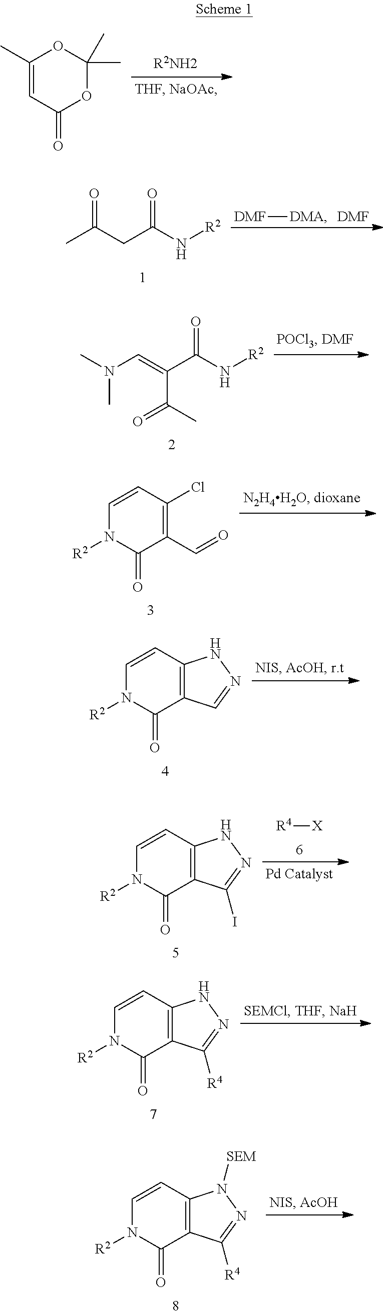

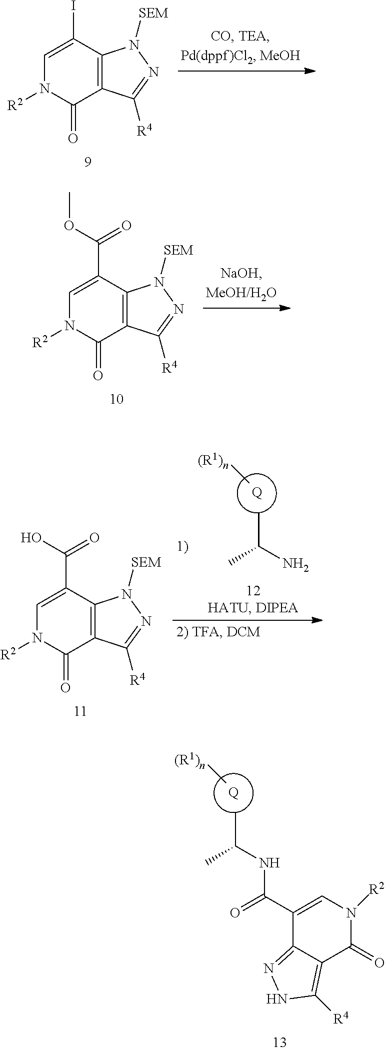

Scheme 1 describes the synthesis of pyrrozolo-pyridone amide 13. 2,2,6-Trimethyl-4H-1,3-dioxin-4-one is reacted with amine R₂—NH₂ to give acetoacetamide 1, which is treated with DMF-DMA to provide intermediate 2. Under POCl₃ condition, 2 is converted to afford pyridine-aldehyde 3. Upon treatment of 3 with hydrazine under elevated temperature, pyrrazolo-pyridone derivative 4 is obtained. 4 is reacted with NIS or NBS to give intermediate 5, which can undergo Suzuki conditions with R₄-boronic acid using appropriate catalyst (e.g. Pd(PPh₃)₄) or nucleophilic aromatic substitution reaction to provide 7. Protection of 7 with a protecting group such as SEM provides 8. Idonation of 8 with NIS to give iodo-intermediate 9, which then undergoes carbonylation reaction using appropriate catalyst such as Pd(dppf)Cl₂ in MeOH to give the corresponding ester 10. Hydrolysis of 10 under basic condition gives acid 11, and then 11 is coupled with amine 12 to afford the desired amide 13 after de-protection with TFA.

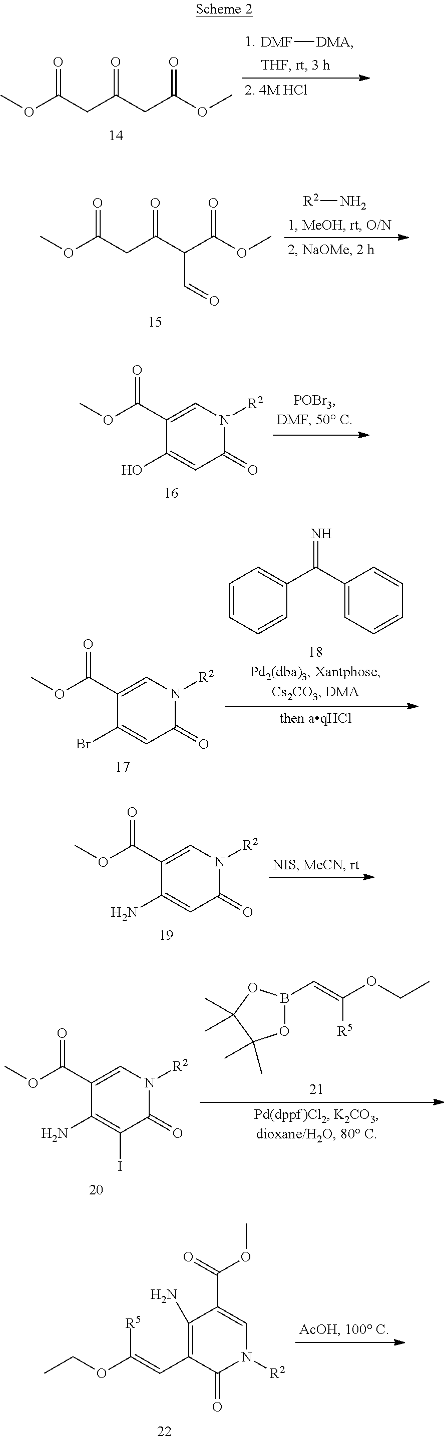

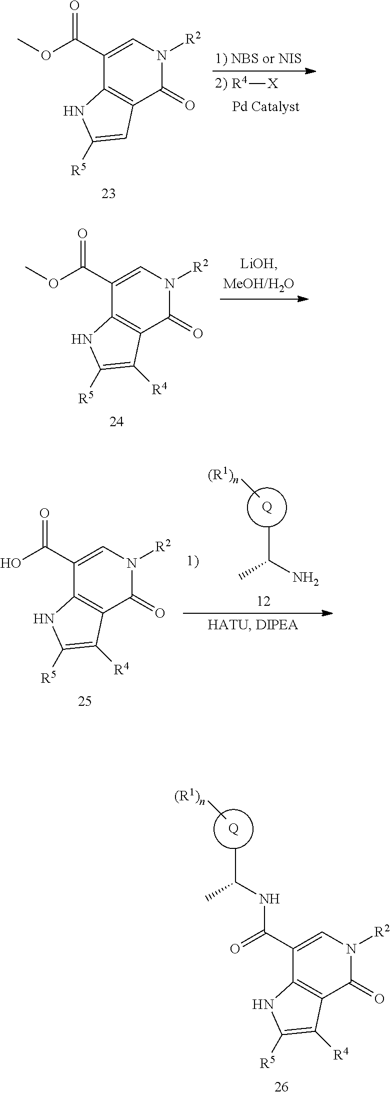

Scheme 2 describes a general synthetic route to pyrrolo-pyridone amide 26. Upon treatment of 14 with DMF-DMA, followed by treatment of acid, aldehyde intermediate 15 is obtained, which is then reacted with amine R₂—NH₂ and subsequently under basic condition to give hydroxyl-pyridone derivative 16. The conversion of 16 to bromo derivative 17 is carried out under POBr₃ condition. Buckwald reaction of 17 with bis-phenyl imine 18 under catalytic condition such as Pd₂(dba)₃/xantphose and followed by acidic treatment, affords amino-pyridone 19. Iodonation of 19 with NIS, subsequently undergoes Suzuki coupling reaction with boronic ester 21, gives intermediate 22. Pyrrolo-pyridone 23 is formed upon treatment of 22 under acidic condition. 23 can be further substituted with R₄ group after treatment of 23 with NBS or NIS, and resulting aryl halide undergoes Suzuki coupling reaction to provide 24. After hydrolysis of ester 24, acid 25 is then coupled with amine 12 under coupling condition such as HATU/DIPEA to give the desired pyrrolo-pyridone amide 26.

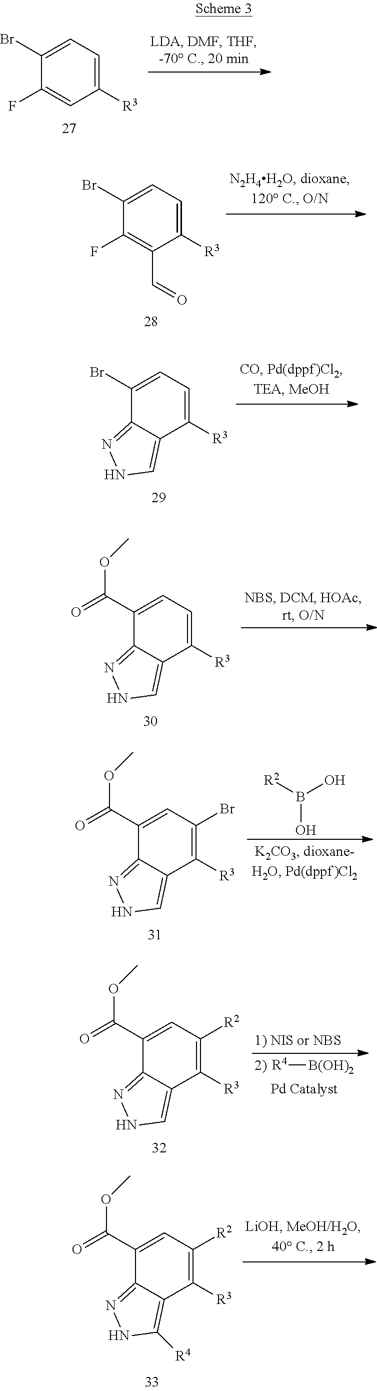

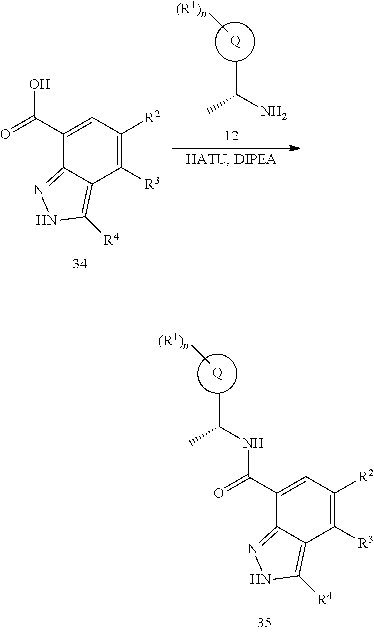

Scheme 3 describes a general synthetic route to indazole amide 35. Ortho-lithiation of 27, followed by treatment with DMF, gives aldehyde 28, which is reacted with hydrazine under elevated temperature to afford indazole 29. Formation of methyl ester 30 is performed by carbonylation of 29 under the condition of Pd(dppf)Cl₂/TEA/MeOH. Bromination of 30 with NBS generates 31, which can undergo Suzuki coupling reaction with R₄-bionic acid to give 33. Hydrolysis of 33 under basic condition, provides acid 34 which is coupled with amine 12 to afford the desired indazole amide 35.

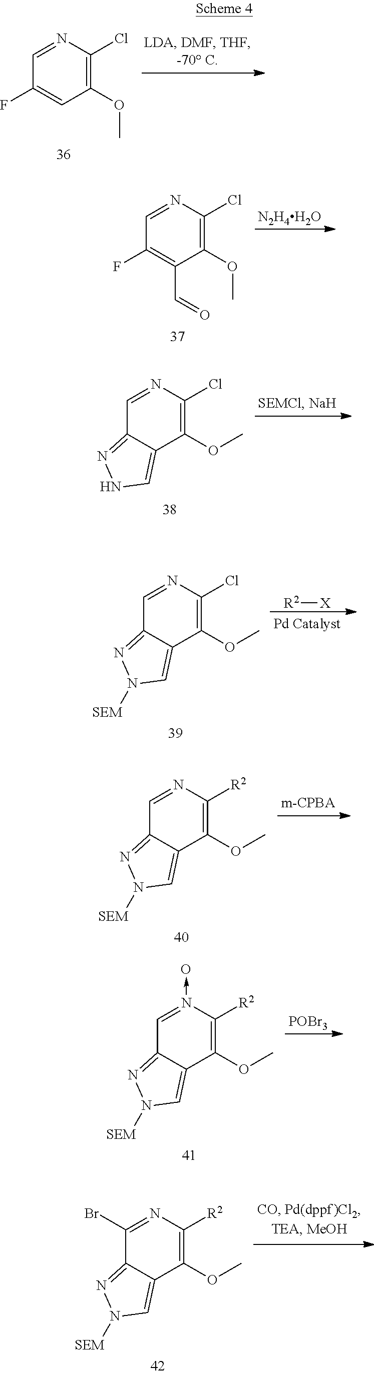

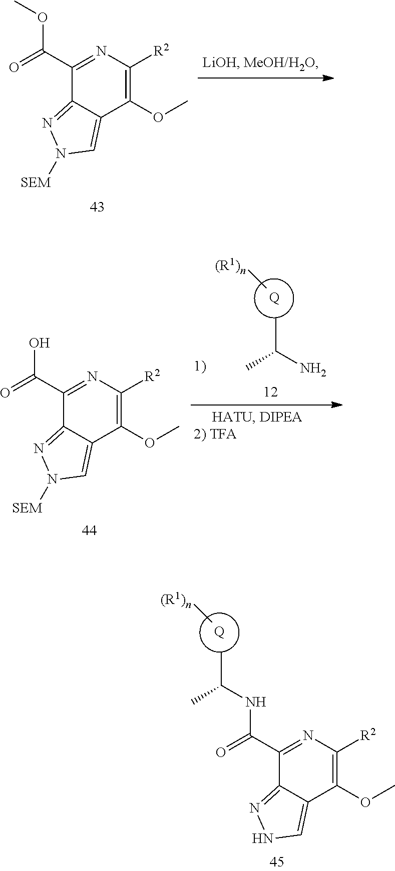

Scheme 4 describes the synthesis of aza-indazole amide derivative 45. Aldehyde intermediate 37 is prepared by ortho-lithiation by LDA at low temperature and subsequent treatment of DMF. Aza-indazole 38 is obtained by treatment of 37 with hydrazine. The protection of 38 by SEM is done by NaH/SEMCl. Aza-indazole chloride 39 can be treated with R₂—NH₂ (Buckwald reaction) or R₂—B(OH)₂ (Suzuki coupling reaction) to give 40. Upon treatment of m-CPBA, 40 is converted to 41, which is reacted with POBr₃ to afford aza-indazole bromide 42. Pd catalyzed carbonylation of 42 in MeOH provides ester 43, which is hydrolyzed under basic condition to yield the acid 44. The desired product 45 is obtained by coupling of acid 44 with amine 12 using coupling reagent such as HATU/DIPEA and followed by de-protection of SEM by TFA.

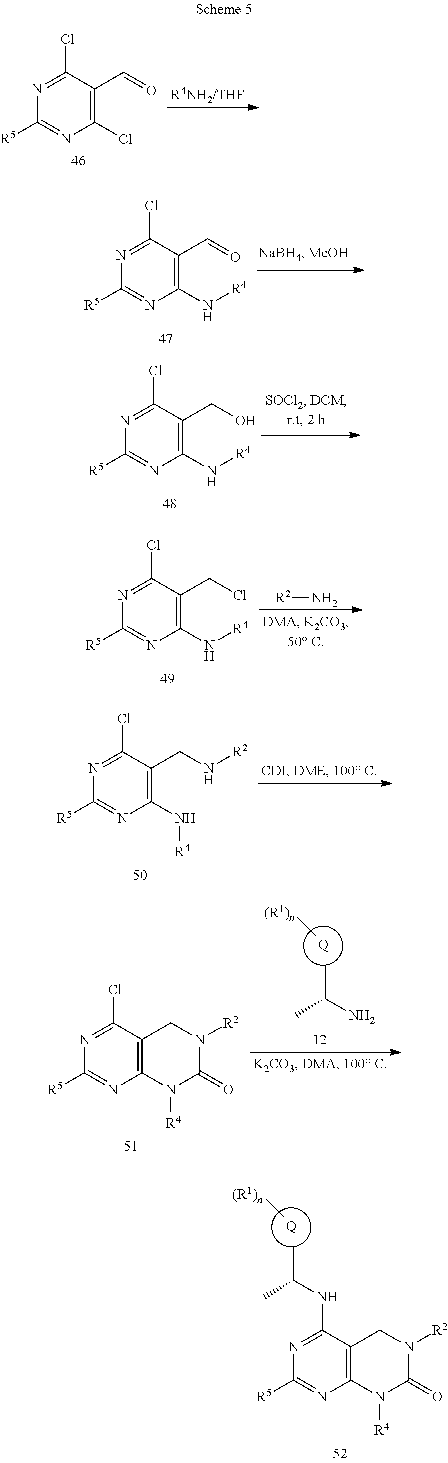

Scheme 5 describes the synthesis of urea containing pyrimidine derivative 52. Nucleophilic substitution of 46 with R₄—NH₂ gives 47, which undergoes aldehyde reduction to afford 48. Chlorination and displacement with R₂—NH₂ provides 50. Treatment of 50 with CDI at elevated temperature gives urea containing pyrimidylchloride derivative 51, which undergoes nucleophilic displacement with amine 12 to provide the desired target 52.

Pharmaceutical Compositions and Methods

The compounds utilized in the methods described herein may be formulated together with a pharmaceutically acceptable carrier or adjuvant into pharmaceutically acceptable compositions prior to be administered to a subject. In another embodiment, such pharmaceutically acceptable compositions further comprise additional therapeutic agents in amounts effective for achieving a modulation of disease or disease symptoms, including those described herein.

The term “pharmaceutically acceptable carrier or adjuvant” refers to a carrier or adjuvant that may be administered to a subject, together with a compound of the present technology, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of the present technology include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of the present technology may be administered orally, parenterally, by inhalation spray, topically, rectally, nasally, buccally, vaginally or via an implanted reservoir, preferably by oral administration or administration by injection. The pharmaceutical compositions of the present technology may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of the present technology may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of the present technology may also be administered in the form of suppositories for rectal administration. These compositions can be prepared by mixing a compound of the present technology with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of the present technology is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of the present technology include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of the present technology may also be topically applied to the lower intestinal tract by rectal suppository formulation or in a suitable enema formulation. Topically-transdermal patches are also included in the present technology.

The pharmaceutical compositions of the present technology may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of the present technology comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of the present technology. Alternatively, those agents may be part of a single dosage form, mixed together with the compounds of the present technology in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the drug. The methods herein contemplate administration of an effective amount of compound or compound composition to achieve the desired or stated effect. Typically, the pharmaceutical compositions of the present technology will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Upon improvement of a subject's condition, a maintenance dose of a compound, composition or combination of the present technology may be administered, if necessary. Subsequently, the dosage or frequency of administration, or both, may be reduced, as a function of the symptoms, to a level at which the improved condition is retained when the symptoms have been alleviated to the desired level. Subjects may, however, require intermittent treatment on a long-term basis upon any recurrence of disease symptoms.

The pharmaceutical compositions described above comprising a compound of formulae (I)-(IV) may further comprise another therapeutic agent for treating or preventing a disease or condition mediated by mammalian Ras family proteins. In particular, such combination may be useful for treating or preventing cancer, including breast cancer, leukemia, prostate cancer, ovarian cancer, pancreatic cancer, colon cancer, lung cancer, endometrial cancer, thyroid cancer, cholangiocarcinoma, multiple myeloma, melanoma, uterine cancer, head and neck squamous cell carcinoma, diffuse large B cell lymphoma, esophageal cancer, hepatocellular cancer, glioblastoma, renal cancer, sarcoma, bladder cancer, urothelial cancer, gastric cancer, or cervical cancer.

All publications, patents, and patent applications mentioned in this specification are herein incorporated by reference to the same extent as if each individual publication, patent, or patent application was specifically and individually indicated to be incorporated by reference. In case of conflict, the present application, including any definitions herein, will control.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### Examples

Abbreviations used herein are as follows:

| Abbrv. | Full Name | Abbrv. | Full Name |
|---|---|---|---|
| anhy. | anhydrous | aq. | aqueous |
| min | minute(s) | satd. | saturated |
| mL | milliliter | hrs | hours |
| mmol | millimole(s) | mol | mole(s) |
| MS | mass spectrometry | NMR | nuclear magnetic |
| | | | resonance |
| TLC | thin layer | HPLC | high-performance |
| | chromatography | | liquid |
| | | | chromatography |
| LCMS | Liquid | PPTS | Pyridinium p- |
| | chromatography- | | Toluenesulfonate |
| | mass spectrometry | | |
| DCE | 1,2-dichloroethane | CHCl₃ | chloroform |
| DCM | dichloromethane | DMF | dimethylformamide |
| Et₂O | diethyl ether | EtOH | ethyl alcohol |
| EtOAc | ethyl acetate | MeOH | methyl alcohol |
| MeCN | acetonitrile | PE | petroleum ether |
| THF | tetrahydrofuran | DMSO | dimethyl sulfoxide |
| AcOH | acetic acid | HCl | hydrochloric acid |
| H₂SO₄ | sulfuric acid | NH₄Cl | ammonium chloride |
| KOH | potassium hydroxide | NaOH | sodium hydroxide |
| K₂CO₃ | potassium carbonate | Na₂CO₃ | sodium carbonate |
| TFA | trifluoroacetic acid | Na₂SO₄ | sodium sulfate |
| NaBH₄ | sodium borohydride | NaHCO₃ | sodium bicarbonate |
| LiHMDS | lithium | NaBH₄ | sodium |
| | hexamethyl- | | borohydride |
| | disilylamide | | |
| Et₃N or | Triethylamine | Py or Pyr | pyridine |
| TEA | | | |
| TBAF | Tetrabutylammonium | MsCl | Methanesulfonyl |
| | fluoride | | chloride |
| BnBr | Benzyl bromide | DHP | 3,4-Dihydro-2H- |
| | | | pyran |
| Cbz | carbobenzyloxy | m-CPBA | 3-Chloroper- |
| | | | oxybenzoic acid |
| Dess- | 1,1,1-Triacetoxy-1,1- | DIAD | Diisopropyl |
| Martin | Dihydro-1,2- | | azodicarboxylate |
| | Benziodoxol-3(1H)-On | | |
| DMAP | 4-(dimethylamino)pyr- | DIPEA | N,N-diisopropyl- |
| | idine | | ethylamine |
| TMSCHN2 | (Trimethylsilyl)diazo- | TMSCH2N3 | Trimethylsilyl- |
| | methane | | methyl azide |

| | |
|---|---|
| Ruphos | 2-Dicyclohexylphosphino-2′,6′-diisopropoxybiphenyl |
| RuPhosPd | Methanesulfonato(2-dicyclohexylphosphino-2′,6′-di-i- |
| G3 | propoxy-1,1′-biphenyl)(2′-amino-1,1′-bipheny1- |
| | 2-yl)palladium(II) |

### General Conditions and Procedures

In the following examples, the chemical reagents were purchased from commercial sources (such as Alfa, Acros, Sigma Aldrich, TCI and Shanghai Chemical Reagent Company), and used without further purification. THE was continuously refluxed and freshly distilled from sodium and benzophenone under nitrogen, dichloromethane was continuously refluxed and freshly distilled from CaH₂ under nitrogen.

Flash chromatography was performed on an Ez Purifier III via column with silica gel particles of 200-300 mesh. Analytical and preparative thin layer chromatography plates (TLC) were HSGF 254 (0.15-0.2 mm thickness, Shanghai Anbang Company, China). Nuclear magnetic resonance (NMR) spectra were recorded using Brucker AMX-300 or AMX-400 NMR (Brucker, Switzerland) at around 20-30° C. unless otherwise specified. The following abbreviations are used: s, singlet; d, doublet; t, triplet; q, quartet; m, multiplet; dd, doublet of doublets; ddd, doublet of doublet of doublet; dt, doublet of triplets; bs, broad signal. Chemical shifts were reported in parts per million (ppm, δ) downfield from tetramethylsilane. Mass spectra were run with electrospray ionization (ESI) from a Waters LCT TOF Mass Spectrometer (Waters, USA). Compound purification was carried out as needed using a variety of traditional methods including, but not limited to, preparative chromatography under acidic, neutral, or basic conditions using either normal phase or reverse phase HPLC or flash columns or Prep-TLC plates.

Preparative HPLC: unless otherwise described, the compounds were purified using a WATERS Fractionlynx system equipped with a YMC Pack Pro d₈ Column (5 μm, 120A, 50×20 mm) and the following solvent system: H₂O, AcCN, and 2% TFA in H₂O. Specific elution gradients were based on the retention times obtained with an analytical LC-MS, however, in general all elution gradients of H₂O and MeCN were run over a 7 minute run time with a flow rate of 35 mL/min. An autoblend method was used to ensure a concentration of 0.1% TFA throughout each run. Specific elution gradients were based on the retention times obtained with an analytical LC-MS, however, in general, all elution gradients of H₂O and MeCN were run over at 8 minute run time with a flow rate of 50 mL/min.

Analytical LC-MS: analytical LC-MS was performed on a WATERS Acquity UPLC-MS instrument equipped with a ACQUITY UPLC BEH Ci₈ Column (2.1×50 mm, 1.7μιη), a column temperature of 45° C. and using the following solvent system: Solvent A: 0.1% HCOOH in H₂O; and Solvent B: 0.1% HCOOH in AcCN. All compounds were run using the same elution gradient, i.e., 5% to 95% Solvent B over a 1.5 min run time with a flow rate of 0.6 mL/min.

Preparative Chiral SFC Separation: stereoisomer mixtures were separated using a Berger Minigram SFC instrument on one of the following columns: ChiralPak AS-H (10×250 mm), ChiralPak IA (10×250 mm), ChiralPak AD-H (21×250 mm), Phenomenex Lux-2 (21.2×250 mm), or ChiralPak IC (10×250 mm); eluting with either 0.1% diethylamine in MeOH/CO₂, or 0.1% diethylamine in EtOH/CO₂ or 0.1% diethylamine in isopropanol/CO₂ with a flow rate of 2.5 mL/min and a column temperature of 35° C.

Analytical Chiral SFC Separation: stereoisomer mixtures or single enantiomers were analyzed using a JASCO analytical SFC instrument on one of the following columns: ChiralPak AS-H (4.6×250 mm), ChiralPak IA (4.6×250 mm), ChiralPak AD-H (4.6×250 mm), Phenomenex Lux-2 (4.6×250 mm), or ChiralPak IC (4.6×250 mm); eluting with either 0.1% diethylamine in MeOH/CO₂, or 0.1% diethylamine in EtOH/CO₂ or 0.1% diethylamine in isopropanol/CO₂, with a flow rate of 6.0 imL/min and a column temperature of 35° C.

### Intermediate 1: Synthesis of (R)-1-(2-methyl-3-(trifluoromethyl)phenyl)ethanamine

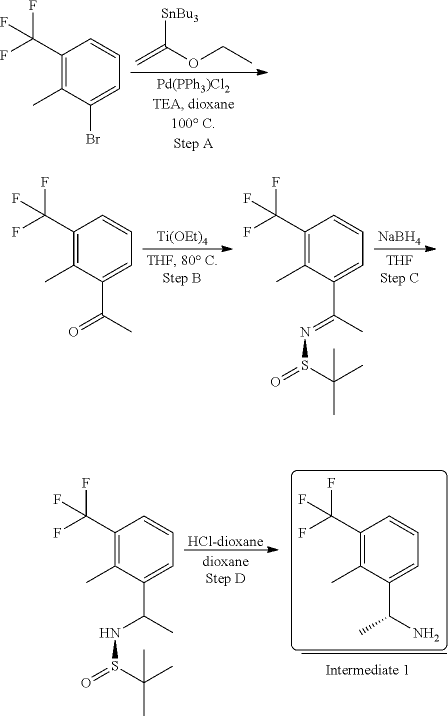

### Step A. 1-(2-methyl-3-(trifluoromethyl)phenyl)ethan-1-one

To a solution of 1-bromo-2-methyl-3-(trifluoromethyl)benzene (1.3 mL, 8.4 mmol) in dry dioxane (20 mL) is added Tributyl(1-ethoxyvinyl)stannane (3.3 g, 9.2 mmol), TEA (3.5 mL, 25.1 mmol) and Pd(PPh₃)₂Cl₂ (0.3 g, 0.4 mmol). After stirring at 100° C. for 2 h, 12 N HCl (15 mL) was added and stirred for 30 min, the cooled mixture was poured into water (50 mL) and extracted with EtOAc (50 mL*2). The combine organic phase was washed with brine and dried over Na₂SO₄ and concentrated. The crude product was purified by chromatography (silica gel, 0-25%, EtOAc in PE) to give 1-[2-methyl-3-(trifluoromethyl)phenyl]ethan-1-one (1.4 g, 7.1 mmol, 85.1%) as a brown oil. LC-MS (ESI) m/z 196 (M+H)⁺.

### Step B. (S,E)-2-methyl-N-(1-(2-methyl-3-(trifluoromethyl)phenyl)ethylidene)propane-2-sulfinamide

To a stirred solution of 1-[2-methyl-3-(trifluoromethyl)phenyl]ethan-1-one (1.44 g, 7.1 mmol) in THF (15 mL) was added (R)-2-methylpropane-2-sulfonamide (1.29 g, 10.7 mmol) and Titanium ethoxide (3 mL, 14.2 mmol) at rt. After stirring at 80° C. overnight, the cooled mixture was poured into water (50 mL) and filtered. The filtrate was extracted with EtOAc (50 mL*2). The combined organic phase was washed with brine, dried over Na₂SO₄, filtered and concentrated. The crude product was purified by chromatography (silica gel, 0-50%, EtOAc in PE) to give 2-methyl-N-[(1E)-1-[2-methyl-3-(trifluoromethyl)phenyl]ethylidene]propane-2-sulfinamide (1.2 g, 3.9 mmol, 55.2%) as a brown oil.

### Step C. (S)-2-methyl-N-(1-(2-methyl-3-(trifluoromethyl)phenyl)ethyl)propane-2-sulfinamide

To a stirred solution of 2-methyl-N-[(1E)-1-[2-methyl-3-(trifluoromethyl)phenyl]ethylidene]propane-2-sulfinamide (1.2 g, 3.9 mmol) in THF (64 mL) was added NaBH₄ (0.26 g, 7.07 mmol) at 0° C. After stirring at rt for 3.5 h, the mixture was poured into water (20 mL) and extracted with EtOAc (20 mL*2). The combined organic phase was washed with brine, dried over Na₂SO₄, filtered and concentrated. The crude product was purified by chromatography (silica gel, 0-60%, EtOAc in PE) to give 2-methyl-N-{1-[2-methyl-3-(trifluoromethyl)phenyl]ethyl}propane-2-sulfinamide (0.6 g, 1.95 mmol, 49.7%) as a yellow oil.

### Step D. (R)-1-(2-methyl-3-(trifluoromethyl)phenyl)ethan-1-amine

To a stirred solution of 2-methyl-N-{1-[2-methyl-3-(trifluoromethyl)phenyl]ethyl}propane-2-sulfinamide (60 mg, 0.2 mmol) in dioxane (5 mL) was added dioxane/HCl (5 mL, 4 mol/L) at rt. After stirring at rt for 1 h, the mixture was concentrated to give crude of (1R)-1-[2-methyl-3-(trifluoromethyl)phenyl]ethan-1-amine (100 mg, 0.15 mmol, 75.6%) as a yellow solid. LC-MS (ESI) m/z 204 (M+H)⁺.

### Intermediate 2

### Synthesis of (1R)-1-[3-(1,1-difluoroethyl)-2-fluorophenyl]ethan-1-amine

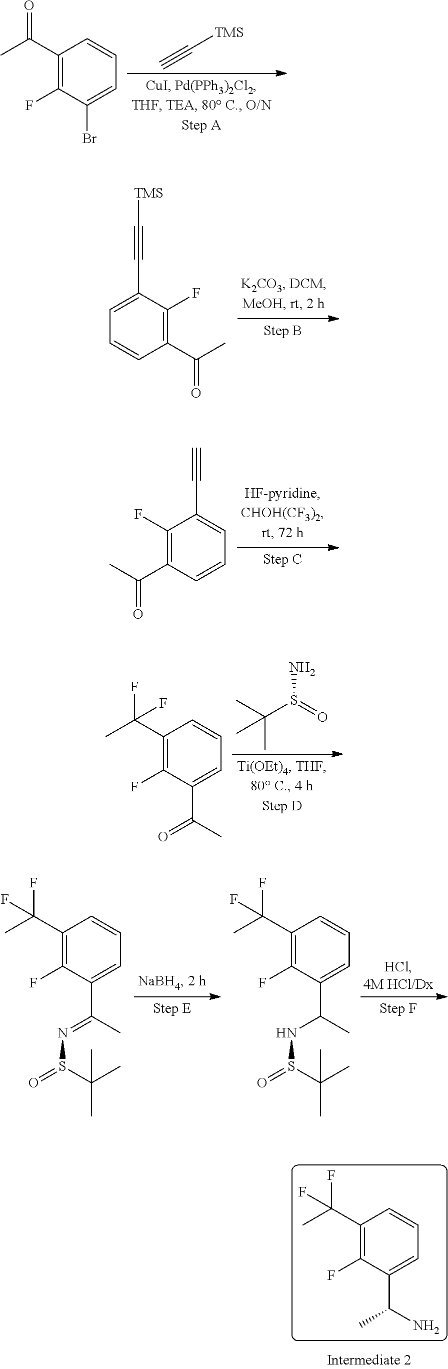

### Step A. 1-{2-fluoro-3-[2-(trimethylsilyl)ethynyl]phenyl}ethan-1-one

To a stirred solution of 1-(3-bromo-2-fluorophenyl)ethan-1-one (5 g, 23.1 mmol) in THF (30 mL) was added ethynyltrimethylsilane (5 mL, 34.5 mmol), CuI (880 mg, 4.6 mmol), Pd(dppf)Cl₂ (1.7 g, 2.3 mmol) and TEA (9.6 mL, 69.1 mmol) at rt. After stirring at 80° C. overnight, the cooled mixture was poured into water (30 ml) and extracted with DCM (20 ml*2). The combined organic phase was washed with brine, dried over Na₂SO₄, filtered and concentrated. The residue was purified by chromatography (silica gel, 0-20%, EtOAc in PE) to give 1-{2-fluoro-3-[2-(trimethylsilyl)ethynyl]phenyl}ethan-1-one (5 g, 21.3 mmol, 92%) as a yellow oil. LCMS: m/z 235 (M+H)⁺.

### Step B. 1-(3-ethynyl-2-fluorophenyl)ethan-1-one

To a stirred solution of 1-{2-fluoro-3-[2-(trimethylsilyl)ethynyl]phenyl}ethan-1-one (5 g, 21.3 mmol) in DCM (20 mL)/MeOH (20 mL) was added K₂CO₃ (8.8 g, 64.1 mmol) at rt. After stirring at rt for 2 h, the cooled mixture was poured into water (30 ml) and extracted with DCM (20 ml*2). The combined organic phase was washed with brine, dried over Na₂SO₄, filtered and concentrated to give 1-(3-ethynyl-2-fluorophenyl)ethan-1-one (3.2 g, 19.7 mmol, 92%) as a brown solid. LCMS: m/z 163 (M+H)⁺.

### Step C. 1-[3-(1,1-difluoroethyl)-2-fluorophenyl]ethan-1-one

To a stirred solution of 1-(3-ethynyl-2-fluorophenyl)ethan-1-one (2.7 g, 16.6 mmol) in CHOH(CF₃)₂ (8 mL) was added HF 70% in pyridine (8 mL) at rt. After stirring at rt over the weekend, the cooled mixture was poured into water (30 ml) and extracted with DCM (20 ml*2). The combined organic phase was washed with brine, dried over Na₂SO₄, filtered and concentrated. The residue was purified by chromatography (silica gel, 0-20%, EtOAc in PE) to give 1-[3-(1,1-difluoroethyl)-2-fluorophenyl]ethan-1-one (1.4 g, 6.9 mmol, 41%) as a brown solid. LCMS: m/z 203 (M+H)⁺.

### Step D. N-[(1E)-1-[3-(1,1-difluoroethyl)-2-fluorophenyl]ethylidene]-2-methylpropane-2-sulfinamide

To a stirred solution of 1-[3-(1,1-difluoroethyl)-2-fluorophenyl]ethan-1-one (700 mg, 3.4 mmol) in THE (10 mL) was added 2-methylpropane-2-sulfinamide (629 mg, 5.2 mmol) and Ti(OEt)₄ (2 mL) at rt. After stirring at 80° C. for 4 h, the cooled mixture was poured into water (30 ml) and extracted with DCM (20 ml*2). The combined organic phase was washed with brine, dried over Na₂SO₄, filtered and concentrated. The residue was purified by chromatography (silica gel, 0-30%, EtOAc in PE) to give N-[(1E)-1-[3-(1,1-difluoroethyl)-2-fluorophenyl]ethylidene]-2-methylpropane-2-sulfinamide (700 mg, 2.3 mmol, 66%) as a yellow oil. LCMS: m/z 236 (M+H)⁺.

### Step E. N-{1-[3-(1,1-difluoroethyl)-2-fluorophenyl]ethyl}-2-methylpropane-2-sulfinamide

To a stirred solution of N-[(1E)-1-[3-(1,1-difluoroethyl)-2-fluorophenyl]ethylidene]-2-methylpropane-2-sulfinamide (700 mg, 2.3 mmol) in THF (5 mL) was added NaBH₄ (116 mg, 3.44 mmol) at 0° C. After stirring at rt for 2 h, the cooled mixture was poured into water (30 ml) and extracted with DCM (20 ml*2). The combined organic phase was washed with brine, dried over Na₂SO₄, filtered and concentrated. The residue was purified by chromatography (silica gel, 0-30%, EtOAc in PE) to give N-{1-[3-(1,1-difluoroethyl)-2-fluorophenyl]ethyl}-2-methylpropane-2-sulfinamide (380 mg, 1.2 mmol, 54%) as a yellow solid. LCMS: m/z 308 (M+H)⁺.

¹H NMR (400 MHz, DMSO) δ 7.70 (s, 1H), 7.45 (d, J=7.3 Hz, 1H), 7.31 (t, J=7.7 Hz, 1H), 5.86 (d, J=7.7 Hz, 1H), 4.75-4.62 (m, 1H), 2.00 (dd, J=21.9, 16.3 Hz, 3H), 1.42 (d, J=6.8 Hz, 3H), 1.11 (s, 9H).

### Step F. (1R)-1-[3-(1,1-difluoroethyl)-2-fluorophenyl]ethan-1-amine

To a stirred mixture of N-{1-[3-(1,1-difluoroethyl)-2-fluorophenyl]ethyl}-2-methylpropane-2-sulfinamide (100 mg, 0.32 mmol) in dioxane (3 mL) was added 4N HCl/dioxane (3 mL) at rt. After stirring at rt for 2 h, the mixture was concentrated to give crude (1R)-1-[3-(1,1-difluoroethyl)-2-fluorophenyl]ethan-1-amine (60 mg, 0.29 mmol, 91%) as a yellow solid. LCMS: m/z 204 (M+H)⁺.

### Intermediate 3

### Synthesis of (R)-1-(6-(trifluoromethyl)pyridin-2-yl)ethanamine

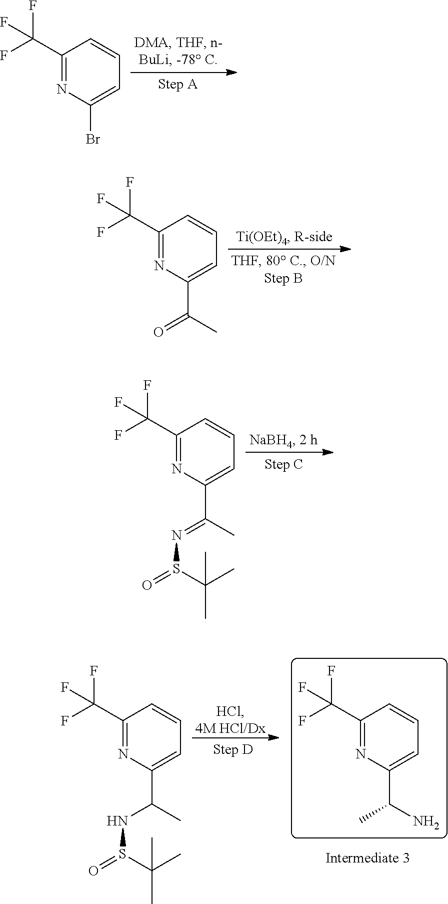

### Step A: 1-(6-(trifluoromethyl)pyridin-2-yl)ethan-1-one

To a solution of 2-bromo-6-(trifluoromethyl)pyridine (0.5 g, 2.2 mmol) in THF (5 mL) was added DMA (0.23 mL, 2.4 mmol) at rt. The mixture was colded to −60° C. and N-BuLi (0.23 g, 3.54 mmol) was dropped wisely. The reaction was stirred at −20° C. over 3 h. After complete conversion of starting material, the mixture was quenched by saturated NH₄Cl solution and extracted with EtOAc. The organic layers was washed with brine and dried over Na₂SO₄ then concentrated under reduced pressure. The crude product was purified by column chromatography on silica gel (gradient elution: 0-10% EtOAc in PE) to give the product of 1-[6-(trifluoromethyl)pyridin-2-yl]ethan-1-one (0.09 g, 0.48 mmol, 21.5%) as a yellow oil. LCMS (ESI) m/z 190 (M+H)⁺

### Step B: (S,E)-2-methyl-N-(1-(6-(trifluoromethyl)pyridin-2-yl)ethylidene)propane-2-sulfinamide

To a stirred solution of 1-[6-(trifluoromethyl)pyridin-2-yl]ethan-1-one (0.7 g, 3.7 mmol) in THE (15 mL) was added (R)-2-methylpropane-2-sulfinamide (0.67 g, 5.55 mmol) and Titanium ethoxide (2.33 mL, 11.1 mmol) at rt. After stirring at 80° C. overnight, the cooled mixture was poured into ice water (25 mL) and filtered. The filtrate was extracted with EtOAc (25 mL*2). The combined organic phase was washed with brine, dried over Na₂SO₄, filtered and concentrated. The crude product was purified by chromatography (silica gel, 0-100%, EtOAc in PE) to give 2-methyl-N-[(1E)-1-[6-(trifluoromethyl)pyridin-2-yl]ethylidene]propane-2-sulfinamide (0.74 g, 2.53 mmol, 68.4%) as a brown oil.

### Step C: (S)-2-methyl-N-(1-(6-(trifluoromethyl)pyridin-2-yl)ethyl)propane-2-sulfinamide

To a stirred solution of 2-methyl-N-[(1E)-1-[6-(trifluoromethyl)pyridin-2-yl]ethylidene]propane-2-sulfinamide (0.7 g, 2.4 mmol) in THE (7 mL) was added NaBH₄ (0.14 mL, 4.31 mmol) at 0° C. After stirring at rt for 3 h, the mixture was poured into water (20 mL) and extracted with EtOAc (20 mL*2). The combined organic phase was washed with brine, dried over Na₂SO₄, filtered and concentrated. The crude product was purified by chromatography (silica gel, 0-60%, EtOAc in PE) to give 2-methyl-N-{1-[6-(trifluoromethyl)pyridin-2-yl]ethyl}propane-2-sulfinamide (0.3 g, 1.0 mmol, 42.6%) as a yellow oil.

### Step D: (R)-1-(6-(trifluoromethyl)pyridin-2-yl)ethan-1-amine

To a solution of 2-methyl-N-{1-[6-(trifluoromethyl)pyridin-2-yl]ethyl}propane-2-sulfinamide (0.08 g, 0.27 mmol) in dioxane (5 mL) was added dioxane/HCl (3 mL, 4 mol/L) at rt. After stirring at rt for 1 h, the mixture was concentrated to give crude of (1R)-1-[6-(trifluoromethyl)pyridin-2-yl]ethan-1-amine (60 mg, 0.25 mmol, 93%) as a yellow solid. LC-MS (ESI) m/z 191 (M+H)⁺.

### Intermediate 4

### Synthesis of (R)-benzyl 2-(5-(1-aminoethyl)thiophen-2-yl)benzyl(methyl)carbamate

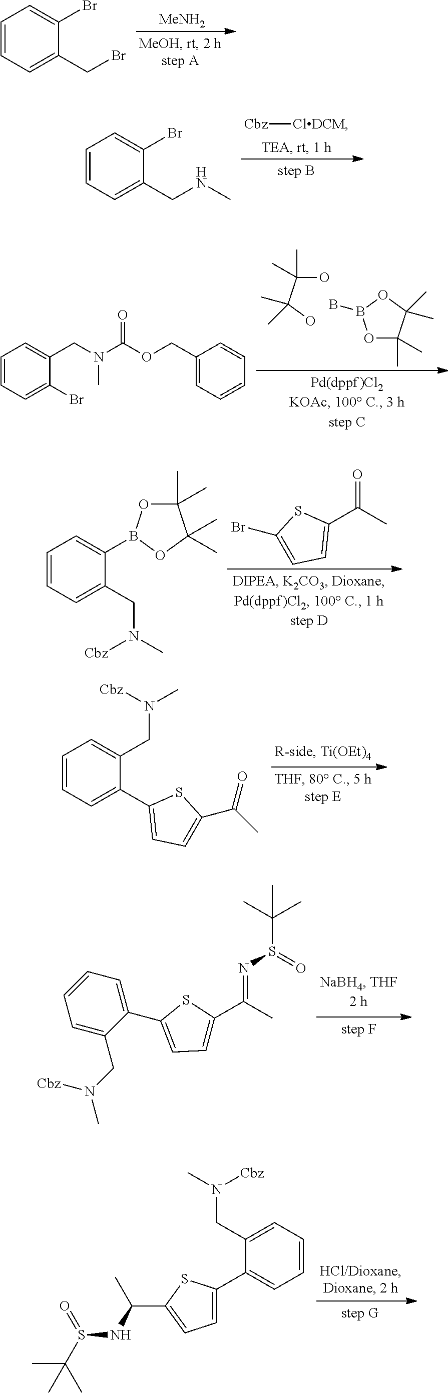

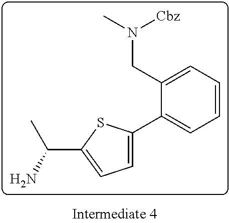 [SMILES: C[C@@H](N)c1ccc(-c2ccccc2CN(C)C(=O)OCc2ccccc2)s1]

### Step A: 1-(2-bromophenyl)-N-methylmethanamine

To a solution of MeNH₂ (10.8 g, 160 mmol) in MeOH was added a solution of 1-bromo-2-(bromomethyl)benzene (10 g, 40.0 mmol) in MeOH over 30 min at rt. After stirring at rt for 2 h, the mixture was concentrated and extracted with EtOAc. The organic layers was washed with brine and dried over Na₂SO₄. The crude product was purified by column chromatography on silica gel (gradient elution: 0-20% MeOH in DCM) to give the product of [(2-bromophenyl)methyl](methyl)amine (2.7 g, 13.5 mmol, 33.7%). LC-MS (ESI) m/z: 249⁺.

### Step B: benzyl N-[(2-bromophenyl)methyl]-N-methylcarbamate

To a solution of [(2-bromophenyl)methyl](methyl)amine (2.7 g, 13.5 mmol) in DCM (30 mL) was added TEA (5.6 mL, 40.5 mmol) at rt. A solution of CbzCl (2.76 g, 16.2 mmol) in DCM (20 mL) was added dropwise. After stirring at rt for 1.5 h, the reaction was quenched by saturated solution of NaHCO₃ and extracted with DCM. The organic layers was washed with brine and dried over Na₂SO₄, then concentrated under reduced pressure. The crude product was purified by column chromatography on silica gel (gradient elution: 0-10% EtOAc in PE) to give the product of benzyl N-[(2-bromophenyl)methyl]-N-methylcarbamate (3 g, 8.976 mmol, 66.5%) as a colourless oil. LC-MS (ESI) m/z: 334⁺

### Step C: benzyl N-methyl-N-{[2-(tetramethyl-1,3,2-dioxaborolan-2-yl)phenyl]methyl}carbamate

To a solution of benzyl N-[(2-bromophenyl)methyl]-N-methylcarbamate (4 g, 11.9 mmol) in dioxane (40 mL) was added 4,4,4′,4′,5,5,5′,5′-octamethyl-2,2′-bi(1,3,2-dioxaborolane) (3.95 g, 15.6 mmol), Pd(dppf)Cl₂ (0.44 g, 0.598 mmol) and KOAc (3.52 g, 35.9 mmol) under N₂ at rt. After stirring at 100° C. overnight, the cooled mixture was filtered. The filtrate was diluted with water and then extracted with EtOAc. The organic layers was washed with brine and dried over Na₂SO₄ then concentrated. The residue was purified by chromatography (silica gel, 0-50%, EtOAc in PE) to give the product of benzyl N-methyl-N-{[2-(tetramethyl-1,3,2-dioxaborolan-2-yl)phenyl]methyl}carbamate (4.35 g, 11.4 mmol, 95.3%) as a green oil. LC-MS (ESI) m/z 382 (M+H)⁺.

### Step D: benzyl N-{[2-(5-acetylthiophen-2-yl)phenyl]methyl}-N-methylcarbamate

To a solution of benzyl N-methyl-N-{[2-(tetramethyl-1,3,2-dioxaborolan-2-yl)phenyl]methyl}carbamate (4.35 g, 11.4 mmol) in dioxane (40 mL) and H₂O (10 mL) was added 1-(5-bromothiophen-2-yl)ethan-1-one (1.87 g, 9.13 mmol), Pd(dppf)Cl₂ (0.83 g, 1.14 mmol) and K₂CO₃ (6.31 g, 45.636 mmol) at rt. After stirring at 100° C. for 1.5 h, the mixture was filtered. The filtrate was extracted with EtOAc. The organic layers was washed with brine and dried over Na₂SO₄ then concentrated. The mixture was purified by chromatography (silica gel, 0-50%, EtOAc in PE) to give the product of benzyl N-{[2-(5-acetylthiophen-2-yl)phenyl]methyl}-N-methylcarbamate (0.38 g, 1.00 mmol, 8.78%) as a white solid. LC-MS (ESI) m/z 380 (M+H)⁺.

### Step E: benzyl N-methyl-N-[(2-{5-[(1E)-1-[(2-methylpropane-2-sulfinyl)imino]ethyl]thiophen-2-yl}phenyl)methyl]carbamate

To a solution of benzyl N-{[2-(5-acetylthiophen-2-yl)phenyl]methyl}-N-methylcarbamate (2.45 g, 6.46 mmol) in THE (30 mL) was added (R)-2-methylpropane-2-sulfinamide (1.17 g, 9.68 mmol), titanium ethoxide (4.061 mL, 19.369 mmol) at rt. After stirring at 80° C. for 5 h, the mixture was quenched with ice water and filtered. The filtrate was extracted with EtOAc. The organic layers was washed with brine and dried over Na₂SO₄, then concentrated under reduced pressure. The mixture was purified by chromatography (silica gel, 0-80%, EtOAc in PE) to give the product of benzyl N-methyl-N-[(2-{5-[(1E)-1-[(2-methylpropane-2-sulfinyl)imino]ethyl]thiophen-2-yl}phenyl)methyl]carbamate (2 g, 4.14 mmol, 64.2%) as a white solid.

### Step F: benzyl N-methyl-N-[(2-{5-[(1R)-1-[(2-methylpropane-2-sulfinyl)amino]ethyl]thiophen-2-yl}phenyl)methyl]carbamate

To a solution of benzyl N-methyl-N-[(2-{5-[(1E)-1-[(2-methylpropane-2-sulfinyl)imino]ethyl]thiophen-2-yl}phenyl)methyl]carbamate (2 g, 4.14 mmol) in THE (20 mL) was added NaBH₄ (0.541 mL, 16.6 mmol) at 0° C. After stirring at rt for 2 h, the mixture was quenched with ice water and extracted with EtOAc. The organic layers was washed with brine and dried over Na₂SO₄ then concentrated under reduced pressure. The mixture was purified by chromatography (silica gel, 0-100%, EtOAc in PE) to give the product of benzyl N-methyl-N-[(2-{5-[(1R)-1-[(2-methylpropane-2-sulfinyl)amino]ethyl]thiophen-2-yl}phenyl)methyl]carbamate (1.76 g, 3.63 mmol, 87.6%) as a white solid.

### Step G: benzyl N-[(2-{5-[(1R)-1-aminoethyl]thiophen-2-yl}phenyl)methyl]-N-methylcarbamate

To a solution of benzyl N-methyl-N-{[2-(5-{1-[(2-methylpropane-2-sulfinyl)amino]ethyl}thiophen-2-yl)phenyl]methyl}carbamate (180 mg, 0.371 mmol) in dioxane (5 mL) was added 4N HCl/dioxane (2 mL) at rt. After stirring at rt for 1 h, the mixture was concentrated under reduced pressure to give the crude of benzyl N-[(2-{5-[(1R)-1-aminoethyl]thiophen-2-yl}phenyl)methyl]-N-methylcarbamate (165 mg, 0.325 mmol, 87.6%) as a white solid. LC-MS (ESI) m/z: 381⁺.

### Example 1: Synthesis of (R)—N-(1-(3-(1,1-difluoro-2-hydroxyethyl)phenyl)ethyl)-4-oxo-5-(tetrahydro-2H-pyran-4-yl)-4,5-dihydro-2H-pyrazolo[4,3-c]pyridine-7-carboxamide

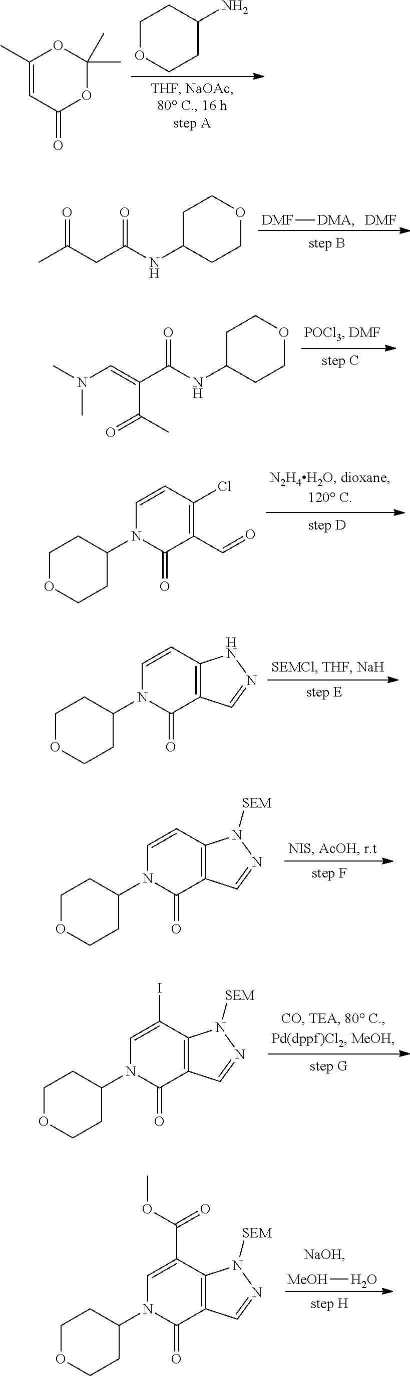

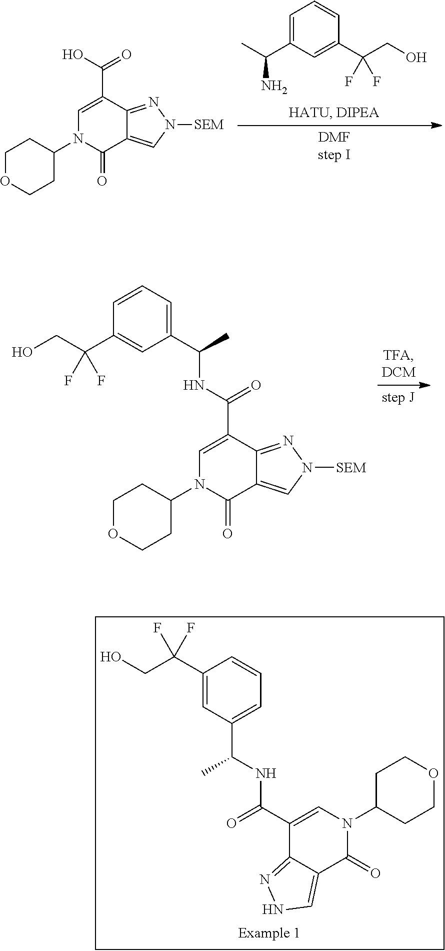 [SMILES: C[Si](C)(C)CCOCn1cc2c(=O)n(C3CCOCC3)cc(C(=O)O)c2n1 ; C[C@H](N)c1cccc(C(F)(F)CO)c1 ; C[C@@H](NC(=O)c1cn(C2CCOCC2)c(=O)c2cn(COCC[Si](C)(C)C)nc12)c1cccc(C(F)(F)CO)c1 ; C[C@@H](NC(=O)c1cn(C2CCOCC2)c(=O)c2c[nH]nc12)c1cccc(C(F)(F)CO)c1]

### Step A. 3-oxo-N-(tetrahydro-2H-pyran-4-yl)butanamide

To a stirred solution of 2,2,6-trimethyl-2,4-dihydro-1,3-dioxin-4-one (2.8 mL, 21.1 mmol) in dry THF (30 mL) was added oxan-4-amine (3.28 mL, 31.7 mmol) and NaOAc (1.90 g, 23.2 mmol). The reaction mixture was stirred at 70° C. for 16 h. The reaction mixture was poured into water, extracted with EtOAc, washed by brine, dried over Na₂SO₄ and concentrated under vacuum under vacuum. The residue was purified by column chromatography (silica gel, 40 g, 0˜100% EtOAc in PE) to give N-(oxan-4-yl)-3-oxobutanamide (3.80 g, 20.5 mmol, 97.1%) as a yellow oil. LCMS: m/z 186 (M+H)⁺.

### Step B. (E)-2-((dimethylamino)methylene)-3-oxo-N-(tetrahydro-2H-pyran-4-yl)butanamide

To a stirred solution of N-(oxan-4-yl)-3-oxobutanamide (3.80 g, 20.5 mmol) in DMF (20 mL) was added DMF-DMA (2.46 g, 20.5 mmol) dropwise over 5 min. The reaction mixture was stirred at room temperature for 16 h. The reaction mixture was concentrated under vacuum to give the residue. The residue was purified by column chromatography (silica gel, 40 g, 0˜10% MeOH in DCM) to give (2E)-2-[(dimethylamino)methylidene]-N-(oxan-4-yl)-3-oxobutanamide (4 g, 16.6 mmol, 81.1%) as a yellow oil. LCMS: m/z 241 (M+H)⁺.

### Step C. 4-chloro-2-oxo-1-(tetrahydro-2H-pyran-4-yl)-1,2-dihydropyridine-3-carbaldehyde

At 0° C., DMF (10 mL) was added into POCl₃ (20 mL) dropwise, the mixture was stirred for 15 min and then (2E)-2-[(dimethylamino)methylidene]-N-(oxan-4-yl)-3-oxobutanamide (4 g, 16.6 mmol) in DMF (40 mL) was added. The reaction mixture was stirred at 100° C. for 1 h. The reaction mixture was concentrated under vacuum. The residue was poured into water and neutralized with saturated aqueous NaHCO₃. The resulting mixture was extracted with EtOAc. The organic layers were washed with brine, dried over Na₂SO₄, concentrated. The residue was purified by column chromatography (silica gel, 40 g, 0-100% EtOAc in PE) to give 4-chloro-1-(oxan-4-yl)-2-oxo-1,2-dihydropyridine-3-carbaldehyde (2.1 g, 8.69 mmol, 52.2%) as a yellow oil. LCMS: m/z 242 (M+H)⁺.

### Step D. 5-(tetrahydro-2H-pyran-4-yl)-1,5-dihydro-4H-pyrazolo[4,3-c]pyridin-4-one

To a solution of 4-chloro-1-(oxan-4-yl)-2-oxo-1,2-dihydropyridine-3-carbaldehyde (2.1 g, 8.69 mmol) in dioxane (20 mL) was added N₂H₄·H₂O (4.34 g, 86.9 mmol). The reaction mixture was stirred at 120° C. for 16 h. The reaction mixture was concentrated under vacuum and purified by column chromatography (silica gel, 24 g, 0˜10% MeOH in DCM) to give 5-(oxan-4-yl)-1H,4H,5H-pyrazolo[4,3-c]pyridin-4-one (1.6 g, 7.3 mmol, 83.9%) as a yellow solid. LCMS: m/z 220 (M+H)⁺.

### Step E. 5-(tetrahydro-2H-pyran-4-yl)-1-((2-(trimethylsilyl)ethoxy)methyl)-1,5-dihydro-4H-pyrazolo[4,3-c]pyridin-4-one

To a stirred solution of 5-(oxan-4-yl)-1H,4H,5H-pyrazolo[4,3-c]pyridin-4-one (1.6 g, 7.3 mmol) in DMF (10 mL) was added NaH (0.53 g, 21.9 mmol) at 0° C. The reaction mixture was stirred at room temperature for 0.5 h and then SEMCl (1.46 g, 8.7 mmol) was added. The reaction mixture was stirred at room temperature for 2 h. The reaction mixture was poured into water, extracted with EtOAc, washed with brine, dried over Na₂SO₄ and concentrated under vacuum to give the residue and the residue purified by column chromatography (silica gel, 24 g, 0˜70% EtOAc in PE) to give 5-(oxan-4-yl)-1-{[2-(trimethylsilyl)ethoxy]methyl}-1H,4H,5H-pyrazolo[4,3-c]pyridin-4-one (2.1 g, 6.0 mmol, 82.3%) as a light yellow solid. LCMS: m/z 350 (M+H)⁺.

### Step F. 7-iodo-5-(tetrahydro-2H-pyran-4-yl)-1-((2-(trimethylsilyl)ethoxy)methyl)-1,5-dihydro-4H-pyrazolo[4,3-c]pyridin-4-one

To a stirred solution of 5-(oxan-4-yl)-1-{[2-(trimethylsilyl)ethoxy]methyl}-1H,4H,5H-pyrazolo[4,3-c]pyridin-4-one (700 mg, 2.003 mmol) in AcOH (10 mL) was added iodo(sulfanylidene)amine (519.6 mg, 3.0 mmol). The reaction mixture was stirred at room temperature for 2 h. The reaction mixture was poured into water and extracted with EtOAc, washed with NaHCO₃, dried over Na₂SO₄ and concentrated in vacuo. The residue was purified by column chromatography (silica gel, 24 g, 0-50% EtOAc in PE) to give 7-iodo-5-(oxan-4-yl)-1-{[2-(trimethylsilyl)ethoxy]methyl}-1H,4H,5H-pyrazolo[4,3-c]pyridin-4-one (400 mg, 0.84 mmol, 42.0%) as a light yellow oil. LCMS: m/z 476 (M+H)⁺.

### Step G. methyl 4-oxo-5-(tetrahydro-2H-pyran-4-yl)-1-((2-(trimethylsilyl)ethoxy)methyl)-4,5-dihydro-1H-pyrazolo[4,3-c]pyridine-7-carboxylate

To a stirred solution of 7-iodo-5-(oxan-4-yl)-1-{[2-(trimethylsilyl)ethoxy]methyl}-1H,4H,5H-pyrazolo[4,3-c]pyridin-4-one (400 mg, 0.841 mmol) in MeOH (10 mL) was added Pd(dppf)Cl₂ (123 mg, 0.17 mmol) and TEA (0.35 mL, 2.5 mmol). The reaction mixture was stirred at 80° C. under CO atmosphere for 16 h. The reaction mixture was filtered and the filtrate was concentrated under vacuum. The residue was purified by column chromatography (silica gel, 24 g, 0˜40% EtOAc in PE) to give methyl 5-(oxan-4-yl)-4-oxo-1-{[2-(trimethylsilyl)ethoxy]methyl}-1H,4H,5H-pyrazolo[4,3-c]pyridine-7-carboxylate (250 mg, 0.61 mmol, 72.9%) as a light yellow oil. LCMS: m/z 408 (M+H)⁺.

### Step H. 4-oxo-5-(tetrahydro-2H-pyran-4-yl)-2-((2-(trimethylsilyl)ethoxy)methyl)-4,5-dihydro-2H-pyrazolo[4,3-c]pyridine-7-carboxylic acid

To a stirred solution of methyl 5-(oxan-4-yl)-4-oxo-1-{[2-(trimethylsilyl)ethoxy]methyl}-1H,4H,5H-pyrazolo[4,3-c]pyridine-7-carboxylate (250 mg, 0.61 mmol) in MeOH (5 mL) was added sodium hydroxide (245 mg, 6.1 mmol) in H₂O (5 mL). The reaction mixture was stirred at room temperature for 2 h. The pH of the reaction mixture was adjusted to 3-4 and extracted with EtOAc. The organic layers were dried over Na₂SO₄ and concentrated under vacuum to give the crude product 5-(oxan-4-yl)-4-oxo-2-{[2-(trimethylsilyl)ethoxy]methyl}-2H,4H,5H-pyrazolo[4,3-c]pyridine-7-carboxylic acid (150 mg, 0.38 mmol, 62.1%) as a light yellow solid. LCMS: m/z 394 (M+H)⁺.

### Step I. (R)—N-(1-(3-(1,1-difluoro-2-hydroxyethyl)phenyl)ethyl)-4-oxo-5-(tetrahydro-2H-pyran-4-yl)-2-((2-(trimethylsilyl)ethoxy)methyl)-4,5-dihydro-2H-pyrazolo[4,3-c]pyridine-7-carboxamide

To a stirred solution of 5-(oxan-4-yl)-4-oxo-2-{[2-(trimethylsilyl)ethoxy]methyl}-2H,4H,5H-pyrazolo[4,3-c]pyridine-7-carboxylic acid (20 mg, 0.05 mmol) in N, N-dimethylformamide (3 mL) was added 2-{3-[(1R)-1-aminoethyl]phenyl}-2,2-difluoroethan-1-ol (11.2 mg, 0.05 mmol), DIPEA (33.0 mg, 0.2 mmol) and HATU (28.9 mg, 0.07 mmol). The reaction mixture was stirred at room temperature for 2 h. The pH of the reaction mixture was poured into water and extracted with EtOAc. The organic layers were washed with brine, dried over Na₂SO₄ and concentrated under vacuum to give the residue and the residue was purified by TLC (100% EtOAc) to give N-[(1R)-1-[3-(1,1-difluoro-2-hydroxyethyl)phenyl]ethyl]-5-(oxan-4-yl)-4-oxo-2-{[2-(trimethylsilyl)ethoxy]methyl}-2H,4H,5H-pyrazolo[4,3-c]pyridine-7-carboxamide (15 mg, 0.02 mmol, 51.1%) as a white solid. LCMS: m/z 577 (M+H)⁺.

### Step J. (R)—N-(1-(3-(1,1-difluoro-2-hydroxyethyl)phenyl)ethyl)-4-oxo-5-(tetrahydro-2H-pyran-4-yl)-4,5-dihydro-2H-pyrazolo[4,3-c]pyridine-7-carboxamide

To a stirred solution of N-[(1R)-1-[3-(1,1-difluoro-2-hydroxyethyl)phenyl]ethyl]-5-(oxan-4-yl)-4-oxo-2-{[2-(trimethylsilyl)ethoxy]methyl}-2H,4H,5H-pyrazolo[4,3-c]pyridine-7-carboxamide (15 mg, 0.03 mmol) in TFA (2 mL). The reaction mixture was stirred at room temperature for 1 h. The reaction mixture was concentrated under vacuum to give the residue and the residue was purified by pre-HPLC (phase A: H₂O (0.1% FA). phase B: MECN) to give N-[(1R)-1-[3-(1,1-difluoro-2-hydroxyethyl)phenyl]ethyl]-5-(oxan-4-yl)-4-oxo-2H,4H,5H-pyrazolo[4,3-c]pyridine-7-carboxamide (3 mg, 0.007 mmol, 25.8%) as a white solid. ¹H NMR (400 MHz, DMSO): δ 13.41 (s, 1H), 8.89 (d, J=7.6 Hz, 1H), 8.14 (d, J=111.7 Hz, 2H), 7.54 (d, J=8.9 Hz, 2H), 7.48 (t, J=7.6 Hz, 1H), 7.41 (d, J=7.6 Hz, 1H), 5.63 (t, J=6.3 Hz, 1H), 5.35-5.18 (m, 1H), 5.08 (s, 1H), 4.04 (d, J=8.0 Hz, 2H), 3.85 (td, J=14.2, 6.1 Hz, 3H), 3.53 (t, J=11.3 Hz, 2H), 2.04 (s, 3H), 1.74 (d, J=9.1 Hz, 3H), 1.55 (d, J=7.0 Hz, 4H). LCMS: m/z 447 (M+H)⁺.

The examples in the following Table 1 were prepared by using a method analogous to that used to prepare the examples as described herein.

TABLE 1

| Ex# | Structure & name | Analytical data | Method |
|---|---|---|---|
| Example 2 | 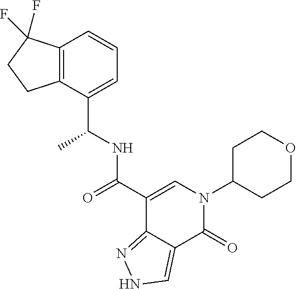 [SMILES: C[C@@H](NC(=O)c1cn(C2CCOCC2)c(=O)c2c[nH]nc12)c1cccc2c1CCC2(F)F] | LCMS: (ESI) m/z 443 (M + H)⁺. ¹H NMR (400 MHz, DMSO) δ 9.37 (s, 1H), 8.19 (s, 1H), 8.11 (s, 1H), 7.62-7.57 (m, 1H), 7.45 (d, J = 6.5 Hz, 3H), 5.26 (t, J = 7.0 Hz, 2H), 5.06 (ddd, J = 12.2, 8.4, 4.0 Hz, 2H), 4.03 (d, J = 11.3 Hz, 3H), 3.59-3.48 (m, 7H), 3.21-3.10 (m, 5H), 2.64 (ddd, J = 21.3, 14.3, 6.8 Hz, 4H), 1.99 (d, J = 9.3 Hz, 3H), 1.73 (d, J = 10.5 Hz, 3H), 1.55 (d, J = 7.0 Hz, 5H). | Example 1 |
| | (R)-N-(1-(1,1-difluoro-2,3-dihydro-1H- | | |
| | inden-4-yl)ethyl)-4-oxo-5-(tetrahydro- | | |
| | 2H-pyran-4-yl)-4,5-dihydro-2H- | | |
| | pyrazolo[4,3-c]pyridine-7-carboxamide | | |
| Example 3 | 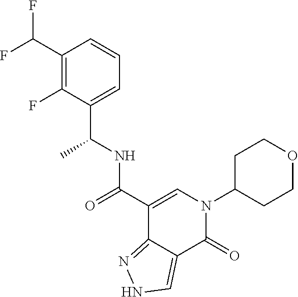 [SMILES: C[C@@H](NC(=O)c1cn(C2CCOCC2)c(=O)c2c[nH]nc12)c1cccc(C(F)F)c1F] | LCMS: (ESI) m/z 435 (M + H)⁺. ¹H NMR (400 MHz, CDCl₃) δ 8.56 (s, 1H), 8.41 (s, 1H), 8.10 (s, 1H), 7.59-7.39 (m, 3H), 7.21 (t, J = 7.8 Hz, 2H), 6.90 (t, J = 55.0 Hz, 2H), 5.61- 5.48 (m, 1H), 5.17 (dd, J = 14.0, 10.1 Hz, 1H), 4.18-4.00 (m, 3H), 3.59 (t, J = 11.7 Hz, 3H), 2.05- 1.88 (m, 3H), 1.82 (s, 3H), 1.64 (d, J = 7.0 Hz, 4H). | Example 1 |
| | (R)-N-(1-(3-(difluoromethyl)-2- | | |
| | fluorophenyl)ethyl)-4-oxo-5- | | |
| | (tetrahydro-2H-pyran-4-yl)-4,5-dihydro- | | |
| | 2H-pyrazolo[4,3-c]pyridine-7- | | |
| | carboxamide | | |
| Example 4 | 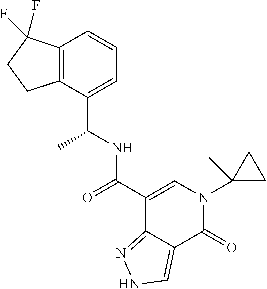 [SMILES: C[C@@H](NC(=O)c1cn(C2(C)CC2)c(=O)c2c[nH]nc12)c1cccc2c1CCC2(F)F] | LCMS: (ESI) m/z 413 (M + H)⁺. ¹H NMR (400 MHz, MeOD) δ 8.42 (d, J = 68.4 Hz, 1H), 8.15 (s, 1H), 7.54 (d, J = 6.2 Hz, 1H), 7.44-7.33 (m, 2H), 5.34 (q, J = 7.0 Hz, 1H), 3.27-3.03 (m, 2H), 2.67-2.49 (m, 2H), 1.59 (t, J = 9.1 Hz, 3H), 1.51 (s, 3H), 1.06 (d, J = 25.4 Hz, 4H). | Example 1 |
| | (R)-N-(1-(1,1-difluoro-2,3-dihydro-1H- | | |
| | inden-4-yl)ethyl)-5-(1- | | |
| | methylcyclopropyl)-4-oxo-4,5-dihydro- | | |
| | 2H-pyrazolo[4,3-c]pyridine-7- | | |
| | carboxamide | | |
| Example 5 | 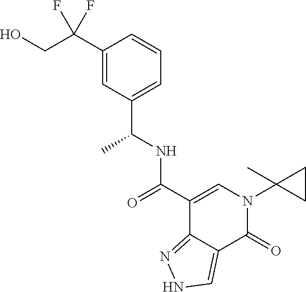 [SMILES: C[C@@H](NC(=O)c1cn(C2(C)CC2)c(=O)c2c[nH]nc12)c1cccc(C(F)(F)CO)c1] | LCMS: (ESI) m/z 417 (M + H)⁺. ¹H NMR (400 MHz, MeOD) δ 8.42 (d, J = 72.6 Hz, 1H), 8.14 (d, J = 17.7 Hz, 1H), 7.55 (d, J = 22.8 Hz, 2H), 7.48- 7.38 (m, 2H), 5.30 (q, J = 7.0 Hz, 1H), 3.88 (t, J = 13.5 Hz, 2H), 1.61 (d, J = 7.0 Hz, 3H), 1.53 (d, J = 16.2 Hz, 3H), 1.14 (d, J = 30.5 Hz, 2H), 1.03 (s, 2H). | Example 1 |
| | (R)-N-(1-(3-(1,1-difluoro-2- | | |
| | hydroxyethyl)phenyl)ethyl)-5-(1- | | |
| | methylcyclopropyl)-4-oxo-4,5-dihydro- | | |
| | 2H-pyrazolo[4,3-c]pyridine-7- | | |
| | carboxamide | | |
| Example 6 | 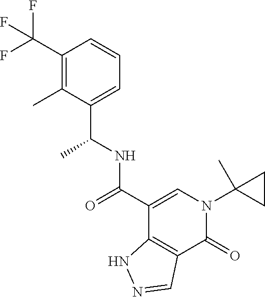 [SMILES: Cc1c([C@@H](C)NC(=O)c2cn(C3(C)CC3)c(=O)c3cn[nH]c23)cccc1C(F)(F)F] | LCMS: (ESI) m/z 419 (M + H)⁺. ¹H NMR (400 MHz, MeOD) δ 8.43 (d, J = 65.1 Hz, 1H), 8.12 (d, J = 12.7 Hz, 1H), 7.66 (d, J = 7.9 Hz, 1H), 7.56 (d, J = 7.7 Hz, 1H), 7.34 (t, J = 7.8 Hz, 1H), 5.57 (d, J = 6.6 Hz, 1H), 2.55 (s, 3H), 1.56 (dd, J = 19.8, 14.0 Hz, 6H), 1.09 (dd, J = 39.4, 26.2 Hz, 4H). | Example 1 |
| | (R)-N-(1-(2-methyl-3- | | |
| | (trifluoromethyl)phenyl)ethyl)-5-(1- | | |
| | methylcyclopropyl)-4-oxo-4,5-dihydro- | | |
| | 1H-pyrazolo[4,3-c]pyridine-7- | | |
| | carboxamide | | |
| Example 7 | 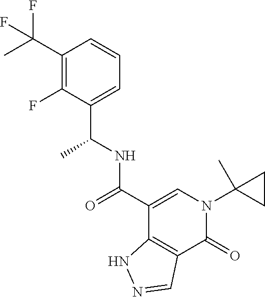 [SMILES: C[C@@H](NC(=O)c1cn(C2(C)CC2)c(=O)c2cn[nH]c12)c1cccc(C(C)(F)F)c1F] | LCMS: (ESI) m/z 419 (M + H)⁺. ¹H NMR (400 MHz, MeOD) δ 8.45 (d, J = 55.8 Hz, 1H), 8.16 (s, 1H), 7.54 (d, J = 6.7 Hz, 1H), 7.46 (t, J = 7.3 Hz, 1H), 7.23 (t, J = 7.7 Hz, 1H), 5.53 (q, J = 6.9 Hz, 1H), 1.98 (t, J = 18.6 Hz, 3H), 1.61 (d, J = 7.0 Hz, 3H), 1.52 (s, 3H), 1.07 (d, J = 25.5 Hz, 4H). | Example 1 |
| | (R)-N-(1-(3-(1,1-difluoroethyl)-2- | | |
| | fluorophenyl)ethyl)-5-(1- | | |
| | methylcyclopropyl)-4-oxo-4,5-dihydro- | | |
| | 1H-pyrazolo[4,3-c]pyridine-7- | | |
| | carboxamide | | |
| Example 8 | 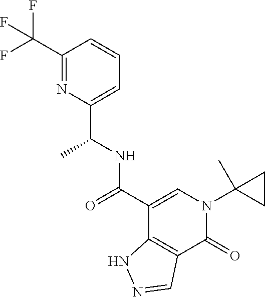 [SMILES: C[C@@H](NC(=O)c1cn(C2(C)CC2)c(=O)c2cn[nH]c12)c1cccc(C(F)(F)F)n1] | LCMS: (ESI) m/z 406 (M + H)⁺. ¹H NMR (400 MHz, MeOD) δ 8.45 (d, J = 43.7 Hz, 1H), 8.15 (d, J = 22.2 Hz, 1H), 7.99 (t, J = 7.8 Hz, 1H), 7.69 (d, J = 7.4 Hz, 2H), 5.44- 5.34 (m, 1H), 1.64 (d, J = 7.0 Hz, 3H), 1.54 (d, J = 18.9 Hz, 3H), 1.10 (dd, J = 41.8, 23.2 Hz, 4H). | Example 1 |
| | (R)-5-(1-methylcyclopropyl)-4-oxo-N- | | |
| | (1-(6-(trifluoromethyl)pyridin-2- | | |
| | yl)ethyl)-4,5-dihydro-1H-pyrazolo[4,3- | | |
| | c]pyridine-7-carboxamide | | |
| Example 9 | 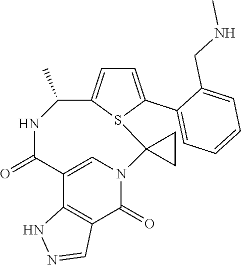 | LC-MS(ESI) m/z: 462⁺. 1H NMR (400 MHz, MeOD) δ 8.53 (s, 1H), 8.38 (s, 1H), 8.26 (s, 1H), 7.54 (s, 1H), 7.47 (dd, J = 6.9, 3.3 Hz, 3H), 7.13 (d, J = 3.7 Hz, 1H), 6.99 (d, J = 3.6 Hz, 1H), 5.58 (d, J = 6.8 Hz, 1H), 4.59 (s, 2H), 4.25 (s, 2H), 2.58 (s, 3H), 1.74 (d, J = 6.9 Hz, 3H), 1.53 (s, 3H), 1.13 (s, 2H), 1.06 (d, J = 4.4 Hz, 2H). | Example 1 (last two step: de-Cbz using THF/Pd- C/H₂/Boc₂O, and then De- Boc) |
| | (R)-N-(1-(5-(2- | | |
| | ((methylamino)methyl)phenyl)thiophen- | | |
| | 2-yl)ethyl)-5-(1-methylcyclopropyl)-4- | | |
| | oxo-4,5-dihydro-1H-pyrazolo[4,3- | | |
| | c]pyridine-7-carboxamide | | |

### Example 10: Synthesis of (R)-5-(4-acetylcyclohexyl)-N-(1-(1,1-difluoro-2,3-dihydro-H-inden-4-yl)ethyl)-4-oxo-4,5-dihydro-2H-pyrazolo[4,3-c]pyridine-7-carboxamide

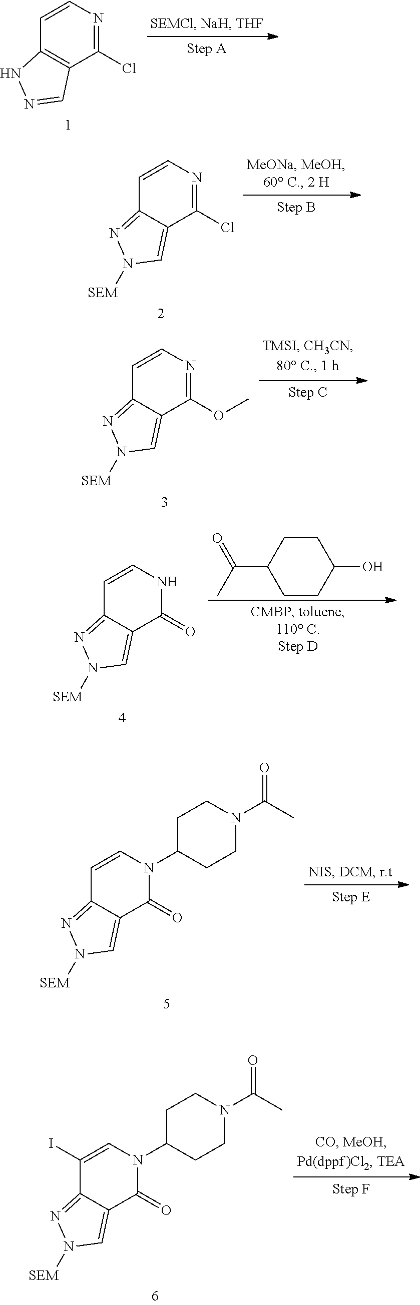

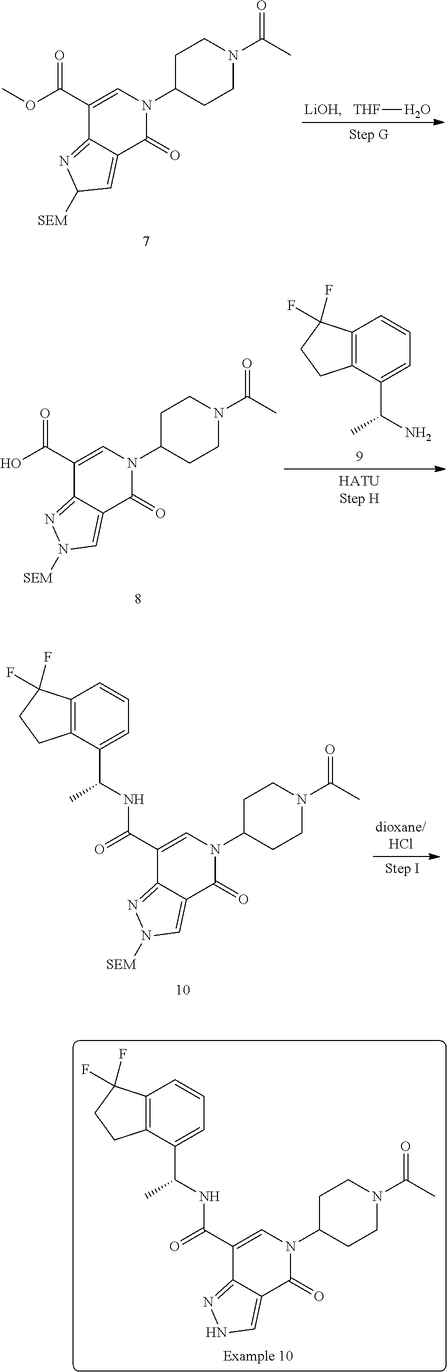

### Step A. 4-chloro-2-{[2-(trimethylsilyl)ethoxy]methyl}-2H-pyrazolo[4,3-c]pyridine

To a stirred solution of 4-chloro-1H-pyrazolo[4,3-c]pyridine (4 g, 26.1 mmol) in THE (50 mL) was added NaH (2.08 g, 52.1 mmol) at 0° C. After stirring at 0° C. for 20 min, SEMCl (6.47 g, 39.1 mmol) was added at 0° C. 2 hr later, the mixture was poured into ice-water (50 mL) and extracted with EtOAc (50 mL*2). The combined organic phase was washed with brine, dried over Na₂SO₄, filtered and concentrated. The crude product was purified by chromatography (silica gel, 0-50%, EtOAc in PE) to give 4-chloro-2-{[2-(trimethylsilyl)ethoxy]methyl}-2H-pyrazolo[4,3-c]pyridine (6.2 g, 21.8 mmol, 83.9%) as a brown solid. LC-MS (ESI) m/z 284 (M+H)⁺.

### Step B. 4-methoxy-2-{[2-(trimethylsilyl)ethoxy]methyl}-2H-pyrazolo[4,3-c]pyridine

To a stirred solution of 4-chloro-2-{[2-(trimethylsilyl)ethoxy]methyl}-2H-pyrazolo[4,3-c]pyridine (6.2 g, 21.8 mmol) in MeOH (100 mL) was added NaOMe (13.1 mL, 65.5 mmol, 5 mol/L in MeOH) at rt. After stirring at 60° C. for 2 h, the cooled mixture was poured into water (100 mL) and extracted with EtOAc (100 mL*2). The combined organic phase was washed with brine, dried over Na₂SO₄, filtered and concentrated. The crude product was purified by chromatography (silica gel, 0-50%, EtOAc in PE) to give 4-methoxy-2-{[2-(trimethylsilyl)ethoxy]methyl}-2H-pyrazolo[4,3-c]pyridine (5.5 g, 19.7 mmol, 90.1%) as a yellow solid. LC-MS (ESI) m/z 280 (M+H)⁺.

### Step C. 2-((2-(trimethylsilyl)ethoxy)methyl)-2,5-dihydro-4H-pyrazolo[4,3-c]pyridin-4-one

To a stirred solution of 4-methoxy-2-{[2-(trimethylsilyl)ethoxy]methyl}-2H-pyrazolo[4,3-c]pyridine (6 g, 21.4 mmol) in MeCN (15 mL) was added TMSI (6.34 mL, 42.9 mmol.) The reaction mixture was stirred at room temperature for 2 h. The reaction mixture was concentrated to dryness. The residue was purified by column chromatography (silica gel, 40 g, 0˜3% MeOH in DCM) to give 2-{[2-(trimethylsilyl)ethoxy]methyl}-2H,4H,5H-pyrazolo[4,3-c]pyridin-4-one (3.5 g, 13.2 mmol, 61.4%) as a yellow solid. LCMS: m/z 266 (M+H)⁺.

### Step D

### 5-(1-acetylpiperidin-4-yl)-2-((2-(trimethylsilyl)ethoxy)methyl)-2,5-dihydro-4H-pyrazolo[4,3-c]pyridin-4-one

To a stirred solution of 2-{[2-(trimethylsilyl)ethoxy]methyl}-2H,4H,5H-pyrazolo[4,3-c]pyridin-4-one (200 mg, 0.75 mmol) in toluene (10 mL) was added 1-(4-hydroxypiperidin-1-yl)ethan-1-one (108 mg, 0.75 mmol) and CMBP (782 mg, 2.26 mmol). The reaction mixture was stirred at room 120° C. for 3 h. The reaction mixture was concentrated under vacuum and extracted with DCM. The organic layer was dried over Na₂SO₄ and concentrated under vacuum to give the residue and the residue was purified by TLC (0˜2% MeOH in DCM) to give 5-(1-acetylpiperidin-4-yl)-2-{[2-(trimethylsilyl)ethoxy]methyl}-2H,4H,5H-pyrazolo[4,3-c]pyridin-4-one (80 mg, 0.20 mmol, 27.2%) as a yellow solid. LCMS: m/z 391 (M+H)⁺.

### Step E. 5-(1-acetylpiperidin-4-yl)-7-iodo-2-((2-(trimethylsilyl)ethoxy)methyl)-2,5-dihydro-4H-pyrazolo[4,3-c]pyridin-4-one

To a stirred solution of 5-(4-acetylcyclohexyl)-2-{[2-(trimethylsilyl)ethoxy]methyl}-2H,4H,5H-pyrazolo[4,3-c]pyridin-4-one (80 mg, 0.20 mmol) in AcOH (10 mL) was added NIS (55.4 mg, 0.24 mmol). The reaction mixture was stirred at room temperature for 2 h. The reaction mixture was poured into water and extracted with EtOAc. The organic phase was washed with NaHCO₃, dried over Na₂SO₄ and concentrated under vacuum to give the residue. The residue was purified by column chromatography (silica gel, 4 g, 0˜100% EtOAc in PE) to give 5-(4-acetylcyclohexyl)-7-iodo-2-{[2-(trimethylsilyl)ethoxy]methyl}-2H,4H,5H-pyrazolo[4,3-c]pyridin-4-one (80 mg, 0.15 mmol, 75.6%) as a light yellow oil. LCMS: m/z 517 (M+H)⁺.

### Step F. methyl 5-(1-acetylpiperidin-4-yl)-4-oxo-2-((2-(trimethylsilyl)ethoxy)methyl)-4,5-dihydro-2H-pyrrolo[3,2-c]pyridine-7-carboxylate

To a stirred solution of 5-(4-acetylcyclohexyl)-7-iodo-2-{[2-(trimethylsilyl)ethoxy]methyl}-2H,4H,5H-pyrazolo[4,3-c]pyridin-4-one (80 mg, 0.15 mmol) in MeOH (5 mL) was added Pd(dppf)Cl₂·CH₂Cl₂ (12.6 mg, 0.01 mmol) and TEA (0.06 mL, 0.46 mmol). The reaction mixture was stirred at 70° C. for 3 h under CO atmosphere. The reaction mixture was filtered and the filtrate was concentrated under vacuum to give the residue and the residue was purified by column chromatography (silica gel, 24 g, 0˜40% EtOAc in PE) to give methyl 5-(4-acetylcyclohexyl)-4-oxo-2-{[2-(trimethylsilyl)ethoxy]methyl}-2H,4H,5H-pyrazolo[4,3-c]pyridine-7-carboxylate (60 mg, 0.13 mmol, 86.5%) as a light yellow solid. LCMS: m/z 448 (M+H)⁺.

### Step G. 5-(1-acetylpiperidin-4-yl)-4-oxo-2-((2-(trimethylsilyl)ethoxy)methyl)-4,5-dihydro-2H-pyrazolo[4,3-c]pyridine-7-carboxylic acid

To a stirred solution of methyl 5-(4-acetylcyclohexyl)-4-oxo-2-{[2-(trimethylsilyl)ethoxy]methyl}-2H,4H,5H-pyrrolo[3,2-c]pyridine-7-carboxylate (60 mg, 0.13 mmol) in MeOH (3 mL) was added NaOH (53.74 mg, 1.34 mmol) in H₂O (2 mL). The reaction mixture was stirred at room temperature for 2 h. The pH of the reaction mixture was adjusted to 3-4 and extracted with EA. The organic layers were dried over Na₂SO₄ and concentrated under vacuum to give the crude product 5-(4-acetylcyclohexyl)-4-oxo-2-{[2-(trimethylsilyl)ethoxy]methyl}-2H,4H,5H-pyrazolo[4,3-c]pyridine-7-carboxylic acid (50 mg, 0.11 mmol, 85.8%) as a white solid. LCMS: m/z 434 (M+H)⁺.

### Step H. (R)-5-(1-acetylpiperidin-4-yl)-N-(1-(1,1-difluoro-2,3-dihydro-1H-inden-4-yl)ethyl)-4-oxo-2-((2-(trimethylsilyl)ethoxy)methyl)-4,5-dihydro-2H-pyrazolo[4,3-c]pyridine-7-carboxamide

To a stirred solution of 5-(4-acetylcyclohexyl)-4-oxo-2-{[2-(trimethylsilyl)ethoxy]methyl}-2H,4H,5H-pyrazolo[4,3-c]pyridine-7-carboxylic acid (50 mg, 0.11 mmol) in DMF (10 mL) was added (1R)-1-(1,1-difluoro-2,3-dihydro-1H-inden-4-yl)ethan-1-amine (27 mg, 0.13 mmol), DIEA (0.057 mL, 0.345 mmol) and HATU (65 mg, 0.17 mmol). The reaction mixture was stirred at room temperature for 2 h. The reaction mixture was poured into water and extracted with DCM. The organic layers were washed with brine, dried over Na₂SO₄ and concentrated under vacuum to give the residue and the residue was purified by column chromatography (silica gel, 24 g, 0˜100% EtOAc in PE) to give 5-(4-acetylcyclohexyl)-N-[(1R)-1-(1,1-difluoro-2,3-dihydro-1H-inden-4-yl)ethyl]-4-oxo-2-{[2-(trimethylsilyl)ethoxy]methyl}-2H,4H,5H-pyrazolo[4,3-c]pyridine-7-carboxamide (50 mg, 0.08 mmol, 70.7%) as a white solid. LCMS: m/z 614 (M+H)⁺.

### Step I. (R)-5-(1-acetylpiperidin-4-yl)-N-(1-(1,1-difluoro-2,3-dihydro-1H-inden-4-yl)ethyl)-4-oxo-4,5-dihydro-2H-pyrazolo[4,3-c]pyridine-7-carboxamide

To a stirred solution of 5-(1-acetylpiperidin-4-yl)-N-[(1R)-1-(1,1-difluoro-2,3-dihydro-1H-inden-4-yl)ethyl]-4-oxo-2-{[2-(trimethylsilyl)ethoxy]methyl}-2H,4H,5H-pyrazolo[4,3-c]pyridine-7-carboxamide (50 mg, 0.081 mmol) in 4N HCl/dioxane (3 mL). The reaction mixture was stirred at room temperature for 1 h. The reaction mixture was concentrated under vacuum to give the residue and the residue was purified by prep-HPLC (phase A: H₂O (0.1% TFA), phase B: MeCN) to give 5-(1-acetylpiperidin-4-yl)-N-[(1R)-1-(1,1-difluoro-2,3-dihydro-1H-inden-4-yl)ethyl]-4-oxo-2H,4H,5H-pyrazolo[4,3-c]pyridine-7-carboxamide (11 mg, 0.023 mmol, 27.9%) as a white solid. ¹H NMR (400 MHz, DMSO) δ 13.45 (s, 1H), 8.86 (t, J=6.9 Hz, 1H), 8.31 (s, 1H), 8.16 (s, 1H), 7.57 (d, J=6.4 Hz, 1H), 7.49-7.37 (m, 3H), 5.24 (dd, J=7.0, 4.8 Hz, 1H), 5.04 (s, 1H), 4.61 (d, J=12.1 Hz, 1H), 4.01 (d, J=15.6 Hz, 1H), 3.17 (dd, J=36.0, 7.2 Hz, 6H), 2.73-2.55 (m, 4H), 2.07 (s, 5H), 1.94-1.74 (m, 6H), 1.53 (d, J=7.0 Hz, 4H). LCMS: m/z 484 (M+H)⁺.

### Example 11: Synthesis of N—((R)-1-(1,1-difluoro-2,3-dihydro-1H-inden-4-yl)ethyl)-5-((1s,4S)-4-((2-hydroxyethyl)(methyl)carbamoyl)cyclohexyl)-4-oxo-4,5-dihydro-2H-pyrazolo[4,3-c]pyridine-7-carboxamide

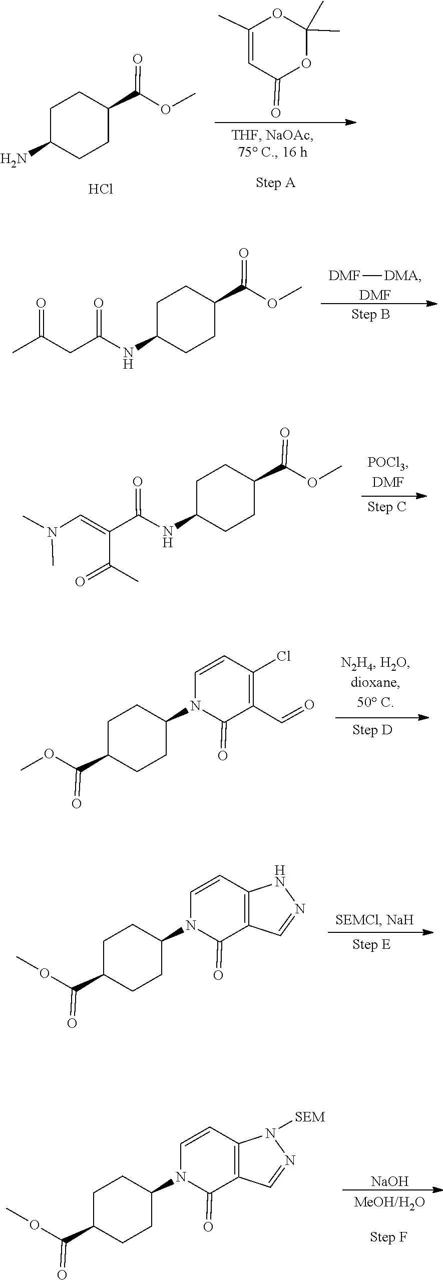

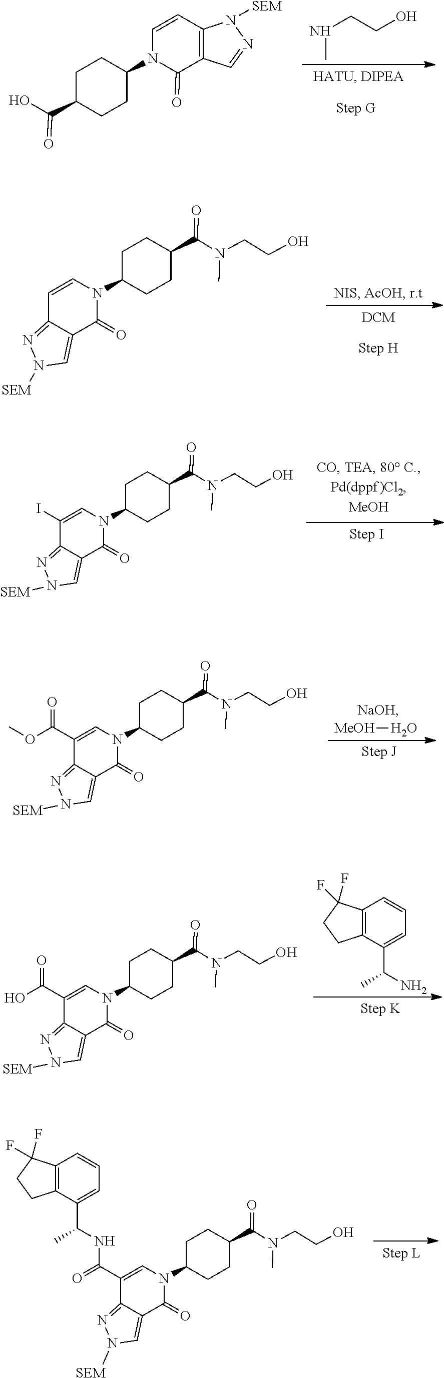

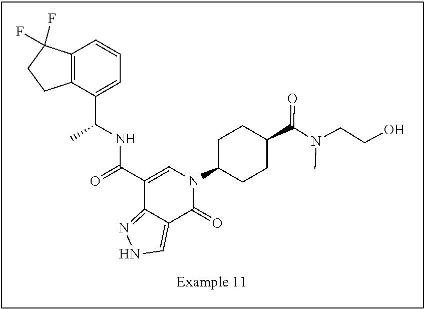 [SMILES: C[C@@H](NC(=O)c1cn([C@H]2CC[C@@H](C(=O)N(C)CCO)CC2)c(=O)c2c[nH]nc12)c1cccc2c1CCC2(F)F]

### Step A. methyl (1s,4s)-4-((E)-2-((dimethylamino)methylene)-3-oxobutanamido)cyclohexane-1-carboxylate

To a stirred solution of methyl (1s,4s)-4-aminocyclohexane-1-carboxylate (5 g, 31.8 mmol) in THF (50 mL) was added 2,2,6-trimethyl-2,4-dihydro-1,3-dioxin-4-one (6.23 mL, 47.7 mmol) and NaOAc (7.83 g, 95.4 mmol). The reaction mixture was stirred at 75° C. for 16 h. The reaction mixture was concentrated, diluted with water, extracted with EtOAc, then washed with brine, dried over anhydrous Na₂SO₄, filtered and concentrated to dryness. The residue was purified by column chromatography (silica gel, 45 g, 0˜100% EtOAc in PE) to give methyl 4-(3-oxobutanamido)cyclohexane-1-carboxylate (4.19 g, 17.3 mmol, 54.6%). LCMS: m/z 242 (M+H)⁺.

### Step B. methyl 4-hydroxy-1-(1-methylcyclopropyl)-6-oxo-1,6-dihydropyridine-3-carboxylate

To a stirred solution of methyl 4-(3-oxobutanamido)cyclohexane-1-carboxylate (3.8 g, 15.7 mmol) in DMF (15 mL) was added DMF-DMA (4.21 mL, 31.4 mmol). The reaction mixture was stirred at room temperature for 16 h. The reaction mixture was concentrated to dryness. The residue was purified by column chromatography (silica gel, 40 g, 0˜5% MeOH in DCM) to give methyl 4-[(2E)-2-[(dimethylamino)methylidene]-3-oxobutanamido]cyclohexane-1-carboxylate (4.2 g, 14.171 mmol, 89.9%) as a yellow solid. LCMS: m/z 297 (M+H)⁺.

### Step C. methyl (1s,4s)-4-(4-chloro-3-formyl-2-oxopyridin-1(2H)-yl)cyclohexane-1-carboxylate

POCl3 (6.605 mL, 70.857 mmol) was added into DMF (15 mL) dropwise at 0° C. and the mixture was stirred for 15 min before methyl 4-[(2E)-2-[(dimethylamino)methylidene]-3-oxobutanamido]cyclohexane-1-carboxylate (4.2 g, 14.1 mmol) in DMF (15 mL) was added. The reaction mixture was stirred at 100° C. for 1 h. The reaction mixture was concentrated under vacuum to remove excess POCl₃ The residue was poured into water and neutralized with saturated aqueous NaHCO₃, and extracted with EtOAc. The organic layers were washed with brine, dried over Na₂SO₄, concentrated under vacuum and purified by column chromatography (silica gel, 40 g, 0˜100% EtOAc in PE) to give methyl (1s,4s)-4-(4-chloro-3-formyl-2-oxo-1,2-dihydropyridin-1-yl)cyclohexane-1-carboxylate (2.3 g, 7.7 mmol, 54.5%) as a yellow oil. LCMS: m/z 298 (M+H)⁺.

### Step D. methyl (1s,4s)-4-(4-oxo-1,4-dihydro-5H-pyrazolo[4,3-c]pyridin-5-yl)cyclohexane-1-carboxylate

To a solution of methyl (1s,4s)-4-(4-chloro-3-formyl-2-oxo-1,2-dihydropyridin-1-yl)cyclohexane-1-carboxylate (2.2 g, 7.3 mmol) in dioxane (20 mL) was added N₂H₄·H₂O (3.69 g, 73.8 mmol). The reaction mixture was stirred at 120° C. for 2 h. The cooled reaction mixture was concentrated under vacuum and purified by column chromatography (silica gel, 24 g, 0˜10% MeOH in DCM) to give methyl (1s,4s)-4-{4-oxo-1H,4H,5H-pyrazolo[4,3-c]pyridin-5-yl}cyclohexane-1-carboxylate (2.0 g, 7.26 mmol, 98.3%) as a yellow solid. LCMS: m/z 276 (M+H)⁺.

### Step E. methyl (1s,4s)-4-(4-oxo-1-((2-(trimethylsilyl)ethoxy)methyl)-1,4-dihydro-5H-pyrazolo[4,3-c]pyridin-5-yl)cyclohexane-1-carboxylate

To a stirred solution of methyl (1s,4s)-4-{4-oxo-1H,4H,5H-pyrazolo[4,3-c]pyridin-5-yl}cyclohexane-1-carboxylate (500 mg, 1.81 mmol) in DMF (5 mL) was added NaH (130.7 mg, 5.4 mmol) at 0° C. The reaction mixture was stirred at room temperature for 0.5 h and then SEM-Cl (0.38 mL, 2.17 mmol) was added. The reaction mixture was stirred at room temperature for 2 h. The reaction mixture was poured into water and extracted with EtOAc washed with brine, dried over Na₂SO₄ and concentrated under vacuum. The residue purified by column chromatography (silica gel, 24 g, 0˜70% EtOAc in PE) to give methyl (1s,4s)-4-(4-oxo-1-{[2-(trimethylsilyl)ethoxy]methyl}-1H,4H,5H-pyrazolo[4,3-c]pyridin-5-yl)cyclohexane-1-carboxylate (600 mg, 1.48 mmol, 81.5%) as a light yellow solid. LCMS: m/z 406 (M+H)⁺.

### Step F. (1s,4s)-4-(4-oxo-1-((2-(trimethylsilyl)ethoxy)methyl)-1,4-dihydro-5H-pyrazolo[4,3-c]pyridin-5-yl)cyclohexane-1-carboxylic acid

To a stirred solution of methyl (1s,4s)-4-(4-oxo-1-{[2-(trimethylsilyl)ethoxy]methyl}-1H,4H,5H-pyrazolo[4,3-c]pyridin-5-yl)cyclohexane-1-carboxylate (600 mg, 1.48 mmol) in MeOH (3 mL) was added NaOH (296 mg, 7.39 mmol) in H₂O (2 mL). The reaction mixture was stirred at room temperature for 2 h. The pH of the reaction mixture was adjusted to 3-4 and extracted with EA. The organic layers were dried over Na₂SO₄ and concentrated under vacuum to give the crude product (1s,4s)-4-(4-oxo-1-{[2-(trimethylsilyl)ethoxy]methyl}-1H,4H,5H-pyrazolo[4,3-c]pyridin-5-yl)cyclohexane-1-carboxylic acid (500 mg, 1.277 mmol, 86.3%) as a light yellow solid. LCMS: m/z 392 (M+H)⁺.

### Step G. (1s,4s)-N-(2-hydroxyethyl)-N-methyl-4-(4-oxo-2-((2-(trimethylsilyl)ethoxy)methyl)-2,4-dihydro-5H-pyrazolo[4,3-c]pyridin-5-yl)cyclohexane-1-carboxamide

To a stirred solution of (1s,4s)-4-(4-oxo-1-{[2-(trimethylsilyl)ethoxy]methyl}-1H,4H,5H-pyrazolo[4,3-c]pyridin-5-yl)cyclohexane-1-carboxylic acid (500 mg, 1.2 mmol) in DCM (10 mL) was added 2-(methylamino)ethan-1-ol (84.42 mg, 1.40 mmol, DIEA (0.42 mL, 2.55 mmol) and HATU (728 mg, 1.91 mmol). The reaction mixture was stirred at room temperature for 2 h. The reaction mixture was poured into water and extracted with DCM. The organic layers were washed with brine, dried over Na₂SO₄ and concentrated under vacuum to give the residue and the residue was purified by column chromatography (silica gel, 24 g, 0-100% EtOAc in PE) to give (1s,4s)-N-(2-hydroxyethyl)-N-methyl-4-(4-oxo-2-{[2-(trimethylsilyl)ethoxy]methyl}-2H,4H,5H-pyrazolo[4,3-c]pyridin-5-yl)cyclohexane-1-carboxamide (550 mg, 1.23 mmol, 96.0%) as a white solid. LCMS: m/z 449 (M+H)⁺.

### Step H. (1s,4s)-N-(2-hydroxyethyl)-4-(7-iodo-4-oxo-2-((2-(trimethylsilyl)ethoxy)methyl)-2,4-dihydro-5H-pyrazolo[4,3-c]pyridin-5-yl)-N-methylcyclohexane-1-carboxamide

To a stirred solution of (1s,4s)-N-(2-hydroxyethyl)-N-methyl-4-(4-oxo-2-{[2-(trimethylsilyl)ethoxy]methyl}-2H,4H,5H-pyrazolo[4,3-c]pyridin-5-yl)cyclohexane-1-carboxamide (600 mg, 1.33 mmol) in AcOH (10 mL) was added NIS (330 mg, 1.47 mmol). The reaction mixture was stirred at room temperature for 2 h. The reaction mixture was poured into water, extracted with EtOAc, washed with NaHCO₃ and dried over Na₂SO₄. Concentrated under vacuum and the residue was purified by column chromatography (silica gel, 24 g, 0˜50% EtOAc in PE) to give (1s,4s)-N-(2-hydroxyethyl)-4-(7-iodo-4-oxo-2-{[2-(trimethylsilyl)ethoxy]methyl}-2H,4H,5H-pyrazolo[4,3-c]pyridin-5-yl)-N-methylcyclohexane-1-carboxamide (400 mg, 0.69 mmol, 52.1%) as a light yellow oil. LCMS: m/z 575 (M+H)⁺.

### Step I. methyl 5-((1s,4s)-4-((2-hydroxyethyl)(methyl)carbamoyl)cyclohexyl)-4-oxo-2-((2-(trimethylsilyl)ethoxy)methyl)-4,5-dihydro-2H-pyrazolo[4,3-c]pyridine-7-carboxylate

To a stirred solution of (1s,4s)-N-(2-hydroxyethyl)-4-(7-iodo-4-oxo-2-{[2-(trimethylsilyl)ethoxy]methyl}-2H,4H,5H-pyrazolo[4,3-c]pyridin-5-yl)-N-methylcyclohexane-1-carboxamide (200 mg, 0.348 mmol) in MeOH (5 mL) was added Pd(dppf)Cl₂·CH₂Cl₂ (20 mg, mmol) and TEA (0.14 mL, 1.04 mmol). The reaction mixture was stirred at 80° C. for 16 h under CO atmosphere. The reaction mixture was filtered and the filtrate was concentrated under vacuum to give the residue and the residue was purified by column chromatography (silica gel, 24 g, 0˜40% EtOAc in PE) to give methyl 4-oxo-5-[(1s,4s)-4-[(2-hydroxyethyl)(methyl)carbamoyl]cyclohexyl]-2-{[2-(trimethylsilyl)ethoxy]methyl}-2H,4H,5H-pyrazolo[4,3-c]pyridine-7-carboxylate (60 mg, 0.11 mmol, 34.0%) as a light yellow solid. LCMS: m/z 507 (M+H)⁺.

### Step J. 5-((1s,4s)-4-((2-hydroxyethyl)(methyl)carbamoyl)cyclohexyl)-4-oxo-2-((2-(trimethylsilyl)ethoxy)methyl)-4,5-dihydro-2H-pyrazolo[4,3-c]pyridine-7-carboxylic acid

To a stirred solution of methyl 4-oxo-5-[(1s,4s)-4-[(2-hydroxyethyl)(methyl) carbamoyl]cyclohexyl]-2-{[2-(trimethylsilyl)ethoxy]methyl}-2H,4H,5H-pyrazolo[4,3-c]pyridine-7-carboxylate (60 mg, 0.118 mmol) in MeOH (3 mL) was added sodium hydroxide (23.6 mg, 0.59 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give the crude product 4-oxo-5-[(1s,4s)-4-[(2-hydroxyethyl)(methyl)carbamoyl]cyclohexyl]-2-{[2-(trimethylsilyl)ethoxy]methyl}-2H,4H,5H-pyrazolo[4,3-c]pyridine-7-carboxylic acid (50 mg, 0.101 mmol, 85.7%) as a light yellow solid. LCMS: m/z 493 (M+H)⁺.

### Step K. N—((R)-1-(1,1-difluoro-2,3-dihydro-1H-inden-4-yl)ethyl)-5-((1s,4S)-4-((2-hydroxyethyl)(methyl)carbamoyl)cyclohexyl)-4-oxo-2-((2-(trimethylsilyl)ethoxy)methyl)-4,5-dihydro-2H-pyrazolo[4,3-c]pyridine-7-carboxamide

To a stirred solution of 4-oxo-5-[(1s,4s)-4-[(2-hydroxyethyl)(methyl)carbamoyl]cyclohexyl]-2-{[2-(trimethylsilyl)ethoxy]methyl}-2H,4H,5H-pyrazolo[4,3-c]pyridine-7-carboxylic acid (50 mg, 0.101 mmol) in DCM (10 mL) was added (1R)-1-(1,1-difluoro-2,3-dihydro-1H-inden-4-yl)ethan-1-amine (30.0 mg, 0.15 mmol), DIEA (0.05 mL, 0.30 mmol) and HATU (38.6 mg, 0.10 mmol). The reaction mixture was stirred at room temperature for 2 h. The reaction mixture was poured into water and extracted with DCM. The organic layers were washed with brine, dried over Na₂SO₄ and concentrated under vacuum to give the residue and the residue was purified by column chromatography (silica gel, 24 g, 0˜100% EtOAc in PE) to give N-[(1S)-1-(1,1-difluoro-2,3-dihydro-1H-inden-4-yl)ethyl]-4-oxo-5-[(1s,4s)-4-[(2-hydroxyethyl)(methyl)carbamoyl]cyclohexyl]-2-{[2-(trimethylsilyl)ethoxy]methyl}-2H,4H,5H-pyrazolo[4,3-c]pyridine-7-carboxamide (50 mg, 0.07 mmol, 73.3%) as a white solid. LCMS: m/z 671 (M+H)⁺.

### Step L. N—((R)-1-(1,1-difluoro-2,3-dihydro-1H-inden-4-yl)ethyl)-5-((1s,4S)-4-((2-hydroxyethyl)(methyl)carbamoyl)cyclohexyl)-4-oxo-4,5-dihydro-2H-pyrazolo[4,3-c]pyridine-7-carboxamide

To a stirred solution of N-[(1S)-1-(1,1-difluoro-2,3-dihydro-1H-inden-4-yl)ethyl]-4-oxo-5-[(1s,4s)-4-[(2-hydroxyethyl)(methyl)carbamoyl]cyclohexyl]-2-{[2-(trimethylsilyl)ethoxy]methyl}-2H,4H,5H-pyrazolo[4,3-c]pyridine-7-carboxamide (50 mg, 0.074 mmol) in 4N HCl/dioxane (3 mL). The reaction mixture was stirred at room temperature for 1 h and concentrated under vacuum to give the residue. The residue was purified by prep-HPLC (phase A: H₂O (0.1% TFA). phase B:MeCN) to give N-[(1R)-1-(1,1-difluoro-2,3-dihydro-1H-inden-4-yl)ethyl]-4-oxo-5-[(1s,4s)-4-[(2-hydroxyethyl)(methyl)carbamoyl]cyclohexyl]-2H,4H,5H-pyrazolo[4,3-c]pyridine-7-carboxamide (10 mg, 0.018 mmol, 24.8%) as a white solid. 1H NMR (400 MHz, MeOD) δ 8.34 (d, J=132.2 Hz, 1H), 8.14 (s, 1H), 7.56 (s, 1H), 7.44-7.34 (m, 2H), 5.39-5.31 (m, 1H), 4.58 (s, 1H), 3.71 (t, J=5.6 Hz, 2H), 3.52 (s, 1H), 3.17 (s, 1H), 3.11 (d, J=17.3 Hz, 1H), 2.97 (s, 1H), 2.60 (dq, J=20.7, 6.8 Hz, 2H), 2.32-2.17 (m, 2H), 2.10 (s, 2H), 1.85 (s, 2H), 1.74 (s, 2H), 1.61 (d, J=7.0 Hz, 2H). LCMS: m/z 542 (M+H)⁺.

### Example 12: Synthesis of (R)—N-(1-(1,1-difluoro-2,3-dihydro-1H-inden-4-yl)ethyl)-5-(1-(methylsulfonyl)piperidin-4-yl)-4-oxo-4,5-dihydro-2H-pyrazolo[4,3-c]pyridine-7-carboxamide

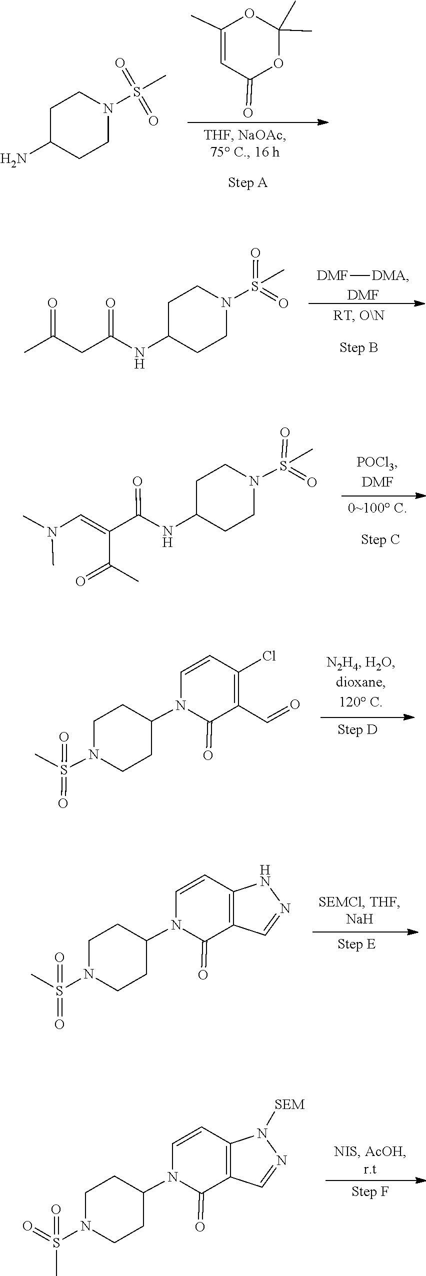

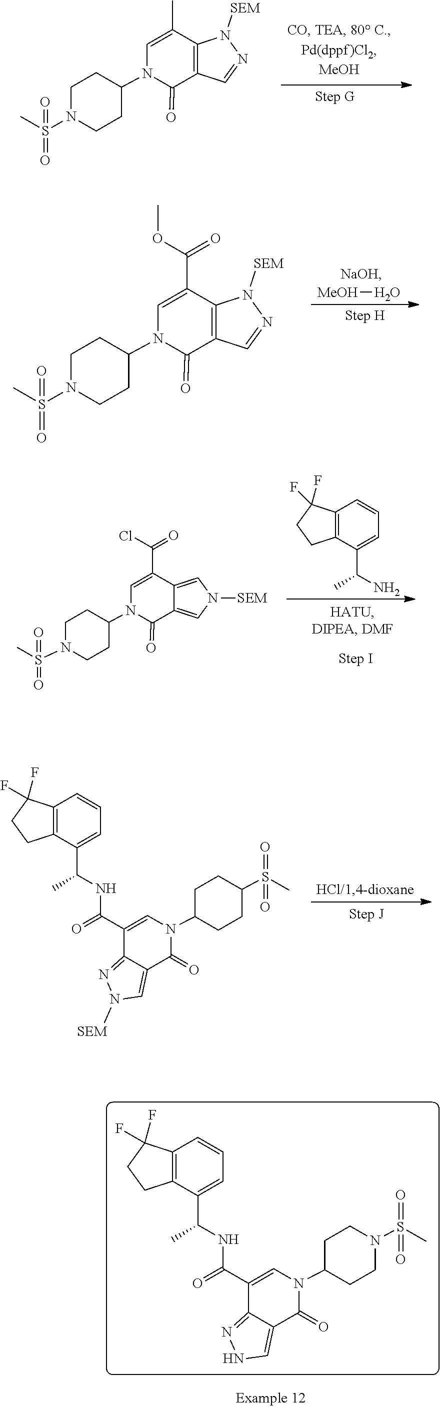

### Step A. N-(1-(methylsulfonyl)piperidin-4-yl)-3-oxobutanamide

To a stirred solution of 1-(methylsulfonyl)piperidin-4-amine (12.0 g, 112 mmol) in dry THF (50 mL) was added 2,2,6-trimethyl-4H-1,3-dioxin-4-one (123 mmol) and NaOAc (18.5 g, 224 mmol). The reaction mixture was stirred at 70° C. for 16 h. The reaction mixture was poured into water, extracted with EtOAc, washed by brine, dried over by Na₂SO₄ and concentrated under vacuum to give the residue. The residue was purified by column chromatography (silica gel, 40 g, 0˜100% EtOAc in PE) to give N-(1-(methylsulfonyl)piperidin-4-yl)-3-oxobutanamide (3.4 g, 21.9 mmol, 18.5%) as a yellow oil. LCMS: m/z 263 (M+H)⁺.

### Step B. (E)-2-((dimethylamino)methylene)-N-(1-(methylsulfonyl)piperidin-4-yl)-3-oxobutanamide

To a stirred solution of N-(1-(methylsulfonyl)piperidin-4-yl)-3-oxobutanamide (3.4 g, 21.9 mmol) in DMF (20 mL) was drop wised DMF-DMA (43.8 mmol) over 5 min The reaction mixture was stirred at room temperature for 16 h. The reaction mixture was concentrated under vacuum to give the residue. The residue was purified by column chromatography (silica gel, 40 g, 0˜10% MeOH in DCM) to give (E)-2-((dimethylamino)methylene)-N-(1-(methylsulfonyl) piperidin-4-yl)-3-oxobutanamide (4.3 g, 20.47 mmol, 97.4%) as a yellow oil. LCMS: m/z 318 (M+H)⁺.

### Step C. 4-chloro-1-(1-(methylsulfonyl)piperidin-4-yl)-2-oxo-1,2-dihydropyridine-3-carbaldehyde

POCl₃ (5.0 eq) was added into DMF (20 mL) dropwise at 0° C. and the mixture was stirred for 15 min. (E)-2-((dimethylamino)methylene)-N-(1-(methylsulfonyl)piperidin-4-yl)-3-oxobutanamide (4.3 g, 20.5 mmol) in DMF (10 mL) was added. The reaction mixture was stirred at 100° C. for 1 h. The cooled reaction mixture was concentrated under vacuum to remove POCl₃. The residue was poured into water and neutralized with saturated aqueous NaHCO₃ and extracted with EtOAc. The organic layers were washed with brine, dried over Na₂SO₄, concentrated under vacuum and purified by column chromatography (silica gel, 40 g, 0˜100% EtOAc in PE) to give 4-chloro-1-(1-(methylsulfonyl)piperidin-4-yl)-2-oxo-1,2-dihydropyridine-3-carbaldehyde (1.3 g, 6.03 mmol, 29.4%) as a yellow oil. LCMS: m/z 319 (M+H)⁺.

### Step D. 5-(1-(methylsulfonyl)piperidin-4-yl)-1,5-dihydro-4H-pyrazolo[4,3-c]pyridin-4-one

To a solution of 4-chloro-1-(1-(methylsulfonyl)piperidin-4-yl)-2-oxo-1,2-dihydropyridine-3-carbaldehyde (1.3 g, 6.03 mmol) in dioxane (20 mL) was added N₂H₄·H₂O (60.3 mmol). The reaction mixture was stirred at 120° C. for 4 h. The reaction mixture was concentrated under vacuum and purified by column chromatography (silica gel, 24 g, 0˜10% MeOH in DCM) to give 5-(1-(methylsulfonyl)piperidin-4-yl)-1,5-dihydro-4H-pyrazolo[4,3-c]pyridin-4-one (900 mg, 4.76 mmol, 78.1%) as a yellow solid. LCMS: m/z 297 (M+H)⁺.

### Step E. 5-(1-(methylsulfonyl)piperidin-4-yl)-1-((2-(trimethylsilyl)ethoxy)methyl)-1,5-dihydro-4H-pyrazolo[4,3-c]pyridin-4-one

To a stirred solution of 5-(1-(methylsulfonyl)piperidin-4-yl)-1,5-dihydro-4H-pyrazolo[4,3-c]pyridin-4-one (900 mg, 4.76 mmol) in DMF (5 mL) was added NaH (571 mg, 14.3 mmol) at 0° C. The reaction mixture was stirred at room temperature for 0.5 h, then SEMCl (948 mg, 5.71 mmol) was added. The reaction mixture was stirred at room temperature for 2 h. The reaction mixture was poured into water and extracted with EtOAc. The organic phase washed with brine, dried over Na₂SO₄ and concentrated under vacuum to give the residue. The residue purified by column chromatography (silica gel, 24 g, 0-70% EtOAc in PE) to give 5-(1-(methylsulfonyl)piperidin-4-yl)-1-((2-(trimethylsilyl)ethoxy)methyl)-1,5-dihydro-4H-pyrazolo[4,3-c]pyridin-4-one (1.2 g, 3.76 mmol, 79.3%) as a light yellow solid. LCMS: m/z 427 (M+H)⁺.

### Step F. 7-iodo-5-(1-(methylsulfonyl)piperidin-4-yl)-1-((2-(trimethylsilyl)ethoxy)methyl)-1,5-dihydro-4H-pyrazolo[4,3-c]pyridin-4-one

To a stirred solution of 5-(1-(methylsulfonyl)piperidin-4-yl)-1-((2-(trimethylsilyl)ethoxy)methyl)-1,5-dihydro-4H-pyrazolo[4,3-c]pyridin-4-one (1.2 g, 3.76 mmol) in AcOH (10 mL) was added NIS (930 mg, 4.13 mmol). The reaction mixture was stirred at room temperature for 2 h. The reaction mixture was poured into water and extracted with EtOAc. The organic phase washed with NaHCO₃, dried over Na₂SO₄ and concentrated. The residue was purified by column chromatography (silica gel, 24 g, 0˜50% EtOAc in PE) to give 7-iodo-5-(1-(methylsulfonyl)piperidin-4-yl)-1-((2-(trimethylsilyl)ethoxy)methyl)-1,5-dihydro-4H-pyrazolo [4,3-c]pyridin-4-one (1.4 g, 3.13 mmol, 83.7%) as a light yellow oil. LCMS: m/z 553 (M+H)⁺.

### Step G. methyl 5-(1-(methylsulfonyl)piperidin-4-yl)-4-oxo-1-((2-(trimethylsilyl)ethoxy) methyl)-4,5-dihydro-1H-pyrazolo[4,3-c]pyridine-7-carboxylate

To a stirred solution of 7-iodo-5-(1-(methylsulfonyl)piperidin-4-yl)-1-((2-(trimethylsilyl) ethoxy)methyl)-1,5-dihydro-4H-pyrazolo[4,3-c]pyridin-4-one (1.4 g, 3.13 mmol) in MeOH (5 mL) was added Pd(dppf)Cl₂ (495 mg, 0.616 mmol) and TEA (6.16 mmol). The reaction mixture was stirred at 80° C. for 16 h under CO atmosphere. The reaction mixture was filtered and the filtrate was concentrated under vacuum to give the residue and the residue was purified by column chromatography (silica gel, 24 g, 0˜40% EtOAc in PE) to give methyl 5-(1-(methylsulfonyl) piperidin-4-yl)-4-oxo-1-((2-(trimethylsilyl)ethoxy)methyl)-4,5-dihydro-1H-pyrazolo[4,3-c]pyridine-7-carboxylate (530 mg, 1.41 mmol, 44.3%) as a light yellow solid. LCMS: m/z 485 (M+H)⁺.

### Step H. 5-(1-(methylsulfonyl)piperidin-4-yl)-4-oxo-2-((2-(trimethylsilyl)ethoxy)methyl)-4,5-dihydro-2H-pyrazolo[4,3-c]pyridine-7-carboxylic acid

To a stirred solution of methyl 5-(1-(methylsulfonyl)piperidin-4-yl)-4-oxo-1-((2-(trimethylsilyl)ethoxy)methyl)-4,5-dihydro-1H-pyrazolo[4,3-c]pyridine-7-carboxylate (50 mg, 0.132 mmol) in MeOH (5 mL) was added NaOH (52.80 mg, 1.32 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give the crude product 5-(1-(methylsulfonyl)piperidin-4-yl)-4-oxo-2-((2-(trimethylsilyl)ethoxy)methyl)-4,5-dihydro-2H-pyrazolo[4,3-c]pyridine-7-carboxylic acid (40 undefined, 0.11 mmol, 80.0%) as a light yellow solid. LCMS: m/z 471 (M+H)⁺.

### Step I. (R)—N-(1-(1,1-difluoro-2,3-dihydro-1H-inden-4-yl)ethyl)-5-(1-(methylsulfonyl) piperidin-4-yl)-4-oxo-2-((2-(trimethylsilyl)ethoxy)methyl)-4,5-dihydro-2H-pyrazolo[4,3-c]pyridine-7-carboxamide

To a stirred solution of 5-(1-(methylsulfonyl)piperidin-4-yl)-4-oxo-2-((2-(trimethylsilyl) ethoxy)methyl)-4,5-dihydro-2H-pyrazolo[4,3-c]pyridine-7-carboxylic acid (20 mg, 0.055 mmol) in DMF (3 mL) was added (1R)-1-(1,1-difluoro-2,3-dihydro-1H-inden-4-yl)ethan-1-amine (16.3 mg, 0.08 mmol), DIPEA (21.46 mg, 0.16 mmol) and HATU (41.84 mg, 0.11 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (100% EtOAc) to give (R)—N-(1-(1,1-difluoro-2,3-dihydro-1H-inden-4-yl)ethyl)-5-(1-(methylsulfonyl) piperidin-4-yl)-4-oxo-2-((2-(trimethylsilyl)ethoxy)methyl)-4,5-dihydro-2H-pyrazolo[4,3-c]pyridine-7-carboxamide (20 mg, 0.04 mmol, 66.9%) as a white solid. LCMS: m/z 650 (M+H)⁺.

### Step J. (R)—N-(1-(1,1-difluoro-2,3-dihydro-1H-inden-4-yl)ethyl)-5-(1-(methylsulfonyl) piperidin-4-yl)-4-oxo-4,5-dihydro-2H-pyrazolo[4,3-c]pyridine-7-carboxamide

To a stirred solution of (R)—N-(1-(1,1-difluoro-2,3-dihydro-1H-inden-4-yl)ethyl)-5-(1-(methylsulfonyl)piperidin-4-yl)-4-oxo-2-((2-(trimethylsilyl)ethoxy)methyl)-4,5-dihydro-2H-pyrazolo[4,3-c]pyridine-7-carboxamide (20 mg, 0.04 mmol) in 4N HCl/dioxane (3 mL). The reaction mixture was stirred at room temperature for 1 h. The reaction mixture was concentrated under vacuum to give the residue and the residue was purified by pre-HPLC (phase A: H₂O (0.1% TFA), phase B: MeCN) to give (R)—N-(1-(1,1-difluoro-2,3-dihydro-1H-inden-4-yl)ethyl)-5-(1-(methylsulfonyl)piperidin-4-yl)-4-oxo-4,5-dihydro-2H-pyrazolo[4,3-c]pyridine-7-carboxamide (5.3 mg, 0.01 mmol, 35.5%) as a white solid. LCMS: m/z 520 (M+H)⁺. ¹H NMR (400 MHz, MeOD) δ 8.54 (s, 1H), 8.28 (s, 1H), 8.14 (s, 1H), 7.54 (d, J=7.3 Hz, 1H), 7.44-7.32 (m, 2H), 5.35 (q, J=6.7 Hz, 1H), 5.05 (d, J=48.2 Hz, 1H), 3.92 (s, 2H), 3.13 (s, 2H), 2.99 (t, J=10.3 Hz, 2H), 2.90 (s, 3H), 2.59 (dq, J=21.2, 7.0 Hz, 2H), 2.00 (s, 4H), 1.61 (d, J=6.7 Hz, 3H).

### Example 13: Synthesis of (R)—N-(1-(1,1-difluoro-2,3-dihydro-1H-inden-4-yl)ethyl)-5-(3,3-difluorocyclobutyl)-4-oxo-4,5-dihydro-1H-pyrazolo[4,3-c]pyridine-7-carboxamide

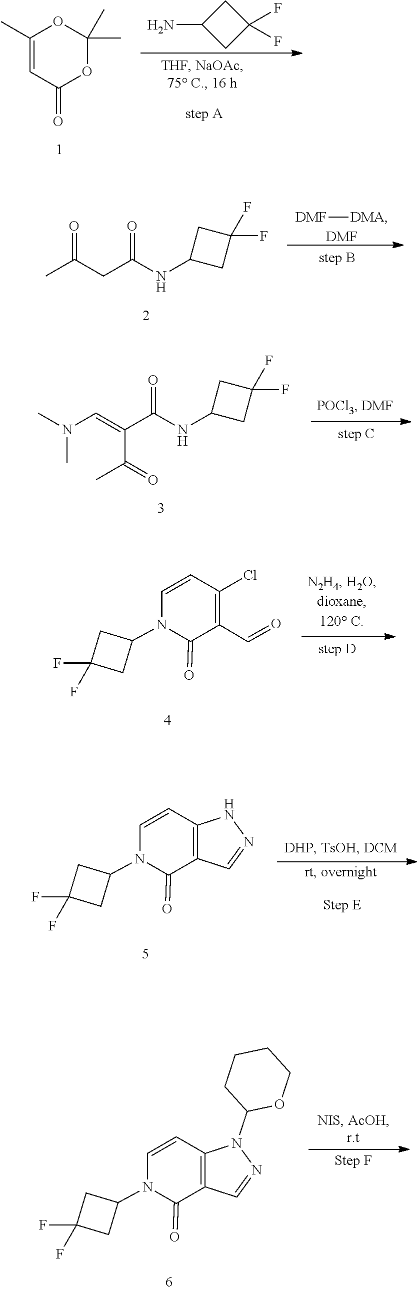

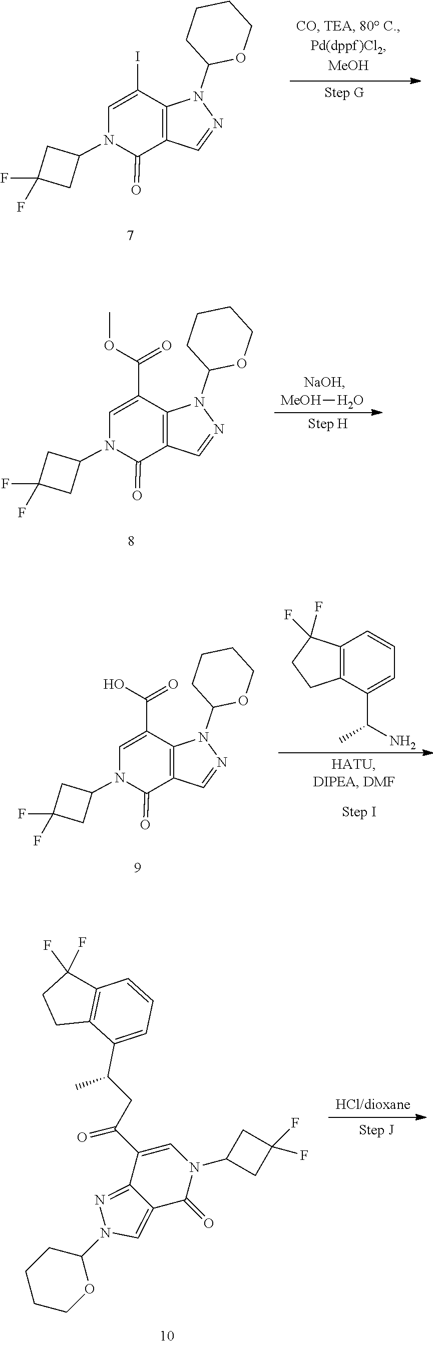

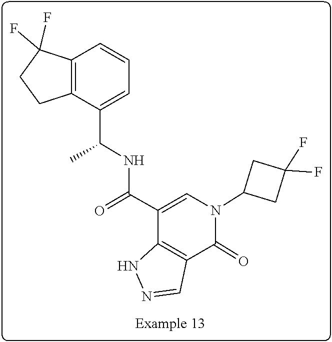 [SMILES: C[C@@H](NC(=O)c1cn(C2CC(F)(F)C2)c(=O)c2cn[nH]c12)c1cccc2c1CCC2(F)F]

### Step A: N-(3,3-difluorocyclobutyl)-3-oxobutanamide

To a solution of 2,2,6-trimethyl-2,4-dihydro-1,3-dioxin-4-one (7.87 mL, 59.8 mmol) in THF (60 mL) was added 3,3-difluorocyclobutan-1-amine (4.56 mL, 49.8 mmol) and NaOAc (12.3 g, 150 mmol) at rt. After stirring at 75° C. overnight, the mixture was quenched with water and extracted with EtOAc. The organic layers was washed with brine and dried over Na₂SO₄ then concentrated under reduced pressure. The residue was purified by chromatography (silica gel, 0-100%, EtOAc in PE) to give the product of N-(3,3-difluorocyclobutyl)-3-oxobutanamide (2.5 g, 13.1 mmol, 26.2%) as a white oil. LC-MS (ESI) m/z: 191⁺.

### Step B: (2E)-N-(3,3-difluorocyclobutyl)-2-[(dimethylamino)methylidene]-3-oxobutanamide

To a solution of N-(3,3-difluorocyclobutyl)-3-oxobutanamide (2.5 g, 13.1 mmol) in DMF (30 mL) was added DMF-DMA (3.5 mL, 26.2 mmol) dropwise at rt. After stirring at rt for overnight, the mixture was concentrated under reduced pressure. The residue was purified by chromatography (silica gel, 0-10%, MeOH in DCM) to give the product of (2E)-N-(3,3-difluorocyclobutyl)-2-[(dimethylamino)methylidene]-3-oxobutanamide (2.5 g, 10.2 mmol, 77.6%) as a white solid. LC-MS (ESI) m/z: 247⁺.

### Step C: 4-chloro-1-(3,3-difluorocyclobutyl)-2-oxo-1,2-dihydropyridine-3-carbaldehyde

To a solution of DMF (70 mL) was added POCl₃ (8.14 mL, 87.3 mmol) dropwise at 0° C. After stirring at 0° C. for 15 min, a solution of (2E)-N-(3,3-difluorocyclobutyl)-2-[(dimethylamino)methylidene]-3-oxobutanamide (4.3 g, 17.5 mmol) in DMF (40 mL) was added at 0° C. The reaction mixture was heated to 100° C. and stirred for 1 h. After complete conversion of situation, the mixture was concentrated under reduced press. The residue was washed with NaHCO₃, extracted with EtOAc. The mixture was purified by chromatography (silica gel, 0-50%, EtOAc in PE) to give the product of 4-chloro-1-(3,3-difluorocyclobutyl)-2-oxo-1,2-dihydropyridine-3-carbaldehyde (2.75 g, 11.1 mmol, 63.6%) as a colourless oil. LC-MS (ESI) m/z: 248⁺.

### Step D: 5-(3,3-difluorocyclobutyl)-1H,4H,5H-pyrazolo[4,3-c]pyridin-4-one

To a solution of 4-chloro-1-(3,3-difluorocyclobutyl)-2-oxo-1,2-dihydropyridine-3-carbaldehyde (2.1 g, 8.48 mmol) in dioxane (37 mL) was added N₂H₄ solution (9.5 g, 190 mmol) at rt. After stirring at 120° C. overnight, the mixture was concentrated and extracted with EtOAc. The organic layers was washed with brine and dried over Na₂SO₄ then concentrated under reduced pressure. The mixture was purified by chromatography (silica gel, 0-20%, DCM in MeOH) to give the product of 5-(3,3-difluorocyclobutyl)-1H,4H,5H-pyrazolo[4,3-c]pyridin-4-one (1.8 g, 8.0 mmol, 94.7%) as a colourless oil. LC-MS (ESI) m/z: 226⁺.

### Step E: 5-(3,3-difluorocyclobutyl)-1-(oxan-2-yl)-1H,4H,5H-pyrazolo[4,3-c]pyridin-4-one

To a solution of 5-(3,3-difluorocyclobutyl)-1H,4H,5H-pyrazolo[4,3-c]pyridin-4-one (1 g, 4.44 mmol) in MeCN (15 mL) was added TsOH·H₂O (0.08 g, 0.444 mmol), 3,4-dihydro-2H-pyran (1.12 g, 13.3 mmol) at rt. After stirring at rt ° C. for 15 min, the mixture was concentrated under reduced pressure. The residue was purified by chromatography (silica gel, 0-50%, EtOAc in PE) to give the product of 5-(3,3-difluorocyclobutyl)-1-(oxan-2-yl)-1H,4H,5H-pyrazolo[4,3-c]pyridin-4-one (0.9 g, 2.91 mmol, 65.5%) as a white oil. LC-MS (ESI) m/z: 310⁺.

### Step F: 5-(3,3-difluorocyclobutyl)-7-iodo-1-(oxan-2-yl)-1H,4H,5H-pyrazolo[4,3-c]pyridin-4-one

To a solution of 5-(3,3-difluorocyclobutyl)-1-(oxan-2-yl)-1H,4H,5H-pyrazolo[4,3-c]pyridin-4-one (0.9 g, 2.91 mmol) in AcOH (5 mL) was added NIS (0.98 g, 4.36 mmol) at rt. After stirring at rt for 1 h, the mixture was quenched with water and extracted with EtOAc. The organic layers was washed with brine and dried over Na₂SO₄ then concentrated under reduced pressure. The mixture was purified by chromatography (silica gel, 0-40%, EtOAc in PE) to give the product of 5-(3,3-difluorocyclobutyl)-7-iodo-1-(oxan-2-yl)-1H,4H,5H-pyrazolo[4,3-c]pyridin-4-one (0.45 g, 1.03 mmol, 35.5%) as a brown solid. LC-MS (ESI) m/z: 436⁺.

### Step G: methyl 5-(3,3-difluorocyclobutyl)-1-(oxan-2-yl)-4-oxo-1H,4H,5H-pyrazolo[4,3-c]pyridine-7-carboxylate

To a solution of 5-(3,3-difluorocyclobutyl)-7-iodo-1-(oxan-2-yl)-1H,4H,5H-pyrazolo[4,3-c]pyridin-4-one (0.4 g, 0.92 mmol) in MeOH (5 mL) was added TEA (0.383 mL, 2.76 mmol) and Pd(dppf)Cl₂ (0.13 g, 0.184 mmol) at rt under CO atmosphere. After stirring at 60° C. overnight, the reaction was filtered. The filtrate was concentrated and purified by chromatography (silica gel, 0-50%, EtOAc in PE) to give the product of methyl 5-(3,3-difluorocyclobutyl)-1-(oxan-2-yl)-4-oxo-1H,4H,5H-pyrazolo[4,3-c]pyridine-7-carboxylate (0.18 g, 0.49 mmol, 53.3%) as a brown oil. LC-MS (ESI) m/z: 368⁺.

### Step H: 5-(3,3-difluorocyclobutyl)-1-(oxan-2-yl)-4-oxo-1H,4H,5H-pyrazolo[4,3-c]pyridine-7-carboxylic acid

To a solution of methyl 5-(3,3-difluorocyclobutyl)-1-(oxan-2-yl)-4-oxo-1H,4H,5H-pyrazolo[4,3-c]pyridine-7-carboxylate (0.07 g, 0.19 mmol) in MeOH (5 mL) was added a solution of NaOH (1.5 mL, 1.27 mol/L) at rt. After stirring at rt for 1.5 h, the mixture was adjusted PH to 3-4 with 2 N HCl (3 mL) and extracted with EtOAc. The organic layers was washed with brine and dried over Na₂SO₄ then concentrated under reduced pressure. The residue was purified by chromatography (silica gel, 0-100%, EtOAc in PE) to give the product of 5-(3,3-difluorocyclobutyl)-1-(oxan-2-yl)-4-oxo-1H,4H,5H-pyrazolo[4,3-c]pyridine-7-carboxylic acid (0.06 g, 0.17 mmol, 89.1%) as a white solid. LC-MS (ESI) m/z: 354⁺.

### Step I: N-[(1R)-1-(1,1-difluoro-2,3-dihydro-1H-inden-4-yl)ethyl]-5-(3,3-difluorocyclobutyl)-2-(oxan-2-yl)-4-oxo-2H,4H,5H-pyrazolo[4,3-c]pyridine-7-carboxamide

To a solution of 5-(3,3-difluorocyclobutyl)-1-(oxan-2-yl)-4-oxo-1H,4H,5H-pyrazolo[4,3-c]pyridine-7-carboxylic acid (0.02 g, 0.057 mmol) in DMF (2 mL) was added (R)-1-(1,1-difluoro-2,3-dihydro-1H-inden-4-yl)ethan-1-amine (0.02 g, 0.102 mmol), DIPEA (0.04 g, 0.283 mmol) and HATU (0.04 g, 0.113 mmol) at rt. After stirring at rt for 1 h, the mixture was concentrated under reduced pressure to give the crude of N-[(1R)-1-(1,1-difluoro-2,3-dihydro-1H-inden-4-yl)ethyl]-5-(3,3-difluorocyclobutyl)-2-(oxan-2-yl)-4-oxo-2H,4H,5H-pyrazolo[4,3-c]pyridine-7-carboxamide (0.03 g, 0.045 mmol, 79.6%) as a white solid. LC-MS (ESI) m/z: 534⁺.

### Step J: N-[(1R)-1-(1,1-difluoro-2,3-dihydro-1H-inden-4-yl)ethyl]-5-(3,3-difluorocyclobutyl)-4-oxo-1H,4H,5H-pyrazolo[4,3-c]pyridine-7-carboxamide

To a stirred solution of N-[(1R)-1-(1,1-difluoro-2,3-dihydro-1H-inden-4-yl)ethyl]-5-(3,3-difluorocyclobutyl)-2-(oxan-2-yl)-4-oxo-2H,4H,5H-pyrazolo[4,3-c]pyridine-7-carboxamide (0.06 g, 0.09 mmol) in dioxane (5 mL) was added 4N HCl/dioxane (2 mL, 8 mmol) at rt. After stirring at rt for 10 min, the mixture was concentrated. The crude product was purified by prep-HPLC (C18, 30˜80% MeCN in H₂O with 0.1% HCOOH) to give N-[(1R)-1-(1,1-difluoro-2,3-dihydro-1H-inden-4-yl)ethyl]-5-(3,3-difluorocyclobutyl)-4-oxo-1H,4H,5H-pyrazolo[4,3-c]pyridine-7-carboxamide (0.02 g, 0.045 mmol, 49.5%) as a white solid. LC-MS (ESI) m/z 449 (M+H)⁺. ¹H NMR (400 MHz, DMSO) δ 8.87 (d, J=7.1 Hz, 1H), 8.08 (s, 1H), 7.58 (d, J=6.8 Hz, 1H), 7.50-7.39 (m, 2H), 5.25 (p, J=7.0 Hz, 1H), 4.96-4.84 (m, 1H), 3.20-3.07 (m, 5H), 2.68-2.54 (m, 2H), 1.54 (d, J=7.0 Hz, 3H).

### Example 14: Synthesis of (R)-5-(3,3-difluorocyclobutyl)-4-oxo-N-(1-(3-(trifluoromethyl) phenyl)ethyl)-4,5-dihydro-1H-pyrazolo[4,3-c]pyridine-7-carboxamide (the Title Compounds was Synthesized from Compound 9 in Example 13.)

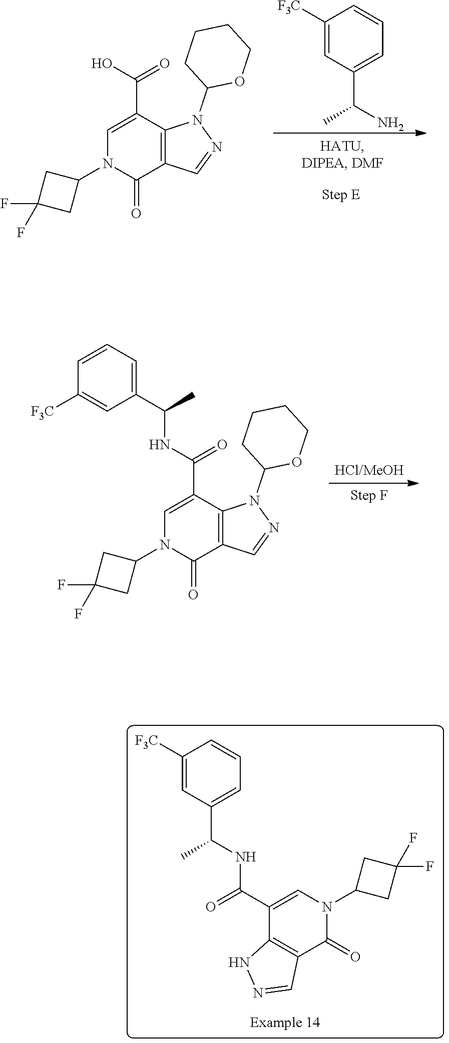 [SMILES: O=C(O)c1cn(C2CC(F)(F)C2)c(=O)c2cnn(C3CCCCO3)c12 ; C[C@@H](N)c1cccc(C(F)(F)F)c1 ; C[C@@H](NC(=O)c1cn(C2CC(F)(F)C2)c(=O)c2cnn(C3CCCCO3)c12)c1cccc(C(F)(F)F)c1 ; C[C@@H](NC(=O)c1cn(C2CC(F)(F)C2)c(=O)c2cn[nH]c12)c1cccc(C(F)(F)F)c1]

### Step E: 5-(3,3-difluorocyclobutyl)-1-(oxan-2-yl)-4-oxo-N-[(1R)-1-[3-(trifluoromethyl) phenyl]ethyl]-1H,4H,5H-pyrazolo[4,3-c]pyridine-7-carboxamide

To a solution of 5-(3,3-difluorocyclobutyl)-1-(oxan-2-yl)-4-oxo-1H,4H,5H-pyrazolo[4,3-c]pyridine-7-carboxylic acid (70 mg, 0.198 mmol) in DMF (0.5 mL) was added (R)-1-(3-(trifluoromethyl)phenyl)ethan-1-amine (67.4 mg, 0.357 mmol), DIPEA (128 mg, 0.991 mmol) and HATU (151 mg, 0.396 mmol) at rt. After stirring at rt for 30 min, the mixture was concentrated under reduced pressure to give the crude of 5-(3,3-difluorocyclobutyl)-1-(oxan-2-yl)-4-oxo-N-[(1R)-1-[3-(trifluoromethyl)phenyl]ethyl]-1H,4H,5H-pyrazolo[4,3-c]pyridine-7-carboxamide (70 mg, 0.08 mmol, 40.4%) as a white solid. LC-MS (ESI) m/z: 534⁺.

### Step F: 5-(3,3-difluorocyclobutyl)-4-oxo-N-[(1R)-1-[3-(trifluoromethyl)phenyl]ethyl]-1H,4H,5H-pyrazolo[4,3-c]pyridine-7-carboxamide

To a stirred solution of 5-(3,3-difluorocyclobutyl)-1-(oxan-2-yl)-4-oxo-N-[(1R)-1-[3-(trifluoromethyl)phenyl]ethyl]-1H,4H,5H-pyrazolo[4,3-c]pyridine-7-carboxamide (70 mg, 0.08 mmol) in dioxane (5 mL) was added 4N HCl/dioxane (4 mol/L, 5 mL) at rt. After stirring at rt for 1 h, the mixture was concentrated. The residue was purified by prep-HPLC (C18, 30˜80% MeCN in H₂O with 0.1% TFA) to give the product of 5-(3,3-difluorocyclobutyl)-4-oxo-N-[(1R)-1-[3-(trifluoromethyl)phenyl]ethyl]-1H,4H,5H-pyrazolo[4,3-c]pyridine-7-carboxamide (15 mg, 0.034 mmol, 42.5%) as a white solid. LC/MS (ESI) m/z: 556 (M+H)⁺. ¹H NMR (400 MHz, MeOD) δ 8.38 (s, 1H), 8.14 (s, 1H), 7.69 (dd, J=8.5, 3.6 Hz, 2H), 7.54 (d, J=6.4 Hz, 2H), 5.33 (q, J=7.0 Hz, 1H), 5.04-4.92 (m, 2H), 3.23-3.02 (m, 4H), 1.63 (d, J=7.0 Hz, 3H).

### Example 15: 5-(3,3-difluorocyclobutyl)-N-[(1R)-1-(4-{2-[(methylamino)methyl]phenyl}thiophen-2-yl)ethyl]-4-oxo-1H,4H,5H-pyrazolo[4,3-c]pyridine-7-carboxamide. (the Title Compounds was Synthesized from Compound 9 in Example 13.)

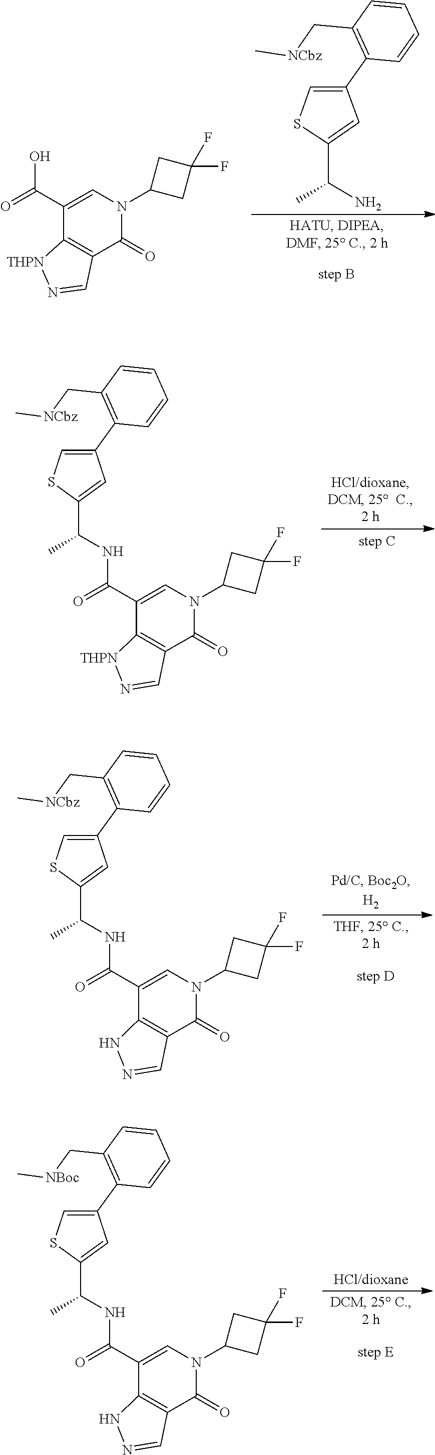

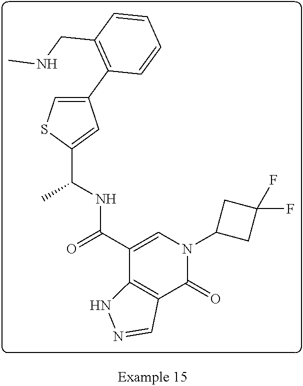 [SMILES: CNCc1ccccc1-c1csc([C@@H](C)NC(=O)c2cn(C3CC(F)(F)C3)c(=O)c3cn[nH]c23)c1]

### Step B: benzyl N-[(2-{5-[(1R)-1-{[5-(3,3-difluorocyclobutyl)-1-(oxan-2-yl)-4-oxo-1H,4H,5H-pyrazolo[4,3-c]pyridin-7-yl]formamido}ethyl]thiophen-3-yl}phenyl)methyl]-N-methylcarbamate

To a stirred solution of 5-(3,3-difluorocyclobutyl)-1-(oxan-2-yl)-4-oxo-1H,4H,5H-pyrazolo[4,3-c]pyridine-7-carboxylic acid (100 mg, 0.283 mmol) in DMF (2 mL) was added benzyl N-[(2-{5-[(1R)-1-aminoethyl]thiophen-3-yl}phenyl)methyl]-N-methylcarbamate (161 mg, 0.425 mmol), HATU (161 mg, 0.425 mmol) and DIPEA (109 mg, 0.849 mmol). The reaction mixture was stirred at 25° C. for 1 h under nitrogen atmosphere. The reaction mixture was poured into ice-water (20 mL), extracted with EtOAc (20 mL×3). The combined organic phase was washed by water and brine, dried over anhydrous Na₂SO₄, filtered and concentrated to give a crude product, which was purified by column chromatography on silica gel (DCM:MeOH=100:1 to 30:1) to obtain benzyl N-[(2-{5-[(1R)-1-{[5-(3,3-difluorocyclobutyl)-1-(oxan-2-yl)-4-oxo-1H,4H,5H-pyrazolo[4,3-c]pyridin-7-yl]formamido}ethyl]thiophen-3-yl}phenyl)methyl]-N-methylcarbamate (170 mg, 83%) as a yellow liquid. LC/MS (ESI) m/z: 716.0 (M+H)⁺.

### Step C: benzyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate

To a stirred solution of benzyl N-[(2-{5-[(1R)-1-{[5-(3,3-difluorocyclobutyl)-1-(oxan-2-yl)-4-oxo-1H,4H,5H-pyrazolo[4,3-c]pyridin-7-yl]formamido}ethyl]thiophen-3-yl}phenyl)methyl]-N-methylcarbamate (80 mg, 0.112 mmol) in DCM (2 mL) was added HCl (2 mL, 4 N). The reaction mixture was stirred at 25° C. for 1 h. The reaction mixture was diluted with water (10 mL). The following mixture was extracted with DCM (10 mL*3). The combined organic phase was washed by water and brine, dried over anhydrous Na₂SO₄, filtered and concentrated to give the crude product which was purified by column chromatography on silica gel (DCM:MeOH=100:1 to 20:1) to obtain benzyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate (60 mg, 85%) as a white solid. LC/MS (ESI) m/z: 632.0 (M+H)⁺

### Step D: tert-butyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate

To a solution of benzyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate (40 mg, 0.063 mmol) in MeOH (5 mL) was added Pd/C (6 mg, 10% wt) and Boc₂O (27 mg, 0.127 mmol). The reaction mixture was concentrated to give a crude product which was purified by column chromatography on silica gel (DCM:MeOH=100:1 to 30:1) to obtain tert-butyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate (20 mg, 53%) as a yellow solid. LC/MS (ESI) m/z: 598 (M+H)⁺.

### Step E: 5-(3,3-difluorocyclobutyl)-N-[(1R)-1-(4-{2-[(methylamino)methyl]phenyl}thiophen-2-yl)ethyl]-4-oxo-1H,4H,5H-pyrazolo[4,3-c]pyridine-7-carboxamide

To a stirred solution of tert-butyl N-[(2-{5-[(1S)-1-{[5-(3,3-difluorocyclobutyl)-4-oxo-1H,4H,5H-pyrazolo[4,3-c]pyridin-7-yl]formamido}ethyl]thiophen-3-yl}phenyl)methyl]-N-methylcarbamate (20 mg, 0.033 mmol) in DCM (2 mL) at 0° C. was added 4N HCl/dioxane (2 mL). The reaction mixture was stirred at 25° C. for 1 h. The mixture was extracted with EtOAc (5 mL×3), the combined organic phase was washed by sat. NaHCO₃ and water and brine, dried over anhydrous Na₂SO₄, filtered and concentrated to give a crude product which was purified by column chromatography on silica gel (DCM:MeOH=10:1) to obtain 5-(3,3-difluorocyclobutyl)-N-[(1R)-1-(4-{2-[(methylamino)methyl]phenyl}thiophen-2-yl)ethyl]-4-oxo-1H,4H,5H-pyrazolo[4,3-c]pyridine-7-carboxamide (10 mg, 60%) as a yellow solid. LC/MS (ESI) m/z: 498 (M+H)⁺. ¹H NMR (400 MHz, CD₃OD-d₄) δ 8.41 (s, 1H), 8.19 (s, 1H), 7.57-7.53 (m, 1H), 7.51-7.41 (m, 3H), 7.14 (dd, J=3.6, 0.8 Hz, 1H), 7.00 (d, J=3.6 Hz, 1H), 5.59 (q, J=6.9 Hz, 1H), 5.05-4.98 (m, 1H), 4.26 (s, 2H), 3.18-3.08 (m, 4H), 2.59 (s, 3H), 1.75 (d, J=6.9 Hz, 3H).

### Example 16: Synthesis of N-[(1R)-1-(1,1-difluoro-2,3-dihydro-1H-inden-4-yl)ethyl]-4-methoxy-5-(oxan-4-yl)-2H-indazole-7-carboxamide

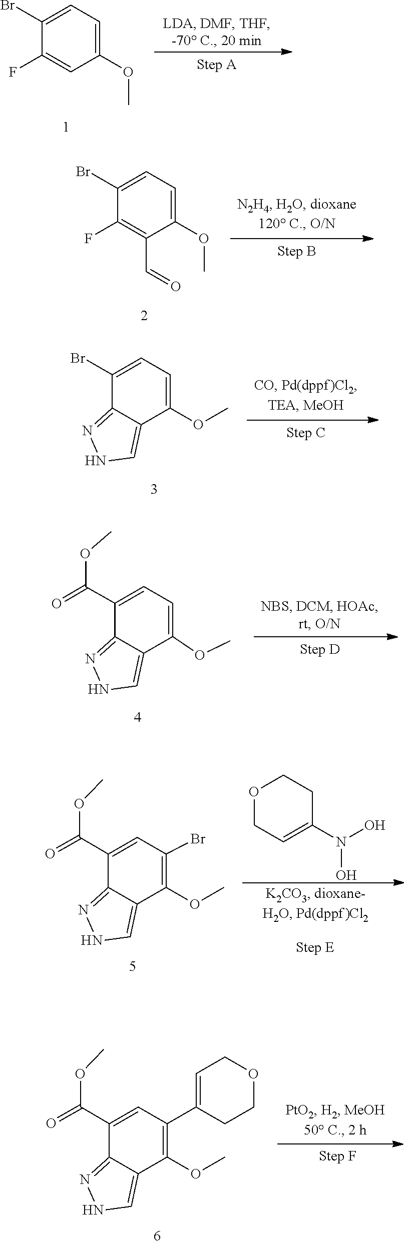

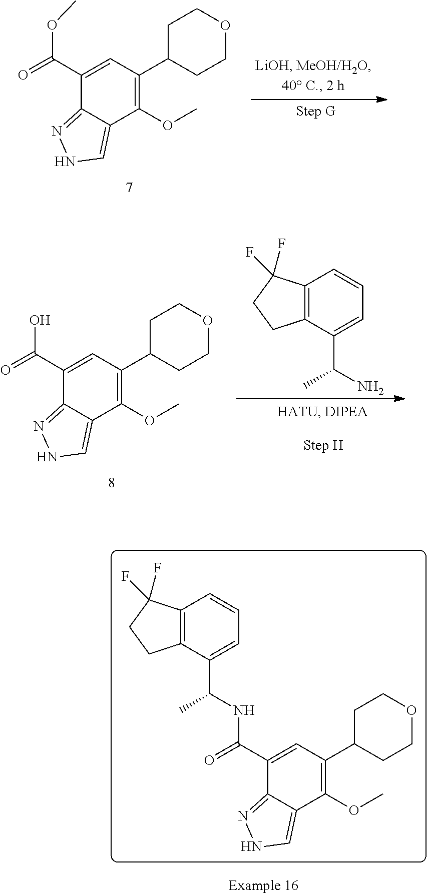 [SMILES: COC(=O)c1cc(C2CCOCC2)c(OC)c2c[nH]nc12 ; COc1c(C2CCOCC2)cc(C(=O)O)c2n[nH]cc12 ; C[C@@H](N)c1cccc2c1CCC2(F)F ; COc1c(C2CCOCC2)cc(C(=O)N[C@H](C)c2cccc3c2CCC3(F)F)c2n[nH]cc12]

### Step A. 3-bromo-2-fluoro-6-methoxybenzaldehyde

To a stirred solution of 1-bromo-2-fluoro-4-methoxybenzene (10 g, 48.7 mmol) in THF (100 mL) was added LDA (30 mL, 60 mmol, 2M in THF) at −70° C. After stirring at −70° C. for 20 min, DMF (5 mL, 64.6 mmol) was added at −70° C. After stirring at −70° C. for 20 min, the mixture was poured into NH₄Cl (aq. 200 mL) and extracted with EtOAc (200 mL*2). The combined organic phase was washed with brine, dried over Na₂SO₄, filtered and concentrated to give 3-bromo-2-fluoro-6-methoxybenzaldehyde (11 g, 47.2 mmol, 96.8%) as a yellow solid. LC-MS (ESI) m/z 234 (M+H)⁺.

### Step B. 7-bromo-4-methoxy-2H-indazole

To a stirred solution of 3-bromo-2-fluoro-6-methoxybenzaldehyde (12 g, 51.4 mmol) in dioxane (50 mL) was added NH₂NH₂·H₂O (36.8 g, 515 mmol, 70% in water) at rt. After stirring at 110° C. overnight, the cooled mixture was concentrated. The crude product was purified by column chromatography (silica gel, 30-100%, EtOAc in PE) to give 7-bromo-4-methoxy-2H-indazole (10 g, 44.04 mmol, 85%) as a white solid. LC-MS (ESI) m/z 228 (M+H)⁺.

### Step C. methyl 4-methoxy-2H-indazole-7-carboxylate

To a stirred solution of 7-bromo-4-methoxy-2H-indazole (200 mg, 0.88 mmol) in MeOH (5 mL) was added Pd(dppf)Cl₂ (64 mg, 0.088 mmol) and TEA (0.4 mL, 2.7 mmol) at rt. After stirring under CO atmosphere at 70° C. overnight, the cooled mixture was filtered and concentrated. The crude product was purified by chromatography (silica gel, 30-100%, EtOAc in PE) to give methyl 4-methoxy-2H-indazole-7-carboxylate (150 mg, 0.72 mmol, 82%) as a white solid. LC-MS (ESI) m/z 207 (M+H)⁺.

### Step D. 5-bromo-4-methoxy-2H-indazole-7-carboxylate

To a stirred solution of methyl 4-methoxy-2H-indazole-7-carboxylate (150 mg, 0.72 mmol) in DCM (5 mL) and HOAc (5 mL) was added NBS (142 mg, 0.80 mmol) at rt. After stirring at rt overnight, the mixture was concentrated. The crude product was poured into NaHCO₃ (aq, 20 mL) and extracted with EtOAc (20 mL*2). The combined organic phase was washed with brine, dried over Na₂SO₄, filtered and concentrated. The crude product was purified by chromatography (silica gel, 50-100%, EtOAc in PE) to give methyl 5-bromo-4-methoxy-2H-indazole-7-carboxylate (130 mg, 0.45 mmol, 62%) as a white solid. LC-MS (ESI) m/z 286 (M+H)⁺.

### Step E. methyl 5-(3,6-dihydro-2H-pyran-4-yl)-4-methoxy-2H-indazole-7-carboxylate

To a stirred solution of methyl 5-bromo-4-methoxy-2H-indazole-7-carboxylate (200 mg, 0.70 mmol) in dioxane (2 mL) and water (0.5 mL) was added (3,6-dihydro-2H-pyran-4-yl)boronic acid (135 mg, 1.052 mmol), Pd(dppf)Cl₂ (103 mg, 0.140 mmol) and K₂CO₃ (291 mg, 2.10 mmol) at rt. After stirring at 100° C. for 2 h, the cooled mixture was filtered and concentrated. The crude product was purified by column chromatography (silica gel, 0-20%, MeOH in DCM) to give methyl 5-(3,6-dihydro-2H-pyran-4-yl)-4-methoxy-2H-indazole-7-carboxylate (150 mg, 0.52 mmol, 74%) as a yellow solid. LC-MS (ESI) m/z 289 (M+H)⁺.

### Step F. methyl 4-methoxy-5-(oxan-4-yl)-2H-indazole-7-carboxylate

A stirred solution of methyl 5-(3,6-dihydro-2H-pyran-4-yl)-4-methoxy-2H-indazole-7-carboxylate (150 mg, 0.52 mmol) in MeOH (10 mL) was added PtO₂ (30 mg, 0.69 mmol) at rt. After stirring at 50° C. under a H₂ balloon for 2 h, the cooled mixture was filtered and concentrated to give methyl 4-methoxy-5-(oxan-4-yl)-2H-indazole-7-carboxylate (120 mg, 0.413 mmol, 79%) as a brown solid. LC-MS (ESI) m/z 291 (M+H)⁺.

### Step G. 4-methoxy-5-(oxan-4-yl)-2H-indazole-7-carboxylic acid

To a stirred solution of methyl 4-methoxy-5-(oxan-4-yl)-2H-indazole-7-carboxylate (120 mg, 0.41 mmol) in MeOH (5 mL) and water (5 mL) was added LiOH (173 mg, 4.13 mmol) at rt. After stirring at 40° C. for 2 h, the cooled mixture was concentrated to give crude 4-methoxy-5-(oxan-4-yl)-2H-indazole-7-carboxylic acid (115 mg, 0.41 mmol, 100%) as a brown solid. LC-MS (ESI) m/z 300 (M+H)⁺.

### Step H. N-[(1R)-1-(1,1-difluoro-2,3-dihydro-1H-inden-4-yl)ethyl]-4-methoxy-5-(oxan-4-yl)-2H-indazole-7-carboxamide

To a stirred solution of 4-methoxy-5-(oxan-4-yl)-2H-indazole-7-carboxylic acid (115 mg, 0.41 mmol) in acetonitrile (10 mL) and DMF (5 mL) was added (1R)-1-(1,1-difluoro-2,3-dihydro-1H-inden-4-yl)ethan-1-amine (123 mg, 0.62 mmol), HATU (316 mg, 0.83 mmol) and DIPEA (0.5 mL, 2.8 mmol) at rt. After stirring at 70° C. overnight, the cooled mixture was poured into water (30 mL) and extracted with EtOAc (30 mL*2). The combined organic phase was washed with brine, dried over Na₂SO₄, filtered and concentrated. The crude product was purified by chromatography (silica gel, 0-20%, MeOH in DCM) and then by prep-HPLC (C18, 30˜80% MeCN in H₂O with 0.1% HCOOH) to give N-[(1R)-1-(1,1-difluoro-2,3-dihydro-1H-inden-4-yl)ethyl]-4-methoxy-5-(oxan-4-yl)-2H-indazole-7-carboxamide (7 mg, 0.015 mmol, 3%) LC-MS (ESI) m/z 456 (M+H)⁺. ¹H NMR (400 MHz, MeOD) δ 8.33 (s, 1H), 7.94 (s, 1H), 7.59 (d, J=6.3 Hz, 1H), 7.38 (d, J=6.7 Hz, 2H), 5.40 (d, J=7.0 Hz, 1H), 4.30 (s, 3H), 4.07 (d, J=8.7 Hz, 2H), 3.61 (t, J=11.6 Hz, 2H), 3.13 (s, 1H), 2.64-2.55 (m, 2H), 2.03-1.94 (m, 2H), 1.71 (d, J=12.6 Hz, 2H), 1.61 (d, J=6.9 Hz, 3H), 1.33 (d, J=25.3 Hz, 2H).

### Example 17: Synthesis of N-[(1R)-1-(1,1-difluoro-2,3-dihydro-1H-inden-4-yl)ethyl]-4-methoxy-5-(1-methyl-6-oxo-1,6-dihydropyridin-3-yl)-2H-indazole-7-carboxamide. (the Title Compounds was Synthesized from Compound 5 in Example 16.)

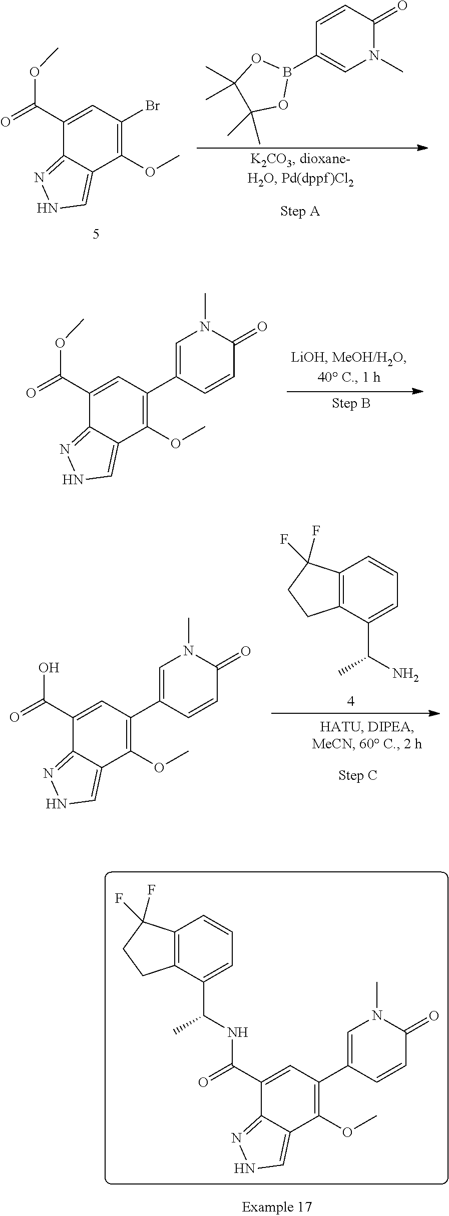

### Step A. methyl 4-methoxy-5-(1-methyl-6-oxo-1,6-dihydropyridin-3-yl)-2H-indazole-7-carboxylate

To a stirred solution of methyl 5-bromo-4-methoxy-2H-indazole-7-carboxylate (130 mg, 0.456 mmol) in dioxane (2 mL) and water (0.5 mL) was added 1-methyl-5-(tetramethyl-1,3,2-dioxaborolan-2-yl)-1,2-dihydropyridin-2-one (161 mg, 0.684 mmol), Pd(dppf)Cl₂ (67 mg, 0.091 mmol) and K₂CO₃ (189 mg, 1.368 mmol) at rt. After stirring at 100° C. for 2 h, the cooled mixture was filtered and concentrated. The crude product was purified by chromatography (silica gel, 0-20%, MeOH in DCM) to give methyl 4-methoxy-5-(1-methyl-6-oxo-1,6-dihydropyridin-3-yl)-2H-indazole-7-carboxylate (120 mg, 0.383 mmol, 84%) as a yellow solid. LC-MS (ESI) m/z 314 (M+H)⁺.

### Step B. 4-methoxy-5-(1-methyl-6-oxo-1,6-dihydropyridin-3-yl)-2H-indazole-7-carboxylic acid

To a stirred solution of methyl 4-methoxy-5-(1-methyl-6-oxo-1,6-dihydropyridin-3-yl)-2H-indazole-7-carboxylate (120 mg, 0.383 mmol) in MeOH (5 mL) and water (5 mL) was added LiOH (160 mg, 3.830 mmol) at rt. After stirring at 40° C. for 2 h, the cooled mixture was concentrated to give crude 4-methoxy-5-(1-methyl-6-oxo-1,6-dihydropyridin-3-yl)-2H-indazole-7-carboxylic acid (115 mg, 0.384 mmol, 100%) as a white solid. LC-MS (ESI) m/z 300 (M+H)⁺.

### Step C. N-[(1R)-1-(1,1-difluoro-2,3-dihydro-1H-inden-4-yl)ethyl]-4-methoxy-5-(1-methyl-6-oxo-1,6-dihydropyridin-3-yl)-2H-indazole-7-carboxamide

To a stirred solution of 4-methoxy-5-(1-methyl-6-oxo-1,6-dihydropyridin-3-yl)-2H-indazole-7-carboxylic acid (115 mg, 0.384 mmol) in acetonitrile (10 mL) and DMF (5 mL) was added (1R)-1-(1,1-difluoro-2,3-dihydro-1H-inden-4-yl)ethan-1-amine (115 mg, 0.576 mmol), HATU (292 mg, 0.768 mmol) and DIPEA (0.5 mL, 2.810 mmol) at rt. After stirring at 70° C. overnight, the cooled mixture was poured into water (30 mL) and extracted with EtOAc (30 mL*2). The combined organic phase was washed with brine, dried over Na₂SO₄, filtered and concentrated. The crude product was purified by chromatography (silica gel, 0-20%, MeOH in DCM) and then by prep-HPLC (C18, 30˜80% MeCN in H₂O with 0.1% HCOOH) to give N-[(1R)-1-(1,1-difluoro-2,3-dihydro-1H-inden-4-yl)ethyl]-4-methoxy-5-(1-methyl-6-oxo-1,6-dihydropyridin-3-yl)-2H-indazole-7-carboxamide (2 mg, 0.004 mmol, 1%). LC-MS (ESI) m/z 479 (M+H)⁺. ¹H NMR (400 MHz, MeOD) δ 8.43 (s, 2H), 7.97 (s, 1H), 7.88-7.78 (m, 2H), 7.59-7.46 (m, 2H), 7.37 (d, J=7.9 Hz, 2H), 6.61 (d, J=9.1 Hz, 1H), 5.39 (d, J=6.8 Hz, 1H), 4.27 (s, 3H), 3.65 (s, 3H), 3.00 (s, 2H), 2.60 (dd, J=14.3, 7.1 Hz, 2H), 1.59 (d, J=6.7 Hz, 3H).

### Example 18: Synthesis of N-[(1R)-1-(1,1-difluoro-2,3-dihydro-1H-inden-4-yl)ethyl]-4-methoxy-5-(morpholin-4-yl)-2H-indazole-7-carboxamide (the Title Compounds was Synthesized from Compound 5 in Example 16.)

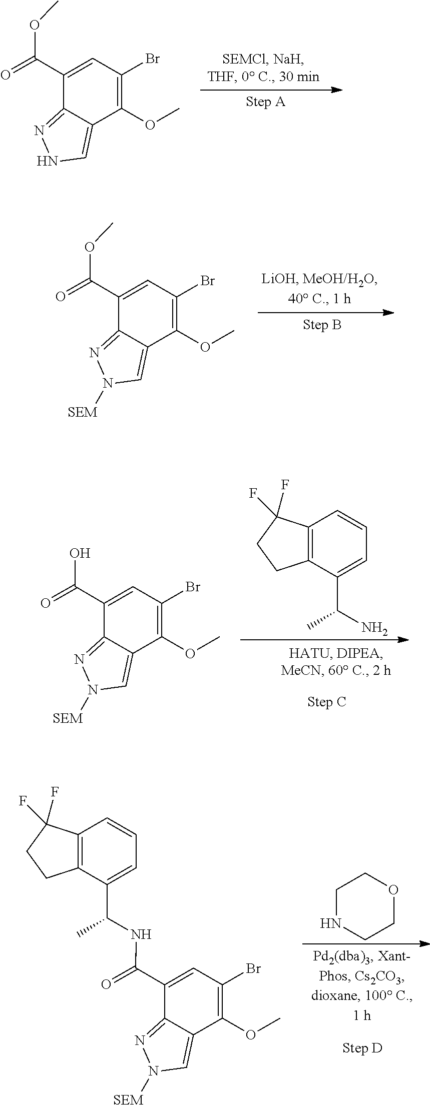 [SMILES: COC(=O)c1cc(Br)c(OC)c2c[nH]nc12 ; COC(=O)c1cc(Br)c(OC)c2cn(COCC[Si](C)(C)C)nc12 ; COc1c(Br)cc(C(=O)O)c2nn(COCC[Si](C)(C)C)cc12 ; C[C@@H](N)c1cccc2c1CCC2(F)F ; COc1c(Br)cc(C(=O)N[C@H](C)c2cccc3c2CCC3(F)F)c2nn(COCC[Si](C)(C)C)cc12 ; C1COCCN1]

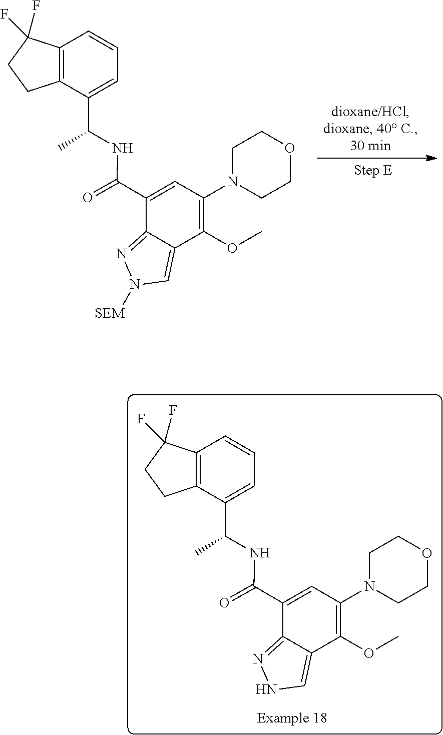 [SMILES: COc1c(N2CCOCC2)cc(C(=O)N[C@H](C)c2cccc3c2CCC3(F)F)c2nn(COCC[Si](C)(C)C)cc12 ; COc1c(N2CCOCC2)cc(C(=O)N[C@H](C)c2cccc3c2CCC3(F)F)c2n[nH]cc12]

### Step A. methyl 5-bromo-4-methoxy-2-{[2-(trimethylsilyl)ethoxy]methyl}-2H-indazole-7-carboxylate

To a stirred solution of methyl 5-bromo-4-methoxy-2H-indazole-7-carboxylate (900 mg, 3.16 mmol) in THF (20 mL) was added NaH (252 mg, 6.31 mmol) at 0° C. After stirring at 0° C. for 20 min, SEM-Cl (0.9 mL, 4.735 mmol) was added at 0° C. After stirring at 0° C. for 1 h, the mixture was poured into ice-water (50 mL) and extracted with EtOAc (50 mL*2). The combined organic phase was washed with brine, dried over Na₂SO₄, filtered and concentrated. The crude product was purified by chromatography (silica gel, 0-50%, EtOAc in PE) to give methyl 5-bromo-4-methoxy-2-{[2-(trimethylsilyl)ethoxy]methyl}-2H-indazole-7-carboxylate (700 mg, 1.68 mmol, 53%) as a white solid. LC-MS (ESI) m/z 415 (M+H)⁺.

### Step B. 5-bromo-4-methoxy-2-{[2-(trimethylsilyl)ethoxy]methyl}-2H-indazole-7-carboxylic acid

To a stirred solution of methyl 5-bromo-4-methoxy-2-{[2-(trimethylsilyl)ethoxy]methyl}-2H-indazole-7-carboxylate (200 mg, 0.48 mmol) in MeOH (5 mL) and water (5 mL) was added LiOH (202 mg, 4.81 mmol) at rt. After stirring at 40° C. for 2 h, the cooled mixture was concentrated to give crude 5-bromo-4-methoxy-2-{[2-(trimethylsilyl)ethoxy]methyl}-2H-indazole-7-carboxylic acid (193 mg, 0.48 mmol, 99.87%) as a white solid. LC-MS (ESI) m/z 401 (M+H)⁺.

### Step C. 5-bromo-N-[(1R)-1-(1,1-difluoro-2,3-dihydro-1H-inden-4-yl)ethyl]-4-methoxy-2-{[2-(trimethylsilyl)ethoxy]methyl}-2H-indazole-7-carboxamide

To a stirred solution of 5-bromo-4-methoxy-2-{[2-(trimethylsilyl)ethoxy]methyl}-2H-indazole-7-carboxylic acid (193 mg, 0.481 mmol) in acetonitrile (10 mL) and DMF (5 mL) was added (1R)-1-(1,1-difluoro-2,3-dihydro-1H-inden-4-yl)ethan-1-amine (142 mg, 0.72 mmol), HATU (365 mg, 0.96 mmol) and DIPEA (0.5 mL, 2.81 mmol) at rt. After stirring at 70° C. overnight, the cooled mixture was poured into water (30 mL) and extracted with EtOAc (30 mL*2). The combined organic phase was washed with brine, dried over Na₂SO₄, filtered and concentrated. The crude product was purified by chromatography (silica gel, 0-20%, MeOH in DCM) to give 5-bromo-N-[(1R)-1-(1,1-difluoro-2,3-dihydro-1H-inden-4-yl)ethyl]-4-methoxy-2-{[2-(trimethylsilyl)ethoxy]methyl}-2H-indazole-7-carboxamide (120 mg, 0.20 mmol, 43%) as a brown solid. LC-MS (ESI) m/z 456 (M+H)⁺.

### Step D. N-[(1R)-1-(1,1-difluoro-2,3-dihydro-1H-inden-4-yl)ethyl]-4-methoxy-5-(morpholin-4-yl)-2-{[2-(trimethylsilyl)ethoxy]methyl}-2H-indazole-7-carboxamide

To a stirred solution of 5-bromo-N-[(1R)-1-(1,1-difluoro-2,3-dihydro-1H-inden-4-yl)ethyl]-4-methoxy-2-{[2-(trimethylsilyl)ethoxy]methyl}-2H-indazole-7-carboxamide (120 mg, 0.20 mmol) in dioxane (5 mL) was added morpholine (0.1 mL, 0.62 mmol), Ru-phos (20 mg, 0.04 mmol), RuPhos Pd G3 (17 mg, 0.02 mmol) and Cs₂CO₃ (135 mg, 0.41 mmol) at rt. After stirring at 100° C. for 1 h, the cooled mixture was filtered and concentrated. The crude product was purified by chromatography (silica gel, 0-20%, MeOH in DCM) to give N-[(1R)-1-(1,1-difluoro-2,3-dihydro-1H-inden-4-yl)ethyl]-4-methoxy-5-(morpholin-4-yl)-2-{[2-(trimethylsilyl) ethoxy]methyl}-2H-indazole-7-carboxamide (55 mg, 0.09 mmol, 45%) as a yellow solid. LC-MS (ESI) m/z 587 (M+H)⁺.

### Step E. N-[(1R)-1-(1,1-difluoro-2,3-dihydro-1H-inden-4-yl)ethyl]-4-methoxy-5-(morpholin-4-yl)-2H-indazole-7-carboxamide

To a stirred solution of N-[(1R)-1-(1,1-difluoro-2,3-dihydro-1H-inden-4-yl)ethyl]-4-methoxy-5-(morpholin-4-yl)-2-{[2-(trimethylsilyl)ethoxy]methyl}-2H-indazole-7-carboxamide (55 mg, 0.094 mmol) in dioxane (5 mL) was added 4N dioxane/HCl (2 mL, 2M) at rt. After stirring at 40° C. for 1 h, the cooled mixture was concentrated. The crude product was purified by prep-HPLC (C18, 30˜80% MeCN in H₂O with 0.1% NH₃·H₂O) to give N-[(1R)-1-(1,1-difluoro-2,3-dihydro-1H-inden-4-yl)ethyl]-4-methoxy-5-(morpholin-4-yl)-2H-indazole-7-carboxamide (17 mg, 0.037 mmol, 40%) as a white solid. LC-MS (ESI) m/z 457 (M+H)⁺. ¹H NMR (400 MHz, DMSO) δ 12.87 (s, 1H), 8.93 (d, J=7.1 Hz, 1H), 8.26 (s, 1H), 7.87 (s, 1H), 7.61 (d, J=5.3 Hz, 1H), 7.43 (d, J=6.2 Hz, 2H), 5.30 (t, J=7.1 Hz, 1H), 4.21 (s, 3H), 3.80-3.76 (m, 4H), 3.15 (d, J=6.0 Hz, 2H), 3.03 (s, 4H), 2.68-2.59 (m, 2H), 1.54 (d, J=7.0 Hz, 3H).

### Example 19: Synthesis of N-[(1R)-1-(1,1-difluoro-2,3-dihydro-1H-inden-4-yl)ethyl]-5-[(dimethylamino)methyl]-4-methoxy-2H-indazole-7-carboxamide. (the Title Compounds was Synthesized from Compound 5 in Example 16.)

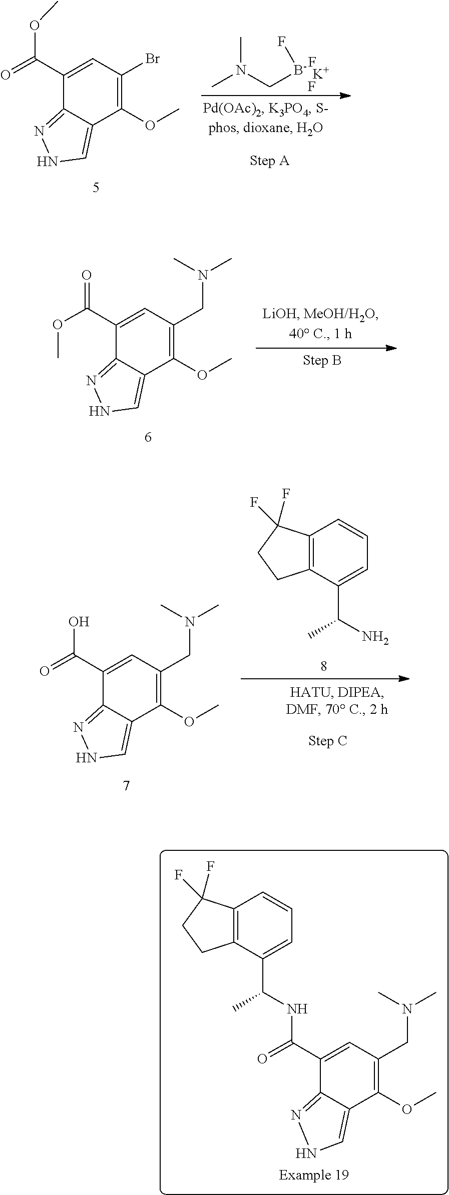

### Step A. methyl 5-[(dimethylamino)methyl]-4-methoxy-2H-indazole-7-carboxylate

To a stirred solution of methyl 5-bromo-4-methoxy-2H-indazole-7-carboxylate (500 mg, 1.75 mmol) in dioxane (4 mL) and water (1 mL) was added 1150655-04-1 (434 mg, 2.63 mmol), S-Phos (144 mg, 0.35 mmol), Pd(OAc)₂ (40 mg, 0.17 mmol) and K₃PO₄ (1.11 g, 5.26 mmol) at rt. After stirring at 100° C. for 2 h, the cooled mixture was filtered and concentrated. The crude product was purified by chromatography (silica gel, 0-40%, MeOH in DCM) to give methyl 5-[(dimethylamino)methyl]-4-methoxy-2H-indazole-7-carboxylate (85 mg, 0.32 mmol, 18%) as a brown solid. LC-MS (ESI) m/z 264 (M+H)⁺.

### Step B. 5-[(dimethylamino)methyl]-4-methoxy-2H-indazole-7-carboxylic acid

To a stirred solution of methyl 5-[(dimethylamino)methyl]-4-methoxy-2H-indazole-7-carboxylate (85 mg, 0.32 mmol) in MeOH (5 mL) and water (5 mL) was added LiOH (135 mg, 3.22 mmol) at rt. After stirring at 40° C. for 2 h, the cooled mixture was concentrated to give crude 5-[(dimethylamino)methyl]-4-methoxy-2H-indazole-7-carboxylic acid (80 mg, 0.32 mmol, 99%) as a white solid. LC-MS (ESI) m/z 250 (M+H)⁺.

### Step C. N-[(1R)-1-(1,1-difluoro-2,3-dihydro-1H-inden-4-yl)ethyl]-5-[(dimethylamino) methyl]-4-methoxy-2H-indazole-7-carboxamide

To a stirred solution of 5-[(dimethylamino)methyl]-4-methoxy-2H-indazole-7-carboxylic acid (40 mg, 0.16 mmol) in DMF (10 mL) was added (1R)-1-(1,1-difluoro-2,3-dihydro-1H-inden-4-yl)ethan-1-amine (47 mg, 0.24 mmol), DIPEA (0.2 mL, 1.12 mmol) and HATU (122 mg, 0.32 mmol) at rt. After stirring at 70° C. for 2 h, the cooled mixture was poured into water (50 mL) and extracted with EtOAc (50 mL*2). The combined organic phase was washed with brine, dried over Na₂SO₄, filtered and concentrated. The crude product was purified by prep-HPLC (C18, 30˜80% MeCN in H₂O with 0.1% HCOOH) to give N-[(1R)-1-(1,1-difluoro-2,3-dihydro-1H-inden-4-yl)ethyl]-5-[(dimethylamino)methyl]-4-methoxy-2H-indazole-7-carboxamide (8 mg, 0.019 mmol, 12%) as a white solid. LC-MS (ESI) m/z 429 (M+H)⁺. ¹H NMR (400 MHz, MeOD) δ 8.51 (s, 1H), 7.95 (s, 1H), 7.59 (d, J=6.9 Hz, 1H), 7.42-7.35 (m, 2H), 5.40 (q, J=7.0 Hz, 1H), 4.46 (s, 3H), 4.30 (s, 2H), 3.25 (d, J=7.0 Hz, 1H), 3.16-3.09 (m, 1H), 2.81 (s, 6H), 2.65-2.54 (m, 2H), 1.61 (d, J=7.0 Hz, 3H).

### Example 20: Synthesis of N-[(1R)-1-[3-(1,1-difluoro-2-hydroxyethyl)phenyl]ethyl]-5-[(dimethylamino)methyl]-4-methoxy-2H-indazole-7-carboxamide. (the Title Compounds was Synthesized from Compound 7 in Example 19.)

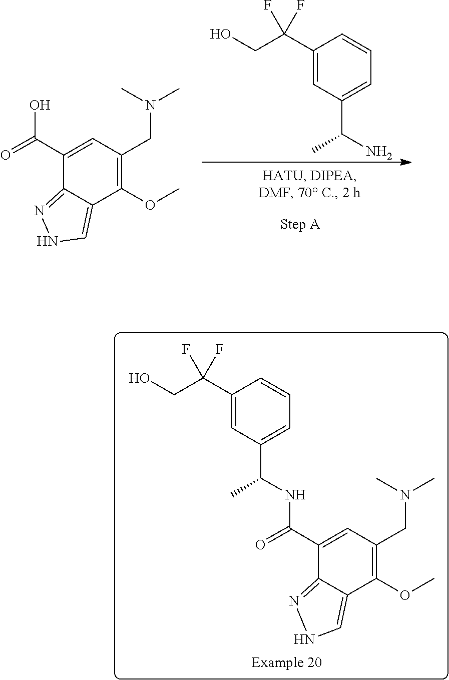 [SMILES: COc1c(CN(C)C)cc(C(=O)O)c2n[nH]cc12 ; C[C@@H](N)c1cccc(C(F)(F)CO)c1 ; COc1c(CN(C)C)cc(C(=O)N[C@H](C)c2cccc(C(F)(F)CO)c2)c2n[nH]cc12]

### Step A. N-[(1R)-1-[3-(1,1-difluoro-2-hydroxyethyl)phenyl]ethyl]-5-[(dimethylamino) methyl]-4-methoxy-2H-indazole-7-carboxamide

To a stirred solution of 5-[(dimethylamino)methyl]-4-methoxy-2H-indazole-7-carboxylic acid (40 mg, 0.16 mmol) in DMF (10 mL) was added 2-{3-[(1R)-1-aminoethyl]phenyl}-2,2-difluoroethan-1-ol (32 mg, 0.16 mmol), DIPEA (0.2 mL, 1.12 mmol) and HATU (122 mg, 0.32 mmol) at rt. After stirring at 70° C. for 2 h, the cooled mixture was poured into water (50 mL) and extracted with EtOAc (50 mL*2). The combined organic phase was washed with brine, dried over Na₂SO₄, filtered and concentrated. The crude product was purified by prep-TLC (10% MeOH in DCM) and then by prep-HPLC (C18, 30˜80% MeCN in H₂O with 0.1% HCOOH) to give N-[(1R)-1-[3-(1,1-difluoro-2-hydroxyethyl)phenyl]ethyl]-5-[(dimethylamino) methyl]-4-methoxy-2H-indazole-7-carboxamide (7 mg, 0.016 mmol, 10%) as a white solid. LC-MS (ESI) m/z 433 (M+H)⁺. ¹H NMR (400 MHz, MeOD) δ 8.53 (s, 1H), 7.92 (s, 1H), 7.61 (s, 1H), 7.57 (d, J=7.0 Hz, 1H), 7.47-7.41 (m, 2H), 5.37 (d, J=7.1 Hz, 1H), 4.48 (s, 3H), 4.38 (s, 2H), 3.88 (t, J=13.5 Hz, 2H), 2.88 (s, 6H), 1.63 (d, J=7.1 Hz, 3H).

### Example 21: Synthesis of (R)—N-(1-(1,1-difluoro-2,3-dihydro-1H-inden-4-yl)ethyl)-3-methyl-5-(1-methylcyclopropyl)-4-oxo-4,5-dihydro-1H-pyrrolo[3,2-c]pyridine-7-carboxamide. (the Title Compounds was Synthesized from Compound 5 in Example 16.)

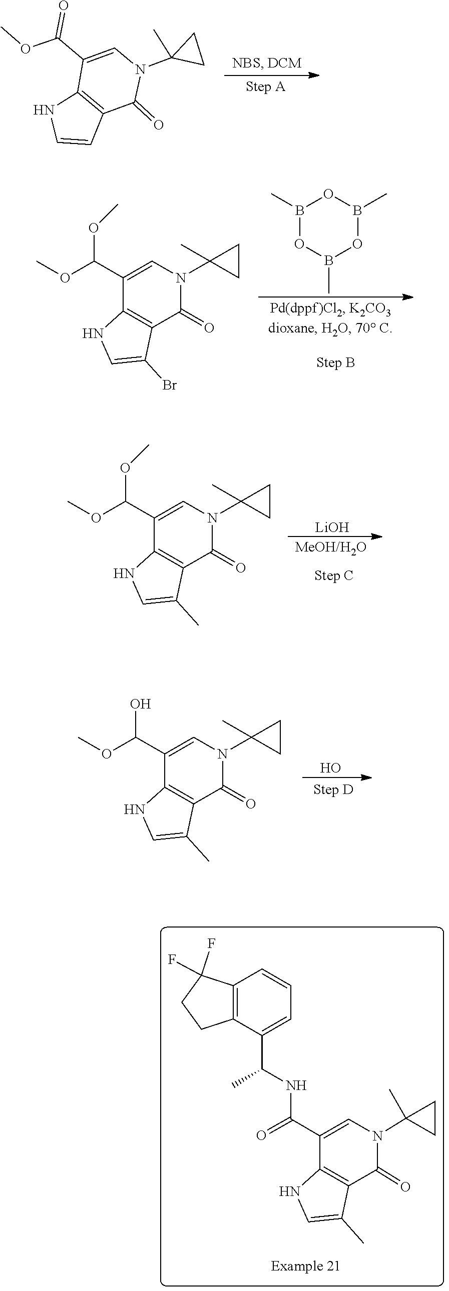

### Step A. methyl 3-bromo-5-(1-methylcyclopropyl)-4-oxo-4,5-dihydro-1H-pyrrolo[3,2-c]pyridine-7-carboxylate

To a stirred solution of methyl 5-(1-methylcyclopropyl)-4-oxo-1H,4H,5H-pyrrolo[3,2-c]pyridine-7-carboxylate (60 mg, 0.24 mmol) in AcOH (1 mL) and DCM (3 mL) was added NBS (47.7 mg, 0.27 mmol). The reaction mixture was stirred at room temperature for 2 h. The reaction mixture was poured into water and extracted with EtOAc. The organic phase was washed with aqueous NaHCO₃, dried over Na₂SO₄ and concentrated under vacuum to give the residue and the residue was purified by column chromatography (silica gel, 24 g, 0-100% EtOAc in PE) to give methyl3-bromo-5-(1-methylcyclopropyl)-4-oxo-1H,4H,5H-pyrrolo[3,2-c]pyridine-7-carboxylate (60 mg, 0.19 mmol, 75.7%) as a colorless solid. LCMS: m/z 326 (M+H)⁺.

### Step B. methyl 3-methyl-5-(1-methylcyclopropyl)-4-oxo-4,5-dihydro-1H-pyrrolo[3,2-c]pyridine-7-carboxylate

To a stirred solution of methyl 3-bromo-5-(1-methylcyclopropyl)-4-oxo-1H,4H,5H-pyrrolo[3,2-c]pyridine-7-carboxylate (60 mg, 0.185 mmol) in dioxane (3 mL) and H₂O (1 mL) was added trimethyl-1,3,5,2,4,6-trioxatriborinane (0.08 mL, 0.55 mmol), Pd(dppf)Cl₂ (13.5 mg, 0.02 mmol) and K₂CO₃ (76.5 mg, 0.55 mmol). The reaction mixture was stirred at 70° C. for 5 h. The reaction mixture was filtered and the filtrate was concentrated under vacuum to give the residue and the residue was purified by column chromatography (silica gel, 24 g, 0-80% EtOAc in PE) to give methyl 3-methyl-5-(1-methylcyclopropyl)-4-oxo-1H,4H,5H-pyrrolo[3,2-c]pyridine-7-carboxylate (10 mg, 0.04 mmol, 20.8%) as a light yellow solid. LCMS: m/z 261 (M+H)⁺.

### Step C. 3-methyl-5-(1-methylcyclopropyl)-4-oxo-4,5-dihydro-1H-pyrrolo[3,2-c]pyridine-7-carboxylic acid

To a stirred solution of methyl 3-methyl-5-(1-methylcyclopropyl)-4-oxo-1H,4H,5H-pyrrolo[3,2-c]pyridine-7-carboxylate (10 mg, 0.04 mmol) in MeOH (3 mL) was added LiOH (15.37 mg, 0.38 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give crude product 3-methyl-5-(1-methylcyclopropyl)-4-oxo-1H,4H,5H-pyrrolo[3,2-c]pyridine-7-carboxylic acid (6 mg, 0.02 mmol, 63.4%) as a light yellow solid. LCMS: m/z 247 (M+H)⁺.

### Step D. (R)—N-(1-(1,1-difluoro-2,3-dihydro-1H-inden-4-yl)ethyl)-3-methyl-5-(1-methylcyclopropyl)-4-oxo-4,5-dihydro-1H-pyrrolo[3,2-c]pyridine-7-carboxamide

To a stirred solution of 3-methyl-5-(1-methylcyclopropyl)-4-oxo-1H,4H,5H-pyrrolo[3,2-c]pyridine-7-carboxylic acid (6 mg, 0.024 mmol) in DMF (3 mL) was added (1R)-1-(1,1-difluoro-2,3-dihydro-1H-inden-4-yl)ethan-1-amine (5.77 mg, 0.03 mmol), DIPEA (15.84 mg, 0.12 mmol) and HATU (13.9 mg, 0.037 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (100% EtOAc) to give the crude product and the crude product was purified by prep-HPLC to give N-[(1R)-1-(1,1-difluoro-2,3-dihydro-1H-inden-4-yl)ethyl]-3-methyl-5-(1-methylcyclopropyl)-4-oxo-1H,4H,5H-pyrrolo[3,2-c]pyridine-7 carboxamide (5 mg, undefined, 0.01 mmol, 48.2%) as a white solid. ¹H NMR (400 MHz, MeOD) δ 8.08 (s, 1H), 7.62-7.51 (m, 1H), 7.48-7.31 (m, 2H), 6.35 (d, J=0.9 Hz, 1H), 5.40-5.14 (m, 1H), 3.10 (ddd, J=16.9, 9.3, 5.3 Hz, 1H), 2.60 (tt, J=14.4, 7.2 Hz, 2H), 2.33 (d, J=0.7 Hz, 3H), 1.58 (d, J=7.1 Hz, 3H), 1.55 (s, 3H), 1.18 (d, J=6.8 Hz, 2H), 1.04 (t, J=5.9 Hz, 2H). LCMS: m/z 426 (M+H)⁺.

### Example 22: Synthesis of N-[(1R)-1-(1,1-difluoro-2,3-dihydro-1H-inden-4-yl)ethyl]-5-(1H-imidazol-5-yl)-4-methoxy-2H-indazole-7-carboxamide. (the Title Compounds was Synthesized from Compound 5 in Example 16.)

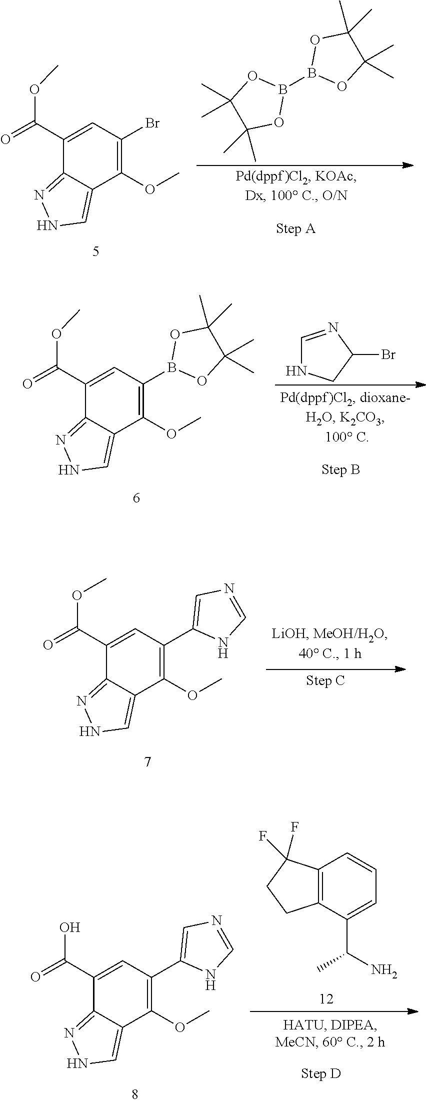

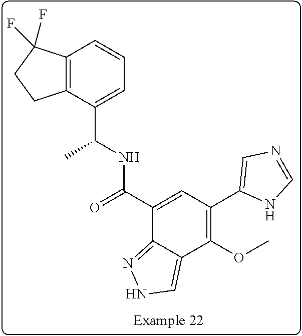 [SMILES: COc1c(-c2cnc[nH]2)cc(C(=O)N[C@H](C)c2cccc3c2CCC3(F)F)c2n[nH]cc12]

### Step A. methyl 4-methoxy-5-(tetramethyl-1,3,2-dioxaborolan-2-yl)-2H-indazole-7-carboxylate

To a stirred solution of methyl 5-bromo-4-methoxy-2H-indazole-7-carboxylate (500 mg, 1.75 mmol) in dioxane (20 mL) was added KOAc (516 mg, 5.26 mmol), Pd(dppf)Cl₂ (128 mg, 0.17 mmol) and 4,4,5,5-tetramethyl-2-(tetramethyl-1,3,2-dioxaborolan-2-yl)-1,3,2-dioxaborolane (0.7 mL, 2.63 mmol) at rt. After stirring at 100° C. overnight, the cooled mixture was concentrated to give crude methyl 4-methoxy-5-(tetramethyl-1,3,2-dioxaborolan-2-yl)-2H-indazole-7-carboxylate (400 mg, 1.21 mmol, 69%) as a brown solid. LCMS: m/z 333 (M+H)⁺.

### Step B. methyl 5-(1H-imidazol-5-yl)-4-methoxy-2H-indazole-7-carboxylate

To a stirred solution of methyl 4-methoxy-5-(tetramethyl-1,3,2-dioxaborolan-2-yl)-2H-indazole-7-carboxylate (300 mg, 0.91 mmol) in H₂O (3 mL)/dioxane (5 mL) was added 4-bromo-1H-imidazole (159 mg, 1.08 mmol), Pd(dppf)Cl₂ (66 mg, 0.09 mmol) and K₂CO₃ (374 mg, 2.71 mmol) at rt. After stirring at 100° C. for 3 h, the cooled mixture was poured into water (20 mL) and extracted with DCM (20 mL*2). The combined organic phase was washed with brine, dried with Na₂SO₄, filtered and concentrated. The residue was purified by chromatography (silica gel, 0-20%, MeOH in DCM) to give methyl 5-(1H-imidazol-5-yl)-4-methoxy-2H-indazole-7-carboxylate (100 mg, 0.367 mmol, 40%) as a brown solid. LCMS: m/z 273 (M+H)⁺.

### Step C. 5-(1H-imidazol-5-yl)-4-methoxy-2H-indazole-7-carboxylic acid

To a stirred mixture of methyl 5-(1H-imidazol-5-yl)-4-methoxy-2H-indazole-7-carboxylate (100 mg, 0.37 mmol) in MeOH (3 mL)/H₂O (3 mL) was added LiOH (88 mg, 3.67 mmol) at rt. After stirring at rt for 2 h, the mixture was concentrated to give crude of 5-(1H-imidazol-5-yl)-4-methoxy-2H-indazole-7-carboxylic acid (90 mg, 0.35 mmol, 95%) as a white solid. LCMS: m/z 259 (M+H)⁺.

### Step D. N-[(1R)-1-(1,1-difluoro-2,3-dihydro-1H-inden-4-yl)ethyl]-5-(1H-imidazol-5-yl)-4-methoxy-2H-indazole-7-carboxamide

To a stirred mixture of 5-(1H-imidazol-5-yl)-4-methoxy-2H-indazole-7-carboxylic acid (40 mg, 0.155 mmol) in DMF (5 mL) was added (1R)-1-(1,1-difluoro-2,3-dihydro-1H-inden-4-yl)ethan-1-amine (36 mg, 0.19 mmol), HATU (118 mg, 0.31 mmol) and DIPEA (0.7 mL, 0.77 mmol) at rt. After stirring at 60° C. for 2 h, the cooled mixture was poured into water (20 mL) and extracted with EtOAc (20 mL*2). The combined organic phase was washed with brine, dried with Na₂SO₄, filtered and concentrated. The residue was purified by prep-TLC (DCM:MeOH=10:1) and then prep-HPLC (C18, 40˜90% MeCN in H₂O with 0.1% HCOOH) to give N-[(1R)-1-(1,1-difluoro-2,3-dihydro-1H-inden-4-yl)ethyl]-5-(1H-imidazol-5-yl)-4-methoxy-2H-indazole-7-carboxamide (2 mg, 0.005 mmol, 3%) as a white solid. LCMS: m/z 438 (M+H)⁺. ¹H NMR (400 MHz, MeOD) δ 8.46 (d, J=17.3 Hz, 2H), 8.14 (s, 1H), 7.66-7.59 (m, 2H), 7.41-7.34 (m, 2H), 5.40 (q, J=7.0 Hz, 1H), 4.42 (s, 3H), 3.27 (s, 1H), 3.12 (dd, J=15.9, 5.8 Hz, 1H), 2.61 (dt, J=20.7, 6.9 Hz, 2H), 1.62 (d, J=7.0 Hz, 3H).

### Example 23: Synthesis of N-[(1R)-1-[3-(1,1-difluoro-2-hydroxyethyl)phenyl]ethyl]-5-(1H-imidazol-5-yl)-4-methoxy-2H-indazole-7-carboxamide. (the Title Compound was Synthesized from Compound 8 in Example 22.)

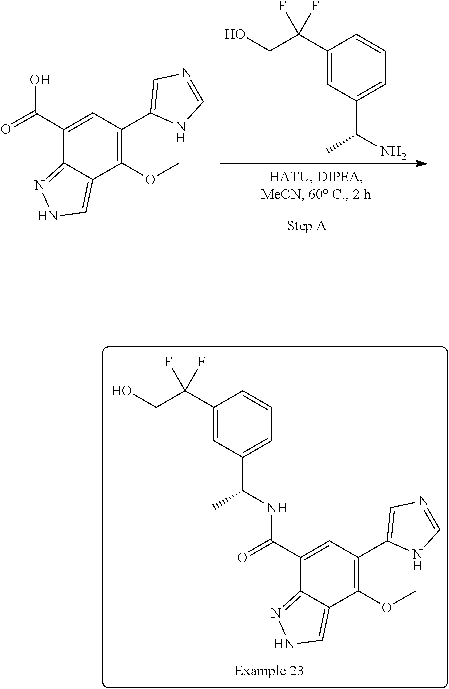 [SMILES: COc1c(-c2cnc[nH]2)cc(C(=O)O)c2n[nH]cc12 ; C[C@@H](N)c1cccc(C(F)(F)CO)c1 ; COc1c(-c2cnc[nH]2)cc(C(=O)N[C@H](C)c2cccc(C(F)(F)CO)c2)c2n[nH]cc12]

### Step A. N-[(1R)-1-[3-(1,1-difluoro-2-hydroxyethyl)phenyl]ethyl]-5-(1H-imidazol-5-yl)-4-methoxy-2H-indazole-7-carboxamide

To a stirred mixture of 5-(1H-imidazol-5-yl)-4-methoxy-2H-indazole-7-carboxylic acid (40 mg, 0.15 mmol, from N201220-7) in DMF (3 mL) was added 2-{3-[(1R)-1-aminoethyl]phenyl}-2,2-difluoroethan-1-ol (37 mg, 0.18 mmol), HATU (118 mg, 0.31 mmol) and DIPEA (0.7 mL, 0.77 mmol) at rt. After stirring at 60° C. for 2 h, the cooled mixture was poured into water (20 mL) and extracted with EtOAc (20 mL*2). The combined organic phase was washed with brine, dried with Na₂SO₄, filtered and concentrated. The residue was purified by prep-TLC (DCM:MeOH=8:1) and then by prep-HPLC (C18, 40˜90% MeCN in H₂O with 0.1% HCOOH) to give N-[(1R)-1-[3-(1,1-difluoro-2-hydroxyethyl)phenyl]ethyl]-5-(1H-imidazol-5-yl)-4-methoxy-2H-indazole-7-carboxamide (2 mg, 0.005 mmol, 3%) as a white solid. LCMS: m/z 442 (M+H)⁺. ¹H NMR (400 MHz, MeOD) δ 8.50 (s, 1H), 8.37 (s, 2H), 7.71 (s, 1H), 7.63 (s, 1H), 7.58 (d, J=7.3 Hz, 1H), 7.47-7.41 (m, 2H), 5.39-5.34 (m, 1H), 4.44 (s, 3H), 3.88 (t, J=13.5 Hz, 2H), 1.64 (d, J=7.0 Hz, 3H).

### Example 24: Synthesis of 5-(1-aminoethyl)-N—((R)-1-(1,1-difluoro-2,3-dihydro-1H-inden-4-yl)ethyl)-4-methoxy-2H-indazole-7-carboxamide. (the Title Compounds was Synthesized from Compound 5 in Example 16.)

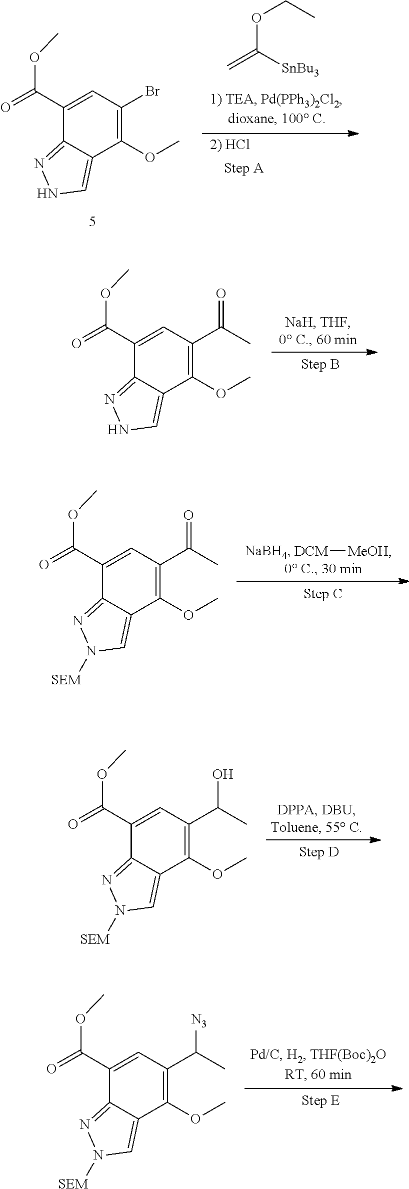

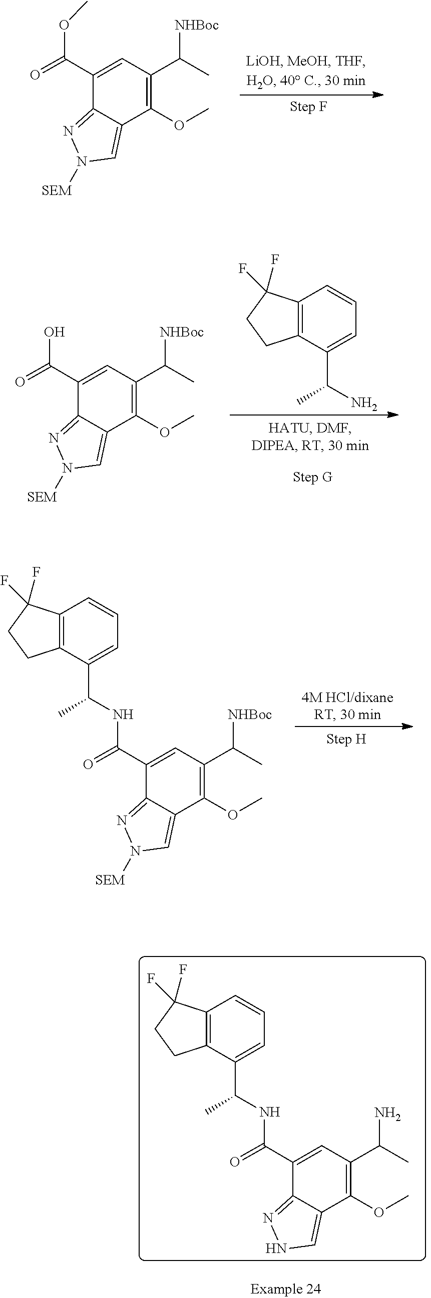 [SMILES: COC(=O)c1cc(C(C)NC(=O)OC(C)(C)C)c(OC)c2cn(COCC[Si](C)(C)C)nc12 ; COc1c(C(C)NC(=O)OC(C)(C)C)cc(C(=O)O)c2nn(COCC[Si](C)(C)C)cc12 ; C[C@@H](N)c1cccc2c1CCC2(F)F ; COc1c(C(C)NC(=O)OC(C)(C)C)cc(C(=O)N[C@H](C)c2cccc3c2CCC3(F)F)c2nn(COCC[Si](C)(C)C)cc12 ; COc1c(C(C)N)cc(C(=O)N[C@H](C)c2cccc3c2CCC3(F)F)c2n[nH]cc12]

### Step A. methyl 5-acetyl-4-methoxy-2H-indazole-7-carboxylate

To a stirred solution of methyl 5-bromo-4-methoxy-2H-indazole-7-carboxylate (1.00 g, 3.51 mmol) in dioxane (15 mL) was added TEA (1.4 mL, 10.5 mmol) and Pd(PPh₃)₂Cl₂ (245 mg, 351 mmol) at rt. The resulting mixture was stirred at 100° C. under nitrogen atmosphere for 4 h. The cooled reaction mixture was diluted with 2 mol/L HCl (50 mL) and extracted with EtOAc (30 mL×3). The combined organic phase was washed with brine, dried over Na₂SO₄, filtered and concentrated. The residue was purified chromatography (silica gel, 0-40%, EtOAc in PE) to give methyl 5-acetyl-4-methoxy-2H-indazole-7-carboxylate (420 mg, 1.69 mmol, 48.2%) as a white solid. LC-MS (ESI) m/z 249 (M+H)⁺.

### Step B. methyl 5-acetyl-4-methoxy-2-((2-(trimethylsilyl)ethoxy)methyl)-2H-indazole-7-carboxylate

To a stirred solution of methyl 5-acetyl-4-methoxy-2H-indazole-7-carboxylate (500 mg, 2.01 mmol) in THE (10 mL) was added NaH (241 mg 6.04 mmol) at 0° C. After stirring at 0° C. for 20 mins. SEMCl (503 mg, 3.02 mmol) was added. The resulting mixture was stirred at 0° C. for 30 mins. The reaction mixture was poured into saturated NaHCO₃ solution, extracted with EtOAc (50 mL*2). The combined organic phase was washed with brine, dried over Na₂SO₄, filtered and concentrated. The crude product was purified by chromatography (silica gel, 0-5% MeOH in DCM with 0.5% TEA) to give methyl 5-acetyl-4-methoxy-2-((2-(trimethylsilyl)ethoxy)methyl)-2H-indazole-7-carboxylate (260 mg, 0.69 mmol. 34.1%) as a brown solid. LC-MS (ESI) m/z 378.9 (M+1)⁺.

### Step C. methyl5-(1-hydroxyethyl)-4-methoxy-2-((2(trimethylsilyl)ethoxy)methyl)-2H-indazole-7-carboxylate

To a stirred solution of methyl-5-acetyl-4-methoxy-2-((2-(trimethylsilyl)ethoxy) methyl)-2H-indazole-7-carboxylate (200 mg, 0.529 mmol) in DCM/MeOH (10 mL/2 mL) was added NaBH₄ (241 mg 6.04 mmol) at 0° C. After stirring at 0° C. for 20 min, the reaction mixture was quenched with water and extracted with DCM (30 mL*2). The combined organic phase was washed with brine, dried over Na₂SO₄, filtered and concentrated to give methyl5-(1-hydroxyethyl)-4-methoxy-2-((2 (trimethylsilyl)ethoxy)methyl)-2H-indazole-7-carboxylate. (200 mg, 0.526 mmol) as a brown oil. LC-MS (ESI) m/z 381.1 (M+1)⁺.

### Step D. methyl 5-(1-azidoethyl)-4-methoxy-2-((2-(trimethylsilyl)ethoxy)methyl)-2H-indazole-7-carboxylate

To a stirred solution of methyl 5-(1-hydroxyethyl)-4-methoxy-2-{[2-(trimethylsilyl) ethoxy] methyl}-2H-indazole-7-carboxylate (200 mg, 0.526 mmol) in toluene (8 mL) was added DBU (240 mg 1.57 mmol) and DPPA (275 mg, 434 mg) at rt. The resulting mixture was stirred at 55° C. for 2 h. The reaction mixture was quenched with water and extracted with EtOAc (50 mL*2). The combined organic phase was washed with brine, dried over Na₂SO₄, filtered and concentrated. The crude product was purified by chromatography (silica gel, 0-30% EtOAc in PE) to give methyl 5-(1-azidoethyl)-4-methoxy-2-((2-(trimethylsilyl)ethoxy)methyl)-2H-indazole-7-carboxylate (35 mg, 0.086 mmol, 16.4%) as a white solid. LC-MS (ESI) m/z 406.1 (M+1)⁺.

### Step E. methyl 5-(1-((tert-butoxycarbonyl)amino)ethyl)-4-methoxy-2-((2-(trimethylsilyl) ethoxy)methyl)-2H-indazole-7-carboxylate

To a stirred solution of methyl 5-(1-azidoethyl)-4-methoxy-2-((2(trimethylsilyl)ethoxy) methyl)-2H-indazole-7-carboxylate (35 mg, 0.086 mmol) in THE (8 mL) was added Boc₂O (94.3 mg 0.432 mmol) and Pd/C (50 mg, 20% wet) at rt. The resulting mixture was stirred at RT for 1 h. The reaction mixture was filtered. The filtrate was concentrated. The crude product was purified by prep-TLC (PE/EA=3/1) to give methyl 5-(1-((tert-butoxycarbonyl)amino)ethyl)-4-methoxy-2-((2-(trimethylsilyl)ethoxy)methyl)-2H-indazole-7-carboxylate (30 mg, 0.063 mmol, 72.4%) as a white solid. LC-MS (ESI) m/z 480.1 (M+1)⁺.

### Step F. 5-(1-((tert-butoxycarbonyl)amino)ethyl)-4-methoxy-2-((2-(trimethylsilyl)ethoxy) methyl)-2H-indazole-7-carboxylic acid

To a stirred solution of methyl 5-(1-((tert-butoxycarbonyl)amino)ethyl)-4-methoxy-2-((2-(trimethylsilyl)ethoxy)methyl)-2H-indazole-7-carboxylate (25 mg, 0.052 mmol) in MeOH/THF/H₂O (2:2:1) (8 mL) was added LiOH (11.0 mg 0.26 mmol) at rt. The resulting mixture was stirred at 40° C. for 40 mins. The reaction mixture was concentrated. The residue was diluted with water (3 mL) and adjust pH to 6 with 1N HCl. The mixture was extracted with EtOAc (20 mL*2). The combined organic phase was washed with brine, dried over Na₂SO₄, filtered and concentrated to give 5-(1-((tert-butoxycarbonyl)amino)ethyl)-4-methoxy-2-((2-(trimethylsilyl)ethoxy)methyl)-2H-indazole-7-carboxylic acid (20 mg, 0.043 mmol, 82.4%) as a white solid. LC-MS (ESI) m/z 466.1 (M+1)⁺.

### Step G. tert-butyl (1-(7-(((R)-1-(1,1-difluoro-2,3-dihydro-1H-inden-4-yl)ethyl)carbamoyl)-4-methoxy-2-((2-(trimethylsilyl)ethoxy)methyl)-2H-indazol-5-yl)ethyl)carbamate

To a stirred solution of 5-(1-((tert-butoxycarbonyl)amino)ethyl)-4-methoxy-2-((2-(trimethylsilyl)ethoxy)methyl)-2H-indazole-7-carboxylic acid (20 mg, 0.043 mmol) in DMF (8 mL) was added DIPEA (17 mg 0.129 mmol) and (1R)-1-(1,1-difluoro-2,3-dihydro-1H-inden-4-yl)ethan-1-amine (13 mg, 0.065 mmol) at rt before HATU (25 mg, 0.065 mmol) was added. The resulting mixture was stirred at rt for 30 min and quenched with saturated NaHCO₃ solution, extracted with EtOAc (20 mL*2). The combined organic phase was washed with brine, dried over Na₂SO₄, filtered and concentrated. The crude product was purified by prep-TLC (PE/EA=3/1) to give tert-butyl (1-(7-(((R)-1-(1,1-difluoro-2,3-dihydro-1H-inden-4-yl)ethyl) carbamoyl)-4-methoxy-2-((2-(trimethylsilyl)ethoxy)methyl)-2H-indazol-5-yl)ethyl)carbamate (18 mg, 0.028 mmol, 65%) as a white solid. LC-MS (ESI) m/z 645.1 (M+1)⁺.

### Step H. 5-(1-aminoethyl)-N—((R)-1-(1,1-difluoro-2,3-dihydro-1H-inden-4-yl)ethyl)-4-methoxy-2H-indazole-7-carboxamide

To a stirred solution of tert-butyl (1-(7-(((R)-1-(1,1-difluoro-2,3-dihydro-1H-inden-4-yl)ethyl)carbamoyl)-4-methoxy-2-((2-(trimethylsilyl)ethoxy)methyl)-2H-indazol-5-yl)ethyl) carbamate (18 mg, 0.028 mmol) in dioxane (2 mL) was added 4 mol/L 4 NHCl/dioxane (0.07 mL, 0.280 mmol). The resulting mixture was stirred at rt for 30 min. The reaction mixture was quenched with saturated NaHCO₃ solution and extracted with EtOAc (20 mL*2). The combined organic phase was washed with brine, dried over Na₂SO₄, filtered and concentrated. The crude product was purified by prep-TLC (DCM/MeOH=10/1) to give 5-(1-aminoethyl)-N-[(1R)-1-(1,1-difluoro-2,3-dihydro-1H-inden-4-yl)ethyl]-4-methoxy-2H-indazole-7-carboxamide (5 mg, 0.012 mmol, 43.2%) as a white solid. LC-MS (ESI) m/z 413.1 (M−1). ¹H NMR (400 MHz, DMSO) δ 13.13 (s, 1H), 8.93 (d, J=14.3 Hz, 1H), 8.49 (s, 1H), 8.28 (d, J=52 Hz, 3H), 7.68 (s, 1H), 7.42 (d, J=6.5 Hz, 2H), 5.39-5.16 (m, 1H), 4.70 (s, 1H), 4.33 (s, 3H), 3.17 (d, J=1.1 Hz, 2H), 2.85-2.54 (m, 2H), 1.74-1.46 (m, 6H),

### Example 25: Synthesis of (R)-5-((1-(3-(1,1-difluoro-2-hydroxyethyl)phenyl)ethyl)amino)-1,7-dimethyl-3-(tetrahydro-2H-pyran-4-yl)-3,4-dihydropyrimido[4,5-d]pyrimidin-2(1H)-one

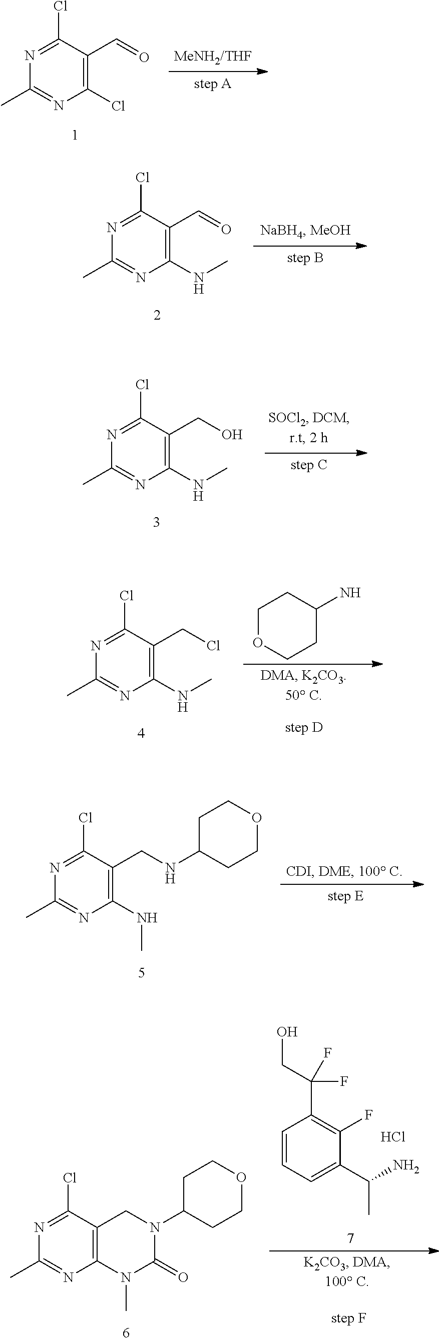

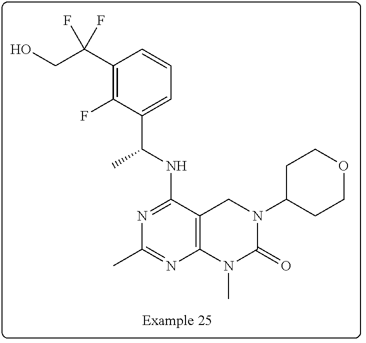 [SMILES: Cc1nc(N[C@H](C)c2cccc(C(F)(F)CO)c2F)c2c(n1)N(C)C(=O)N(C1CCOCC1)C2]

### Step A. 4-chloro-2-methyl-6-(methylamino)pyrimidine-5-carbaldehyde

To a stirred solution of 4,6-dichloro-2-methylpyrimidine-5-carbaldehyde (4 g, 20.9 mmol) in THE (40 mL) was added 2 mol/l MeNH₂/THF (11.6 ml, 23.2 mmol) at rt. After stirring at rt for 1 h, the reaction mixture was diluted water (100 mL). The following mixture was extracted with EtOAc (30 mL×2). The combined organic phase was washed with brine (50 ml), dried with anhydrous Na₂SO₄ and concentrated to afford 4-chloro-2-methyl-6-(methylamino)pyrimidine-5-carbaldehyde (3.8 g, 19.4 mmol, 92.9%) as a yellow solid. LC-MS (ESI) m/z 186 (M+H)⁺.

### Step B. (4-chloro-2-methyl-6-(methylamino)pyrimidin-5-yl)methanol

To a stirred solution of 4-chloro-2-methyl-6-(methylamino)pyrimidine-5-carbaldehyde (3.8 g, 19.4 mmol) in MeOH (40 mL) was added NaBH₄ (810 mg, 21.4 mmol) at rt. After stirring at rt for 60 mins, the mixture was quenched with water (10 mL) and concentrated. The residue was extracted with DCM (50 mL×2). The combined organic phase was washed with brine, dried over Na₂SO₄, filtered and concentrated to afford (4-chloro-2-methyl-6-(methylamino)pyrimidin-5-yl)methanol (3.6 g, 0.184 mmol, 98.2%) as a yellow solid. LC-MS (ESI) m/z 188 (M+H)⁺.

### Step C. 6-chloro-5-(chloromethyl)-N,2-dimethylpyrimidin-4-amine

To a stirred solution of (4-chloro-2-methyl-6-(methylamino)pyrimidin-5-yl)methanol (120 mg, 0.428 mmol) in DCM (30 mL) was added SOCl₂ (2.3 mL, 31.9 mmol) at rt. After stirring at rt for 2 h, the mixture was poured into NaHCO₃ (30 mL) and extracted with DCM (50 mL×2). The combined organic phase was washed with brine, dried over Na₂SO₄, filtered and concentrated. The crude product was purified by chromatography (silica gel, 0-10%, MeOH in DCM) to give 6-chloro-5-(chloromethyl)-N,2-dimethylpyrimidin-4-amine (3.00 g, 14.557 mmol, 91.0%) as a yellow solid. LC-MS (ESI) m/z 206 (M+H)⁺.

### Step D. 6-chloro-N,2-dimethyl-5-(((tetrahydro-2H-pyran-4-yl)amino)methyl)pyrimidin-4-amine

To a stirred solution of 6-chloro-5-(chloromethyl)-N,2-dimethylpyrimidin-4-amine (3 g, 14.5 mmol) in DMA (30 mL) was added K₂CO₃ (2.3 mL, 31.9 mmol) and tetrahydro-2H-pyran-4-amine (1.55 g, 15.2 mmol) at rt. The resulting mixture was stirred at 50° C. for 2 h. After cooled down to rt, the mixture was diluted with water (100 mL), extracted with EtOAc (30 mL×3). The combined organic phase was washed with brine, dried over Na₂SO₄, filtered and concentrated. The crude product was purified by chromatography (silica gel, 0-10%, MeOH in DCM) to give 6-chloro-N,2-dimethyl-5-(((tetrahydro-2H-pyran-4-yl)amino)methyl)pyrimidin-4-amine (1.8 g, 6.65 mmol, 45.6%) as a brown solid. LC-MS (ESI) m/z 271 (M+H)⁺.

### Step E. 5-chloro-1,7-dimethyl-3-(tetrahydro-2H-pyran-4-yl)-3,4-dihydropyrimido[4,5-d]pyrimidin-2(1H)-one

To a stirred solution of 6-chloro-N,2-dimethyl-5-(((tetrahydro-2H-pyran-4-yl)amino) methyl)pyrimidin-4-amine (1.8 g, 6.65 mmol) in anhydrous DME (20 mL) was added CDI (4.68 g, 33.2 mmol) at rt. The resulting mixture was stirred at 100° C. overnight. After cooled down to rt, the mixture was poured into NaHCO₃ (aq) and extracted with EtOAc (30 mL*3). The combined organic phase was washed with brine, dried over Na₂SO₄, filtered and concentrated. The crude product was purified by chromatography (silica gel, 0-7%, MeOH in DCM) to give 5-chloro-1,7-dimethyl-3-(tetrahydro-2H-pyran-4-yl)-3,4-dihydropyrimido[4,5-d]pyrimidin-2(1H)-one (600 mg, 2.02 mmol, 30.4%) as a white solid. LC-MS (ESI) m/z 297 (M+H)⁺.

### Step F. (R)-5-((1-(3-(1,1-difluoro-2-hydroxyethyl)phenyl)ethyl)amino)-1,7-dimethyl-3-(tetrahydro-2H-pyran-4-yl)-3,4-dihydropyrimido[4,5-d]pyrimidin-2(1H)-one

To a stirred solution of 5-chloro-1,7-dimethyl-3-(tetrahydro-2H-pyran-4-yl)-3,4-dihydropyrimido[4,5-d]pyrimidin-2(1H)-one (60 mg, 0.202 mmol) in anhydrous DMA (3 mL) was added K₂CO₃ (30.7 mg, 0.222 mmol) and (R)-2-(3-(1-aminoethyl)-2-fluorophenyl)-2,2-difluoroethanol (60 mg, 0.298 mmol) at rt. The resulting mixture was stirred at 100° C. under nitrogen atmosphere overnight. The cooled reaction mixture was diluted with water (10 mL) and extracted with EtOAc (30 mL*3). The combined organic phase was washed with brine, dried over Na₂SO₄, filtered and concentrated. The residue was purified by prep-TLC (DCM/MeOH=15/1) to give the crude product which was purified further by prep-HPLC (C18, 20% acetonitrile in H₂O with 0.1% formic acid) to give (R)-5-((1-(3-(1,1-difluoro-2-hydroxyethyl)phenyl)ethyl)amino)-1,7-dimethyl-3-(tetrahydro-2H-pyran-4-yl)-3,4-dihydropyrimido[4,5-d]pyrimidin-2(1H)-one (3 mg, 0.007 mmol, 3.22%) as a white solid. LC-MS (ESI) m/z 462 (M+H)⁺. ¹H NMR (400 MHz, DMSO) δ8.32 (s, 1H), 7.68 (s, 1H), 7.58 (d, J=6.6 Hz, 1H), 7.54-7.46 (m, 2H), 4.95 (t, J=13.1 Hz, 2H), 4.33 (dd, J=14.0, 10.0 Hz, 1H), 4.21 (d, J=6.7 Hz, 1H), 4.10 (s, 2H), 3.94 (dd, J=11.1, 3.8 Hz, 2H), 3.36 (s, 2H), 3.21 (s, 3H), 2.42 (s, 3H), 2.42 (s, 3H), 1.76 (qd, J=12.2, 4.6 Hz, 2H), 1.52 (d, J=10.0 Hz, 2H), 1.33 (d, J=6.6 Hz, 3H).

### Example 26: Synthesis of (R)-5-((1-(1,1-difluoro-2,3-dihydro-1H-inden-4-yl)ethyl)amino)-1,7-dimethyl-3-(tetrahydro-2H-pyran-4-yl)-3,4-dihydropyrimido[4,5-d]pyrimidin-2(1H)-one. (the Title Compounds was Synthesized from Compound 6 in Example 25.)

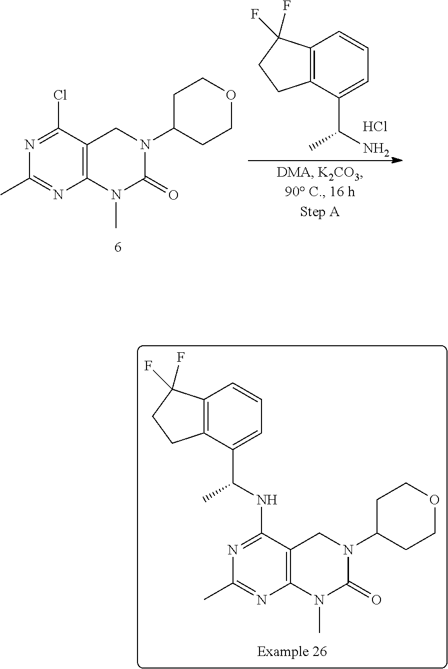

### Step A. 7-(R)-5-((1-(1,1-difluoro-2,3-dihydro-1H-inden-4-yl)ethyl)amino)-1,7-dimethyl-3-(tetrahydro-2H-pyran-4-yl)-3,4-dihydropyrimido[4,5-d]pyrimidin-2(1H)-one

To a stirred solution of 5-chloro-1,7-dimethyl-3-(tetrahydro-2H-pyran-4-yl)-3,4-dihydropyrimido[4,5-d]pyrimidin-2(1H)-one (50 mg, 0.168 mmol) in anhydrous DMA (3 mL) was added K₂CO₃ (57.9 mg, 0.420 mmol) & (R)-1-(1,1-difluoro-2,3-dihydro-1H-inden-4-yl)ethanamine (50 mg, 0.254 mol) at rt. The resulting mixture was stirred at 90° C. under nitrogen atmosphere overnight. The cooled reaction mixture was diluted with water (10 ml) and extracted with EtOAc (30 mL*3). The combined organic phase was washed with brine, dried over Na₂SO₄, filtered and concentrated. The residue was purified by prep-TLC (DCM/MeOH=15/1) to give the crude product which was purified further by prep-HPLC (C18, 30% MeOH in H₂O with 0.1% formic acid) to give (R)-5-((1-(1,1-difluoro-2,3-dihydro-1H-inden-4-yl)ethyl)amino)-1,7-dimethyl-3-(tetrahydro-2H-pyran-4-yl)-3,4-dihydropyrimido[4,5-d]pyrimidin-2(1H)-one (1.5 mg, 0.003 mmol, 1.95%) as a white solid. LC-MS (ESI) m/z 458 (M+H)⁺. ¹H NMR (400 MHz, DMSO) δ 7.71˜7.51 (m, 1H), 7.39 (d, J=5.3 Hz, 2H), 7.01 (d, J=7.5 Hz, 1H), 5.66-5.25 (m, 1H), 4.45 (t, J=12.0 Hz, 1H), 4.17 (s, 2H), 3.98 (dd, J=11.1, 3.9 Hz, 2H), 3.43 (d, J=11.4 Hz, 2H), 3.14 (s, 3H), 2.68 (s, 3H), 2.35˜2.19 (m, 5H), 1.96 (dd, J=12.3, 4.5 Hz, 6H), 1.49 (d, J=7.0 Hz, 6H), 0.86. (t, J=6.8 Hz, 1H).

### Example 27: Synthesis of 4-methoxy-5-(((S)-tetrahydrofuran-3-yl)oxy)-N—((R)-1-(3-(trifluoromethyl)phenyl)ethyl)-2H-indazole-7-carboxamide. (the Title Compounds was Synthesized from Compound 5 in Example 16.)

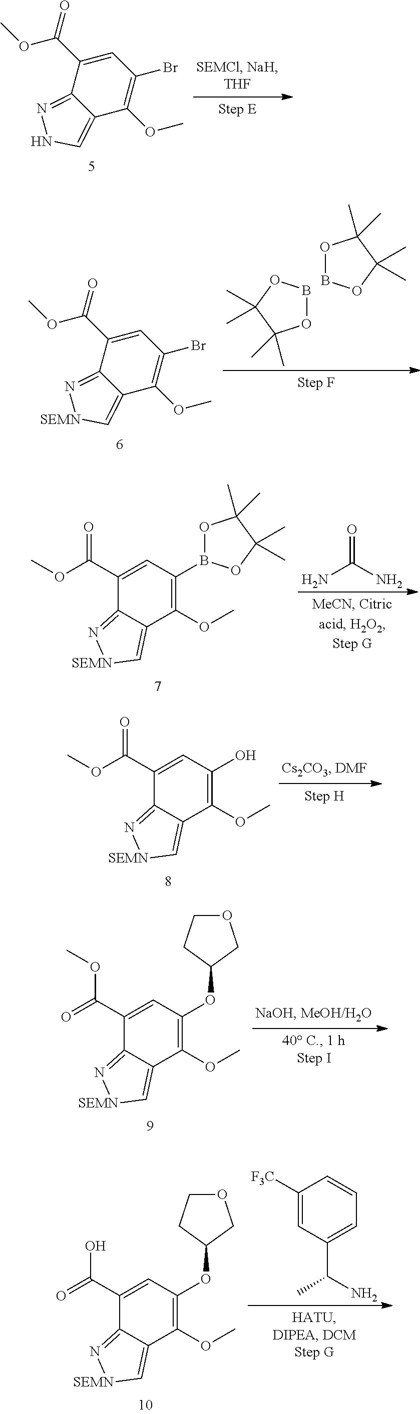

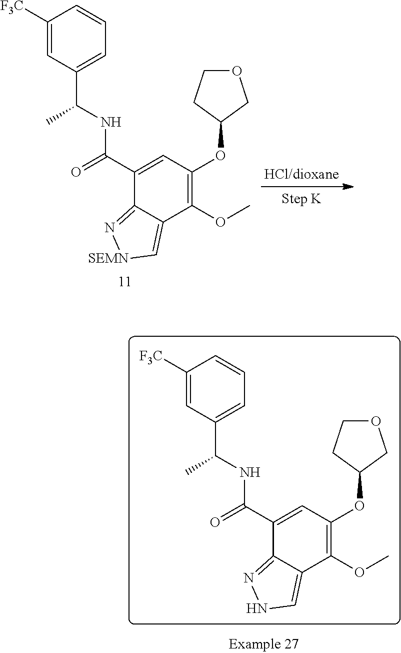 [SMILES: COc1c(O[C@H]2CCOC2)cc(C(=O)N[C@H](C)c2cccc(C(F)(F)F)c2)c2nn(COCC[Si](C)(C)C)cc12 ; COc1c(O[C@H]2CCOC2)cc(C(=O)N[C@H](C)c2cccc(C(F)(F)F)c2)c2n[nH]cc12]

### Step E. methyl 5-bromo-4-methoxy-2-((2-(trimethylsilyl)ethoxy)methyl)-2H-indazole-7-carboxylate

To a stirred solution of methyl 5-bromo-4-methoxy-2H-indazole-7-carboxylate (2 g, 7.0 mmol) in THE (20 mL) was added NaH (0.51 g, 21.0 mmol) at 0° C. After stirring at 0° C. for 20 min, SEM-Cl (1.4 mL, 8.4 mmol) was added. The reaction mixture was stirred at room temperature for 1 h The mixture was poured into NH₄Cl (aq, 50 mL) and extracted with EtOAc (50 mL*2). The combined organic phase was washed with brine, dried over Na₂SO₄, filtered and concentrated to give methyl 5-bromo-4-methoxy-2-{[2-(trimethylsilyl)ethoxy]methyl}-2H-indazole-7-carboxylate (2.2 g, 5.2 mmol, 75.5%) as a yellow solid. LCMS: m/z 415 (M+H)⁺.

### Step F. methyl 4-methoxy-5-(4,4,5,5-tetramethyl-1,3,2-dioxaborolan-2-yl)-2-((2-(trimethylsilyl)ethoxy)methyl)-2H-indazole-7-carboxylate

To a stirred solution of methyl 5-bromo-4-methoxy-2-{[2-(trimethylsilyl)ethoxy]methyl}-2H-indazole-7-carboxylate (1.5 g, 3.6 mmol) in dioxane (5 mL) was added 4,4,5,5-tetramethyl-2-(tetramethyl-1,3,2-dioxaborolan-2-yl)-1,3,2-dioxaborolane (1.8 mL, 7.2 mmol) Pd(dppf)Cl₂ (2.6 g, 3.6 mmol) and AcOK (1.1 g, 10.8 mmol). The reaction mixture was stirred at 100° C. for 16 h under N₂ atmosphere. The cooled reaction mixture was filtered and the filtrate was concentrated under vacuum. The residue was purified by column chromatography (silica gel, 25 g, 0-50% EtOAc in PE) to give methyl 4-methoxy-5-(tetramethyl-1,3,2-dioxaborolan-2-yl)-2-{[2-(trimethylsilyl)ethoxy]methyl}-2H-indazole-7-carboxylate (1.2 g, 2.5 mmol, 71.8%) as a white solid. LCMS: m/z 463 (M+H)⁺.

### Step G. methyl 5-hydroxy-4-methoxy-2-((2-(trimethylsilyl)ethoxy)methyl)-2H-indazole-7-carboxylate

To a stirred solution of methyl 4-methoxy-5-(tetramethyl-1,3,2-dioxaborolan-2-yl)-2-{[2-(trimethylsilyl)ethoxy]methyl}-2H-indazole-7-carboxylate (1.2 g, 2.5 mmol) in MeCN (4 mL) was added urea hydrogen peroxide (0.3 g) and citric acid (0.1 g, 0.52 mmol) in H₂O (1 mL). The reaction mixture was stirred at 25° C. for 2 h. The reaction mixture was poured into water, extracted with EtOAc. The organic layer was washed with brine, dried over Na₂SO₄ and concentrated under vacuum to give the residue and the residue was purified by column chromatography (silica gel, 5 g, 0˜4% MeOH in DCM) to give methyl 5-hydroxy-4-methoxy-2-{[2-(trimethylsilyl)ethoxy]methyl}-2H-indazole-7-carboxylate (320 mg, 0.9 mmol, 34.9%) as a light yellow solid. LCMS: m/z 353 (M+H)⁺.

### Step H. methyl (S)-4-methoxy-5-((tetrahydrofuran-3-yl)oxy)-2-((2-(trimethylsilyl) ethoxy)methyl)-2H-indazole-7-carboxylate

To a stirred solution of methyl 5-hydroxy-4-methoxy-2-{[2-(trimethylsilyl)ethoxy]methyl}-2H-indazole-7-carboxylate (320 mg, 0.9 mmol) in DMF (10 mL) was added oxolan-3-yl 4-methylbenzene-1-sulfonate (329 mg, 1.3 mmol) and Cs₂CO₃ (887 mg, 2.7 mmol). The reaction mixture was stirred at 90° C. for 2 h. The reaction mixture was poured into water, extracted with EtOAc. The organic layer was washed with brine, dried over Na₂SO₄ and concentrated under vacuum to give the residue and the residue was purified by column chromatography (silica gel, 25 g, 0˜50% PE in EtOAc) to give methyl 4-methoxy-5-(oxolan-3-yloxy)-2-{[2-(trimethylsilyl)ethoxy]methyl}-2H-indazole-7-carboxylate (300 mg, 0.71 mmol, 78.2%) as a white solid. LCMS: m/z 423 (M+H)⁺.

### Step I. (S)-4-methoxy-5-((tetrahydrofuran-3-yl)oxy)-2-((2-(trimethylsilyl)ethoxy)methyl)-2H-indazole-7-carboxylic acid

To a stirred solution of methyl 4-methoxy-5-(oxolan-3-yloxy)-2-{[2-(trimethylsilyl)ethoxy]methyl}-2H-indazole-7-carboxylate (300 mg, 0.71 mmol) in MeOH (3 mL) was added NaOH (142 mg, 3.5 mmol) in H₂O (2 mL). The reaction mixture was stirred at room temperature for 2 h. The pH of the reaction mixture was adjusted to 3-4 and extracted with EA. The organic layers were dried over Na₂SO₄ and concentrated under vacuum to give the crude product 4-methoxy-5-(oxolan-3-yloxy)-2-{[2-(trimethylsilyl)ethoxy]methyl}-2H-indazole-7-carboxylic acid (240 mg, 0.58 mmol, 82.7%) as a white solid. LCMS: m/z 409 (M+H)⁺.

### Step G. 4-methoxy-5-(((S)-tetrahydrofuran-3-yl)oxy)-N—((R)-1-(3-(trifluoromethyl) phenyl)ethyl)-2-((2-(trimethylsilyl)ethoxy)methyl)-2H-indazole-7-carboxamide

To a stirred solution of 4-methoxy-5-(oxolan-3-yloxy)-2-{[2-(trimethylsilyl) ethoxy]methyl}-2H-indazole-7-carboxylic acid (50 mg, 0.12 mmol) in DMF (3 mL) was added (1R)-1-[3-(trifluoromethyl)phenyl]ethan-1-amine (27.7 mg, 0.15 mmol), DIEA (0.06 mL, 0.36 mmol) and HATU (69.8 mg, 0.18 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (50% EtOAc in PE) to give the crude product and the crude product was purified by prep-HPLC to give 4-methoxy-5-(oxolan-3-yloxy)-N-[(1R)-1-[3-(trifluoromethyl)phenyl]ethyl]-2-{[2-(trimethylsilyl)ethoxy]methyl}-2H-indazole-7-carboxamide (60 mg, 0.10 mmol, 84.5%) as a white solid. LCMS: m/z 580 (M+H)⁺.

### Step K. 4-methoxy-5-(((S)-tetrahydrofuran-3-yl)oxy)-N—((R)-1-(3-(trifluoromethyl)phenyl) ethyl)-2H-indazole-7-carboxamide

To a stirred solution of 4-methoxy-5-(oxolan-3-yloxy)-N-[(1R)-1-[3-(trifluoromethyl) phenyl]ethyl]-2-{[2-(trimethylsilyl)ethoxy]methyl}-2H-indazole-7-carboxamide (50 mg, 0.086 mmol) in 4N HCl/dioxane (3 mL). The reaction mixture was stirred at room temperature for 1 h. The reaction mixture was concentrated under vacuum to give the residue and the residue was purified by pre-HPLC (phase A:H2O (0.1% FA). phase B:MeCN) to give 4-methoxy-5-(oxolan-3-yloxy)-N-[(1R)-1-[3-(trifluoromethyl)phenyl]ethyl]-2H-indazole-7-carboxamide (11 mg, 0.02 mmol, 28.3%) as a white solid. LCMS: m/z 450 (M+H)⁺. ¹H NMR (400 MHz, MeOD) δ 8.26 (s, 1H), 7.85 (s, 1H), 7.75-7.66 (m, 2H), 7.57-7.49 (m, 2H), 5.44-5.31 (m, 1H), 5.04 (t, J=4.6 Hz, 1H), 4.26 (s, 3H), 4.11-4.01 (m, 2H), 3.92 (td, J=8.3, 3.8 Hz, 1H), 3.85 (dd, J=10.1, 4.1 Hz, 1H), 2.22 (dd, J=12.6, 6.1 Hz, 1H), 2.17-2.06 (m, 1H), 1.63 (d, J=7.1 Hz, 3H).

The examples in the following Table 2 were prepared by using a method analogous to that used to prepare the examples as described herein.

TABLE 2

| Ex# | Structure & name | Analytical data | Method |
|---|---|---|---|
| Example 28 | 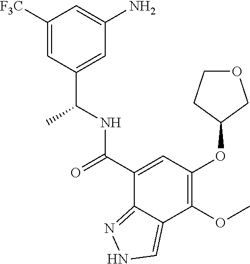 [SMILES: COc1c(O[C@H]2CCOC2)cc(C(=O)N[C@H](C)c2cc(N)cc(C(F)(F)F)c2)c2n[nH]cc12] | LCMS: m/z 465 (M + H)⁺. ¹H NMR (400 MHz, MeOD) δ 8.15 (d, J = 86.0 Hz, 1H), 7.85 (s, 1H), 6.95 (s, 2H), 6.81 (s, 1H), 5.24 (q, J = 7.0 Hz, 1H), 5.05 (s, 2H), 4.58 (s, 2H), 4.25 (s, 3H), 4.16-3.99 (m, 2H), 3.97-3.79 (m, 2H), 2.29- 2.03 (m, 2H), 1.58 (d, J = 7.0 Hz, 3H). | Example 27 |
| | N-((R)-1-(3-amino-5- | | |
| | (trifluoromethyl)phenyl)ethyl)-4- | | |
| | methoxy-5-(((S)-tetrahydrofuran- | | |
| | 3-yl)oxy)-2H-indazole-7- | | |
| | carboxamide | | |
| Example 29 | 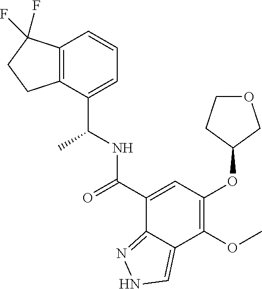 [SMILES: COc1c(O[C@H]2CCOC2)cc(C(=O)N[C@H](C)c2cccc3c2CCC3(F)F)c2n[nH]cc12] | LCMS: m/z 458 (M + H)⁺. ¹H NMR (400 MHz, MeOD) δ 8.25 (s, 1H), 7.84 (s, 1H), 7.57 (d, J = 6.7 Hz, 1H), 7.42-7.31 (m, 2H), 5.38 (q, J = 7.0 Hz, 1H), 5.03 (s, 1H), 4.25 (s, 3H), 4.06 (dd, J = 13.8, 8.9 Hz, 2H), 3.96-3.77 (m, 2H), 3.26-3.03 (m, 2H), 2.59 (tt, J = 13.6, 6.8 Hz, 2H), 2.28-2.00 (m, 3H), 1.60 (d, J = 7.0 Hz, 4H). | Example 27 |
| | N-((R)-1-(1,1-difluoro-2,3- | | |
| | dihydro-1H-inden-4-yl)ethyl)-4- | | |
| | methoxy-5-(((S)-tetrahydrofuran- | | |
| | 3-yl)oxy)-2H-indazole-7- | | |
| | carboxamide | | |
| Example 30 | 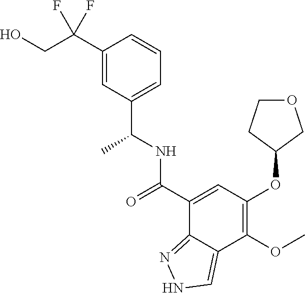 [SMILES: COc1c(O[C@H]2CCOC2)cc(C(=O)N[C@H](C)c2cccc(C(F)(F)CO)c2)c2n[nH]cc12] | LCMS: m/z 462 (M + H)⁺. ¹H NMR (400 MHz, MeOD) δ 8.25 (s, 1H), 7.85 (s, 1H), 7.61 (s, 1H), 7.54 (d, J = 6.8 Hz, 1H), 7.46- 7.38 (m, 2H), 5.36 (q, J = 6.8 Hz, 1H), 5.03 (s, 1H), 4.24 (s, 3H), 4.06 (dd, J = 18.2, 9.4 Hz, 2H), 3.94-3.76 (m, 5H), 2.39-1.96 (m, 3H), 1.61 (d, J = 7.0 Hz, 3H). | Example 27 |
| | N-((R)-1-(3-(1,1-difluoro-2- | | |
| | hydroxyethyl)phenyl)ethyl)-4- | | |
| | methoxy-5-(((S)-tetrahydrofuran- | | |
| | 3-yl)oxy)-2H-indazole-7- | | |
| | carboxamide | | |
| Example 31 | 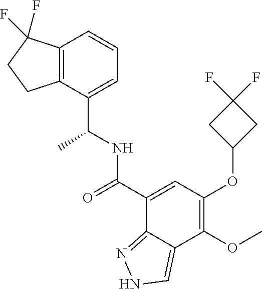 [SMILES: COc1c(OC2CC(F)(F)C2)cc(C(=O)N[C@H](C)c2cccc3c2CCC3(F)F)c2n[nH]cc12] | LCMS: m/z 478 (M + H)⁺. ¹H NMR (400 MHz, MeOD) δ 8.26 (s, 1H), 7.79 (s, 1H), 7.57 (d, J = 6.6 Hz, 1H), 7.43-7.33 (m, 2H), 5.38 (q, J = 7.0 Hz, 1H), 4.76 (dd, J = 7.0, 5.5 Hz, 1H), 3.28-3.08 (m, 2H), 3.05-2.94 (m, 2H), 2.82 (ddd, J = 18.4, 13.4,5.4 Hz, 2H), 2.59 (dq, = 21.0, 7.0 Hz, 2H), 1.60 (d, J = 7.1 Hz, 4H). LCMS: m/z 478 (M + H)⁺. | Example 27 |
| | (R)-N-(1-(1,1-difluoro-2,3- | | |
| | dihydro-1H-inden-4-yl)ethyl)-5- | | |
| | (3,3-difluorocyclobutoxy)-4- | | |
| | methoxy-2H-indazole-7- | | |
| | carboxamide | | |

### Example 32: Synthesis of N-[(1R)-1-[3-(1,1-difluoro-2-hydroxyethyl)phenyl]ethyl]-4-methoxy-5-(4-methylpiperazin-1-yl)-1H-indazole-7-carboxamide. (the Title Compounds was Synthesized from Compound 5 in Example 16.)

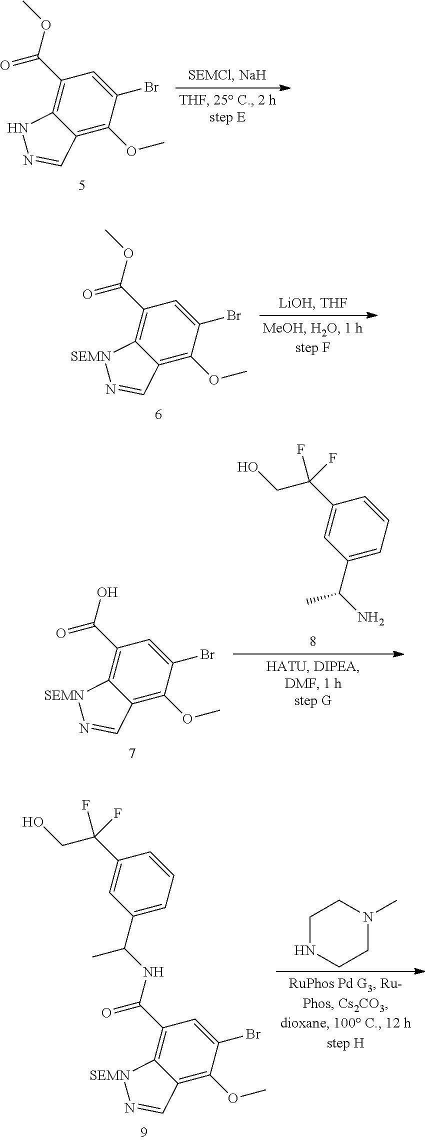

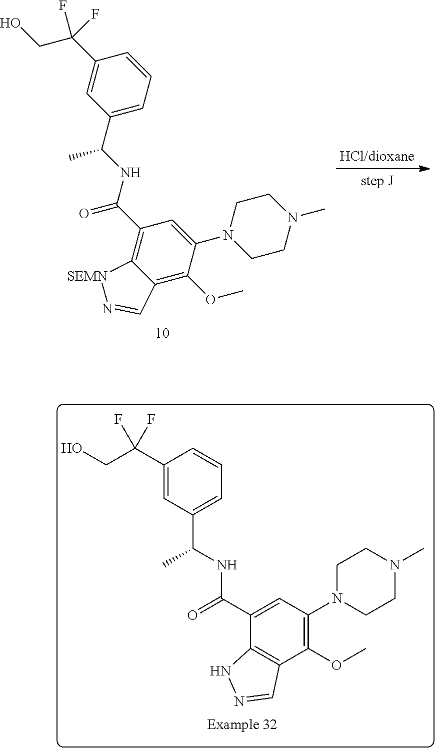 [SMILES: COc1c(N2CCN(C)CC2)cc(C(=O)N[C@H](C)c2cccc(C(F)(F)CO)c2)c2c1cnn2COCC[Si](C)(C)C ; COc1c(N2CCN(C)CC2)cc(C(=O)N[C@H](C)c2cccc(C(F)(F)CO)c2)c2[nH]ncc12]

### Step E. 5-bromo-4-methoxy-1-{[2-(trimethylsilyl)ethoxy]methyl}-1H-indazole-7-carboxylate

To a solution of methyl 5-bromo-4-methoxy-1H-indazole-7-carboxylate (3 g, 10.5 mmol) in THF (30 mL) was added NaH (0.63 g, 15.7 mmol) and 2-(Trimethylsilyl)ethoxymethyl Chloride (2.79 mL, 15.7 mmol) and the mixture was stirred at room temperature for 2 hours. The mixture was quenched saturated aq·NH₄Cl solution, extracted with EtOAc. The organic phase was washed with brine, dried over anhydrous Na₂SO₄, filtered and concentrated to dryness. The residue was purified by chromatography on silica gel (DCM:MeOH=20:1) to give methyl 5-bromo-4-methoxy-1-{[2-(trimethylsilyl)ethoxy]methyl}-1H-indazole-7-carboxylate (3 g, 68%) as yellow solid. LC/MS (ESI) (m/z): 415 (M+H)⁺.

### Step F. 5-bromo-4-methoxy-1-{[2-(trimethylsilyl)ethoxy]methyl}-1H-indazole-7-carboxylic acid

To a solution of methyl 5-bromo-4-methoxy-1-{[2-(trimethylsilyl)ethoxy]methyl}-1H-indazole-7-carboxylate (1 g, 2.408 mmol) in H₂O (10 mL)/MeOH (10 mL) was added NaOH (4.815 mL, 1 N) at 0° C. The reaction mixture was stirred at 25° C. for 2 hours. The mixture was concentrated to dryness and the residue was washed with diethyl ether and dried under vacuum to give 5-bromo-4-methoxy-1-{[2-(trimethylsilyl)ethoxy]methyl}-1H-indazole-7-carboxylic acid (900 mg, 2.24 mmol, 93.2%) as brown solid, which was used directly in the next step. LC/MS (ESI) (m/z): 401 (M+H)⁺.

### Step G. 5-bromo-N-{1-[3-(1,1-difluoro-2-hydroxyethyl)phenyl]ethyl}-4-methoxy-1-{[2-(trimethylsilyl)ethoxy]methyl}-1H-indazole-7-carboxamide

To a mixture of 5-bromo-4-methoxy-1-{[2-(trimethylsilyl)ethoxy]methyl}-1H-indazole-7-carboxylic acid (1 g, 2.49 mmol) and 2-{3-[(1R)-1-aminoethyl]phenyl}-2,2-difluoroethan-1-ol (1.00 g, 4.98 mmol) in DMF (20 mL) was added HATU (1.89 g, 4.98 mmol) and DIEA (1.64 mL, 9.96 mmol) at 0° C. The mixture was stirred at room temperature under N₂ atmosphere for 2 hours. The mixture was diluted with EtOAc and washed with saturated aq·NH₄Cl solution and brine, dried over anhydrous Na₂SO₄, filtered and concentrated to dryness. The residue was purified by chromatography on silica gel (DCM:MeOH=97:3) to give 5-bromo-N-{1-[3-(1,1-difluoro-2-hydroxyethyl)phenyl]ethyl}-4-methoxy-1-{[2-(trimethylsilyl)ethoxy]methyl}-1H-indazole-7-carboxamide (500 mg, 34%) as brown solid. LC/MS (ESI) (m/z): 584 (M+H)⁺.

### Step H. N-[(1S)-1-[3-(1,1-difluoro-2-hydroxyethyl)phenyl]ethyl]-4-methoxy-5-(4-methylpiperazin-1-yl)-1-{[2-(trimethylsilyl)ethoxy]methyl}-1H-indazole-7-carboxamide

To a mixture of 5-bromo-N-{1-[3-(1,1-difluoro-2-hydroxyethyl)phenyl]ethyl}-4-methoxy-1-{[2-(trimethylsilyl)ethoxy]methyl}-1H-indazole-7-carboxamide (100 mg, 0.171 mmol) and 1-methylpiperazine (34 mg, 0.34 mmol) in dioxane (10 mL) was added RuPhos-Pd-G3 (14 mg, 0.017 mmol), RuPhos (16 mg, 0.034 mmol) and Cs₂CO₃ (167 mg, 0.513 mmol) under N₂ atmosphere. The mixture was degassed under N₂ atmosphere for three times and stirred at 120° C. under N₂ atmosphere for 12 hours. The mixture was diluted with EtOAc, washed with water and brine, dried and concentrated to dryness to give crude product, which was purified on flash chromatography (DCM:MeOH=20:1 to 10:1) to give N-[(1S)-1-[3-(1,1-difluoro-2-hydroxyethyl)phenyl]ethyl]-4-methoxy-5-(4-methylpiperazin-1-yl)-1-{[2-(trimethylsilyl)ethoxy]methyl}-1H-indazole-7-carboxamide (10 mg, 9%) as yellow solid. LC/MS (ESI) (m/z): 604 (M+H)^{m}.

### Step J. N-[(1R)-1-[3-(1,1-difluoro-2-hydroxyethyl)phenyl]ethyl]-4-methoxy-5-(4-methylpiperazin-1-yl)-1H-indazole-7-carboxamide

To a solution of N-[(1S)-1-[3-(1,1-difluoro-2-hydroxyethyl)phenyl]ethyl]-4-methoxy-5-(4-methylpiperazin-1-yl)-1-{[2-(trimethylsilyl)ethoxy]methyl}-1H-indazole-7-carboxamide (10 mg, 0.017 mmol) in DCM (3 mL) was added HCl (1 mL, 4 N) and the mixture was stirred at room temperature for 1 hours. The mixture was diluted with DCM, washed with saturated aq·NaHCO₃ solution and brine, dried over anhydrous Na₂SO₄, filtered and concentrated to dryness. The residue was purified by chromatography on silica gel (DCM:MeOH=10:1) to give N-[(1R)-1-[3-(1,1-difluoro-2-hydroxyethyl)phenyl]ethyl]-4-methoxy-5-(4-methylpiperazin-1-yl)-1H-indazole-7-carboxamide (2.6 mg, 0.005 mmol, 33.2%) as white solid. LC/MS (ESI) (m/z): 474 (M+H)⁺. ¹HNMR (400 MHz, MeOD-d₄) δ 8.45 (s, 1H), 8.32 (s, 1H), 7.84 (s, 1H), 7.62 (s, 1H), 7.56 (d, J=7.1 Hz, 11H), 7.43 (p, J=7.8 Hz, 2H), 5.37 (q, J=7.0 Hz, 11H), 4.31 (s, 3H), 3.88 (t, J=13.5 Hz, 2H), 3.31-3.27 (m, 4H), 3.25-3.10 (M, 4H), 2.76 (s, 3H), 1.63 (d, J=7.1 Hz, 3H).

### Example 33: N-[(1R)-1-[3-(1,1-difluoro-2-hydroxyethyl)phenyl]ethyl]-4-methoxy-5-(4-methylpiperazin-1-yl)-1H-indazole-7-carboxamide. (the Title Compounds was Synthesized from Compound 9 in Example 32.)

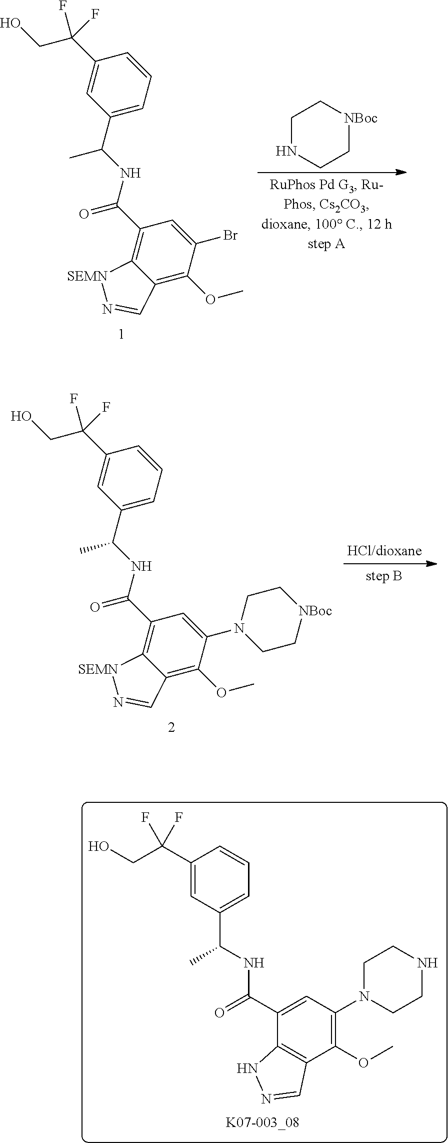 [SMILES: COc1c(Br)cc(C(=O)NC(C)c2cccc(C(F)(F)CO)c2)c2c1cnn2COCC[Si](C)(C)C ; CC(C)(C)OC(=O)N1CCNCC1 ; COc1c(N2CCN(C(=O)OC(C)(C)C)CC2)cc(C(=O)N[C@H](C)c2cccc(C(F)(F)CO)c2)c2c1cnn2COCC[Si](C)(C)C ; COc1c(N2CCNCC2)cc(C(=O)N[C@H](C)c2cccc(C(F)(F)CO)c2)c2[nH]ncc12]

### Step A. tert-butyl 4-(7-{[(1S)-1-[3-(1,1-difluoro-2-hydroxyethyl)phenyl]ethyl]carbamoyl}-4-methoxy-1-{[2-(trimethylsilyl)ethoxy]methyl}-1H-indazol-5-yl)piperazine-1-carboxylate

To a mixture of 5-bromo-N-{1-[3-(1,1-difluoro-2-hydroxyethyl)phenyl]ethyl}-4-methoxy-1-{[2-(trimethylsilyl)ethoxy]methyl}-1H-indazole-7-carboxamide (100 mg, 0.171 mmol) and tert-butyl piperazine-1-carboxylate (63 mg, 0.342 mmol) in dioxane (5 mL) was added RuPhos-Pd-G3 (14 mg, 0.017 mmol), RuPhos (15 mg, 0.034 mmol) and Cs₂CO₃ (167 mg, 0.513 mmol) under N₂ atmosphere. The mixture was degassed under N₂ atmosphere for three times and stirred at 120° C. under N₂ atmosphere for 12 hours. The mixture was diluted with EtOAc, washed with water and brine, dried and concentrated to dryness to give crude product, which was purified on flash chromatography (DCM:MeOH=20:1 to 10:1) to give tert-butyl 4-(7-{[(1S)-1-[3-(1,1-difluoro-2-hydroxyethyl)phenyl]ethyl]carbamoyl}-4-methoxy-1-{[2-(trimethylsilyl)ethoxy]methyl}-1H-indazol-5-yl)piperazine-1-carboxylate (10 mg, 0.014 mmol, 8.47%) as yellow solid. LC/MS (ESI) (m/z): 689 (M+H)⁺.

### Step B: N-[(1R)-1-[3-(1,1-difluoro-2-hydroxyethyl)phenyl]ethyl]-4-methoxy-5-(4-methylpiperazin-1-yl)-1H-indazole-7-carboxamide

To a solution of tert-butyl 4-(7-{[(1S)-1-[3-(1,1-difluoro-2-hydroxyethyl) phenyl]ethyl]carbamoyl}-4-methoxy-1-{[2-(trimethylsilyl)ethoxy]methyl}-1H-indazol-5-yl)piperazine-1-carboxylate (10 mg, 0.014 mmol) in DCM (3 mL) was added HCl (1 mL, 4.00 mmol). The reaction mixture was stirred at room temperature for 1 hours. The mixture was diluted with DCM, washed with saturated aq·NaHCO₃ solution and brine, dried over anhydrous Na₂SO₄, filtered and concentrated to dryness. The residue was purified by chromatography on silica gel (DCM:MeOH=10:1) to give N-[(1R)-1-[3-(1,1-difluoro-2-hydroxyethyl) phenyl]ethyl]-4-methoxy-5-(4-methylpiperazin-1-yl)-1H-indazole-7-carboxamide (2.6 mg, 37%) as white solid. LC/MS (ESI) (m/z): 460 (M+H)⁺. ¹HNMR (400 MHz, MeOD-d₄) δ 8.53 (s, 1H), 8.36 (s, 1H), 7.85 (s, 1H), 7.68-7.62 (m, 1H), 7.60-7.55 (m, 1H), 7.50-7.40 (m, 2H), 5.39 (m, 1H), 4.35 (s, 3H), 3.90 (t, J=13.5 Hz, 2H), 3.42-3.35 (m, 8H), 1.66 (d, J=7.0 Hz, 3H).

### Example 34: N-[(1R)-1-[3-(1,1-difluoro-2-hydroxyethyl)phenyl]ethyl]-4-methoxy-5-(1-methylpiperidin-4-yl)-1H-indazole-7-carboxamide. (the Title Compounds was Synthesized from Compound 5 in Example 16.)

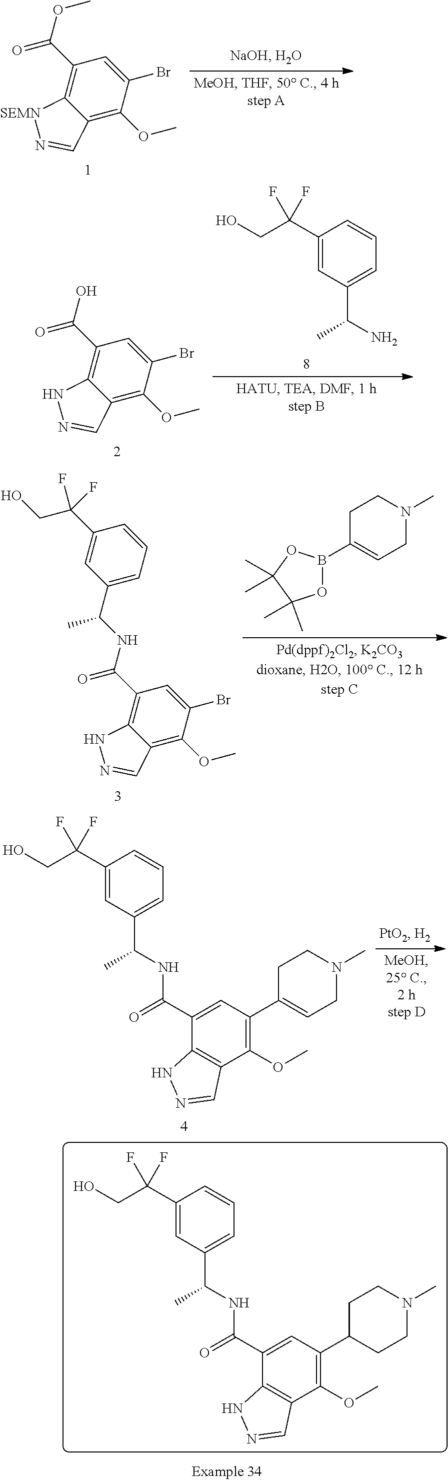

### Step A: 5-bromo-4-methoxy-1H-indazole-7-carboxylic acid

To a solution of methyl 5-bromo-4-methoxy-1-{[2-(trimethylsilyl)ethoxy]methyl}-1H-indazole-7-carboxylate (400 mg, 0.963 mmol) in MeOH (5 mL) and water (1 mL) was added NaOH (77 mg, 1.92 mmol) and THF (5 mL). The mixture was stirred at room temperature for 2 hours. The mixture was concentrated to 1/5 volume, diluted with water and washed with MTBE twice. The aqueous layer was acidified with 1N aq·HCl to pH-3 and extracted with DCM twice. The combined organic layers were washed with brine, dried over anhydrous Na₂SO₄, and concentrated under reduced pressure to give 5-bromo-4-methoxy-1H-indazole-7-carboxylic acid (230 mg, 88%) as white solid. LC/MS (ESI) (m/z): 271 (M+H)⁺.

### Step B: 5-bromo-N-[(1R)-1-[3-(1,1-difluoro-2-hydroxyethyl)phenyl]ethyl]-4-methoxy-1H-indazole-7-carboxamide

To a mixture of 5-bromo-4-methoxy-1H-indazole-7-carboxylic acid (230 mg, 0.848 mmol) and 2-{3-[(1R)-1-aminoethyl]phenyl}-2,2-difluoroethan-1-ol (170 mg, 0.848 mmol) in DMF (5 mL) was added DIEA (0.42 mL, 2.54 mmol), HATU (483 mg, 1.27 mmol) at 25° C. The mixture was stirred at room temperature under N₂ atmosphere for 2 hours. The mixture was diluted with EtOAc and washed with saturated aq·NH₄Cl solution and brine, dried over anhydrous Na₂SO₄, filtered and concentrated to dryness. The residue was purified by chromatography on silica gel (DCM:MeOH=97:3) to give 5-bromo-N-[(1R)-1-[3-(1,1-difluoro-2-hydroxyethyl)phenyl]ethyl]-4-methoxy-1H-indazole-7-carboxamide (230 mg, 59%) as white solid. LC/MS (ESI) (m/z): 454 (M+H)⁺.

### Step C: N-[(1R)-1-[3-(1,1-difluoro-2-hydroxyethyl)phenyl]ethyl]-4-methoxy-5-(1-methyl-1,2,3,6-tetrahydropyridin-4-yl)-1H-indazole-7-carboxamide

To a mixture of 5-bromo-N-[(1R)-1-[3-(1,1-difluoro-2-hydroxyethyl)phenyl]ethyl]-4-methoxy-1H-indazole-7-carboxamide (60 mg, 0.132 mmol) and 1-methyl-4-(tetramethyl-1,3,2-dioxaborolan-2-yl)-1,2,3,6-tetrahydropyridine (58 mg, 0.264 mmol) in 1,4-dioxane (2.4 mL) and water (0.3 mL) was added Pd(dppf)Cl₂ (9 mg, 0.013 mmol), Na₂CO₃ (42 mg, 0.396 mmol) under N₂ atmosphere. The mixture was degassed under N₂ atmosphere for three times and stirred at 95° C. under N₂ atmosphere for 12 hours. The mixture was diluted with EtOAc, washed with water and brine, dried and concentrated to dryness to give crude product, which was purified on flash chromatography (DCM:MeOH=20:1 to 10:2) to give N-[(1R)-1-[3-(1,1-difluoro-2-hydroxyethyl)phenyl]ethyl]-4-methoxy-5-(1-methyl-1,2,3,6-tetrahydropyridin-4-yl)-1H-indazole-7-carboxamide (40 mg, 64%) as white solid. LC/MS (ESI) (m/z): 471 (M+H)⁺.

### Step D: N-[(1R)-1-[3-(1,1-difluoro-2-hydroxyethyl)phenyl]ethyl]-4-methoxy-5-(1-methylpiperidin-4-yl)-1H-indazole-7-carboxamide

To a solution of N-[(1R)-1-[3-(1,1-difluoro-2-hydroxyethyl)phenyl]ethyl]-4-methoxy-5-(1-methyl-1,2,3,6-tetrahydropyridin-4-yl)-1H-indazole-7-carboxamide (30 mg, 0.064 mmol) in MeOH (5 mL) was added PtO₂ (3 mg) at 25° C. and the mixture was degassed under N₂ atmosphere for three times and stirred under a H₂ balloon at room temperature for 1 hour. The mixture was filtered and the filtrate was concentrated to dryness to a residue. The residue was purified by chromatography on silica gel (DCM:MeOH=10:1) to give N-[(1R)-1-[3-(1,1-difluoro-2-hydroxyethyl)phenyl]ethyl]-4-methoxy-5-(1-methylpiperidin-4-yl)-1H-indazole-7-carboxamide (15 mg, 0.032 mmol, 49.7%) as white solid. LC/MS (ESI) (m/z): 473 (M+H)⁺.

¹HNMR (400 MHz, MeOD) δ 8.38 (s, 1H), 7.84 (s, 1H), 7.62 (s, 1H), 7.57 (d, J=7.0 Hz, 1H), 7.49-7.37 (m, 2H), 5.42-5.31 (m, 1H), 4.35 (s, 3H), 3.88 (t, J=13.5 Hz, 2H), 3.60 (d, J=11.9 Hz, 2H), 3.38-3.35 (m, 1H), 3.20-3.13 (m, 2H), 2.91 (s, 3H), 2.28-2.16 (m, 2H), 2.15-2.03 (d, 2H), 1.64 (d, J=7.1 Hz, 3H).

### Example 35: N-[(1R)-1-[3-(1,1-difluoro-2-hydroxyethyl)phenyl]ethyl]-4-methoxy-5-(piperidin-4-yl)-1H-indazole-7-carboxamide. (the Title Compounds was Synthesized from Compound 9 in Example 32.)

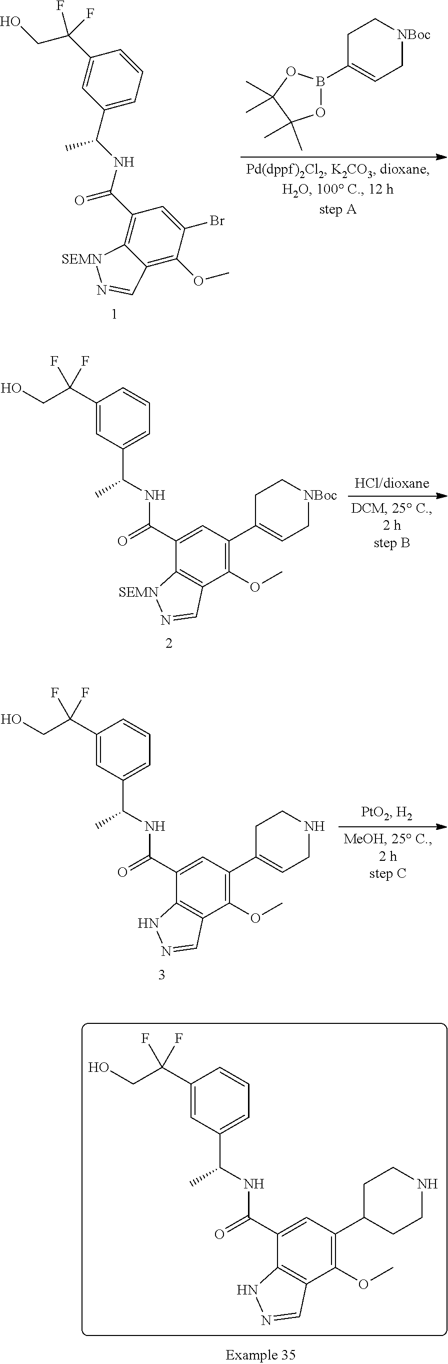 [SMILES: COc1c(Br)cc(C(=O)N[C@H](C)c2cccc(C(F)(F)CO)c2)c2c1cnn2COCC[Si](C)(C)C ; CC(C)(C)OC(=O)N1CC=C(B2OC(C)(C)C(C)(C)O2)CC1 ; COc1c(C2=CCN(C(=O)OC(C)(C)C)CC2)cc(C(=O)N[C@H](C)c2cccc(C(F)(F)CO)c2)c2c1cnn2COCC[Si](C)(C)C ; COc1c(C2=CCNCC2)cc(C(=O)N[C@H](C)c2cccc(C(F)(F)CO)c2)c2[nH]ncc12 ; COc1c(C2CCNCC2)cc(C(=O)N[C@H](C)c2cccc(C(F)(F)CO)c2)c2[nH]ncc12]

### Step A. tert-butyl 4-(7-{[(1R)-1-[3-(1,1-difluoro-2-hydroxyethyl)phenyl]ethyl]carbamoyl}-4-methoxy-1-{[2-(trimethylsilyl)ethoxy]methyl}-1H-indazol-5-yl)-1,2,3,6-tetrahydropyridine-1-carboxylate

To a mixture of 5-bromo-N-[(1S)-1-[3-(1,1-difluoro-2-hydroxyethyl)phenyl]ethyl]-4-methoxy-1-{[2-(trimethylsilyl)ethoxy]methyl}-1H-indazole-7-carboxamide (80 mg, 0.137 mmol) and tert-butyl 4-(tetramethyl-1,3,2-dioxaborolan-2-yl)-1,2,3,6-tetrahydropyridine-1-carboxylate (63.5 mg, 0.20 mmol) in 1,4-dioxane (5 mL) and water (1 mL) was added Pd(dppf)Cl₂ (5 mg, 0.007 mmol), K₂CO₃ (56 mg, 0.411 mmol) under N₂ atmosphere. The mixture was degassed under N₂ atmosphere for three times and stirred at 95° C. under N₂ atmosphere for 12 hours. The mixture was diluted with EtOAc, washed with water and brine, dried and concentrated to dryness to give crude product, which was purified on flash chromatography (DCM:MeOH=20:1) to give tert-butyl 4-(7-{[(1R)-1-[3-(1,1-difluoro-2-hydroxyethyl)phenyl]ethyl]carbamoyl}-4-methoxy-1-{[2-(trimethylsilyl)ethoxy]methyl}-1H-indazol-5-yl)-1,2,3,6-tetrahydropyridine-1-carboxylate (80 mg, 85%) as yellow solid. LC/MS (ESI) (m/z): 687 (M+H)⁺.

### Step B: N-[(1R)-1-[3-(1,1-difluoro-2-hydroxyethyl)phenyl]ethyl]-4-methoxy-5-(1,2,3,6-tetrahydropyridin-4-yl)-1H-indazole-7-carboxamide

To a solution of tert-butyl 4-(7-{[(1S)-1-[3-(1,1-difluoro-2-hydroxyethyl) phenyl]ethyl]carbamoyl}-4-methoxy-1-{[2-(trimethylsilyl)ethoxy]methyl}-1H-indazol-5-yl)-1,2,3,6-tetrahydropyridine-1-carboxylate (80 mg, 0.11 mmol) in DCM (2 mL) was added 4N HCl/dioxane (1 mL, 0.116 mmol) at 0° C. and the mixture was stirred at 25° C. for 2 hours. The mixture was concentrated to dryness and the residue was washed with diethyl ether and dried under vacuum to give N-[(1R)-1-[3-(1,1-difluoro-2-hydroxyethyl)phenyl]ethyl]-4-methoxy-5-(1,2,3,6-tetrahydropyridin-4-yl)-1H-indazole-7-carboxamide (50 mg, 94%) as brown solid, which was used directly in the next step. LC/MS (ESI) (m/z): 457 (M+H)⁺.

### Step C: N-[(1R)-1-[3-(1,1-difluoro-2-hydroxyethyl)phenyl]ethyl]-4-methoxy-5-(piperidin-4-yl)-1H-indazole-7-carboxamide

To a solution of N-[(1R)-1-[3-(1,1-difluoro-2-hydroxyethyl)phenyl]ethyl]-4-methoxy-5-(1,2,3,6-tetrahydropyridin-4-yl)-1H-indazole-7-carboxamide (50 mg, 0.11 mmol) in CF₃CH₂OH (5 mL) was added PtO₂ (5 mg) at 25° C. and the mixture was degassed under N₂ atmosphere for three times and stirred under a H₂ balloon at room temperature for 2 hour. The mixture was filtered and the filtrate was concentrated to dryness to give N-[(1R)-1-[3-(1,1-difluoro-2-hydroxyethyl)phenyl]ethyl]-4-methoxy-5-(piperidin-4-yl)-1H-indazole-7-carboxamide (5 mg, 10%) as white solid. LC/MS (ESI) (m/z): 459 (M+H)⁺. ¹H NMR (400 MHz, CD₃OD-d₄) δ 8.56 (s, 1H), 8.42 (s, 1H), 7.85 (s, 1H), 7.62 (s, 1H), 7.56 (d, J=7.0 Hz, 1H), 7.48-7.35 (m, 2H), 5.37 (q, J=7.0 Hz, 1H), 4.34 (s, 3H), 3.88 (t, J=13.5 Hz, 2H), 3.50 (d, J=12.7 Hz, 2H), 3.40-3.30 (m, 1H), 3.14 (td, J=12.4, 3.6 Hz, 2H), 2.17-1.96 (m, 4H), 1.64 (d, J=7.1 Hz, 3H).

### Example 36: Synthesis of N—((R)-1-(3-(1,1-difluoro-2-hydroxyethyl)phenyl)ethyl)-4-methoxy-5-(piperidin-3-yl)-1H-indazole-7-carboxamide. (the Title Compound was Synthesized from Compound 3 in Example 34.)

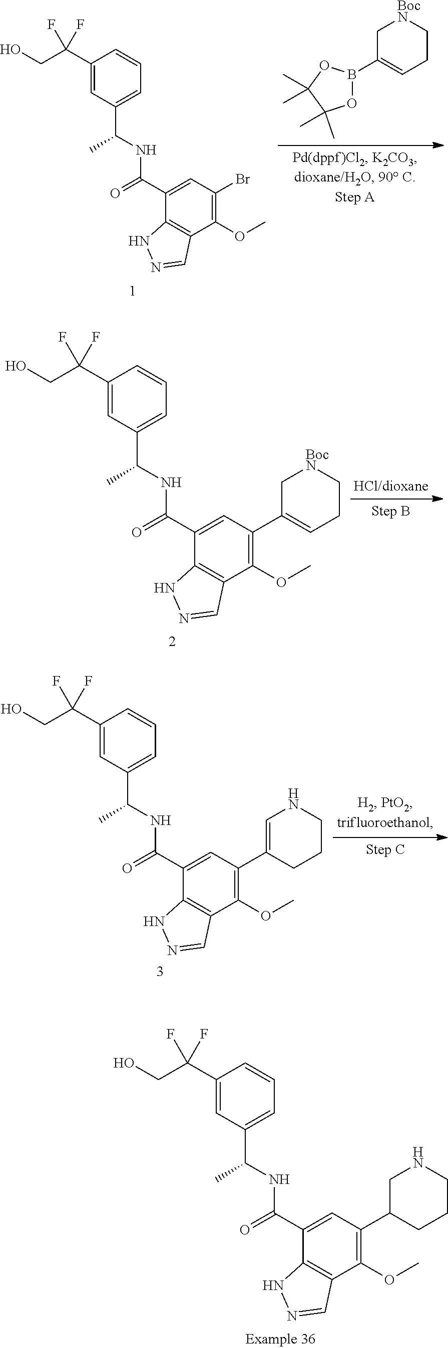 [SMILES: COc1c(Br)cc(C(=O)N[C@H](C)c2cccc(C(F)(F)CO)c2)c2[nH]ncc12 ; CC(C)(C)OC(=O)N1CCC=C(B2OC(C)(C)C(C)(C)O2)C1 ; COc1c(C2=CCCN(C(=O)OC(C)(C)C)C2)cc(C(=O)N[C@H](C)c2cccc(C(F)(F)CO)c2)c2[nH]ncc12 ; COc1c(C2=CNCCC2)cc(C(=O)N[C@H](C)c2cccc(C(F)(F)CO)c2)c2[nH]ncc12 ; COc1c(C2CCCNC2)cc(C(=O)N[C@H](C)c2cccc(C(F)(F)CO)c2)c2[nH]ncc12]

### Step A. tert-butyl (R)-5-(7-((1-(3-(1,1-difluoro-2-hydroxyethyl)phenyl)ethyl)carbamoyl)-4-methoxy-1H-indazol-5-yl)-3,6-dihydropyridine-1(2H)-carboxylate

To a stirred solution of 5-bromo-N-[(1R)-1-[3-(1,1-difluoro-2-hydroxyethyl) phenyl]ethyl]-4-methoxy-1H-indazole-7-carboxamide (100 mg, 0.2 mmol) in dioxane (2 mL) and H₂O (0.5 mL) was added tert-butyl 5-(4,4,5,5-tetramethyl-1,3,2-dioxaborolan-2-yl)-3,6-dihydropyridine-1(2H)-carboxylate (81.6 mg, 0.264 mmol) Pd(dppf)Cl₂ (32.1 mg, 0.04 mmol) and K₂CO₃ (91.2 mg, 0.6 mmol). The reaction mixture was stirred at 90° C. for 2 h. The reaction mixture was filtered and the filtrate was concentrated under vacuum to give the residue and the residue was purified [symbol] prep-TLC (50% EtOAc in PE) to give tert-butyl (R)-5-(7-((1-(3-(1,1-difluoro-2-hydroxyethyl)phenyl)ethyl)carbamoyl)-4-methoxy-1H-indazol-5-yl)-3,6-dihydropyridine-1(2H)-carboxylate (80 mg, 0.14 mmol, 65.4%) as a white solid. LCMS: m/z 557 (M+H)⁺.

### Step B. (R)—N-(1-(3-(1,1-difluoro-2-hydroxyethyl)phenyl)ethyl)-4-methoxy-5-(1,4,5,6-tetrahydropyridin-3-yl)-1H-indazole-7-carboxamide

To a stirred solution of tert-butyl (R)-5-(7-((1-(3-(1,1-difluoro-2-hydroxyethyl)phenyl)ethyl)carbamoyl)-4-methoxy-1H-indazol-5-yl)-3,6-dihydropyridine-1(2H)-carboxylate (80 mg, 0.2 mmol) in 4N HCl/dioxane (3 mL). The reaction mixture was stirred at room temperature for 2 h. The reaction mixture was concentrated under vacuum to give the residue N-[(1R)-1-[3-(1,1-difluoro-2-hydroxyethyl)phenyl]ethyl]-4-methoxy-5-(1,4,5,6-tetrahydropyridin-3-yl)-1H-indazole-7-carboxamide (60 mg, 0.13 mmol, 91.4%) as a colorless oil. LCMS: m/z 457 (M+H)⁺.

### Step C. N—((R)-1-(3-(1,1-difluoro-2-hydroxyethyl)phenyl)ethyl)-4-methoxy-5-(piperidin-3-yl)-1H-indazole-7-carboxamide

To a stirred solution of N-[(1R)-1-[3-(1,1-difluoro-2-hydroxyethyl)phenyl]ethyl]-4-methoxy-5-(1,4,5,6-tetrahydropyridin-3-yl)-1H-indazole-7-carboxamide (30 mg, 0.1 mmol) in 2,2,2-trifluoroethanol (3 mL) was added Platinum dioxide (15 mg, 0.1 mmol). The reaction mixture was stirred at 60° C. for 5 h using a H₂ balloon. The reaction mixture was filtered and the filtrate was concentrated under vacuum to give the residue and the residue was purified by prep-HPLC to give N-[(1R)-1-[3-(1,1-difluoro-2-hydroxyethyl)phenyl]ethyl]-4-methoxy-5-(piperidin-3-yl)-1H-indazole-7-carboxamide (3.1 mg, 0.01 mmol, 10.3%) as a white solid. LCMS: m/z 459 (M+H)⁺. ¹H NMR (400 MHz, MeOD) δ 8.36 (d, J=5.6 Hz, 1H), 7.97-7.83 (m, 1H), 7.62 (s, 1H), 7.56 (d, J=7.2 Hz, 1H), 7.47-7.39 (m, 2H), 5.44-5.28 (m, 1H), 3.88 (t, J=13.5 Hz, 2H), 3.14 (t, J=12.4 Hz, 2H), 2.78 (dt, J=23.0, 12.3 Hz, 2H), 1.90 (dd, J=15.6, 6.3 Hz, 4H), 1.75 (d, J=7.7 Hz, 2H), 1.63 (t, J=8.6 Hz, 4H).

### Example 37: Synthesis of N—((R)-1-(3-(1,1-difluoro-2-hydroxyethyl)phenyl)ethyl)-4-methoxy-5-(1-methylpiperidin-3-yl)-1H-indazole-7-carboxamide. (the Title Compounds was Synthesized from Compound 3 in Example 36.)

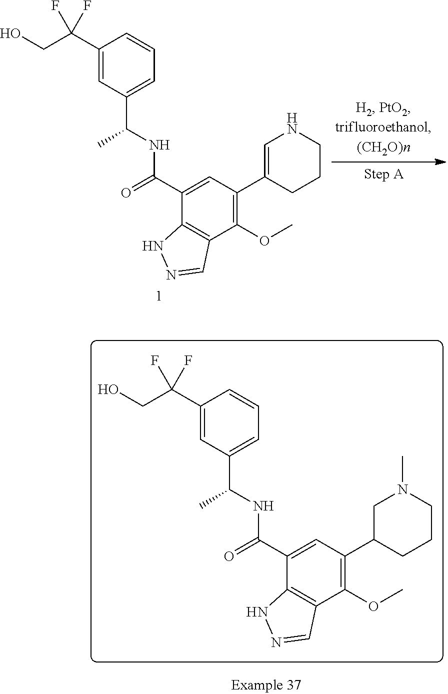 [SMILES: COc1c(C2=CNCCC2)cc(C(=O)N[C@H](C)c2cccc(C(F)(F)CO)c2)c2[nH]ncc12 ; COc1c(C2CCCN(C)C2)cc(C(=O)N[C@H](C)c2cccc(C(F)(F)CO)c2)c2[nH]ncc12]

### Step A. N—((R)-1-(3-(1,1-difluoro-2-hydroxyethyl)phenyl)ethyl)-4-methoxy-5-(1-methylpiperidin-3-yl)-1H-indazole-7-carboxamide

To a stirred solution of N-[(1R)-1-[3-(1,1-difluoro-2-hydroxyethyl)phenyl]ethyl]-4-methoxy-5-(1,4,5,6-tetrahydropyridin-3-yl)-1H-indazole-7-carboxamide (30 mg, 0.1 mmol) in 2,2,2-trifluoroethanol (3 mL) was added PtO₂ (7.5 mg, 0.1 mmol) and paraformaldehyde (10 mg, 0.1 mmol). The reaction mixture was stirred at 60° C. for 5 h under a H₂ balloon. The reaction mixture was filtered and the filtrate was concentrated under vacuum to give the residue and the residue was purified by prep-HPLC to give N-[(1R)-1-[3-(1,1-difluoro-2-hydroxyethyl)phenyl]ethyl]-4-methoxy-5-(1-methylpiperidin-3-yl)-1H-indazole-7-carboxamide (2.6 mg, 0.006 mmol, 8.3%) as a white solid. LCMS: m/z 473 (M+H)⁺. ¹H NMR (400 MHz, MeOD) δ 8.45 (s, 1H), 7.89 (s, 1H), 7.62 (s, 1H), 7.56 (d, J=7.1 Hz, 1H), 7.44 (q, J=7.8 Hz, 2H), 5.37 (dt, J=17.3, 8.6 Hz, 1H), 4.37 (s, 3H), 3.88 (t, J=13.5 Hz, 2H), 3.57 (dd, J=23.4, 11.5 Hz, 3H), 3.12 (td, J=12.0, 7.6 Hz, 1H), 2.97 (d, J=26.7 Hz, 1H), 2.91 (d, J=2.4 Hz, 3H), 2.18 (dd, J=16.2, 8.3 Hz, 1H), 1.97 (dd, J=18.7, 8.5 Hz, 4H), 1.64 (dd, J=7.0, 1.2 Hz, 4H).

### Example 38: Synthesis of (R)—N-(1-(1,1-difluoro-2,3-dihydro-1H-inden-4-yl)ethyl)-4-oxo-5-(tetrahydro-2H-pyran-4-yl)-4,5-dihydro-1H-pyrrolo[3,2-c]pyridine-7-carboxamide

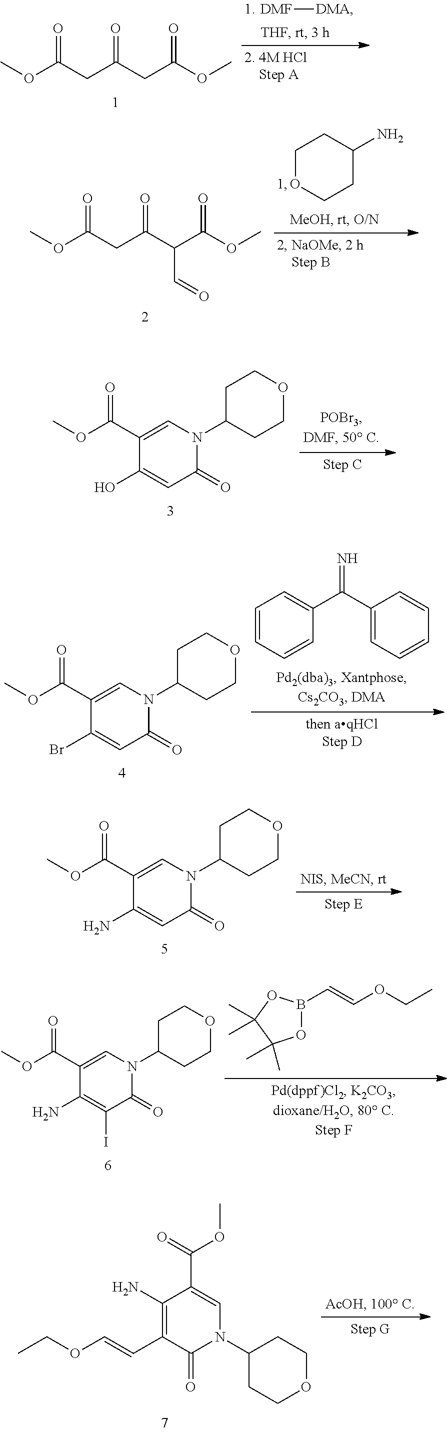

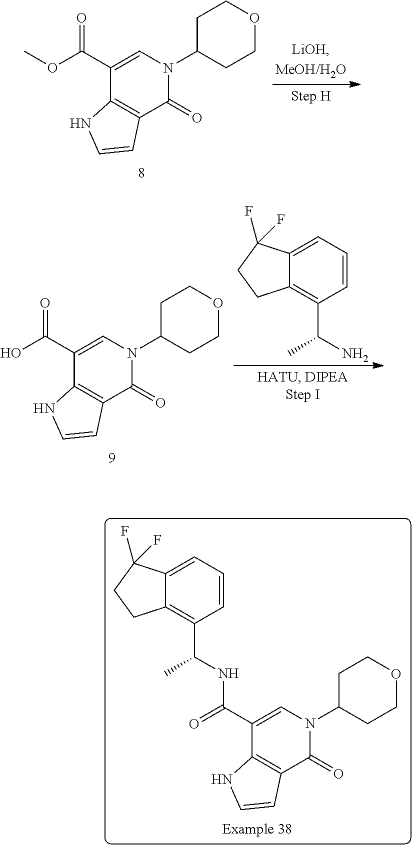

### Step A. dimethyl 2-formyl-3-oxopentanedioate

To a stirred solution of 1,5-dimethyl 3-oxopentanedioate (10 g, 57.4 mmol) in 2-Methyltetrahydrofuran (100 mL) at 0° C. was added DMF-DMA (6.8 g, 57.4 mmol). The reaction mixture was stirred at 0° C. for 1.5 h under nitrogen atmosphere before 4M HCl (28 mL, 13.1 mmol) was added. The result solution was allowed to warm to room temperature and stirred for 2 h. The reaction mixture was diluted with water (100 mL). The following mixture was extracted with EtOAc (10 mL*3). The combined organic phase was washed by water and brine, dried over anhydrous Na₂SO₄, filtered and concentrated to give the crude product which was purified by column chromatography on silica gel (PE:EtOAc=10:1 to 3:1) to obtain 1,5-dimethyl 2-formyl-3-oxopentanedioate (9.6 g, 47.5 mmol, yield 82.7%) as a colorless oil. LC/MS (ESI) m/z: 203.0 (M+H)⁺

### Step B: methyl 4-hydroxy-6-oxo-1-(tetrahydro-2H-pyran-4-yl)-1,6-dihydropyridine-3-carboxylate

To a stirred solution of 1,5-dimethyl 2-formyl-3-oxopentanedioate (12.0 g, 59.3 mmol) in CH₃₀H (80 mL) at 0° C. was added oxan-4-amine (5.0 g, 49.4 mmol). The reaction mixture was stirred at room temperature for 16 h under nitrogen atmosphere before MeONa (40 mL, 59.3 mmol, 30% in CH3OH) was added. The result solution was stirred at room temperature for 2 h. The reaction mixture was poured into ice-water (100 mL). CH₃OH was removed by reduce power. The precipitate was filtered and dried to give a crude desire product. The filtrate was extracted with DCM (80 mL×3), the combined organic phase was washed by water and brine, the organic phase was dried over anhydrous Na₂SO₄, filtered and concentrated to give a crude product. The combined crude product was triturated with 100 mL PE:DCM=10:1 to give the desire product (11 g, 43.5 mmol, 73.3%) as an off-white solid. LC/MS (ESI) m/z: 253.9 (M+H)⁺.

### Step C. methyl 4-bromo-6-oxo-1-(tetrahydro-2H-pyran-4-yl)-1,6-dihydropyridine-3-carboxylate

to a solution of methyl 4-hydroxy-1-(oxan-4-yl)-6-oxo-1,6-dihydropyridine-3-carboxylate (7 g, 27.64 mmol) in DMF (20 mL) was added into POBr₃ (11.9 g, 41.40 mmol). The reaction mixture was stirred at 100° C. for 5 h. The cooled reaction mixture was poured into water and extracted with DCM. The organic layer was dried over Na₂SO₄ and concentrated under vacuum to give the residue. The residue was purified by column chromatography (silica gel, 24 g, 0˜60% EA in PE) to give methyl 4-bromo-1-(oxan-4-yl)-6-oxo-1,6-dihydropyridine-3-carboxylate (5 g, 15.8 mmol, 57.2%) as a light yellow solid. LCMS: m/z 316 (M+H)⁺.

### Step D. methyl 4-amino-6-oxo-1-(tetrahydro-2H-pyran-4-yl)-1,6-dihydropyridine-3-carboxylate

To a stirred solution of methyl 4-bromo-1-(oxan-4-yl)-6-oxo-1,6-dihydropyridine-3-carboxylate (5 g, 15.82 mmol) in dioxane (30 mL) was added diphenylmethanimine (2.65 mL, 15.82 mmol), Pd₂(dba)₃ (1.45 g, 1.58 mmol), Xant-phos (1.76 g, 3.16 mmol) and Cs₂CO₃ (12.8 g, 39.5 mmol). The reaction mixture was stirred at 95° C. for 5 h and then cooled to r.t. HCl (2M, 20 mL) was added and stirred for 0.5 h. The pH of the reaction mixture was adjusted to 8-9 by adding saturated sodium bicarbonate and extracted with DCM. The organic layers were washed with brine, dried over Na₂SO₄ and concentrated under vacuum to give the residue. The residue was purified by column chromatography (silica gel, 24 g, 0˜10% MeOH in DCM) to give methyl 4-amino-1-(oxan-4-yl)-6-oxo-1,6-dihydropyridine-3-carboxylate (2.2 g, 8.72 mmol, 55.14%) as a light yellow solid. LCMS: m/z 253 (M+H)⁺.

### Step E. methyl 4-amino-5-iodo-6-oxo-1-(tetrahydro-2H-pyran-4-yl)-1,6-dihydropyridine-3-carboxylate

To a stirred solution of methyl 4-amino-1-(oxan-4-yl)-6-oxo-1,6-dihydropyridine-3-carboxylate (2.2 g, 8.72 mmol) in MeCN (20 mL) and DCM (10 mL) was added into(sulfanylidene)amine (1.66 g, 9.59 mmol). The reaction mixture was stirred at room temperature for 2 h. The reaction mixture was poured into water and extracted with EtOAc. The organic phase was washed with NaHCO₃, dried over Na₂SO₄ and concentrated under vacuum to give the residue. The residue purified by column chromatography (silica gel, 24 g, 0˜100% EtOAc in PE) to give methyl 4-amino-5-iodo-1-(oxan-4-yl)-6-oxo-1,6-dihydropyridine-3-carboxylate (1.9 g, 5.02 mmol, 57.6%) as a white solid. LCMS: m/z 379 (M+H)⁺.

### Step F. methyl (E)-4-amino-5-(2-ethoxyvinyl)-6-oxo-1-(tetrahydro-2H-pyran-4-yl)-1,6-dihydropyridine-3-carboxylate

To a stirred solution of methyl 4-amino-5-iodo-1-(oxan-4-yl)-6-oxo-1,6-dihydropyridine-3-carboxylate (1.9 g, 5.02 mmol) in dioxane (30 mL) and H₂O (5 mL) was added 2-[(E)-2-ethoxyethenyl]-4,4,5,5-tetramethyl-1,3,2-dioxaborolane (1.17 mL, 5.52 mmol), Pd(dppf)Cl₂ (0.37 g, 0.50 mmol) and K₂CO₃ (1.39 g, 10.04 mmol). The reaction mixture was stirred at 95° C. for 16 h. The reaction mixture was filtered and the filtrate was concentrated under vacuum to give the residue. The residue was purified by column chromatography (silica gel, 24 g, 0˜100% EtOAc in PE) to give methyl 4-amino-1-(oxan-4-yl)-6-oxo-1,6-dihydropyridine-3-carboxylate (2.20 g, 8.72 mmol, 55.1%) as a light yellow solid. LCMS: m/z 323 (M+H)⁺.

### Step G. methyl 4-oxo-5-(tetrahydro-2H-pyran-4-yl)-4,5-dihydro-1H-pyrrolo[3,2-c]pyridine-7-carboxylate

A stirred mixture of methyl 4-amino-5-[(E)-2-ethoxyethenyl]-1-(oxan-4-yl)-6-oxo-1,6-dihydropyridine-3-carboxylate (380 mg, 1.179 mmol) in AcOH (5 mL) was stirred at 100° C. for 2 h. The reaction mixture was concentrated under vacuum to give the residue. The residue was purified by column chromatography (silica gel, 24 g, 0˜60% EtOAc in PE) to give methyl 5-(oxan-4-yl)-4-oxo-1H,4H,5H-pyrrolo[3,2-c]pyridine-7-carboxylate (120 undefined, 0.43 mmol, 36.3%) as a light yellow solid. LCMS: m/z 277 (M+H)⁺.

### Step H. 4-oxo-5-(tetrahydro-2H-pyran-4-yl)-4,5-dihydro-1H-pyrrolo[3,2-c]pyridine-7-carboxylic acid

To a stirred solution of methyl 5-(oxan-4-yl)-4-oxo-1H,4H,5H-pyrrolo[3,2-c]pyridine-7-carboxylate (120 mg, 0.43 mmol) in MeOH (3 mL) was added a solution of LiOH (173 mg, 4.34 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give the crude product 5-(oxan-4-yl)-4-oxo-1H,4H,5H-pyrrolo[3,2-c]pyridine-7-carboxylic acid (90 mg, 0.34 mmol, 79.0%) as a light yellow solid. LCMS: m/z 263 (M+H)⁺.

### Step I. (R)—N-(1-(1,1-difluoro-2,3-dihydro-1H-inden-4-yl)ethyl)-4-oxo-5-(tetrahydro-2H-pyran-4-yl)-4,5-dihydro-1H-pyrrolo[3,2-c]pyridine-7-carboxamide

To a stirred solution of 5-(oxan-4-yl)-4-oxo-1H,4H,5H-pyrrolo[3,2-c]pyridine-7-carboxylic acid (30 mg, 0.114 mmol) in DMF (3 mL) was added (1R)-1-(1,1-difluoro-2,3-dihydro-1H-inden-4-yl)ethan-1-amine (26.98 mg, 0.14 mmol), DIPEA (74.10 mg, 0.57 mmol) and HATU (65.2 mg, 0.17 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (100% EtOAc) to give the crude product and the crude product was purified by prep-HPLC to give N-[(1R)-1-(1,1-difluoro-2,3-dihydro-1H-inden-4-yl)ethyl]-5-(oxan-4-yl)-4-oxo-1H,4H,5H-pyrrolo[3,2-c]pyridine-7-carboxamide (3.0 mg, 0.007 mmol, 5.94%) as a white solid. LCMS: m/z 408 (M+H)⁺. ¹H NMR (400 MHz, DMSO) δ 11.35 (s, 1H), 8.82 (d, J=7.0 Hz, 1H), 8.25 (d, J=15.4 Hz, 1H), 7.66-7.50 (m, 1H), 7.47-7.39 (m, 2H), 7.06-6.88 (m, 1H), 6.51 (dd, J=2.9, 2.3 Hz, 1H), 5.33-5.06 (m, 2H), 4.06 (dd, J=11.2, 3.9 Hz, 2H), 3.53 (dd, J=24.2, 12.9 Hz, 2H), 3.19-3.05 (m, 2H), 2.73-2.56 (m, 2H), 2.20-2.03 (m, 2H), 1.71 (d, J=9.6 Hz, 2H), 1.53 (d, J=7.1 Hz, 3H).

### Example 39: Synthesis of (R)—N-(1-(3-(1,1-difluoro-2-hydroxyethyl)phenyl)ethyl)-4-oxo-5-(tetrahydro-2H-pyran-4-yl)-4,5-dihydro-1H-pyrrolo[3,2-c]pyridine-7-carboxamide. (the Title Compounds was Synthesized from Compound 9 in Example 38.)

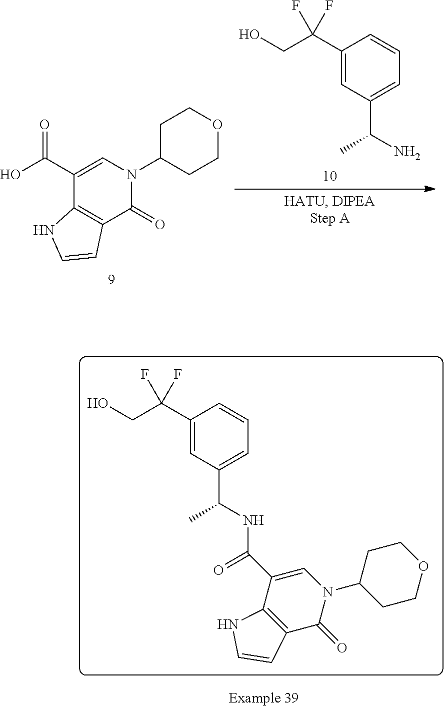 [SMILES: O=C(O)c1cn(C2CCOCC2)c(=O)c2cc[nH]c12 ; C[C@@H](N)c1cccc(C(F)(F)CO)c1 ; C[C@@H](NC(=O)c1cn(C2CCOCC2)c(=O)c2cc[nH]c12)c1cccc(C(F)(F)CO)c1]

### Step A. of (R)—N-(1-(3-(1,1-difluoro-2-hydroxyethyl)phenyl)ethyl)-4-oxo-5-(tetrahydro-2H-pyran-4-yl)-4,5-dihydro-1H-pyrrolo[3,2-c]pyridine-7-carboxamide

To a stirred solution of 5-(oxan-4-yl)-4-oxo-1H,4H,5H-pyrrolo[3,2-c]pyridine-7-carboxylic acid (30 mg, 0.11 mmol) in DMF (3 mL) was added 2-{3-[(1R)-1-aminoethyl]phenyl}-2,2-difluoroethan-1-ol (27.53 mg, 0.14 mmol), DIEA (0.09 mL, 0.57 mmol) and HATU>=98.0% (CHN) (86.7 mg, 0.23 mmol). The reaction mixture was stirred at room temperature for 2 h. The reaction mixture was poured into water and extracted with EtOAc. The organic layers were washed with brine, dried over Na₂SO₄ and concentrated under vacuum. The residue was purified by prep-TLC (100% EtOAc) to give the crude product and the crude product was purified by prep-HPLC to give N-[(1R)-1-[3-(1,1-difluoro-2-hydroxyethyl)phenyl]ethyl]-5-(oxan-4-yl)-4-oxo-1H,4H,5H-pyrrolo[3,2-c]pyridine-7-carboxamide (2.1 mg, 0.005 mmol, 4.14%) as a white solid. ¹H NMR (400 MHz, DMSO) δ 11.35 (s, 1H), 8.82 (d, J=7.0 Hz, 1H), 8.25 (d, J=15.4 Hz, 1H), 7.66-7.50 (m, 1H), 7.47-7.39 (m, 2H), 7.06-6.88 (m, 1H), 6.51 (dd, J=2.9, 2.3 Hz, 1H), 5.33-5.06 (m, 2H), 4.06 (dd, J=11.2, 3.9 Hz, 2H), 3.53 (dd, J=24.2, 12.9 Hz, 2H), 3.19-3.05 (m, 2H), 2.73-2.56 (m, 2H), 2.20-2.03 (m, 2H), 1.71 (d, J=9.6 Hz, 2H), 1.53 (d, J=7.1 Hz, 3H). LCMS: m/z 446 (M+H)⁺.

### Example 40: Synthesis of (R)—N-(1-(3-(difluoromethyl)-2-fluorophenyl)ethyl)-4-oxo-5-(tetrahydro-2H-pyran-4-yl)-4,5-dihydro-1H-pyrrolo[3,2-c]pyridine-7-carboxamide. (the Title Compounds was Synthesized from Compound 9 in Example 38.)

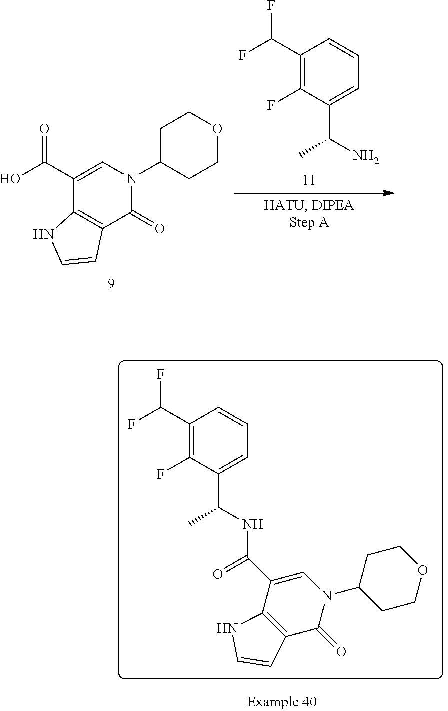 [SMILES: O=C(O)c1cn(C2CCOCC2)c(=O)c2cc[nH]c12 ; C[C@@H](N)c1cccc(C(F)F)c1F ; C[C@@H](NC(=O)c1cn(C2CCOCC2)c(=O)c2cc[nH]c12)c1cccc(C(F)F)c1F]

### Step A. of (R)—N-(1-(3-(difluoromethyl)-2-fluorophenyl)ethyl)-4-oxo-5-(tetrahydro-2H-pyran-4-yl)-4,5-dihydro-1H-pyrrolo[3,2-c]pyridine-7-carboxamide

To a stirred solution of 5-(oxan-4-yl)-4-oxo-1H,4H,5H-pyrrolo[3,2-c]pyridine-7-carboxylic acid (30 mg, 0.11 mmol) in DMF (3 mL) was added (1R)-1-[3-(difluoromethyl)-2-fluorophenyl]ethan-1-amine (21.6 mg, 0.11 mmol), DIEA (0.10 mL, 0.57 mmol) and HATU>=98.0% (CHN) (65.0 mg, 0.171 mmol). The reaction mixture was stirred at room temperature for 2 h. The reaction mixture was poured into water and extracted with EtOAc. The organic layers were washed with brine, dried over Na₂SO₄ and concentrated. The residue was purified by TLC (100% EtOAc) to give the crude product and the crude product was purified by prep-HPLC to give N-[(1R)-1-[3-(difluoromethyl)-2-fluorophenyl]ethyl]-5-(oxan-4-yl)-4-oxo-1H,4H,5H-pyrrolo[3,2-c]pyridine-7-carboxamide (16.8 mg, 0.04 mmol, 34.0%) as a white solid. ¹H NMR (400 MHz, DMSO) δ 11.37 (s, 1H), 8.88 (d, J=6.8 Hz, 1H), 8.26 (s, 1H), 7.66 (t, J=7.1 Hz, 1H), 7.52 (t, J=6.9 Hz, 1H), 7.36 (d, J=8.0 Hz, 1H), 7.23 (t, J=45.7 Hz, 1H), 7.01-6.96 (m, 1H), 6.54-6.48 (m, 1H), 5.47-5.35 (m, 1H), 5.26-5.05 (m, 1H), 4.07 (dd, J=11.1, 3.7 Hz, 2H), 3.55 (t, J=11.3 Hz, 2H), 2.13 (tt, J=16.6, 8.3 Hz, 2H), 1.72 (d, J=10.4 Hz, 2H), 1.55 (d, J=7.1 Hz, 3H). LCMS: m/z 434 (M+H)⁺.

### Example 41: Synthesis of (R)—N-(1-(1,1-difluoro-2,3-dihydro-1H-inden-4-yl)ethyl)-5-(1-methylcyclopropyl)-4-oxo-4,5-dihydro-1H-pyrrolo[3,2-c]pyridine-7-carboxamide

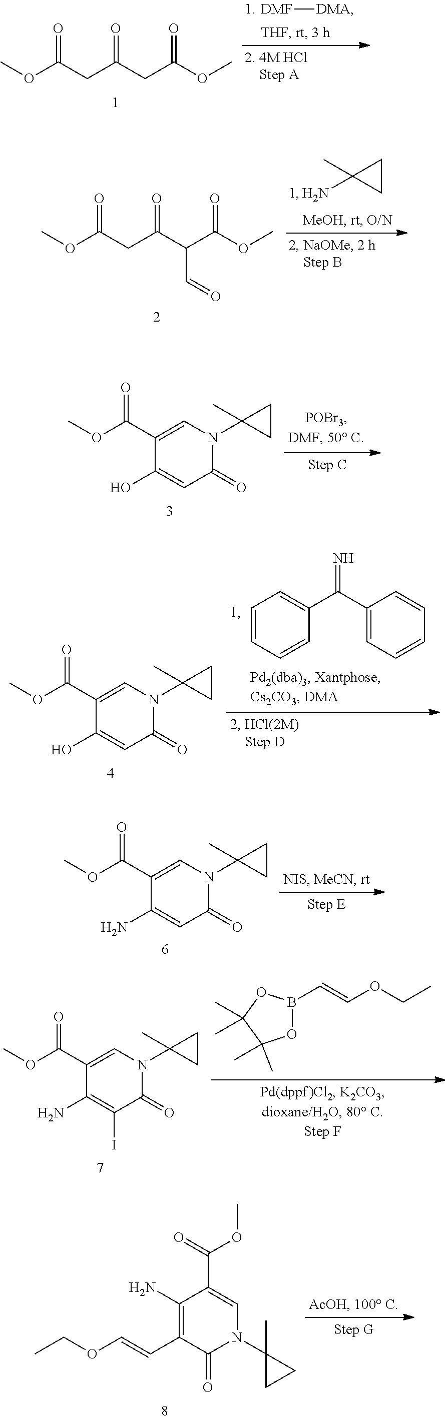

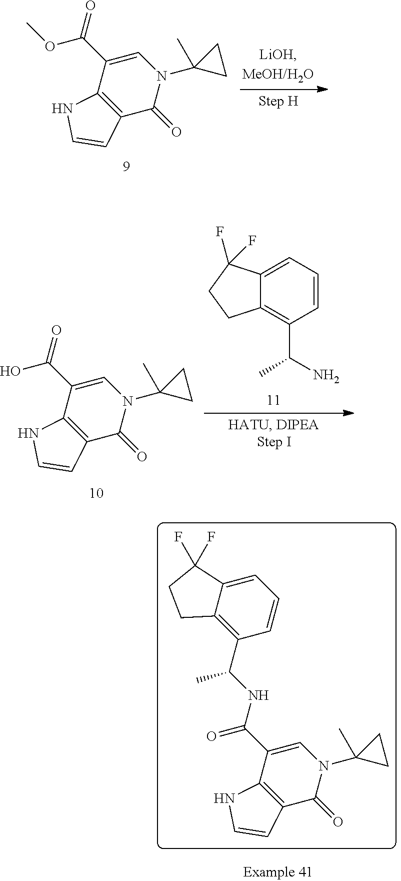 [SMILES: COC(=O)c1cn(C2(C)CC2)c(=O)c2cc[nH]c12 ; CC1(n2cc(C(=O)O)c3[nH]ccc3c2=O)CC1 ; C[C@@H](N)c1cccc2c1CCC2(F)F ; C[C@@H](NC(=O)c1cn(C2(C)CC2)c(=O)c2cc[nH]c12)c1cccc2c1CCC2(F)F]

### Step A. dimethyl 2-formyl-3-oxopentanedioate

To a stirred solution of 1,5-dimethyl 3-oxopentanedioate (7.63 mL, 51.7 mmol) in THF (50 mL) was added DMF-DMA (13.9 mL, 103 mmol) dropwise over 5 min. The reaction mixture was stirred at room temperature for 3 h. The pH of the reaction mixture was adjusted to 3-4 by adding HCl (5 mL, 4M). The reaction mixture was poured into water and extracted with DCM. The organic layer was washed with brine, dried over by Na₂SO₄ and concentrated under vacuum to give the residue. The residue was purified by column chromatography (silica gel, 40 g, 0˜2% MeOH in DCM) to give 1,5-dimethyl 2-formyl-3-oxopentanedioate (10 g, 49.46 mmol, 95.7%) as a yellow oil. LCMS: m/z 203 (M+H)⁺.

### Step B. methyl 4-hydroxy-1-(1-methylcyclopropyl)-6-oxo-1,6-dihydropyridine-3-carboxylate

To a stirred solution of 1,5-dimethyl 2-formyl-3-oxopentanedioate (3 g, 14.8 mmol) in MeOH (20 mL) was added 1-methylcyclopropan-1-amine (1.58 g, 22.26 mmol). The reaction mixture was stirred at room temperature for 16 h. Then MeONa (7.4 mL, 29.6 mmol) was added. The reaction mixture was poured into water and extracted with DCM. The organic layer was washed with brine, dried over by Na₂SO₄ and concentrated under vacuum to give the residue. The residue was purified by column chromatography (silica gel, 40 g, 0˜5% MeOH in DCM) to give methyl 4-hydroxy-1-(1-methylcyclopropyl)-6-oxo-1,6-dihydropyridine-3-carboxylate (800 undefined) as a yellow solid. LCMS: m/z 224 (M+H)⁺.

### Step C. methyl 4-bromo-1-(1-methylcyclopropyl)-6-oxo-1,6-dihydropyridine-3-carboxylate

To a solution of methyl 4-hydroxy-1-(1-methylcyclopropyl)-6-oxo-1,6-dihydropyridine-3-carboxylate (3 g, 13.4 mmol) in DMF (50 mL) was added POBr₃ (2.73 mL, 26.8 mmol). The reaction mixture was stirred at 60° C. for 2 h. The reaction mixture was poured into NaHCO₃ (a.q) and extracted with DCM. The organic layers were washed with brine, dried over Na₂SO₄, concentrated under vacuum and purified by column chromatography (silica gel, 40 g, 0-50% EtOAc in PE) to give methyl 4-bromo-1-(1-methylcyclopropyl)-6-oxo-1,6-dihydropyridine-3-carboxylate (3.1 g, 10.8 mmol, 80.6%) as a yellow oil. LCMS: m/z 286 (M+H)⁺.

### Step D. methyl 4-amino-1-(1-methylcyclopropyl)-6-oxo-1,6-dihydropyridine-3-carboxylate

To a stirred solution of methyl 4-bromo-1-(1-methylcyclopropyl)-6-oxo-1,6-dihydropyridine-3-carboxylate (3.1 g, 10.8 mmol) in dioxane (30 mL) was added diphenylmethanimine (2.0 mL, 11.9 mmol), Pd₂(dba)₃ (1.02 g, 1.08 mmol), Xant-Phos (1.20 g, 2.17 mmol) and Cs₂CO₃ (8.8 g, 27.1 mmol). The reaction mixture was stirred at 95° C. for 5 h. Then HCl (2M, 20 mL) was added and stirred for 0.5 h. The pH of the reaction mixture was adjusted to 8-9 by adding saturated Sodium bicarbonate and extracted with DCM. The organic layers were washed with brine, dried over Na₂SO₄ and concentrated under vacuum to give the residue and the residue was purified by (silica gel, 20 g, 0˜5% MeOH in DCM) to give methyl 4-amino-1-(1-methylcyclopropyl)-6-oxo-1,6-dihydropyridine-3-carboxylate (2 g, 8.99 mmol, 83.1%) as a light yellow solid. LCMS: m/z 223 (M+H)⁺.

### Step E. methyl 4-amino-5-iodo-1-(1-methylcyclopropyl)-6-oxo-1,6-dihydropyridine-3-carboxylate

To a stirred solution of methyl 4-amino-1-(1-methylcyclopropyl)-6-oxo-1,6-dihydropyridine-3-carboxylate (1.8 g, 8.10 mmol in AcOH (5 mL) and DCM (10 mL) was added iodo(sulfanylidene)amine (1.5 g, 8.90 mmol). The reaction mixture was stirred at room temperature for 2 h. The reaction mixture was poured into water and extracted with EtOAc washed with NaHCO₃ and dried over Na₂SO₄ and concentrated under vacuum to give the residue and the residue purified by column chromatography (silica gel, 24 g, 0˜100% EtOAc in PE) to give methyl4-amino-5-iodo-1-(1-methylcyclopropyl)-6-oxo-1,6-dihydropyridine-3-carboxylate (1.2 g, 3.45 mmol, 42.5%) as a colorless solid. LCMS: m/z 349 (M+H)⁺.

### Step F. methyl (E)-4-amino-5-(2-ethoxyvinyl)-1-(1-methylcyclopropyl)-6-oxo-1,6-dihydropyridine-3-carboxylate

To a stirred solution of methyl 4-amino-5-iodo-1-(1-methylcyclopropyl)-6-oxo-1,6-dihydropyridine-3-carboxylate (700 mg, 2.01 mmol) in dioxane (10 mL) and H₂O (2 mL) was added 2-[(E)-2-ethoxyethenyl]-4,4,5,5-tetramethyl-1,3,2-dioxaborolane (0.47 mL, 2.21 mmol), Pd(dppf)Cl₂ (147.1 mg, 0.20 mmol) and K₂CO₃ (555.8 mg, 4.02 mmol). The reaction mixture was stirred at 95° C. for 16 h. The reaction mixture was filtered and the filtrate was concentrated under vacuum to give the residue and the residue was purified by column chromatography (silica gel, 24 g, 0˜100% EtOAc in PE) to give methyl4-amino-5-[(E)-2-ethoxyethenyl]-1-(1-methylcyclopropyl)-6-oxo-1,6-dihydropyridine-3-carboxylate (500 mg, 1.71 mmol, 85.0%) as a light yellow solid. LCMS: m/z 292 (M+H)⁺.

### Step G. methyl 5-(1-methylcyclopropyl)-4-oxo-4,5-dihydro-1H-pyrrolo[3,2-c]pyridine-7-carboxylate

A reaction mixture of methyl4-amino-5-[(E)-2-ethoxyethenyl]-1-(1-methylcyclopropyl)-6-oxo-1,6-dihydropyridine-3-carboxylate (500 mg, 1.71 mmol) in AcOH (10 mL) was stirred at 100° C. for 2 h. The reaction mixture was concentrated under vacuum to give the residue and the residue was purified by column chromatography (silica gel, 24 g, 0˜60% EtOAc in PE) to give methyl 5-(1-methylcyclopropyl)-4-oxo-1H,4H,5H-pyrrolo[3,2-c]pyridine-7-carboxylate (150 mg, 0.61 mmol, 35.6%) as a light yellow solid. LCMS: m/z 247 (M+H)⁺.

### Step H. 5-(1-methylcyclopropyl)-4-oxo-4,5-dihydro-1H-pyrrolo[3,2-c]pyridine-7-carboxylic acid

To a stirred solution of methyl 5-(1-methylcyclopropyl)-4-oxo-1H,4H,5H-pyrrolo[3,2-c]pyridine-7-carboxylate (90 mg, 0.36 mmol) in MeOH (3 mL) was added LiOH (174 mg, 4.3 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give the crude product 5-(1-methylcyclopropyl)-4-oxo-1H,4H,5H-pyrrolo[3,2-c]pyridine-7-carboxylic acid (70 mg, 0.301 mmol, 82.5%) as a light yellow solid. LCMS: m/z 233 (M+H)⁺.

### Step I. (R)—N-(1-(1,1-difluoro-2,3-dihydro-1H-inden-4-yl)ethyl)-5-(1-methylcyclopropyl)-4-oxo-4,5-dihydro-1H-pyrrolo[3,2-c]pyridine-7-carboxamide

To a stirred solution of 5-(1-methylcyclopropyl)-4-oxo-1H,4H,5H-pyrrolo[3,2-c]pyridine-7-carboxylic acid (35 mg, 0.151 mmol) in DMF (3 mL) was added (1R)-1-(1,1-difluoro-2,3-dihydro-1H-inden-4-yl)ethan-1-amine (35.6 mg, 0.18 mmol), DIPEA (97.9 mg, 0.75 mmol) and HATU (114 mg, 0.30 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (100% EtOAc) to give the crude product and the crude product was purified by prep-HPLC to give N-[(1R)-1-(1,1-difluoro-2,3-dihydro-1H-inden-4-yl)ethyl]-5-(1-methylcyclopropyl)-4-oxo-1H,4H,5H-pyrrolo[3,2-c]pyridine-7-carboxamide (15 mg, 0.036 mmol, 24.2%) as a white solid. ¹H NMR (400 MHz, DMSO) δ 11.20 (s, 1H), 8.72 (d, J=7.1 Hz, 1H), 8.15 (s, 1H), 7.51 (dd, J=8.1, 5.1 Hz, 1H), 7.39-7.31 (m, 2H), 6.89 (t, J=2.6 Hz, 1H), 6.44-6.36 (m, 1H), 5.15 (p, J=6.9 Hz, 1H), 3.15-2.98 (m, 2H), 2.63-2.47 (m, 2H), 1.44 (t, J=6.0 Hz, 3H), 1.42 (s, 3H), 1.00 (t, J=8.2 Hz, 2H), 0.91 (s, 2H LCMS: m/z 412 (M+H)⁺.

### Example 42: Synthesis of (R)—N-(1-(3-(1,1-difluoro-2-hydroxyethyl)phenyl)ethyl)-5-(1-methylcyclopropyl)-4-oxo-4,5-dihydro-1H-pyrrolo[3,2-c]pyridine-7-carboxamide. (the Title Compounds was Synthesized from Compound 10 According to the Same Sequence Used for the Synthesis of Example 41.)

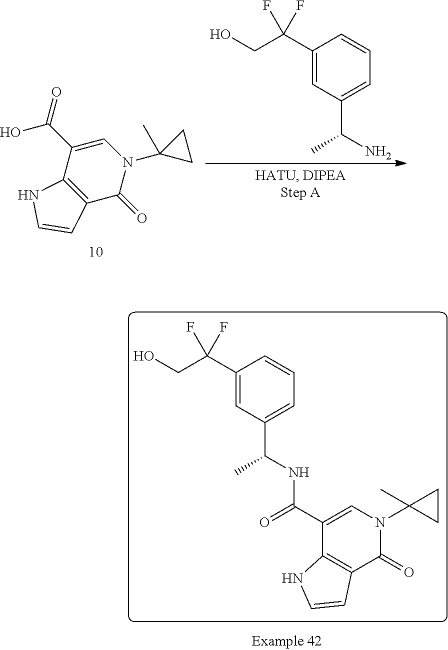 [SMILES: CC1(n2cc(C(=O)O)c3[nH]ccc3c2=O)CC1 ; C[C@@H](N)c1cccc(C(F)(F)CO)c1 ; C[C@@H](NC(=O)c1cn(C2(C)CC2)c(=O)c2cc[nH]c12)c1cccc(C(F)(F)CO)c1]

### Step A. (R)—N-(1-(3-(1,1-difluoro-2-hydroxyethyl)phenyl)ethyl)-5-(1-methylcyclopropyl)-4-oxo-4,5-dihydro-1H-pyrrolo[3,2-c]pyridine-7-carboxamide

To a stirred solution of 5-(1-methylcyclopropyl)-4-oxo-1H,4H,5H-pyrrolo[3,2-c]pyridine-7-carboxylic acid (35 mg, 0.151 mmol) in DMF (3 mL) was added 2-{3-[(1R)-1-aminoethyl]phenyl}-2,2-difluoroethan-1-ol (36.4 mg, 0.181 mmol), DIEA (0.125 mL, 0.754 mmol) and HATU>=98.0% (CHN) (114 mg, 0.30 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by prep-TLC (100% EtOAc) to give the crude product and the crude product was purified by prep-HPLC to give N-[(1R)-1-[3-(1,1-difluoro-2-hydroxyethyl)phenyl]ethyl]-5-(1-methylcyclopropyl)-4-oxo-1H,4H,5H-pyrrolo[3,2-c]pyridine-7-carboxamide (12 mg, 0.029 mmol, 19.17%) as a white solid. ¹H NMR (400 MHz, DMSO) δ 11.21 (s, 1H), 8.71 (d, J=7.6 Hz, 1H), 8.12 (s, 1H), 7.47 (d, J=8.9 Hz, 2H), 7.39 (t, J=7.6 Hz, 1H), 7.32 (d, J=7.7 Hz, 1H), 6.97-6.85 (m, 1H), 6.45-6.34 (m, 1H), 5.57 (t, J=6.3 Hz, 1H), 5.16 (p, J=7.0 Hz, 1H), 3.78 (td, J=14.2, 6.3 Hz, 2H), 1.46 (d, J=7.1 Hz, 3H), 1.41 (s, 3H), 1.05-0.96 (m, 2H), 0.91 (s, 2H). LCMS: m/z 416 (M+H)⁺.

### Example 43: Synthesis of (R)—N-(1-(1,1-difluoro-2,3-dihydro-1H-inden-4-yl)ethyl)-3-methyl-5-(1-methylcyclopropyl)-4-oxo-4,5-dihydro-1H-pyrrolo[3,2-c]pyridine-7-carboxamide. (the Title Compounds was Synthesized from Compound 9 in Example 41.)

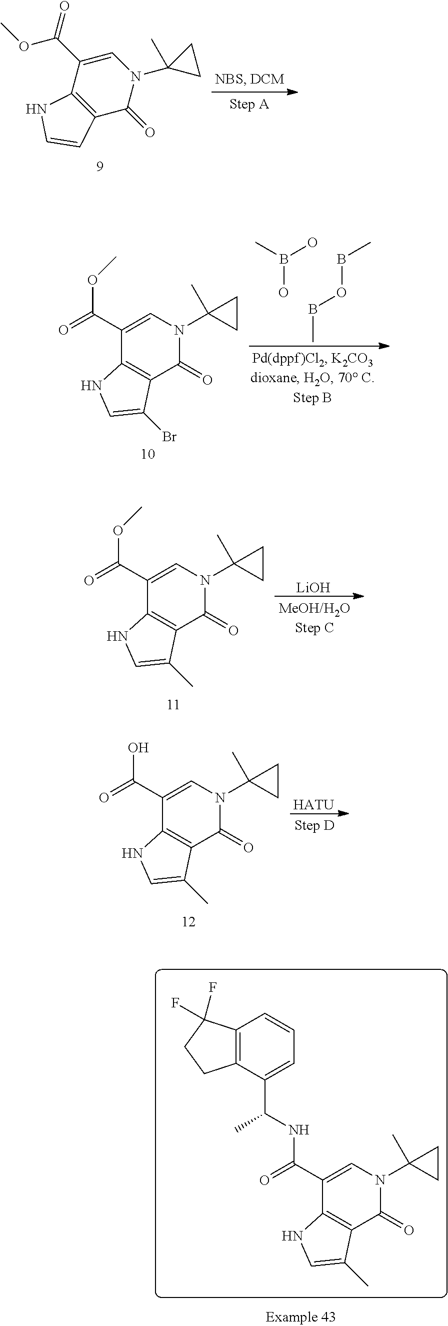

### Step A. methyl 3-bromo-5-(1-methylcyclopropyl)-4-oxo-4,5-dihydro-1H-pyrrolo[3,2-c]pyridine-7-carboxylate

To a stirred solution of methyl 5-(1-methylcyclopropyl)-4-oxo-1H,4H,5H-pyrrolo[3,2-c]pyridine-7-carboxylate (60 mg, 0.24 mmol) in AcOH (1 mL) and DCM (3 mL) was added NBS (47.7 mg, 0.27 mmol). The reaction mixture was stirred at room temperature for 2 h. The reaction mixture was poured into water and extracted with EtOAc. The organic phase was washed with aqueous NaHCO₃, dried over Na₂SO₄ and concentrated under vacuum to give the residue and the residue was purified by column chromatography (silica gel, 24 g, 0˜100% EtOAc in PE) to give methyl3-bromo-5-(1-methylcyclopropyl)-4-oxo-1H,4H,5H-pyrrolo[3,2-c]pyridine-7-carboxylate (60 mg, 0.19 mmol, 75.7%) as a colorless solid. LCMS: m/z 326 (M+H)⁺.

### Step B. methyl 3-methyl-5-(1-methylcyclopropyl)-4-oxo-4,5-dihydro-1H-pyrrolo[3,2-c]pyridine-7-carboxylate

To a stirred solution of methyl 3-bromo-5-(1-methylcyclopropyl)-4-oxo-1H,4H,5H-pyrrolo[3,2-c]pyridine-7-carboxylate (60 mg, 0.185 mmol) in dioxane (3 mL) and H₂O (1 mL) was added trimethyl-1,3,5,2,4,6-trioxatriborinane (0.08 mL, 0.55 mmol), Pd(dppf)Cl₂ (13.5 mg, 0.02 mmol) and K₂CO₃ (76.5 mg, 0.55 mmol). The reaction mixture was stirred at 70° C. for 5 h. The reaction mixture was filtered and the filtrate was concentrated under vacuum to give the residue and the residue was purified by column chromatography (silica gel, 24 g, 0-80% EtOAc in PE) to give methyl 3-methyl-5-(1-methylcyclopropyl)-4-oxo-1H,4H,5H-pyrrolo[3,2-c]pyridine-7-carboxylate (10 mg, 0.04 mmol, 20.8%) as a light yellow solid. LCMS: m/z 261 (M+H)⁺.

### Step C. 3-methyl-5-(1-methylcyclopropyl)-4-oxo-4,5-dihydro-1H-pyrrolo[3,2-c]pyridine-7-carboxylic acid

To a stirred solution of methyl 3-methyl-5-(1-methylcyclopropyl)-4-oxo-1H,4H,5H-pyrrolo[3,2-c]pyridine-7-carboxylate (10 mg, 0.04 mmol) in MeOH (3 mL) was added LiOH (15.37 mg, 0.38 mmol) in H₂O (2 mL). The reaction mixture was stirred at room temperature for 2 h. The pH of the reaction mixture was adjusted to 3-4 and extracted with EtOAc. The organic layers were dried over Na₂SO₄ and concentrated under vacuum to give crude product 3-methyl-5-(1-methylcyclopropyl)-4-oxo-1H,4H,5H-pyrrolo[3,2-c]pyridine-7-carboxylic acid (6 mg, 0.02 mmol, 63.4%) as a light yellow solid. LCMS: m/z 247 (M+H)⁺.

### Step D. (R)—N-(1-(1,1-difluoro-2,3-dihydro-1H-inden-4-yl)ethyl)-3-methyl-5-(1-methylcyclopropyl)-4-oxo-4,5-dihydro-1H-pyrrolo[3,2-c]pyridine-7-carboxamide

To a stirred solution of 3-methyl-5-(1-methylcyclopropyl)-4-oxo-1H,4H,5H-pyrrolo[3,2-c]pyridine-7-carboxylic acid (6 mg, 0.024 mmol) in DMF (3 mL) was added (1R)-1-(1,1-difluoro-2,3-dihydro-1H-inden-4-yl)ethan-1-amine (5.77 mg, 0.03 mmol), DIPEA (15.84 mg, 0.12 mmol) and HATU (13.9 mg, 0.037 mmol). The reaction mixture was stirred at room temperature for 2 h. The reaction mixture was poured into water and extracted with EtOAc. The organic layers were washed with brine, dried over Na₂SO₄ and concentrated under vacuum to give the residue and the residue was purified by TLC (100% EtOAc) to give the crude product and the crude product was purified by prep-HPLC to give N-[(1R)-1-(1,1-difluoro-2,3-dihydro-1H-inden-4-yl)ethyl]-3-methyl-5-(1-methylcyclopropyl)-4-oxo-1H,4H,5H-pyrrolo[3,2-c]pyridine-7 carboxamide (5 mg, undefined, 0.01 mmol, 48.2%) as a white solid. LCMS: m/z 426 (M+H)⁺. ¹H NMR (400 MHz, MeOD) δ 8.08 (s, 1H), 7.62-7.51 (m, 1H), 7.48-7.31 (m, 2H), 6.35 (d, J=0.9 Hz, 1H), 5.40-5.14 (m, 1H), 3.10 (ddd, J=16.9, 9.3, 5.3 Hz, 1H), 2.60 (tt, J=14.4, 7.2 Hz, 2H), 2.33 (d, J=0.7 Hz, 3H), 1.58 (d, J=7.1 Hz, 3H), 1.55 (s, 3H), 1.18 (d, J=6.8 Hz, 2H), 1.04 (t, J=5.9 Hz, 2H).

### Example 44: Synthesis of (R)—N-(1-(1,1-difluoro-2,3-dihydro-1H-inden-4-yl)ethyl)-4-methoxy-5-morpholino-2H-pyrazolo[3,4-c]pyridine-7-carboxamide

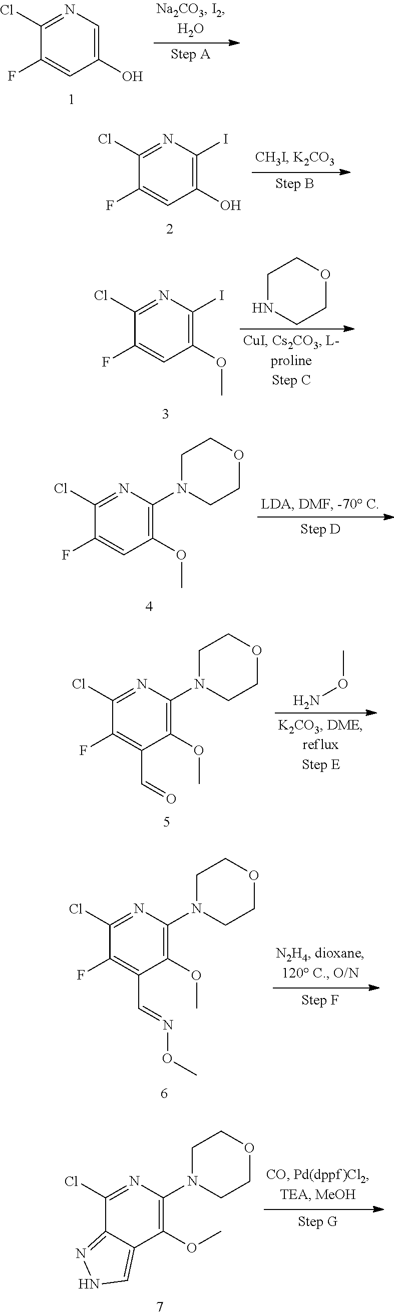

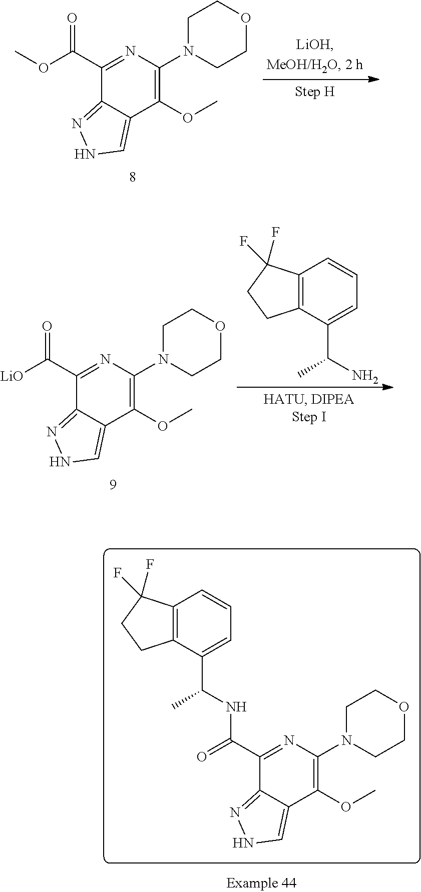

### Step A: 6-chloro-5-fluoro-2-iodopyridin-3-ol

To a stirred solution of 6-chloro-5-fluoropyridin-3-ol (2.5 g, 16.9 mmol) and Na₂CO₃ (3.6 g, 33.9 mmol) in H₂O (30 mL) at 0° C. was added iodine (4.30 g, 16.9 mmol) slowly. The reaction mixture was stirred at room temperature for 2 h. The reaction mixture was diluted with ice-water (50 mL), adjust pH to 5-6 with 1N HCl. The following mixture was extracted with EtOAc (50 mL×3). The combined organic phase was washed by water and brine, dried over anhydrous Na₂SO₄, filtered and concentrated to give a crude product 6-chloro-5-fluoro-2-iodopyridin-3-ol (3.7 g, 13.5 mmol, 79.8%) as a brown solid. The crude product was used directly for next step without purification. LC/MS (ESI) m/z: 274 (M+H)⁺.

### Step B: 2-chloro-3-fluoro-6-iodo-5-methoxypyridine

To a stirred mixture of 6-chloro-5-fluoro-2-iodopyridin-3-ol (2.1 g, 7.7 mmol) and K₂CO₃ (1.6 g, 11.5 mmol) in DMF (30 mL) was added iodomethane (1.2 g, 8.4 mmol) slowly. The reaction mixture was stirred at room temperature for 2 h. The reaction mixture was diluted with ice-water (50 mL). The mixture was extracted with EtOAc (50 mL×3). The combined organic phase was washed by water and brine, dried over anhydrous Na₂SO₄, filtered and concentrated to give a crude product which was purified by column chromatography on silica gel (EtOAc in PE: 0˜30%) to obtain 2-chloro-3-fluoro-6-iodo-5-methoxypyridine (1.8 g, 6.3 mmol, 81.5%) as a white solid. LC/MS (ESI) m/z: 287 (M+H)⁺.

### Step C: 4-(6-chloro-5-fluoro-3-methoxypyridin-2-yl)morpholine

To a stirred mixture of 2-chloro-3-fluoro-6-iodo-5-methoxypyridine (2.8 g, 9.7 mmol), morpholine (2.6 g, 29.2 mmol) and Cs₂CO₃ (9.5 g, 29.2 mmol) in DMSO (30 mL) was added CuI (370 mg, 1.9 mmol) and L-proline (220 mg, 1.9 mmol). The reaction mixture was stirred at 90° C. for 2 h under nitrogen atmosphere. The reaction mixture was cooled to room temperature and diluted with water (50 mL). The following mixture was extracted with EtOAc (50 mL×3). The combined organic phase was washed by water and brine, dried over anhydrous Na₂SO₄, filtered and concentrated to give the crude product which was purified by column chromatography on silica gel (EtOAc in PE: 0˜30%) to obtain 4-(6-chloro-5-fluoro-3-methoxypyridin-2-yl)morpholine (1.3 g, 5.3 mmol, 54.1%) as a white solid. LC/MS (ESI) m/z: 247 (M+H)⁺.

### Step D: 2-chloro-3-fluoro-5-methoxy-6-(morpholin-4-yl)pyridine-4-carbaldehyde

To a stirred solution of 4-(6-chloro-5-fluoro-3-methoxypyridin-2-yl)morpholine (1.3 g, 5.3 mmol) in anhydrous THE (20 mL) at −78° C. was added Lithium diisopropylamide (3.4 mL, 2.0 M solution in THF) slowly. The reaction mixture was stirred at −70° C. for 30 min under nitrogen atmosphere before DMF (1.9 g, 26. mmol) was added. The result solution was stirred at room temperature for 2 h. The reaction mixture was diluted with Sat·NH₄Cl (50 mL) at 0° C. The resulting mixture was extracted with EtOAc (50 mL×3). The combined organic phase was washed by water and brine, dried over anhydrous Na₂SO₄, filtered and concentrated to give a crude product which was purified by column chromatography on silica gel (EtOAc in PE: 0˜40%) to obtain 2-chloro-3-fluoro-5-methoxy-6-(morpholin-4-yl)pyridine-4-carbaldehyde (1.1 g, 4 mmol, 76%) as a yellow solid. LC/MS (ESI) m/z: 275 (M+H)⁺.

### Step E: (E)-{[2-chloro-3-fluoro-5-methoxy-6-(morpholin-4-yl)pyridin-4-yl]methylidene}(methoxy)amine

A mixture of 2-chloro-3-fluoro-5-methoxy-6-(morpholin-4-yl)pyridine-4-carbaldehyde (1.1 g, 4.0 mmol), Methoxyammonium chloride (350 mg, 4.2 mmol) and K₂CO₃ (1.4 g, 10.0 mmol) in 1,2-dimethoxyethane (20 mL) was stirred at reflux for 2 h under nitrogen atmosphere. The reaction mixture was cooled to room temperature and filtered. The filtrate was concentrated to give a crude product (E)-{[2-chloro-3-fluoro-5-methoxy-6-(morpholin-4-yl)pyridin-4-yl]methylidene}(methoxy)amine (1.1 g, 3.6 mmol, 90.5%) as a yellow solid, which was used directly for next step. LC/MS (ESI) m/z: 304 (M+H)⁺.

### Step F: 4-{7-chloro-4-methoxy-1H-pyrazolo[3,4-c]pyridin-5-yl}morpholine

To a stirred solution of (E)-{[2-chloro-3-fluoro-5-methoxy-6-(morpholin-4-yl)pyridin-4-yl]methylidene}(methoxy)amine (130 mg, 0.43 mmol) in 1,4-dioxane (5 mL) was added hydrazine hydrate (214 mg, 3.4 mmol). The reaction mixture was stirred at 120° C. for 16 h under nitrogen atmosphere. The reaction mixture was diluted with water (20 mL). The mixture was extracted with EtOAc (20 mL×3). The combined organic phase was washed by water and brine, dried over anhydrous Na₂SO₄, filtered and concentrated to give the crude product which was purified by column chromatography on silica gel (EtOAc in PE: 0˜40%) to obtain 4-{7-chloro-4-methoxy-1H-pyrazolo[3,4-c]pyridin-5-yl}morpholine (35 mg, 0.13 mmol, 30.4%) as a yellow solid (40 mg of (E)-{[2-chloro-3-fluoro-5-methoxy-6-(morpholin-4-yl)pyridin-4-yl]methylidene}(methoxy)amine was recovered). LC/MS (ESI) m/z: 269 (M+H)⁺.

### Step G: methyl 4-methoxy-5-morpholino-2H-pyrazolo[3,4-c]pyridine-7-carboxylate

To a stirred solution of 4-{7-chloro-4-methoxy-1H-pyrazolo[3,4-c]pyridin-5-yl}morpholine (35 mg, 0.13 mmol), triethylamine (39 mg, 0.4 mmol) in MeOH (5 mL) was added Pd(dppf)Cl₂ (9 mg, 0.013 mmol). The reaction mixture was stirred at 80° C. for 3 h under carbon monoxide atmosphere. The reaction mixture was filtered and concentrated to give a crude product which was purified by column chromatography on silica gel (EtOAc in PE: 0˜60%) to obtain methyl 4-methoxy-5-(morpholin-4-yl)-1H-pyrazolo[3,4-c]pyridine-7-carboxylate (30 mg, 0.1 mmol, 78.8%) as a yellow solid. LC/MS (ESI) m/z: 293 (M+H)⁺.

### Step G: lithium 4-methoxy-5-morpholino-2H-pyrazolo[3,4-c]pyridine-7-carboxylate

To a stirred solution of methyl 4-methoxy-5-(morpholin-4-yl)-1H-pyrazolo[3,4-c]pyridine-7-carboxylate (30 mg, 0.1 mmol) in MeOH (4 mL) and H₂O (4 mL) was added LiOH (13 mg, 0.3 mmol). The reaction mixture was stirred at room temperature for 2 h. The reaction mixture was concentrated to dryness to afford a crude lithium 4-methoxy-5-morpholino-2H-pyrazolo[3,4-c]pyridine-7-carboxylate (30 mg, 0.1 mmol, 105. %) as a yellow solid which was used directly for next step. LC/MS (ESI) m/z: 279 (M+H)⁺.

### Step H: (R)—N-(1-(1,1-difluoro-2,3-dihydro-1H-inden-4-yl)ethyl)-4-methoxy-5-morpholino-2H-pyrazolo[3,4-c]pyridine-7-carboxamide

To a stirred solution of lithium 4-methoxy-5-morpholino-2H-pyrazolo[3,4-c]pyridine-7-carboxylate (30 mg, 0.1 mmol), (1R)-1-(1,1-difluoro-2,3-dihydro-1H-inden-4-yl)ethan-1-amine (42 mg, 0.2 mmol), (30 mg, 0.1 mmol) and HATU (61 mg, 0.16 mmol) in DMF (4 mL) was added N,N-Diisopropylethylamine (28 mg, 0.2 mmol) slowly. The reaction mixture was stirred at room temperature for 2 h. The reaction mixture was diluted with water (20 mL). The following mixture was extracted with EtOAc (20 mL×3). The combined organic phase was washed by water and brine, dried over anhydrous Na₂SO₄, filtered and concentrated to give a crude product which was purified by column chromatography on silica gel (MeOH in DCM: 0˜8%) and further purified by prep-TLC (DCM:MeOH=25:1) to obtain N-[(1R)-1-(1,1-difluoro-2,3-dihydro-1H-inden-4-yl)ethyl]-4-methoxy-5-(morpholin-4-yl)-2H-pyrazolo[3,4-c]pyridine-7-carboxamide (13 mg, 0.03 mmol, 26%) as a yellow solid. LC/MS (ESI) m/z: 458 (M+H)⁺. ¹H NMR (400 MHz, DMSO) δ 13.37 (s, 1H), 8.57 (s, 1H), 8.51 (d, J=7.9 Hz, 1H), 7.75 (d, J=6.9 Hz, 1H), 7.51 (d, J=7.2 Hz, 2H), 5.42-5.30 (m, 1H), 4.37 (s, 3H), 3.92-3.85 (m, 4H), 3.37-3.33 (m, 4H), 3.30-3.25 (m, 2H), 2.77-2.67 (m, 2H), 1.68 (d, J=7.0 Hz, 3H).

Biological Assays

### KRAS-WT (or G12C/G12D/G12V)/SOS1 Binding Assay

The KRAS-WT (or G12C/G12D/G12V)/SOS1 binding assay is designed to measure the potency with which compounds inhibit the protein interaction between KRAS-WT (or G12C/G12D/G12V) and SOS1 proteins using a HTRF (Homogeneous Time-resolved Fluorescence) methodology. Low IC50 values are indicative of high potency of the SOS1 inhibitor compounds in this assay setting.

The KRAS-WT (or G12C/G12D/G12V)/SOS1 binding assay is performed using an assay kit (Cisbio, Cat #63ADK100CB15PEG or 63ADK000CB16PEG/63ADK000CB17PEG/63ADK000CB18PEG) according to the procedure recommended by the manufacturer.
1) Thaw and prepare the working solution of Tag1-SOS1 protein and Tag2-KRAS-WT (or G12C/G12D/G12V) protein with final concentration of 10 μM GTP (Sigma, Cat #V900868).
2) Thaw and prepare the working solution of anti-Tag1-Tb³⁺ anti-Tag2-XL665.
3) Dispense 2 μL of serially diluted compound, 4 μL of Tag1-SOS1 protein and 4 μL of Tag2-KRAS-WT (or G12C/G12D/G12V) protein with GTP into a ProxiPlate-384 Plus, white 384-shallow well microplate (PerkinElmer, Cat #6008280).
4) Incubate the plate for 15 minutes at room temperature.
5) Dispense 10 μL of pre-mixed anti-Tag1-Tb³⁺ and anti-Tag2-XL665 into the plate.
6) Seal the plate and incubate for 2 hours at room temperature.
7) Remove the plate sealer and read on PHERAstar FS (BMG LABTECH, PHERAstar FS).
8) Calculate the ratio of the acceptor and donor emission signals for each individual well using the following equation.

$$Ratio = \frac{Signal 665 nm}{Signal 620 nm} \times {10}^{4}$$

9) IC50 values are fitted and calculated by HTRF signal values and log of compound concentrations to nonlinear regression [Log (inhibitor) vs. Response—Variable Slope (four parameters)] using the GraphPad Prism 8.0.

TABLE 3
Activity Data in Kras: SOS1 Assay*

| Example No. | Wild Type | G12C | G12D |
|---|---|---|---|
| 1 | | A | A |
| 2 | A | A | A |
| 3 | A | A | A |
| 4 | A | | A |
| 5 | A | | A |
| 6 | A | | B |
| 7 | A | | B |
| 8 | C | | C |
| 9 | A | | A |
| 10 | A | | A |
| 11 | C | | C |
| 12 | B | | B |
| 13 | A | | A |
| 14 | B | | B |
| 15 | A | | A |
| 16 | | B | |
| 17 | | A | |
| 18 | A | | A |
| 19 | B | | B |
| 20 | B | | B |
| 21 | A | | B |
| 22 | B | | B |
| 23 | B | | B |
| 24 | B | | B |
| 25 | B | B | |
| 26 | | B | |
| 27 | B | | B |
| 28 | B | | B |
| 29 | A | | B |
| 30 | B | | B |
| 31 | C | | C |
| 32 | A | | A |
| 33 | A | | A |
| 34 | A | | A |
| 35 | A | | A |
| 36 | A | | A |
| 37 | A | | A |
| 38 | | A | |
| 39 | | A | |
| 40 | | A | |
| 41 | A | | A |
| 42 | A | | A |
| 43 | A | | B |
| 44 | C | | C |

*A: <100 nM; B: 100~500 nM; C: 500~5 μM; D: >5 μM

We claim:

1. A compound of Formulae (I)-(IV), 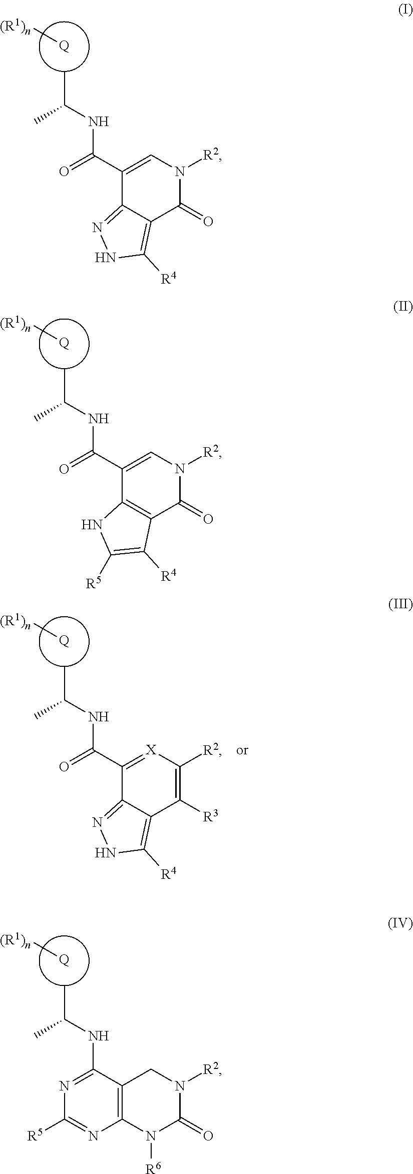
or a pharmaceutically acceptable salt, tautomer or stereoisomer thereof,
wherein:
Q at each occurrence is independently a ring selected from phenyl or a 5- or 6-membered heteroaryl group, wherein the heteroaryl group comprises at least one carbon atom and 1-4 additional heteroatoms independently selected from nitrogen, oxygen and sulfur;
X is CH or N;
R¹ at each occurrence is independently hydrogen, halogen, C₁₋₆alkyl, haloC₁₋₆alkyl, C₁₋₆alkenyl, C₁₋₆alkynyl, —NR^{a}R^{b}, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, —S(O)ₜ—C₁₋₆alkyl, —S(O)ₜ—NR^{a}R^{b}, phenyl, or 3-7-membered heterocyclyl, wherein the phenyl and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b}, or two adjacent R¹ groups, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄ alkyl-NR^{a}R^{b}, and oxo group (═O);
R² at each occurrence is independently hydrogen, halogen, CN, —OR^{a}, —NR^{a}R^{b}, C₁₋₆alkyl, haloC₁₋₆alkyl, C₂₋₆alkenyl, C₂₋₆alkynyl, C₃₋₇cycloalkyl, 3-7-membered heterocylyl, phenyl, or 5-6-membered heteroaryl, wherein each of the C₁₋₆alkyl, haloC₁₋₆alkyl, C₂₋₆alkenyl, C₂₋₆alkynyl, C₃₋₇cycloalkyl, 3-7-membered heterocylyl, phenyl, and 5-6-membered heteroaryl is optionally substituted with 1-5 R⁸;
R³ at each occurrence is independently hydrogen, halogen, C₁₋₆alkyl, haloC₁₋₆alkyl, C₁₋₆alkoxy, haloC₁₋₆alkoxy, C₁₋₆alkyl-OH, CN, C₃₋₇cycloalkyl, C₃₋₇cycloalkyl-OH, C₃₋₇cycloalkoyx, —NH₂, —NHC₁₋₄alkyl, —N(C₁₋₄alkyl)₂, or 3-7-membered cyclic amine;
R⁴ at each occurrence is independently hydrogen, halogen, C₁₋₆alkyl, haloC₁₋₆alkyl, C₁₋₆alkoxy, haloC₁₋₆alkoxy, CN, NH₂, C₃₋₇cycloalkyl or C₃₋₇cycloalkoxy;
R⁵ at each occurrence is independently hydrogen, C₁₋₄alkyl, or haloC₁₋₄alkyl;
R⁶ at each occurrence is independently hydrogen, C₁₋₆alkyl, haloC₁₋₆alkyl, or C₃₋₇cycloalkyl;
R⁸ at each occurrence is independently hydrogen, halogen, C₁₋₄alkyl, haloC₁₋₄alkyl, C₁₋₄alkoxy, C₂₋₄alkenyl, C₂₋₄alkynyl, C₃₋₇cycloalkyl, C₃₋₇cycloalkoxy, 3-7-membered heterocyclyl, phenyl, 5-6-membered heteroaryl, —OR^{a}, —SR^{a}, S(O)ₜR^{a}, —S(O)ₜNR^{a}R^{b}, —OC(O)—R^{a}, —NR^{a}R^{b}, —C(O)R^{a}, —C(O)OR^{a}, —OC(O)NR^{a}R^{b}₂, —C(O)NR^{a}R^{b}, —N(R^{a})C(O)OR^{a}, —N(R^{a})C(O)R^{a}, —N(R^{a})C(O)NR^{a}R^{b}, —N(R^{a})C(NR^{a})NR^{a}R^{b}, —N(R^{a})S(O)ₜNR^{a}R^{b}, —P(═O)(R^{a})(R), —O—P(═O)(OR^{a})(OR^{b}), or oxo group (═O);
R^{a} and R^{b} at each occurrence are independently hydrogen, C₁₋₆alkyl, haloC₁₋₆alkyl, C₁₋₆alkyl-OH, C₁₋₆alkoxy, C₃₋₇cycloalkyl, 3-7-membered heterocyclyl, C₁₋₆alkyl-NH₂, C₁₋₆alkyl-NHC₁₋₄alkyl, C₁₋₆alkyl-N(C₁₋₄alkyl)₂, or C₁₋₆alkyl-(3-7-membered cyclic amine), wherein each of the foregoing groups may be optionally substituted by one to three substituents independently selected from the group consisting of C₁₋₄alkyl, haloC₁₋₄alkyl, halogen, OH, NH₂, C₁₋₄alkoxy, haloC₁₋₄alkoxy, CN, and —C(O)C₁₋₄alkyl; or R^{a} and R^{b}, together with the nitrogen atom to which they are attached, form a saturated or unsaturated heterocyclic ring containing from three to seven ring atoms, which ring may optionally contain an additional one or two heteroatoms independently selected from the group consisting of nitrogen, oxygen and sulfur and may be optionally substituted by from one to three substituents independently selected from the group consisting of C₁₋₄alkyl, —C(O)C₁₋₄alkyl, phenyl and benzyl;
n at each occurrence is independently 1, 2 or 3, and
t at each occurrence is independently 1 or 2.

2. The compound of claim 1, having the structure of Formula (I):

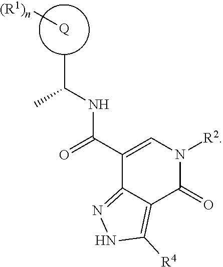

3. The compound of claim 1, having the structure of Formula (II):

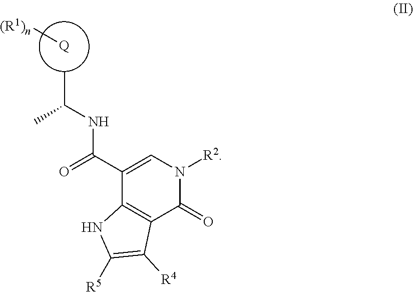

4. The compound of claim 1, having the structure of Formula (III):

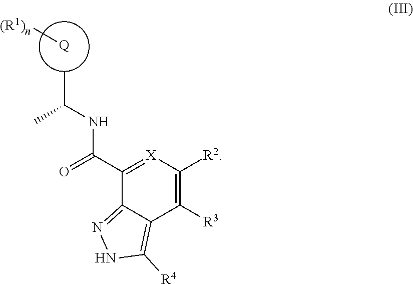

5. The compound of claim 1, having the structure of Formula (IV):

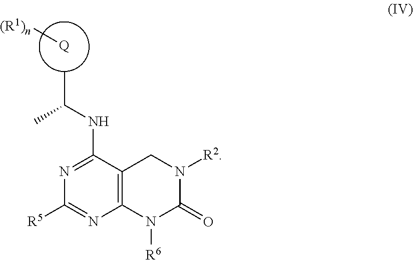

6. The compound of any of claims 1-5, wherein Q at each occurrence is phenyl.

7. The compound of any of claims 1-5, wherein Q at each occurrence is independently a 5-membered heteroaryl group.

8. The compound of any of claims 1-5, wherein Q at each occurrence is independently a 6-membered heteroaryl group.

9. The compound of any of claims 1-5, wherein Q at each occurrence is thiophenyl.

10. The compound of any of claims 1-5, wherein Q at each occurrence is pyridinyl.

11. The compound of any of claims 1-10, wherein R¹ at each occurrence is independently hydrogen, halogen, C₁₋₆alkyl, haloC₁₋₆alkyl, —NR^{a}R^{b}, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy or —S(O)ₜ—C₁₋₆alkyl.

12. The compound of any of claims 1-10, wherein R¹ at each occurrence is independently phenyl or 3-7-membered heterocyclyl, wherein the phenyl and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜNR^{a}R^{b}, —NR^{a}R^{b} and C₁₋₄alkyl-NR^{a}R^{b}.

13. The compound of any of claims 1-10, wherein two adjacent R¹ groups at each occurrence, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b}, and oxo group (═O).

14. The compound of any of claims 1-10, wherein two adjacent R¹ groups at each occurrence, together with the carbon atoms to which they are attached, form a 5-6-membered carbocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b} and oxo group (═O).

15. The compound of any of claims 1-10, wherein two adjacent R¹ groups at each occurrence, together with the carbon atoms to which they are attached, form a 5-6-membered heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b} and oxo group (═O).

16. The compound of claim 1, wherein Q together with its substituents (R¹)ₙ, 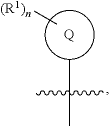
at each occurrence independently has the structure of 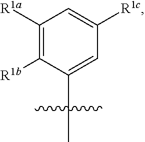
wherein:
R^{1a} at each occurrence is independently hydrogen, halogen, C₁₋₆alkyl, haloC₁₋₆alkyl, C₁₋₆alkenyl, C₁₋₆alkynyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, —S(O)ₜ—C₁₋₆alkyl, —S(O)ₜ—NR^{a}R^{b}, or 3-7-membered heterocyclyl, wherein the 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b};
R^{1b} at each occurrence is independently hydrogen, halogen, C₁₋₄alkyl or C₃₋₆cycloalkyl;
or R^{1a} and R^{1b}, at each occurrence, together with the carbon atoms to which they are attached, form a 5-7-membered carbocyclic or heterocyclic ring optionally substituted with 1-3 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b}, C₁₋₄alkyl-NR^{a}R^{b}, and oxo group (═O); and
R^{1c} at each occurrence is independently hydrogen, halogen, NH₂, or C₁₋₄alkyl.

17. The compound of claim 16, having the structure of Formula (Ia),

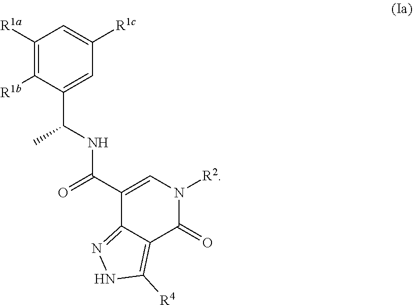

18. The compound of claim 16, having the structure of Formula (IIa),

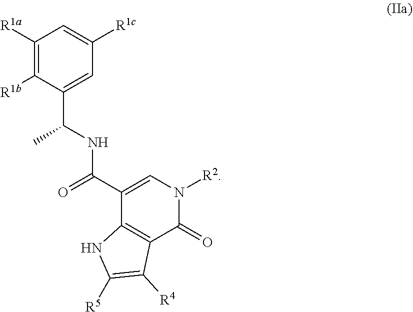

19. The compound of claim 16, having the structure of Formula (IIIa),

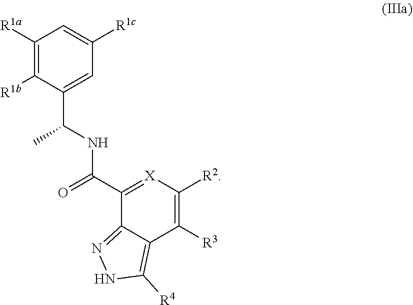

20. The compound of claim 16, having the structure of Formula (IVa),

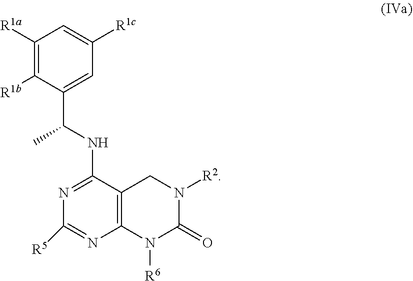

21. The compound of any one of claims 16-20, wherein R^{1a} at each occurrence is independently hydrogen, halogen, C₁₋₆alkyl, haloC₁₋₆alkyl, C₁₋₆alkenyl, C₁₋₆alkynyl, OH, C₁₋₆alkyl-OH, haloC₁₋₆alkyl-OH, C₁₋₆alkoxy, haloC₁₋₆alkoxy, CN, C₃₋₇cycloalkyl, C₃₋₇cycloalkyl-OH, C₃₋₇cycloalkoxy, or —S(O)ₜ—C₁₋₆alkyl.

22. The compound of any one of claims 16-20, wherein R^{1a} at each occurrence is independently 3-7-membered heterocyclyl optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —S(O)ₜ—C₁₋₆alkyl, —S(O)ₜ—NR^{a}R^{b}, —NR^{a}R^{b}, and C₁₋₄alkyl-NR^{a}R^{b}.

23. The compound of any one of claims 16-20, wherein R^{1a} at each occurrence is independently CHF₂, CH₂F, CF₃, CF₂CH₃ or CF₂CH₂OH.

24. The compound of any one of claims 16-20, wherein R^{1a} and R^{1b}, at each occurrence, together with the carbon atoms to which they are attached, form a 5- or 6-membered carbocyclic ring optionally substituted with 1-3 halogen.

25. The compound of any one of claims 16-20, wherein R^{1a} and R^{1b}, at each occurrence, together with the carbon atoms to which they are attached, form a carbocyclic ring having the structure of

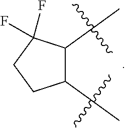 [SMILES: *C1CCC(F)(F)C1*]

26. The compound of any one of claims 16-23, wherein R^{1b} at each occurrence is hydrogen.

27. The compound of any one of claims 16-23, wherein R^{1b} at each occurrence is independently halogen.

28. The compound of any one of claims 16-23, wherein R^{1b} at each occurrence is independently C₁₋₄alkyl.

29. The compound of any one of claims 16-23, wherein R^{1b} at each occurrence is independently F or methyl.

30. The compound of any one of claims 16-29, wherein R^{1c} at each occurrence is hydrogen.

31. The compound of any one of claims 16-29, wherein R^{1c} at each occurrence is independently halogen.

32. The compound of any one of claims 16-29, wherein R^{1c} at each occurrence is NH₂.

33. The compound of any one of claims 16-29, wherein R^{1c} at each occurrence is independently C₁₋₄alkyl.

34. The compound of claim 1, wherein Q together with its substituents (R¹)ₙ, 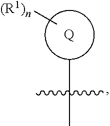
at each occurrence independently has the structure of 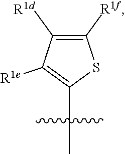
wherein:
R^{1d} and R^{1e} at each occurrence are independently hydrogen, halogen, C₁₋₄alkyl, or haloC₁₋₄alkyl; and
R^{1f} at each occurrence is independently phenyl, 5-6-membered heteroaryl, or 3-7-membered heterocyclyl, wherein the phenyl, 5-6-membered heteroaryl, and 3-7-membered heterocyclyl are optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b} and C₁₋₄alkyl-NR^{a}R^{b}.

35. The compound of claim 34, having the structure of Formula (Ib),

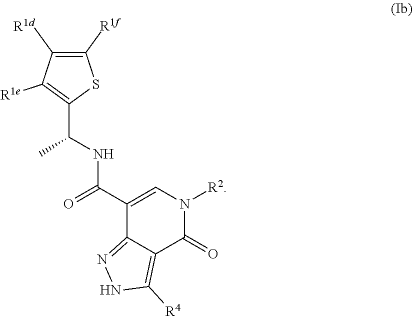

36. The compound of claim 34, having the structure of Formula (IIb),

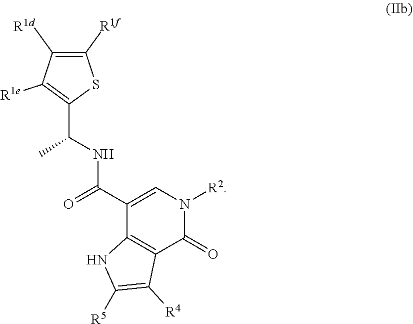

37. The compound of claim 34, having the structure of Formula (IIIb),

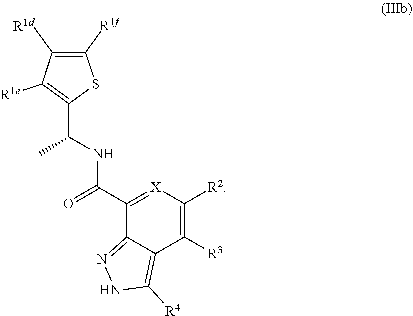

38. The compound of claim 34, having the structure of Formula (IVb),

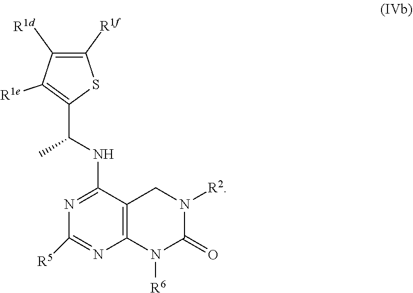

39. The compound of any one of claims 34-38, wherein R^{1d} and R^{1e} at each occurrence are independently hydrogen and methyl.

40. The compound of any one of claims 34-38, wherein R^{1d} and R^{1e} at each occurrence are hydrogen.

41. The compound of any one of claims 34-40, wherein R^{1f} at each occurrence is independently phenyl optionally substituted with 1-4 substituents independently selected from C₁₋₄alkyl, haloC₁₋₄alkyl, C₁₋₄alkoxy, haloC₁₋₄alkoxy, C₁₋₄alkyl-OH, haloC₁₋₄alkyl-OH, OH, halogen, CN, —NR^{a}R^{b} and C₁₋₄alkyl-NR^{a}R^{b}.

42. The compound of any one of claims 1-41, wherein R² at each occurrence is independently hydrogen, halogen, CN, —OR^{a}, —NR^{a}R^{b}, C₁₋₆alkyl, or haloC₁₋₆alkyl, wherein each of the C₁₋₆alkyl and haloC₁₋₆alkyl is optionally substituted with 1-5 R⁸.

43. The compound of any one of claims 1-41, wherein R² at each occurrence is independently C₃₋₇cycloalkyl optionally substituted with 1-5 R⁸.

44. The compound of any one of claims 1-41, wherein R² at each occurrence is independently 3-7-membered heterocylyl optionally substituted with 1-5 R⁸.

45. The compound of any one of claims 1-41, wherein R² at each occurrence is independently phenyl optionally substituted with 1-5 R⁸.

46. The compound of any one of claims 1-41, wherein R² at each occurrence is independently 5-membered heteroaryl optionally substituted with 1-5 R⁸.

47. The compound of any one of claims 1-41, wherein R² at each occurrence is independently 6-membered heteroaryl optionally substituted with 1-5 R⁸.

48. The compound of any one of claims 1, 4, 19 and 37, wherein R³ at each occurrence is independently hydrogen, halogen, C₁₋₆alkyl, haloC₁₋₆alkyl, or CN.

49. The compound of any one of claims 1, 4, 19 and 37, wherein R³ at each occurrence is independently C₁₋₆alkoxy, haloC₁₋₆alkoxy, or C₁₋₆alkyl-OH.

50. The compound of any one of claims 1, 4, 19 and 37, wherein R³ at each occurrence is independently C₃₋₇cycloalkyl or C₃₋₇cycloalkyl-OH.

51. The compound of any one of claims 1, 4, 19 and 37, wherein R³ at each occurrence is independently —NH₂, —NHC₁₋₄alkyl, —N(C₁₋₄alkyl)₂, or 3-7-membered cyclic amine.

52. The compound of any one of claims 1, 4, 19, 37 and 48-51, wherein X is CH.

53. The compound of any one of claims 1, 4, 19, 37 and 48-51, wherein X is N.

54. The compound of any one of claims 1-53, wherein R⁴ at each occurrence is hydrogen.

55. The compound of any one of claims 1-53, wherein R⁴ at each occurrence is independently halogen or C₁₋₆alkyl.

56. The compound of any one of claims 1, 3, 5, 18, 20, 36 and 38, wherein R⁵ at each occurrence is independently hydrogen or C₁₋₄alkyl.

57. The compound of any one of claims 1, 3, 5, 18, 20, 36 and 38, wherein R⁵ at each occurrence is hydrogen.

58. The compound of any one of claims 1, 3, 5, 18, 20, 36 and 38, wherein R⁵ at each occurrence is methyl.

59. The compound of any one of claims 1, 5, 20 and 38, wherein R⁶ at each occurrence is independently hydrogen or C₁₋₄alkyl.

60. The compound of any one of claims 1-59, wherein R⁸ at each occurrence is independently hydrogen, halogen, C₁₋₄alkyl, haloC₁₋₄alkyl, or C₁₋₄alkoxy.

61. The compound of any one of claims 1-59, wherein R⁸ at each occurrence is independently C₃₋₇cycloalkyl or 3-7-membered heterocyclyl.

62. The compound of any one of claims 1-59, wherein R⁸ at each occurrence is independently phenyl or 5-6-membered heteroaryl.

63. The compound of any one of claims 1-59, wherein R⁸ at each occurrence is independently —OR^{a}, —SR^{a}, —S(O)ₜR^{a}, —S(O)ₜ—NR^{a}R^{b}, —OC(O)—R^{a}, —NR^{a}R^{b}, —C(O)R^{a}, —C(O)OR^{a}, —OC(O)NR^{a}R^{b}, —C(O)NR^{a}R^{b}, —N(R^{a})C(O)OR^{a}, —N(R^{a})C(O)R^{a}, —N(R^{a})C(O)NR^{a}R^{b}, —N(R^{a})C(NR^{a})NR^{a}R^{b}, —N(R^{a})S(O)ₜNR^{a}R^{b}, —P(═O)(R^{a})(R), —O—P(═O)(OR^{a})(OR^{b}), or oxo group (═O).

64. The compound of any one of claims 1-59, wherein R⁸ at each occurrence is oxo group (═O).

65. The compound of any one of claims 1-59, wherein R⁸ at each occurrence is independently —NR^{a}R^{b}.

66. The compound of claim 65, wherein said —NR^{a}R^{b} is independently NH₂, —NHC₁₋₄alkyl, —N(C₁₋₄alkyl)₂, or 3-7-membered cyclic amine.

67. The compound of any one of claims 1-59, wherein R⁸ at each occurrence is independently —S(O)ₜ—C₁₋₄alkyl.

68. The compound of claim 1, selected from:

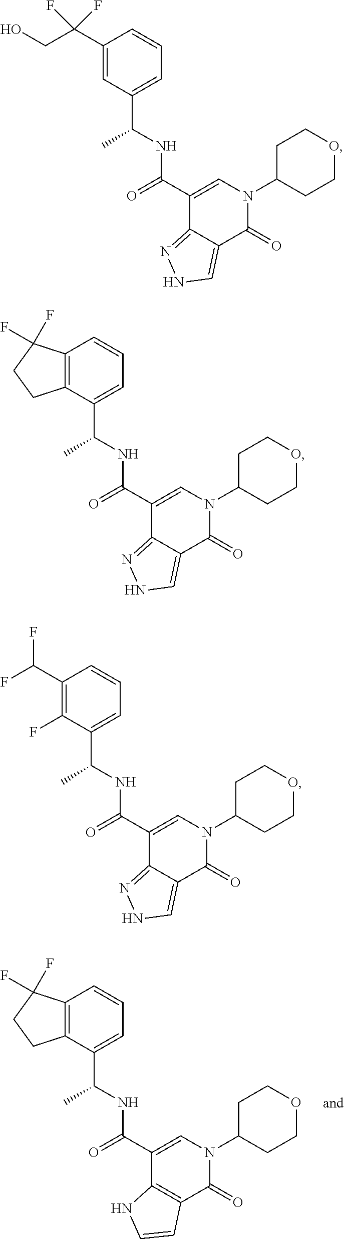 [SMILES: C[C@@H](NC(=O)c1cn(C2CCOCC2)c(=O)c2c[nH]nc12)c1cccc(C(F)(F)CO)c1 ; C[C@@H](NC(=O)c1cn(C2CCOCC2)c(=O)c2c[nH]nc12)c1cccc2c1CCC2(F)F ; C[C@@H](NC(=O)c1cn(C2CCOCC2)c(=O)c2c[nH]nc12)c1cccc(C(F)F)c1F ; C[C@@H](NC(=O)c1cn(C2CCOCC2)c(=O)c2cc[nH]c12)c1cccc2c1CCC2(F)F ; "and"]

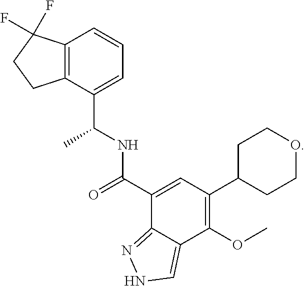 [SMILES: COc1c(C2CCOCC2)cc(C(=O)N[C@H](C)c2cccc3c2CCC3(F)F)c2n[nH]cc12]

69. The compound of claim 1, selected from:

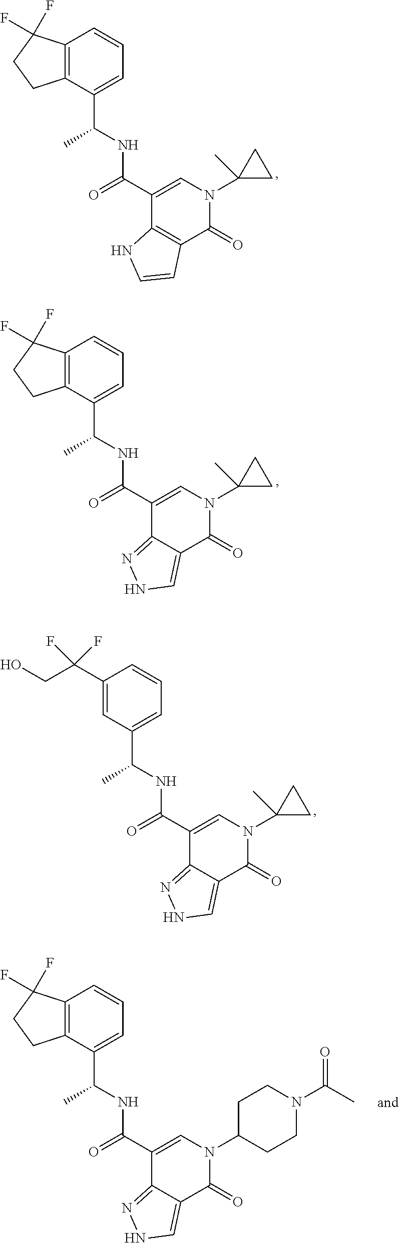 [SMILES: C[C@@H](NC(=O)c1cn(C2(C)CC2)c(=O)c2cc[nH]c12)c1cccc2c1CCC2(F)F ; C[C@@H](NC(=O)c1cn(C2(C)CC2)c(=O)c2c[nH]nc12)c1cccc2c1CCC2(F)F ; C[C@@H](NC(=O)c1cn(C2(C)CC2)c(=O)c2c[nH]nc12)c1cccc(C(F)(F)CO)c1 ; CC(=O)N1CCC(n2cc(C(=O)N[C@H](C)c3cccc4c3CCC4(F)F)c3n[nH]cc3c2=O)CC1 ; "and"]

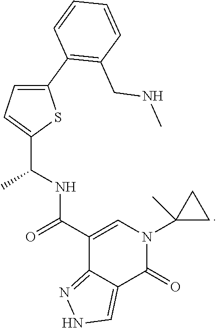 [SMILES: CNCc1ccccc1-c1ccc([C@@H](C)NC(=O)c2cn(C3(C)CC3)c(=O)c3c[nH]nc23)s1]

70. A pharmaceutical composition comprising the compound of any one of claims 1-69, and a pharmaceutically acceptable carrier.

71. A method for treating or preventing a disease or condition mediated by mammalian Ras family proteins in a subject in need thereof, comprising administering an effective amount of a compound of any one of claims 1-69 to the subject.

72. A method for treating or preventing cancer in a subject in need thereof, comprising administering an effective amount of a compound of any one of claims 1-69 to the subject.

73. The method of claim 72, wherein the cancer is breast cancer, leukemia, prostate cancer, ovarian cancer, pancreatic cancer, colon cancer, lung cancer, endometrial cancer, thyroid cancer, cholangiocarcinoma, multiple myeloma, melanoma, uterine cancer, head and neck squamous cell carcinoma, diffuse large B cell lymphoma, esophageal cancer, hepatocellular cancer, glioblastoma, renal cancer, sarcoma, bladder cancer, urothelial cancer, gastric cancer, or cervical cancer.
